(19) United States
(12) Patent Application Publication
(10) Pub. No.: US 2022/0281870 A1
(43) Pub. Date: Sep. 8, 2022

(54) COMPOUND, MATERIAL FOR AN ORGANIC ELECTROLUMINESCENT DEVICE AND APPLICATION THEREOF

(71) Applicant: Shanghai Tianma AM-OLED Co., Ltd., Shanghai (CN)

(72) Inventors: Wenpeng DAI, Shanghai (CN); Wei GAO, Shanghai (CN); Lei ZHANG, Shanghai (CN); Quan RAN, Shanghai (CN); Lu ZHAI, Shanghai (CN)

(73) Assignee: Shanghai Tianma AM-OLED Co., Ltd., Shanghai (CN)

(21) Appl. No.: 17/364,855

(22) Filed: Jun. 30, 2021

(30) Foreign Application Priority Data
Mar. 1, 2021 (CN) 202110227782.2

(51) Int. Cl. C07D 487/04; H01L 51/00; C07D 241/44; C07D 231/12; C07D 285/12; C07D 271/10; C07D 413/14; C07D 471/04; C07D 277/66; C07D 263/57; C07D 417/12; C07D 403/12; C07D 285/14; C07D 285/10; C07D 513/04; C07D 519/00; C07D 413/12

(52) U.S. Cl. CPC C07D 487/04; H01L 51/0074; H01L 51/0067; H01L 51/0072; H01L 51/0058; H01L 51/0069; H01L 51/006; C07D 241/44; C07D 231/12; C07D 285/12; C07D 271/10; C07D 413/14; C07D 471/04; C07D 277/66; C07D 263/57; C07D 417/12; C07D 403/12; C07D 285/14; C07D 285/10; C07D 513/04; C07D 519/00; C07D 413/12

(57) ABSTRACT

A compound, a material for an organic electroluminescent device and an application thereof are disclosed herein. The compound has a structure represented by Formula (1). The compound has a relatively high refractive index and can effectively improve the light extraction efficiency. The external quantum efficiency of the compound makes it suitable for use as a capping layer when used in an organic electroluminescent device. The compound has a relatively high refractive index in the region of visible light (400-750 nm), which is advantageous for improving the light-emitting efficiency. The compound has a relatively large extinction coefficient in the ultraviolet region (less than 400 nm), which is advantageous for absorbing harmful light and protecting eyesight. Meanwhile, a deuterium atom may be introduced into the compound to significantly improve the device lifetime.

### CROSS-REFERENCE TO RELATED APPLICATION(S)

This application claims priority to Chinese Patent Application No. 202110227782.2 filed on Mar. 1, 2021, the disclosure of which is incorporated herein by reference in its entirety.

### TECHNICAL FIELD

The present disclosure relates to the technical field of organic electroluminescence and in particular, to a compound, a material for an organic electroluminescent device and an application thereof.

### BACKGROUND

After decades of development, organic electroluminescence (such as organic light-emitting diode, OLED) has gained considerable progress. The OLED has an internal quantum efficiency of approximately 100% and an external quantum efficiency of only about 20%. Most light is confined inside a light-emitting device due to factors such as loss of a substrate mode, surface plasmon loss and waveguide effect, resulting in the loss of a large amount of energy.

In a top emitting device, an organic capping layer (CPL) is deposited through evaporation on a translucent metal electrode Al so that an optical interference distance is adjusted, the reflection of external light is suppressed, and the extinction caused by the movement of surface plasmon is suppressed, thereby improving light extraction efficiency and light-emitting efficiency.

High requirements are imposed on the performance of a material for CPL: no absorption within the wavelength range (400 nm to 700 nm) of visible light, a high refractive index (generally, n>2.1), a low extinction coefficient (k≤0.00) within the wavelength range of 400 nm to 600 nm, a high glass transition temperature, a high molecular thermal stability, and an ability to be deposited through evaporation without thermal decomposition.

Materials for CPL in the related art still have many problems, for example, (1) the refractive index is generally below 1.9 and cannot meet the requirement for high refractive index; (2) in the case where the refractive index meets the requirement, the materials have relatively strong absorption or a relatively large extinction coefficient in the region of visible light; (3) amine derivatives with a particular structure and a high refractive index and the use of materials that have particular parameters have improved the light extraction efficiency, while the problems of light-emitting efficiency and chromaticity are still to be solved especially for blue light-emitting elements; (4) to increase the density of molecules and achieve high thermal stability, a molecular structure is designed to be large and loose so that molecules cannot be tightly packed, resulting in too many molecular gel holes during evaporation and incomplete coverage; (5) a simple design of an electron-type capping layer material to achieve the effects of electron transmission and light extraction saves a preparation cost of the device to a certain extent so that multiple effects are achieved, while the design is not conducive to light extraction and improves the light-emitting efficiency only slightly and the problem of chromaticity is not solved.

Therefore, more kinds of CPL materials with higher performance are to be developed in the art.

### SUMMARY

In view of defects in the related art, a first aspect of the present disclosure is to provide a compound, and in particular an organic electroluminescent material. The compound has a relatively high refractive index and can effectively improve the external quantum efficiency (EQE) of an organic electroluminescent device when used as a material for a capping layer. Meanwhile, the compound has a relatively small extinction coefficient in the region of blue light (400-450 nm) and hardly absorbs blue light, thereby improving the light-emitting efficiency.

To achieve the above advantages, the present disclosure adopts a solution described below.

The present disclosure provides a compound, which has a structure represented by Formula (1):

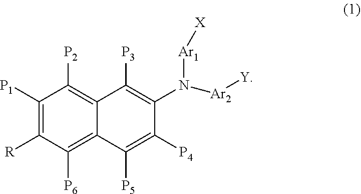

In Formula (1), R is selected from any one of substituted or unsubstituted C10-C60 fused-ring aryl and substituted or unsubstituted C6-C60 fused-ring heteroaryl.

In Formula (1), P₁, P₂, P₃, P₄, P₅ and P₆ are each independently selected from any one of a hydrogen atom, a deuterium atom, CD₃, CD₂CH₃ and CD₂CD₃.

In Formula (1), Ar₁ and Ar₂ are each independently selected from any one of a single bond, substituted or unsubstituted C6-C60 arylene, substituted or unsubstituted C3-C60 heteroarylene, substituted or unsubstituted C10-C60 fused-ring arylene and substituted or unsubstituted C6-C60 fused-ring heteroarylene.

In Formula (1), X and Y are each independently selected from substituted or unsubstituted C6-C60 aryl and substituted or unsubstituted C3-C60 heteroaryl, and at least one of X and Y is selected from substituted or unsubstituted C3-C60 electron withdrawing heteroaryl.

Substituted groups in R, Ar₁, Ar₂, X and Y are each independently selected from any one or a combination of at least two of protium, deuterium, tritium, halogen, C1-C10 alkyl, C1-C10 haloalkyl, C1-C10 alkoxy, C6-C60 aryl and C3-C60 heteroaryl.

A second aspect of the present disclosure provides a material for an organic electroluminescent device. The material for an organic electroluminescent device includes any one or a combination of at least two of the compound as described herein.

A third aspect of the present disclosure provides an organic electroluminescent device. The organic electroluminescent device includes a first electrode layer, an organic function layer and a second electrode layer which are stacked in sequence.

The organic function layer includes the material as described herein.

A fourth aspect of the present disclosure provides another organic electroluminescent device. The organic electroluminescent device includes a first capping layer, a first electrode layer, an organic function layer and a second electrode layer which are stacked in sequence.

The first capping layer includes the material as described herein.

A fifth aspect of the present disclosure provides a display panel. The display panel includes the organic electroluminescent device as described herein.

A sixth aspect of the present disclosure is to provide a display device. The display device includes the display panel as described herein.

Compared with the related art, the present disclosure has beneficial effects described below.

The compound provided by the present disclosure has a relatively high refractive index and can effectively improve the light extraction efficiency and the external quantum efficiency (EQE) of an organic electroluminescent device when used in the organic electroluminescent device especially as a material for a capping layer. Meanwhile, the compound has a relatively small extinction coefficient in the region of blue light (400-450 nm) and hardly absorbs blue light, thereby improving the light-emitting efficiency.

### BRIEF DESCRIPTION OF DRAWINGS

FIG. 1 is a structure diagram of an organic electroluminescent device according to an embodiment of the present disclosure.

FIG. 2 is a structure diagram of an organic electroluminescent device according to another embodiment of the present disclosure.

### REFERENCE LIST

1 substrate
2 anode
3 hole injection layer
4 first hole transport layer
5 second hole transport layer
6 light-emitting layer
7 first electron transport layer
8 second electron transport layer
9 cathode
10 first capping layer
11 second capping layer

### DETAILED DESCRIPTION

In accordance with a first aspect of the present disclosure, a compound is provided. The compound has a structure represented by Formula (1):

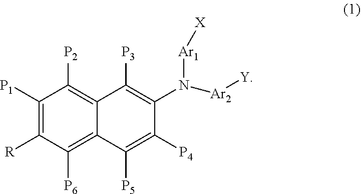

In Formula (1), R is selected from any one of substituted or unsubstituted C10-C60 (for example, C12, C14, C16, C18, C20, C22, C24, C26, C28, C30, C32, C34, C36, C38, C40, C42, C44, C46, C48, C50, C52, C54, C56, C58 or the like) fused-ring aryl and substituted or unsubstituted C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) fused-ring heteroaryl.

In Formula (1), P₁, P₂, P₃, P₄, P₅ and P₆ are each independently selected from any one of a hydrogen atom, a deuterium atom, CD₃, CD₂CH₃ and CD₂CD₃.

In Formula (1), Ar₁ and Ar₂ are each independently selected from any one of a single bond, substituted or unsubstituted C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) arylene, substituted or unsubstituted C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroarylene, substituted or unsubstituted C10-C60 (for example, C12, C14, C16, C18, C20, C22, C24, C26, C28, C30, C32, C34, C36, C38, C40, C42, C44, C46, C48, C50, C52, C54, C56, C58 or the like) fused-ring arylene and substituted or unsubstituted C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) fused-ring heteroarylene.

In Formula (1), X and Y are each independently selected from substituted or unsubstituted C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl and substituted or unsubstituted C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl, and at least one of X and Y is selected from substituted or unsubstituted C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) electron withdrawing heteroaryl.

Substituted groups in R, Ar₁, Ar₂, X and Y are each independently selected from any one or a combination of at least two of protium, deuterium, tritium, halogen, C1-C10 (for example, C2, C3, C4, C5, C6, C7, C8, C9 or the like) alkyl, C1-C10 (for example, C2, C3, C4, C5, C6, C7, C8, C9 or the like) haloalkyl, C1-C10 (for example, C2, C3, C4, C5, C6, C7, C8, C9 or the like) alkoxy, C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl and C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl.

As described hereinbelow, in accordance with one aspect of the present disclosure, in the compound structure formed through the substitution of an arylamino group at position 2 and the substitution of a fused ring group (R) at position 6 on a naphthalene ring, an naphthalene conjugation is the longest, smallest molecular distortion formed, and a film formed through molecular evaporation has a smoother arrangement, which facilitates the internal extraction of visible light. The compound has a higher refractive index, is suitable for use as a material for a capping layer of an organic electroluminescent device, and can effectively improve the light extraction efficiency and the external quantum efficiency. Moreover, the compound has a relatively large extinction coefficient in the ultraviolet region (less than 400 nm), thereby facilitating the absorption of harmful light.

In an embodiment, R is selected from groups represented by Formula (2), Formula (3) and Formula (4):

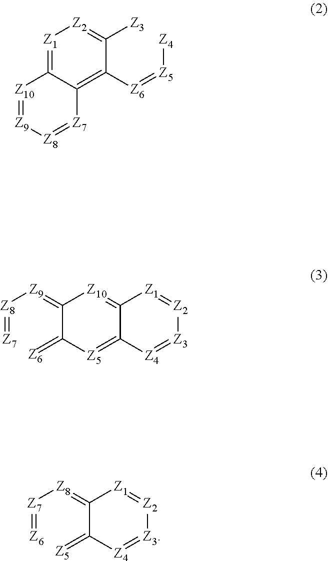

Z₁ to Z₁₀ are each independently selected from a N atom, CH and CR₁, where R₁ is selected from any one or a combination of at least two of protium, deuterium, tritium, halogen, C1-C10 (for example, C2, C3, C4, C5, C6, C7, C8, C9 or the like) alkyl, C1-C10 (for example, C2, C3, C4, C5, C6, C7, C8, C9 or the like) haloalkyl, C1-C10 (for example, C2, C3, C4, C5, C6, C7, C8, C9 or the like) alkoxy, C6-C60 (for example, C8, C10, C12, C14, C16, C18, C20, C22, C24, C26, C28, C30, C32, C34, C36, C38, C40, C42, C44, C46, C48, C50, C52, C54, C56, C58 or the like) aryl and C3-C60 (for example, C4, C5, C6, C7, C8, C10, C12, C14, C16, C18, C20, C22, C24, C26, C28, C30, C32, C34, C36, C38, C40, C42, C44, C46, C48, C50, C52, C54, C56, C58 or the like) heteroaryl.

In accordance with one embodiment of the present disclosure, R is a structure where at most three rings are fused so that not only a good light extraction effect but also a high thermal stability can be achieved. A structure with too many fused rings results in thermal decomposition and poor solubility, which is unfavorable for cleaning a MASK during mass production.

In an embodiment, Z₁ to Z₁₀ are each independently selected from CH and CR₁.

In an embodiment, R is selected from any one of the following groups:

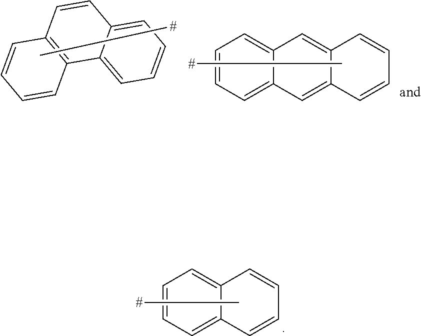

# represents a linkage site of the group.

In an embodiment, R is selected from any one of the following groups:

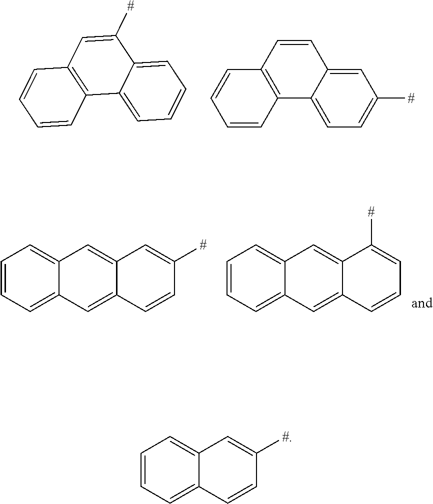

# represents a linkage site of the group.

In one embodiment, naphthyl, anthracenyl and phenanthrenyl are linked at the preceding sites so that the molecule of the compound has a high refractive index due to the longest conjugated chain.

In an embodiment, R is selected from any one of the following groups:

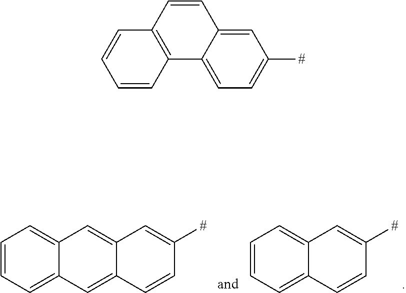

where # represents a linkage site of the group.

Furthermore, in one embodiment, anthracenyl, phenanthrenyl and naphthyl are linked at the preceding particular sites so that the relative volume of the molecule can be further reduced and the refractive index of the molecule can be improved.

In an embodiment, in Formula (2), at least one of Z₁ to Z₁₀ is selected from N.

In Formula (3), at least one of Z₁ to Z₁₀ is selected from N.

In Formula (4), at least one of Z₁ to Z₈ is selected from N.

In accordance with another embodiment, R is a N-containing fused-ring heteroaryl group and can provide a better light-emitting effect than a fused-ring heteroaryl group containing no nitrogen since the molecular polarity is improved.

In an embodiment, R is selected from any one of the following groups:

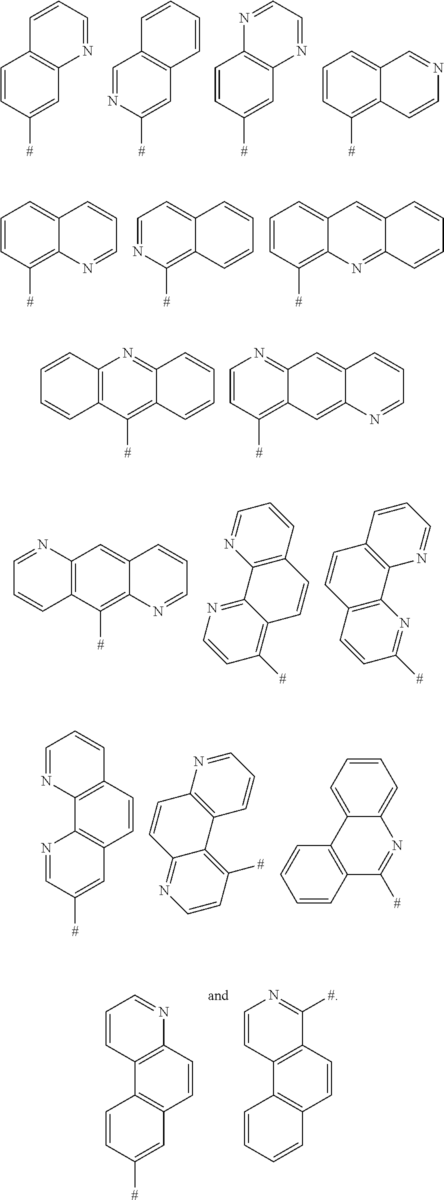

# represents a linkage site of the group.

In an embodiment, in Formula (2), at least one of Z₁ to Z₁₀ is selected from CR₁, and R₁ is selected from any one or a combination of at least two of deuterium, halogen and C1-C10 haloalkyl.

In Formula (3), at least one of Z₁ to Z₁₀ is selected from CR₁, and R₁ is selected from any one or a combination of at least two of deuterium, halogen and C1-C10 haloalkyl.

In Formula (4), at least one of Z₁ to Z₈ is selected from CR₁, and R₁ is selected from any one or a combination of at least two of deuterium, halogen and C1-C10 haloalkyl.

“A combination of at least two” refers to that in the presence of at least two R₁ in the structure, the at least two R₁ may be selected from different substituents.

In another embodiment, R includes the substitution of deuterium, halogen or C1-C10 haloalkyl. A structure with the substitution of deuterium increases the lifetime of the device, and a structure with the substitution of halogen or C1-C10 haloalkyl increases the molecular polarity so that the device can have better efficiency.

In an embodiment, R is selected from any one of the following groups:

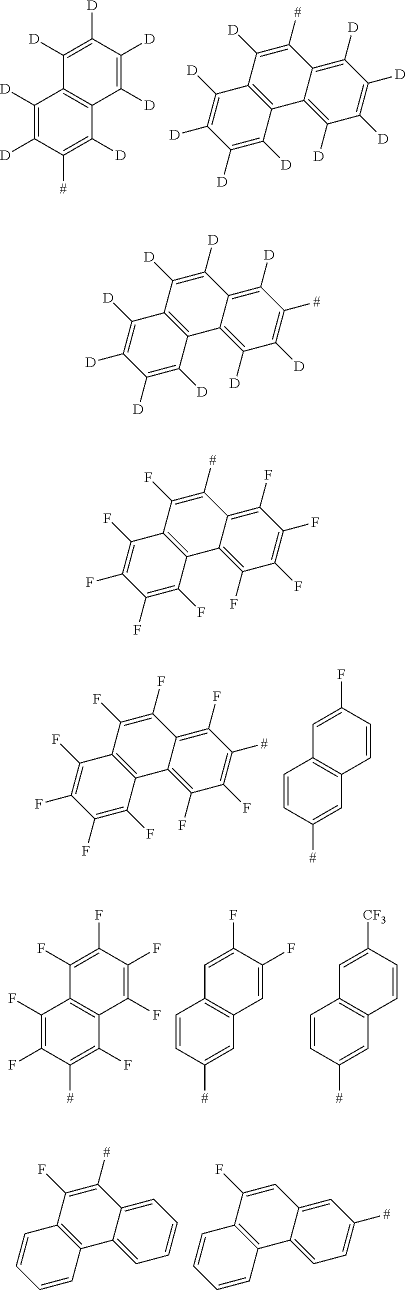

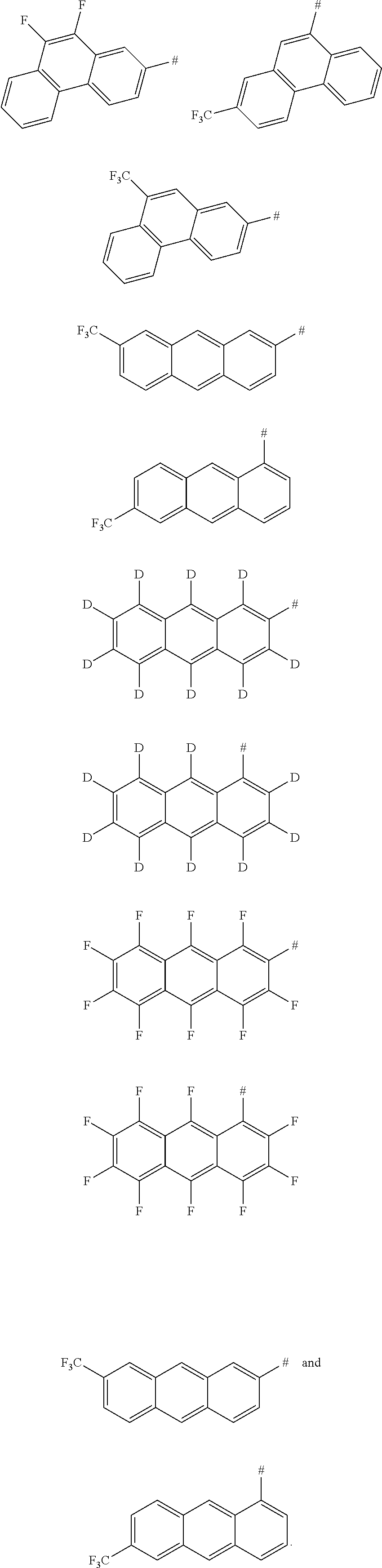

Where # represents a linkage site of the group.

In an embodiment, X and Y are each independently selected from substituted or unsubstituted C3-C60 electron withdrawing heteroaryl.

In an embodiment, X and Y are each independently selected from any one of the following substituted or unsubstituted groups:

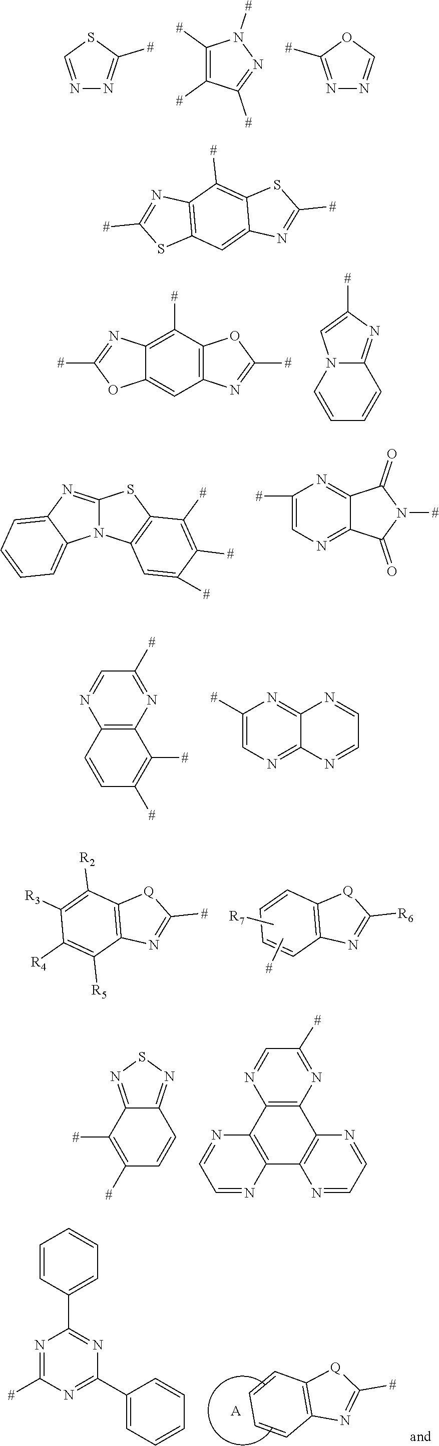

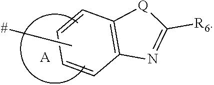

Where # represents a linkage site of the group.

Q is selected from an O atom, a S atom and NR₈.

R₂ to R₇ are each independently selected from any one of hydrogen, protium, deuterium, tritium, halogen, C1-C10 alkyl, C1-C10 alkoxy, C6-C60 aryl and C3-C60 heteroaryl.

The ring A is fused at any position of a benzene ring where the ring A can be fused and the ring A is selected from substituted or unsubstituted C6-C30 aromatic rings and substituted or unsubstituted C3-C30 heteroaromatic rings.

In an embodiment, X and Y are each independently selected from any one of the following groups:

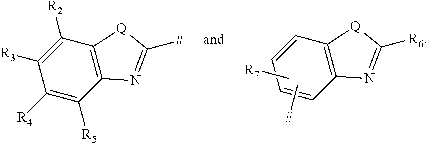

Where # represents a linkage site of the group.

Q and R₂ to R₇ each have the same ranges as defined above.

In an embodiment, X and Y are each independently selected from any one of the following groups:

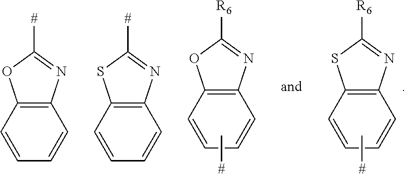

Where # represents a linkage site of the group.

R₆ has the same range as defined above.

In an embodiment, Ar₁ is selected from substituted or unsubstituted C6-C60 arylene, substituted or unsubstituted C3-C60 heteroarylene, substituted or unsubstituted C10-C60 fused-ring arylene and substituted or unsubstituted C6-C60 fused-ring heteroarylene, and X is selected from

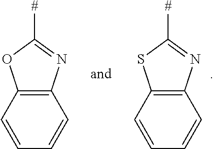

Additionally and alternatively, Ar₂ is selected from substituted or unsubstituted C6-C60 arylene, substituted or unsubstituted C3-C60 heteroarylene, substituted or unsubstituted C10-C60 fused-ring arylene and substituted or unsubstituted C6-C60 fused-ring heteroarylene, and Y is selected from

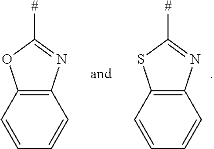

Where # represents a linkage site of the group.

In one embodiment, if Ar₁ and Ar₂ are the preceding aromatic groups, X and Y are linked to a side of a five-membered ring. This structure has the longest conjugation length and a lone pair on the N atom has the largest conjugation area so that the largest refractive index is obtained and the best device performance is achieved.

In an embodiment, Ar₁ is selected from a single bond, and X is selected from

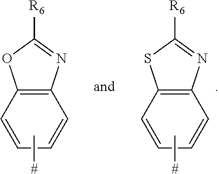

Additionally and alternatively, Ar₂ is selected from a single bond, and Y is selected from

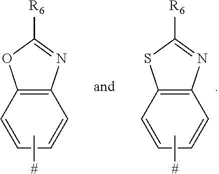

R₆ has the same range as defined above.

Where # represents a linkage site of the group.

In another embodiment, if Ar₁ and Ar₂ are single bonds, X and Y are linked to a side of a benzene ring. This structure has a more uniform arrangement of electron clouds in the molecule than that formed by a direct linkage via R₆ so that the largest refractive index is obtained and the best device performance is achieved.

In an embodiment, Ar₁ and Ar₂ are each independently selected from any one of phenylene, biphenylene, terphenylene, naphthylene, anthrylene, phenanthrylene, pyridinylene, pyrimidinylene, triazinylene, furylene, pyrrolidene, thienylene, quinolylene, isoquinolylene, benzofurylene and benzothienylene.

In an embodiment, Ar₁ and Ar₂ are each independently selected from

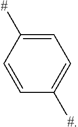

In one embodiment, Ar₁ and Ar₂ are p-phenylene. When Ar₁ and Ar₂ match R with a fused ring structure, the longest linear conjugated chain can be obtained so that the refractive index of the compound is further improved and device efficiency is improved.

In an embodiment, Ar₁ and Ar₂ are each independently selected from naphthylene and biphenylene.

In one embodiment, Ar₁ and Ar₂ are selected from naphthylene and biphenylene, which enable the compound to have a greater conjugation length than phenylene, thereby further improving the performance.

In an embodiment, Ar₁ and Ar₂ are each independently selected from

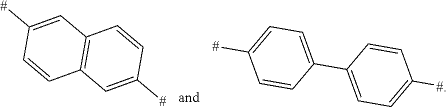

Where # represents a linkage site of the group.

In one embodiment, naphthylene and phenylene are linked through the preceding sites so that the compound has the longest linear conjugation, thereby further improving the device performance.

In an embodiment, the compound has a structure represented by Formula (2):

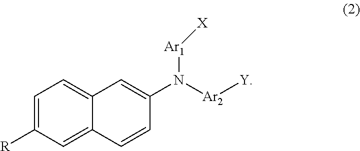

R, Ar₁, Ar₂, X and Y each have the same ranges as defined in Formula (1).

In an embodiment, at least one of P₁, P₂, P₃, P₄, P₅ and P₆ is selected from a deuterium atom.

When a naphthalene ring includes the substitution of at least one deuterium atom, the compound has more stable chemical properties, thereby obtaining a longer device lifetime.

In an embodiment, P₂, P₃, P₅ and P₆ are each selected from a deuterium atom.

Further, a better effect is achieved when four positions P₂, P₃, P₅ and P₆ are all substituted with deuterium atoms. This is because hydrogen at the positions of P₂, P₃, P₅ and P₆ is the most active and when these four positions are all substituted, the compound does not easily break down at a high temperature.

In an embodiment, the difference between the refractive index of the compound at a wavelength of 460 nm and the refractive index of the compound at a wavelength of 530 nm is 0.10-0.17, the difference between the refractive index of the compound at a wavelength of 530 nm and the refractive index of the compound at a wavelength of 620 nm is 0.03-0.10, and the difference between the refractive index of the compound at a wavelength of 460 nm and the refractive index of the compound at a wavelength of 620 nm is 0.15-0.40.

The compound of the present disclosure can satisfy the differences between the refractive indexes at the preceding wavelength bands, which means that a display panel prepared from the compound of the present disclosure can improve a color cast when performing display at multiple angles.

In an embodiment, a film with a thickness of 700 angstroms and prepared from the compound has an absorbance greater than 0.3 at a wavelength of 250 nm and at a wavelength of 380 nm.

Light at the wavelengths of 250 nm and 380 nm is harmful. The compound of the present disclosure can absorb light at the wavelengths of 250 nm and 380 nm to avoid damage to human eyes.

In an embodiment, the compound has any one of the following structures represented by P1 to P305:

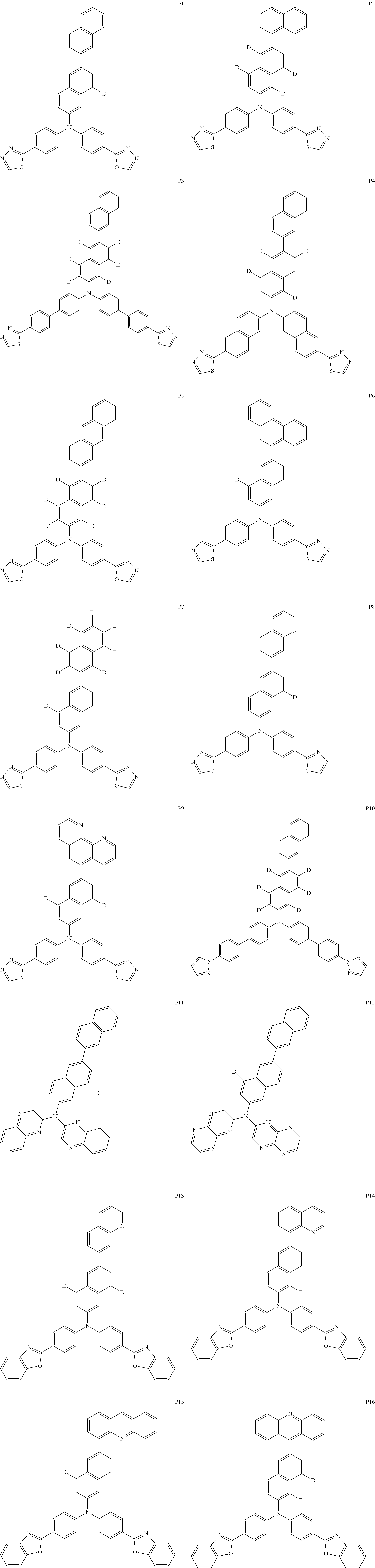

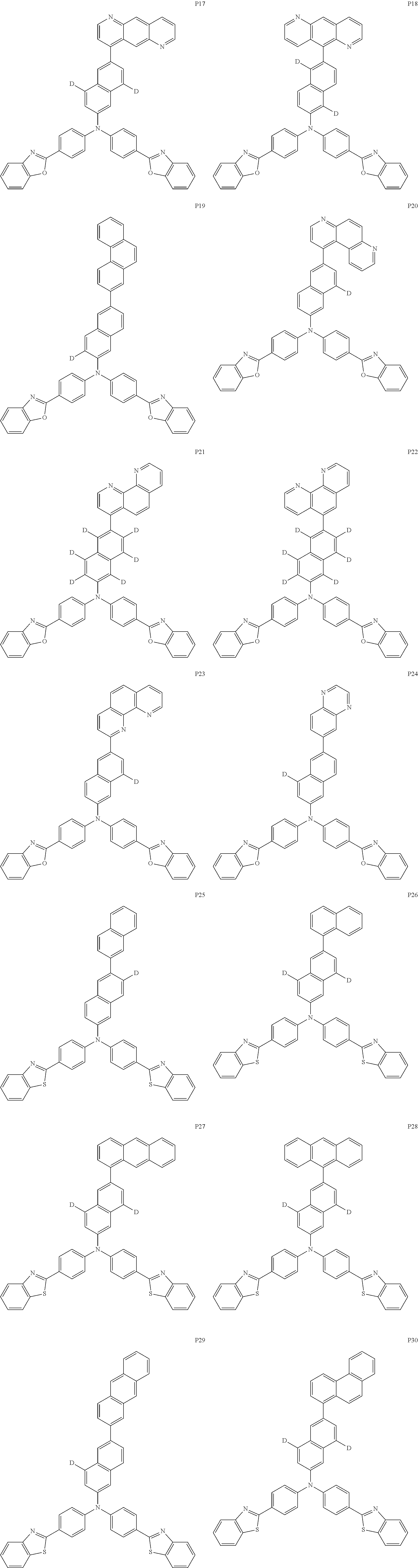

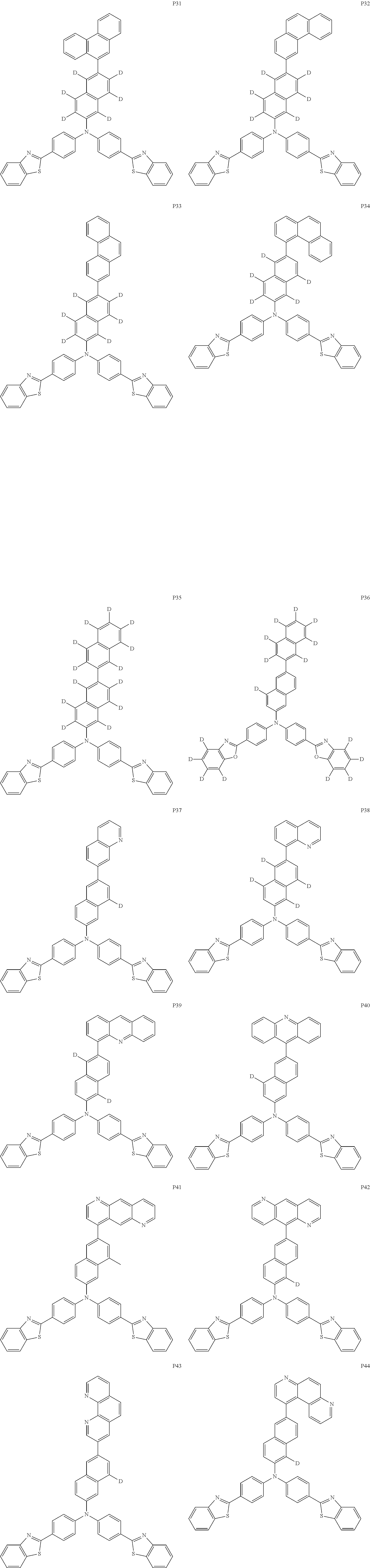

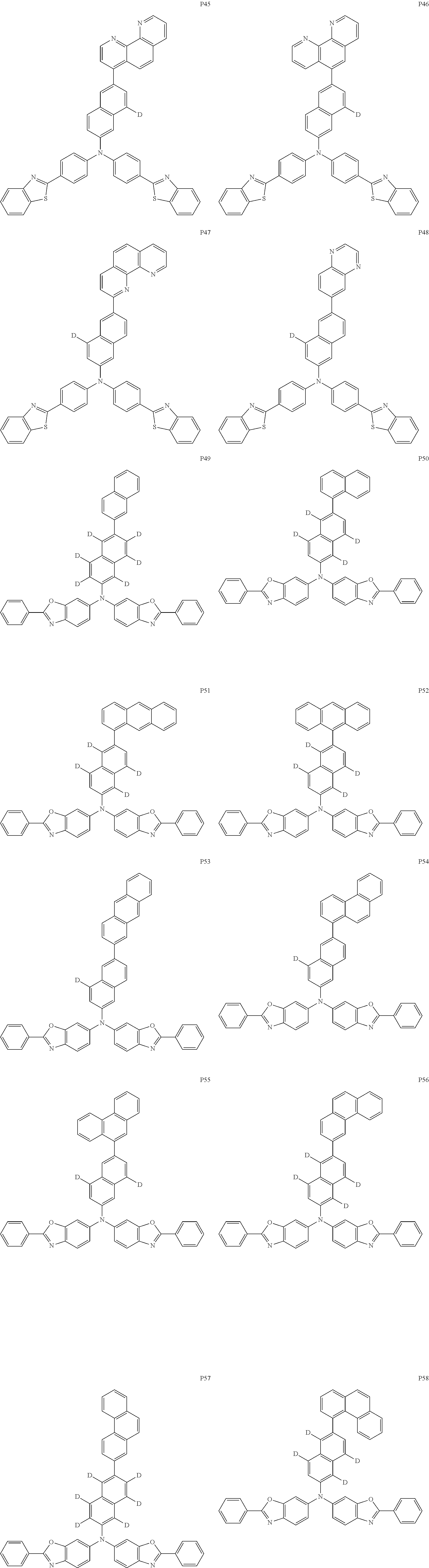

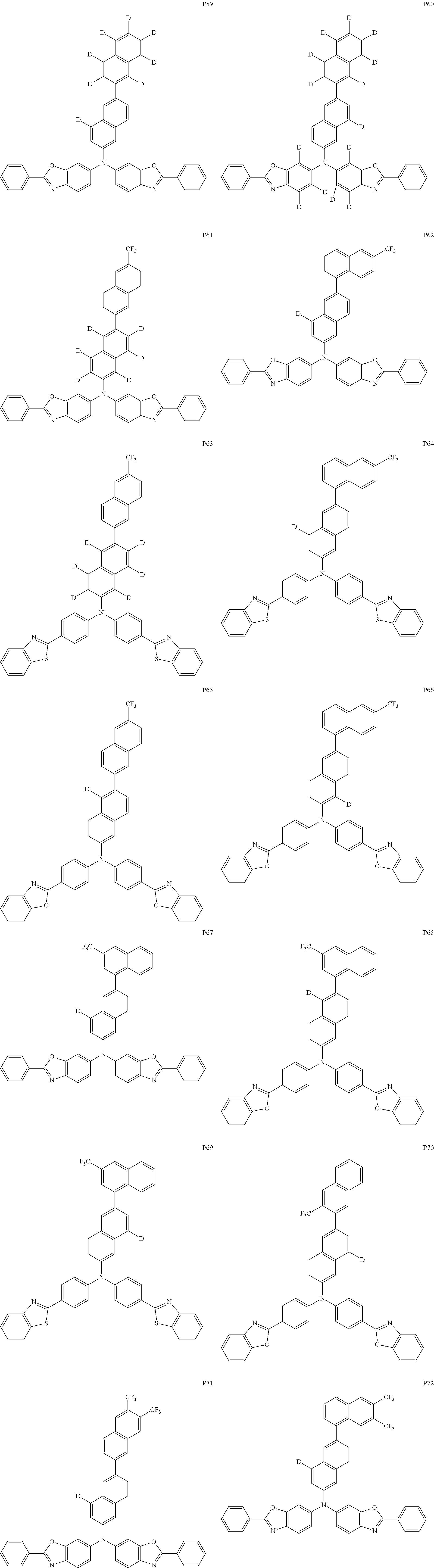

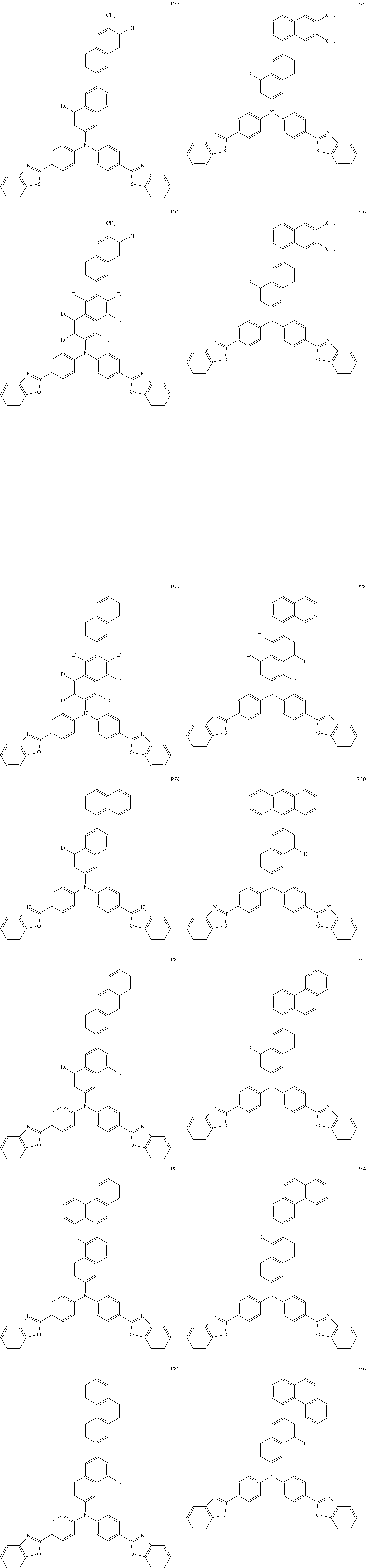

[chemical structure]

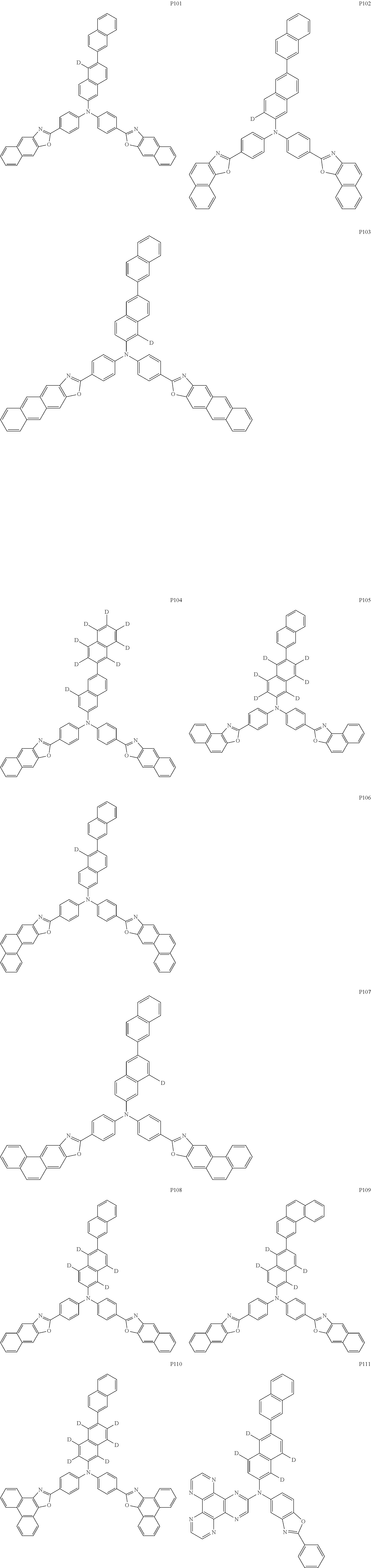

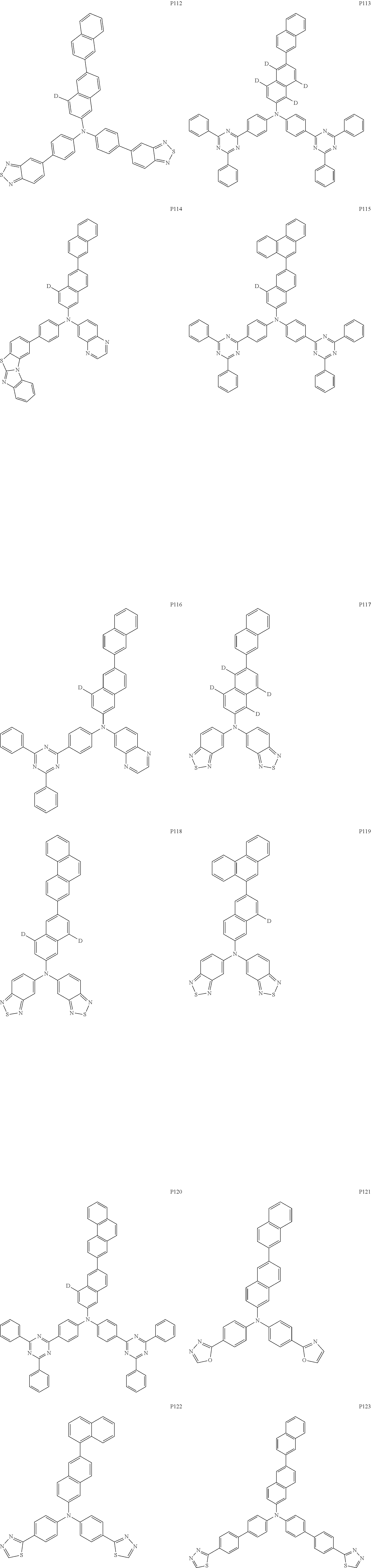

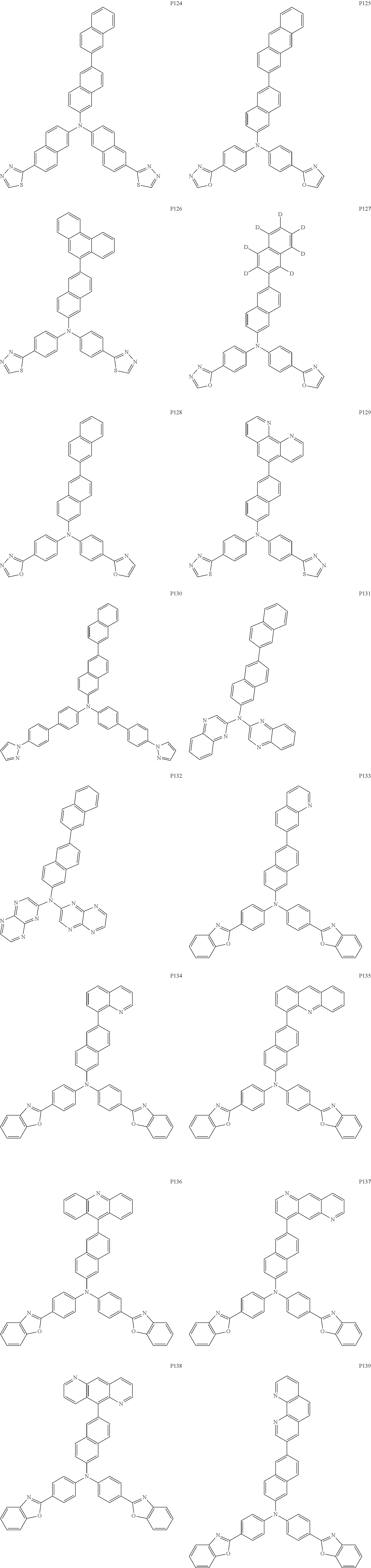

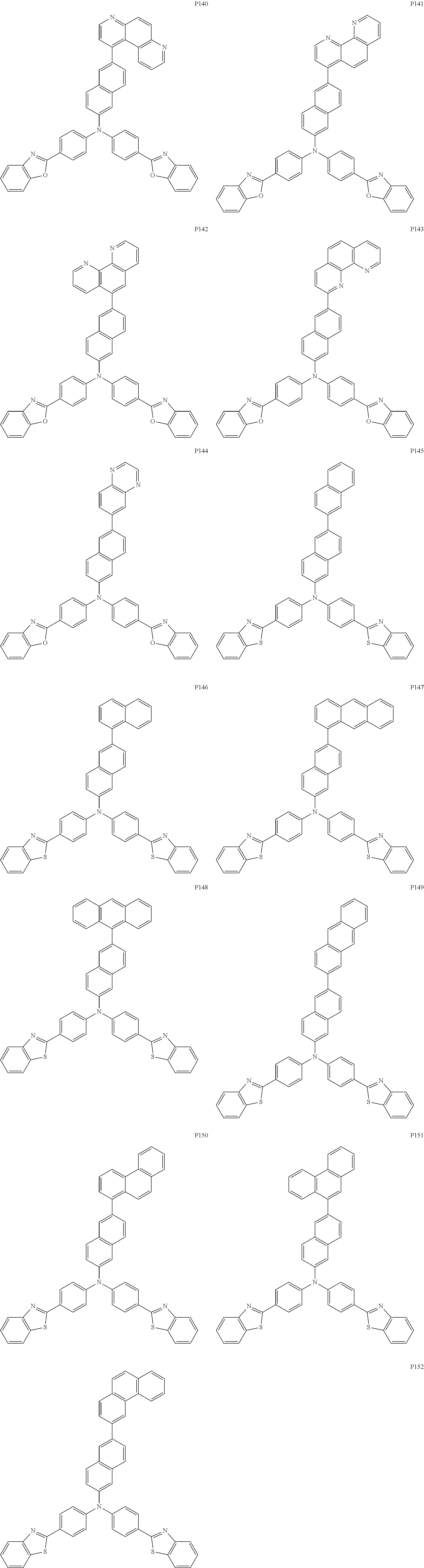

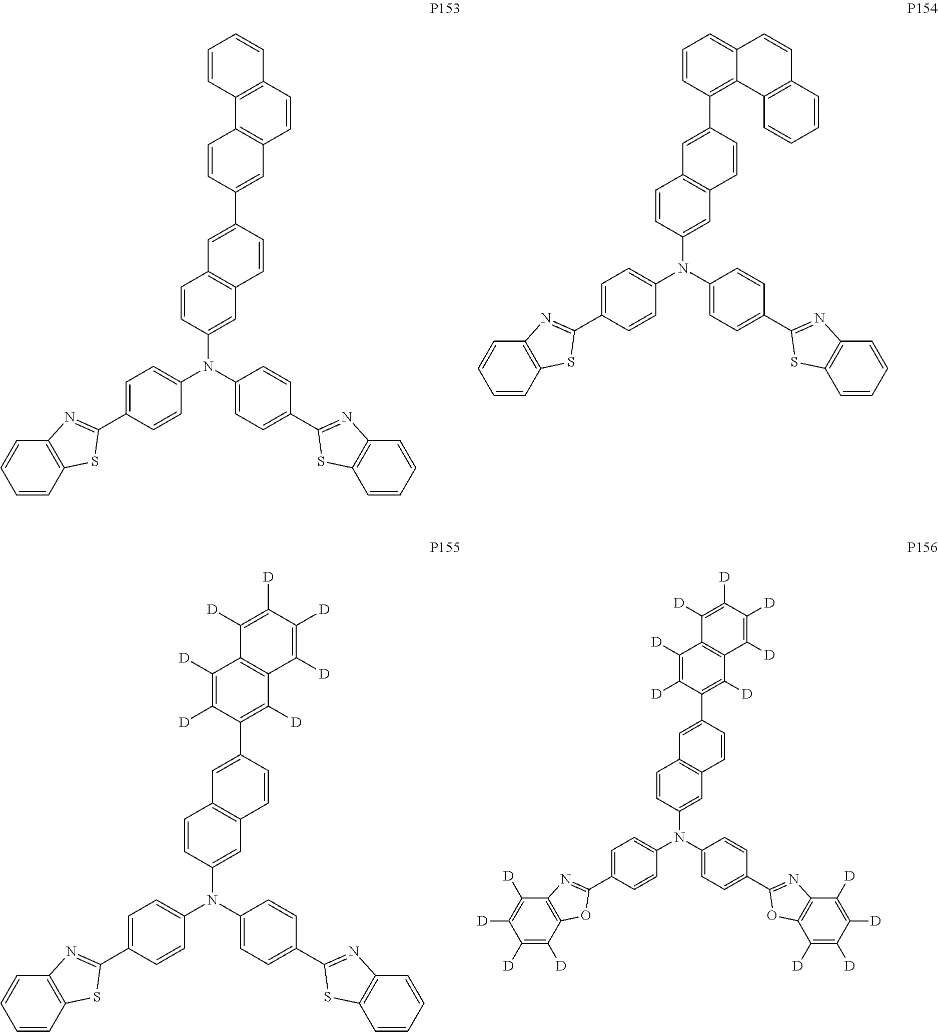 [SMILES: c1ccc2c(c1)ccc1cc(-c3ccc4cc(N(c5ccc(-c6nc7ccccc7s6)cc5)c5ccc(-c6nc7ccccc7s6)cc5)ccc4c3)ccc12 ; c1ccc2c(c1)ccc1cccc(-c3ccc4cc(N(c5ccc(-c6nc7ccccc7s6)cc5)c5ccc(-c6nc7ccccc7s6)cc5)ccc4c3)c12 ; [2H]c1c([2H])c([2H])c2c([2H])c(-c3ccc4cc(N(c5ccc(-c6nc7ccccc7s6)cc5)c5ccc(-c6nc7ccccc7s6)cc5)ccc4c3)c([2H])c([2H])c2c1[2H] ; [2H]c1c([2H])c([2H])c2oc(-c3ccc(N(c4ccc(-c5nc6c([2H])c([2H])c([2H])c([2H])c6o5)cc4)c4ccc5cc(-c6c([2H])c([2H])c7c([2H])c([2H])c([2H])c([2H])c7c6[2H])ccc5c4)cc3)nc2c1[2H]]

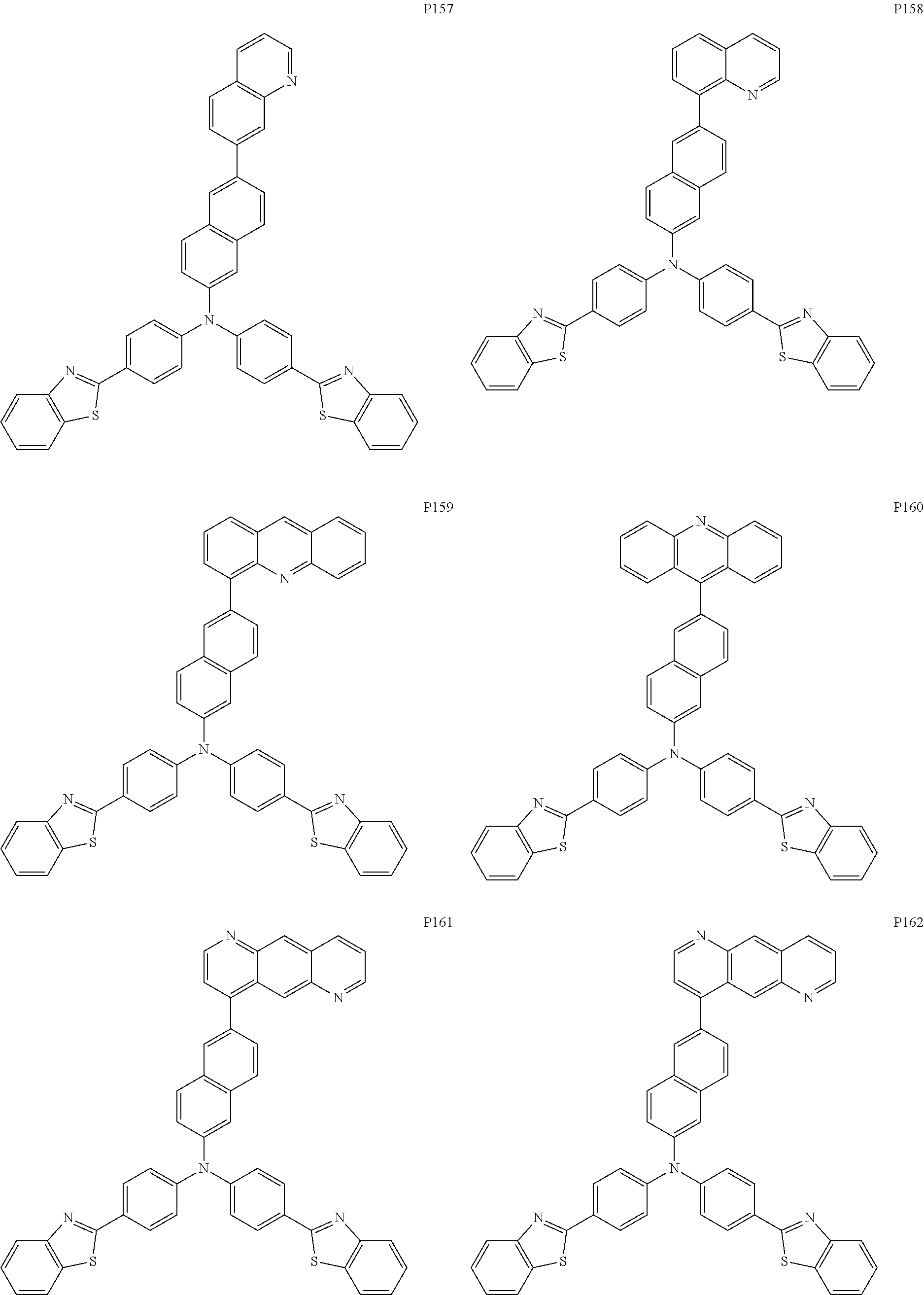

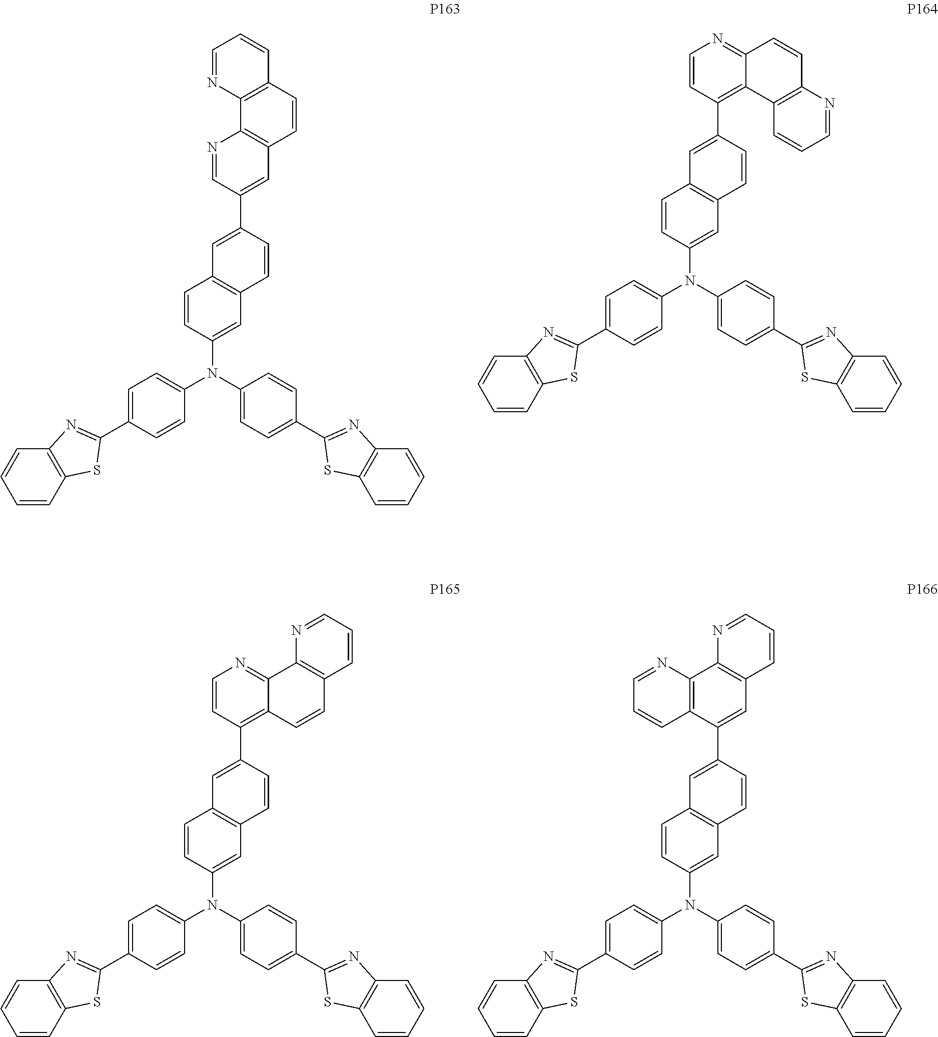 [SMILES: c1cnc2c(c1)ccc1cc(-c3ccc4cc(N(c5ccc(-c6nc7ccccc7s6)cc5)c5ccc(-c6nc7ccccc7s6)cc5)ccc4c3)cnc12 ; c1ccc2sc(-c3ccc(N(c4ccc(-c5nc6ccccc6s5)cc4)c4ccc5cc(-c6ccnc7ccc8ncccc8c67)ccc5c4)cc3)nc2c1 ; c1cnc2c(c1)ccc1c(-c3ccc4cc(N(c5ccc(-c6nc7ccccc7s6)cc5)c5ccc(-c6nc7ccccc7s6)cc5)ccc4c3)ccnc12 ; c1cnc2c(c1)cc(-c1ccc3cc(N(c4ccc(-c5nc6ccccc6s5)cc4)c4ccc(-c5nc6ccccc6s5)cc4)ccc3c1)c1cccnc12]

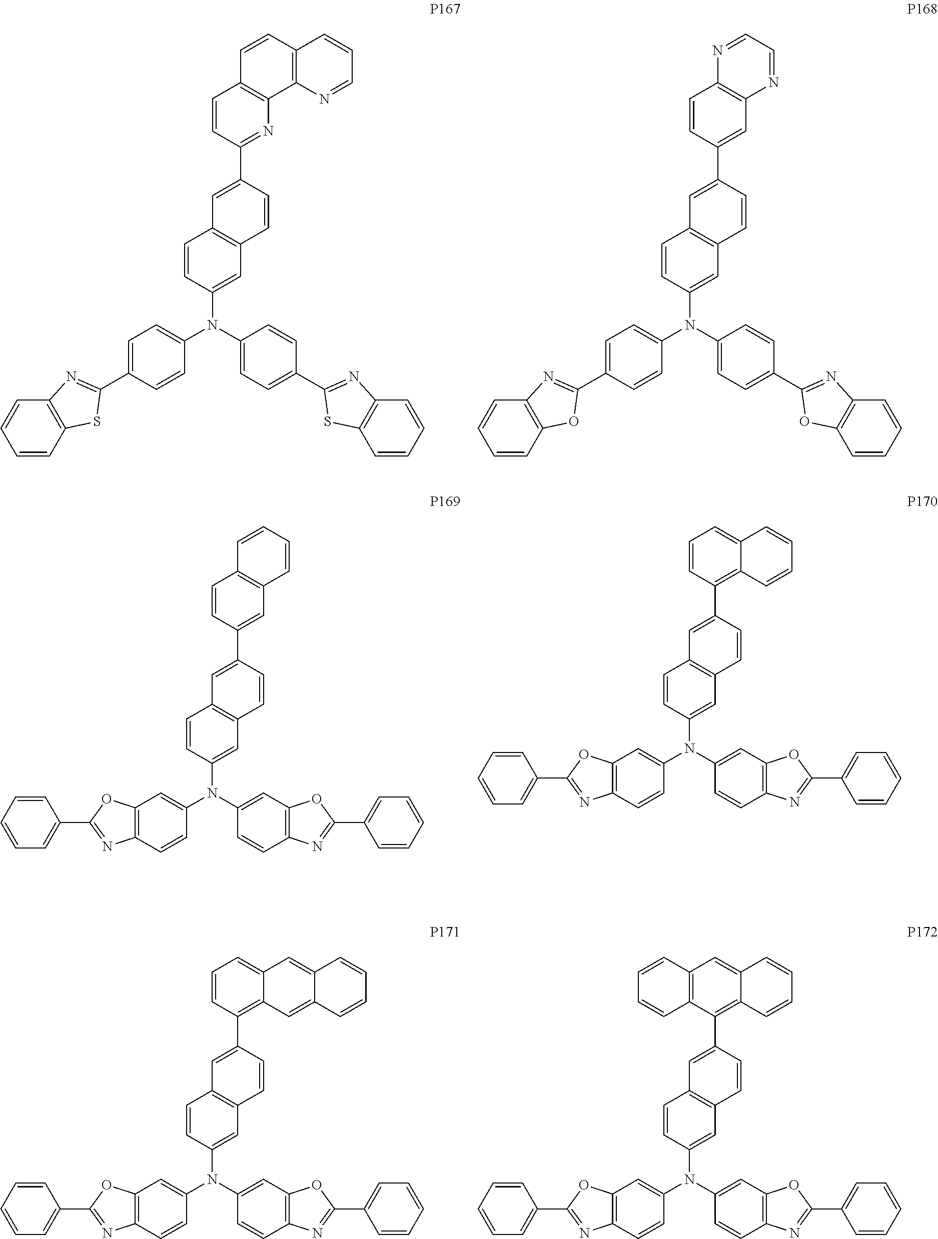

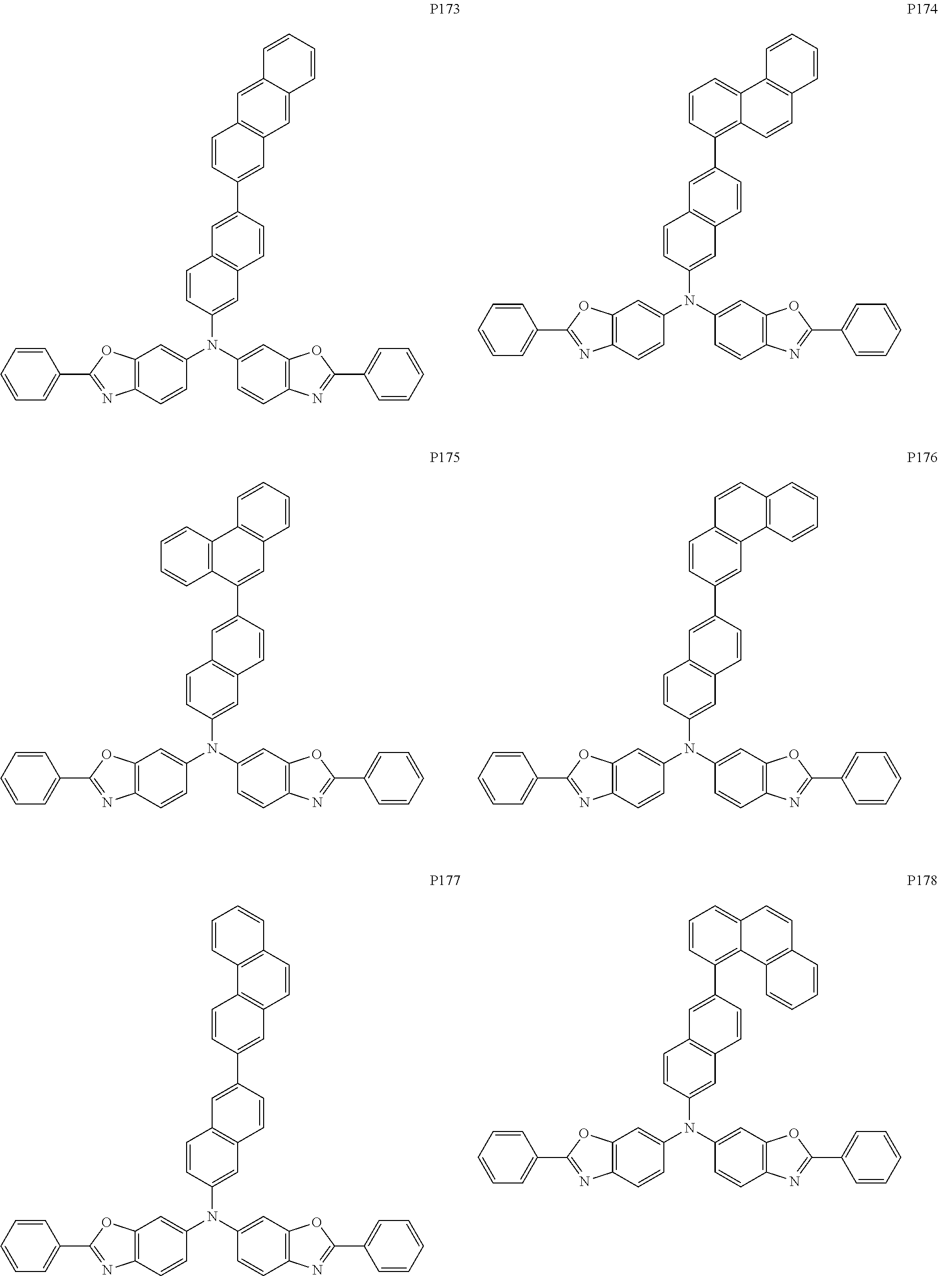

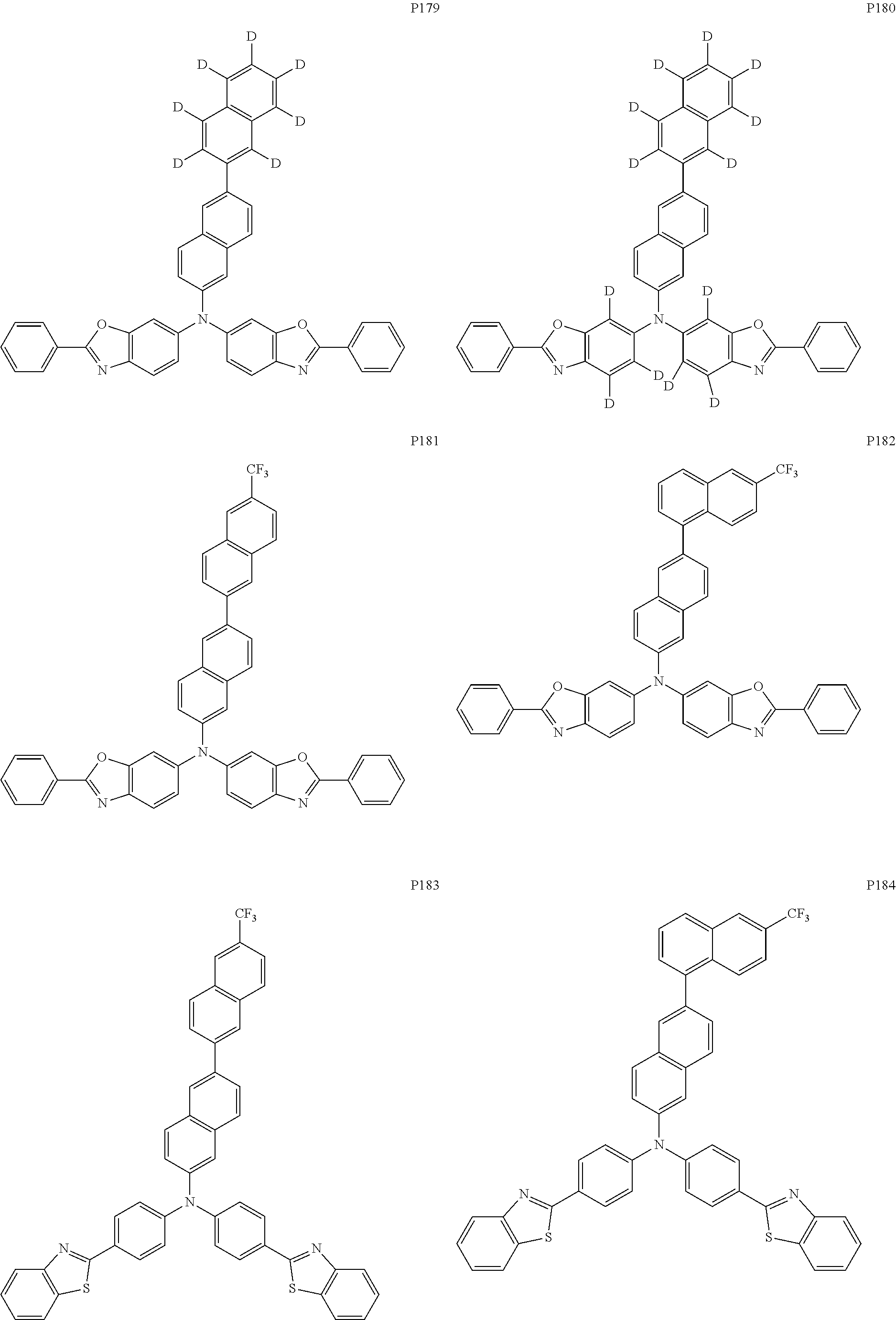

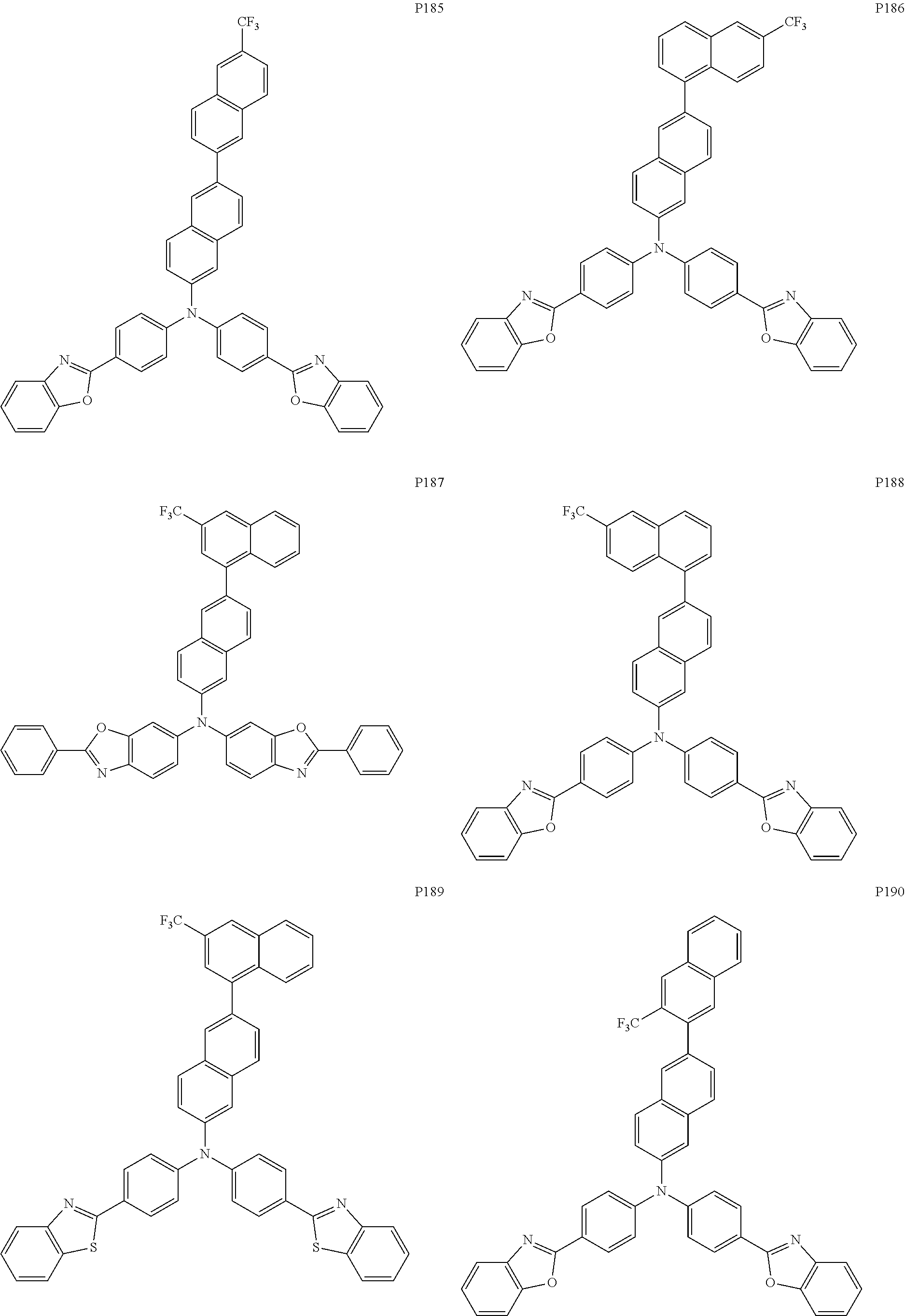

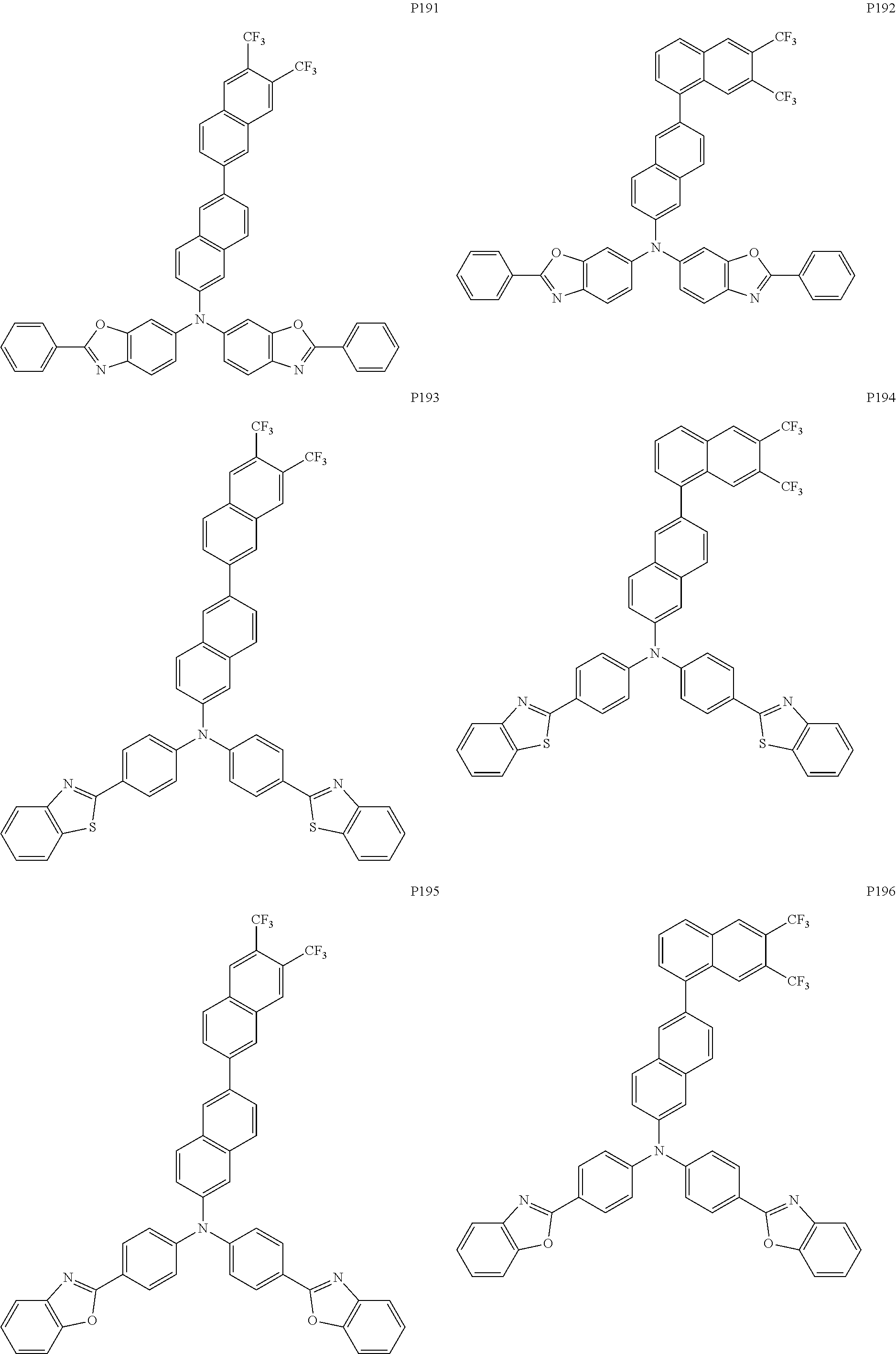

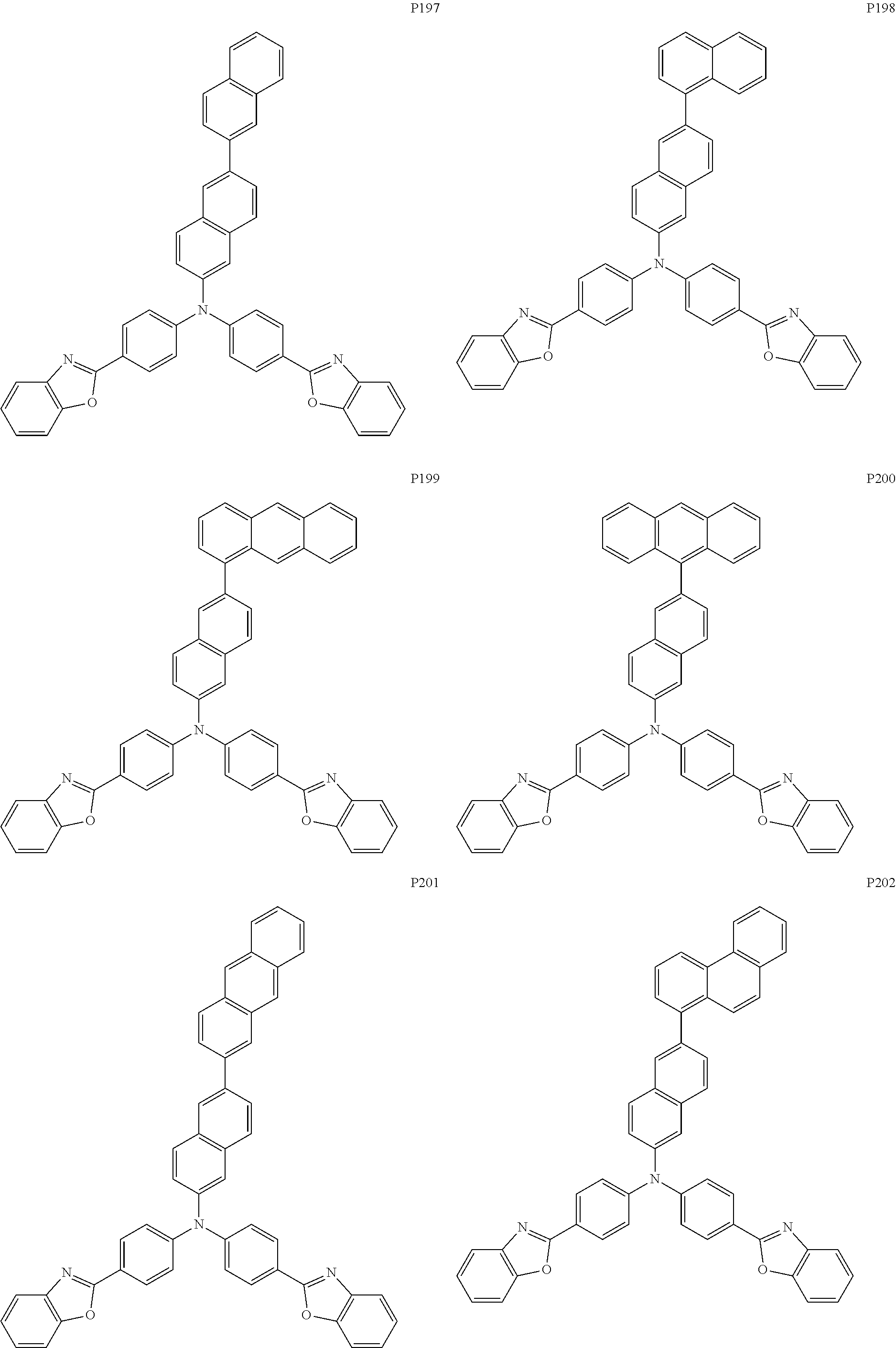

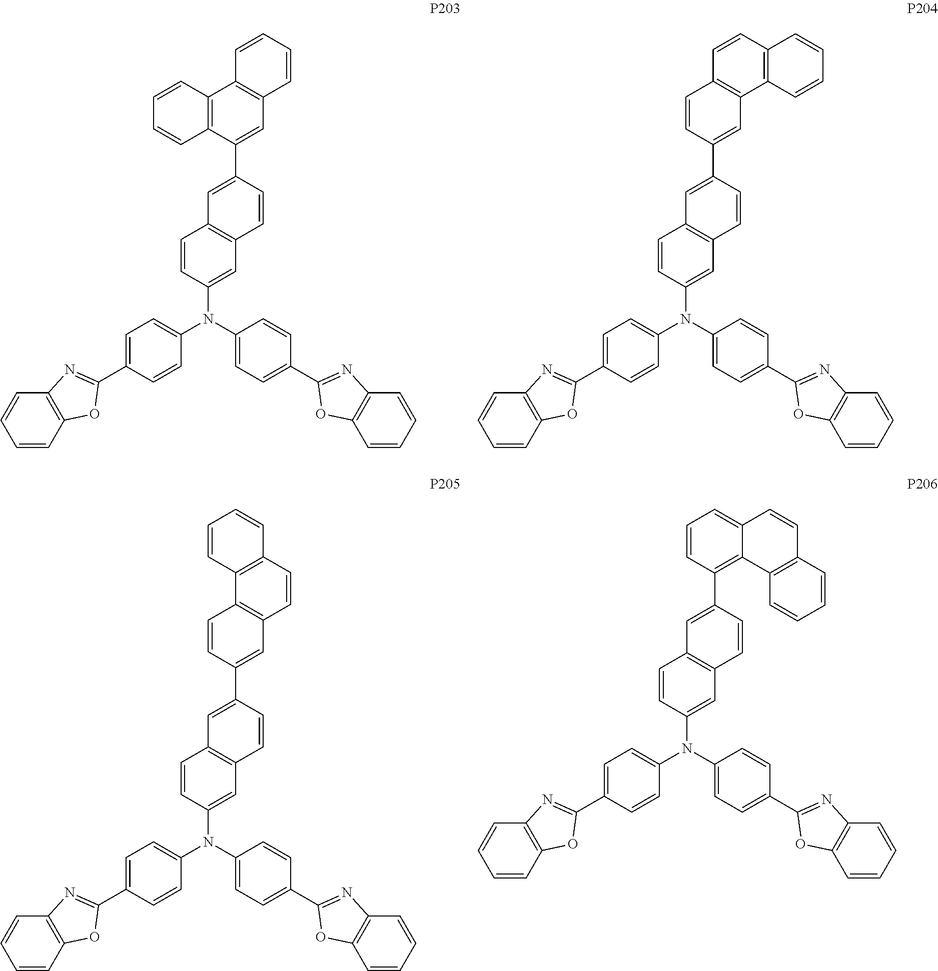 [SMILES: c1ccc2c(c1)cc(-c1ccc3cc(N(c4ccc(-c5nc6ccccc6o5)cc4)c4ccc(-c5nc6ccccc6o5)cc4)ccc3c1)c1ccccc12 ; c1ccc2c(c1)ccc1ccc(-c3ccc4cc(N(c5ccc(-c6nc7ccccc7o6)cc5)c5ccc(-c6nc7ccccc7o6)cc5)ccc4c3)cc12 ; c1ccc2c(c1)ccc1cc(-c3ccc4cc(N(c5ccc(-c6nc7ccccc7o6)cc5)c5ccc(-c6nc7ccccc7o6)cc5)ccc4c3)ccc12 ; c1ccc2c(c1)ccc1cccc(-c3ccc4cc(N(c5ccc(-c6nc7ccccc7o6)cc5)c5ccc(-c6nc7ccccc7o6)cc5)ccc4c3)c12]

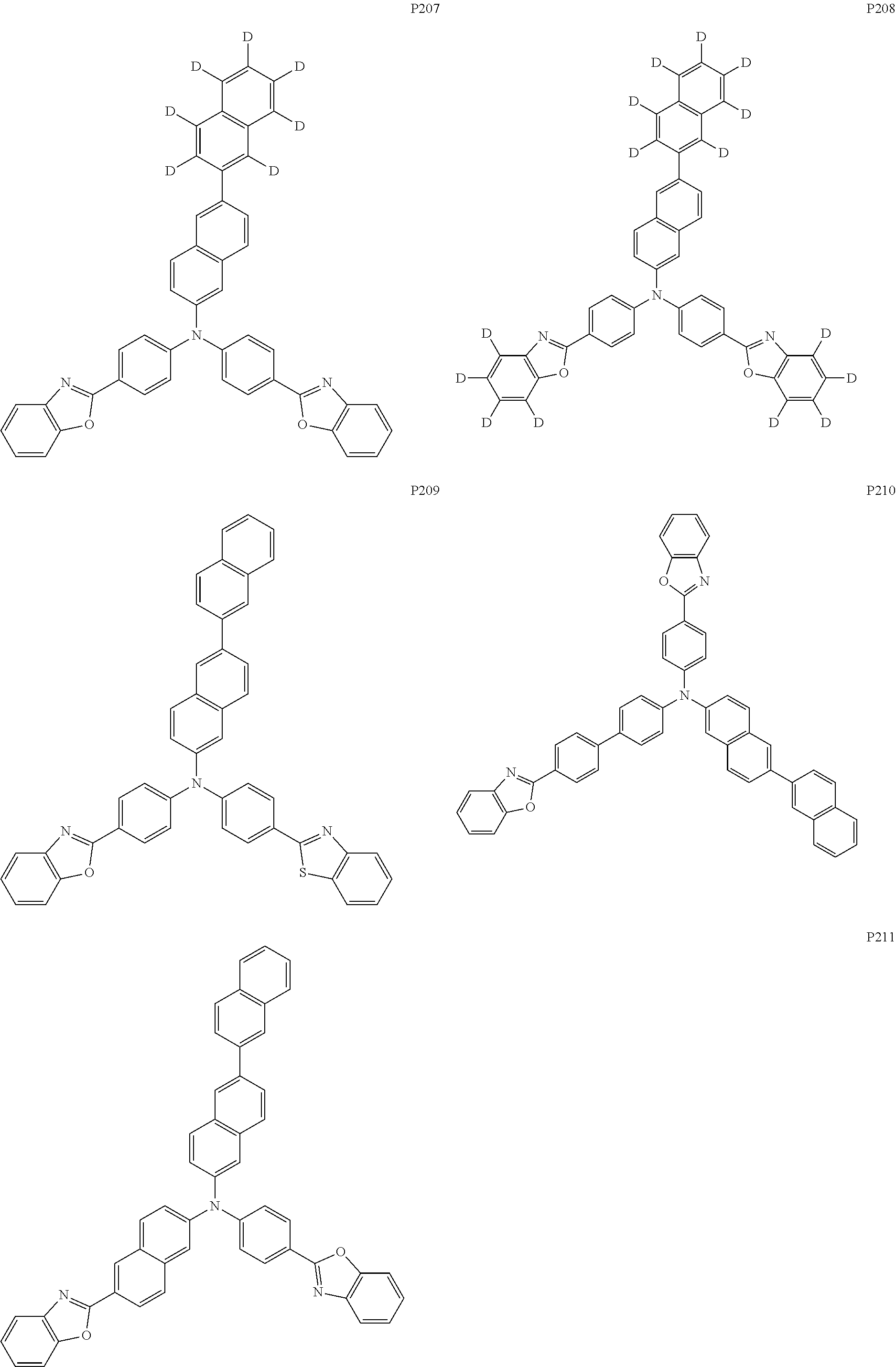

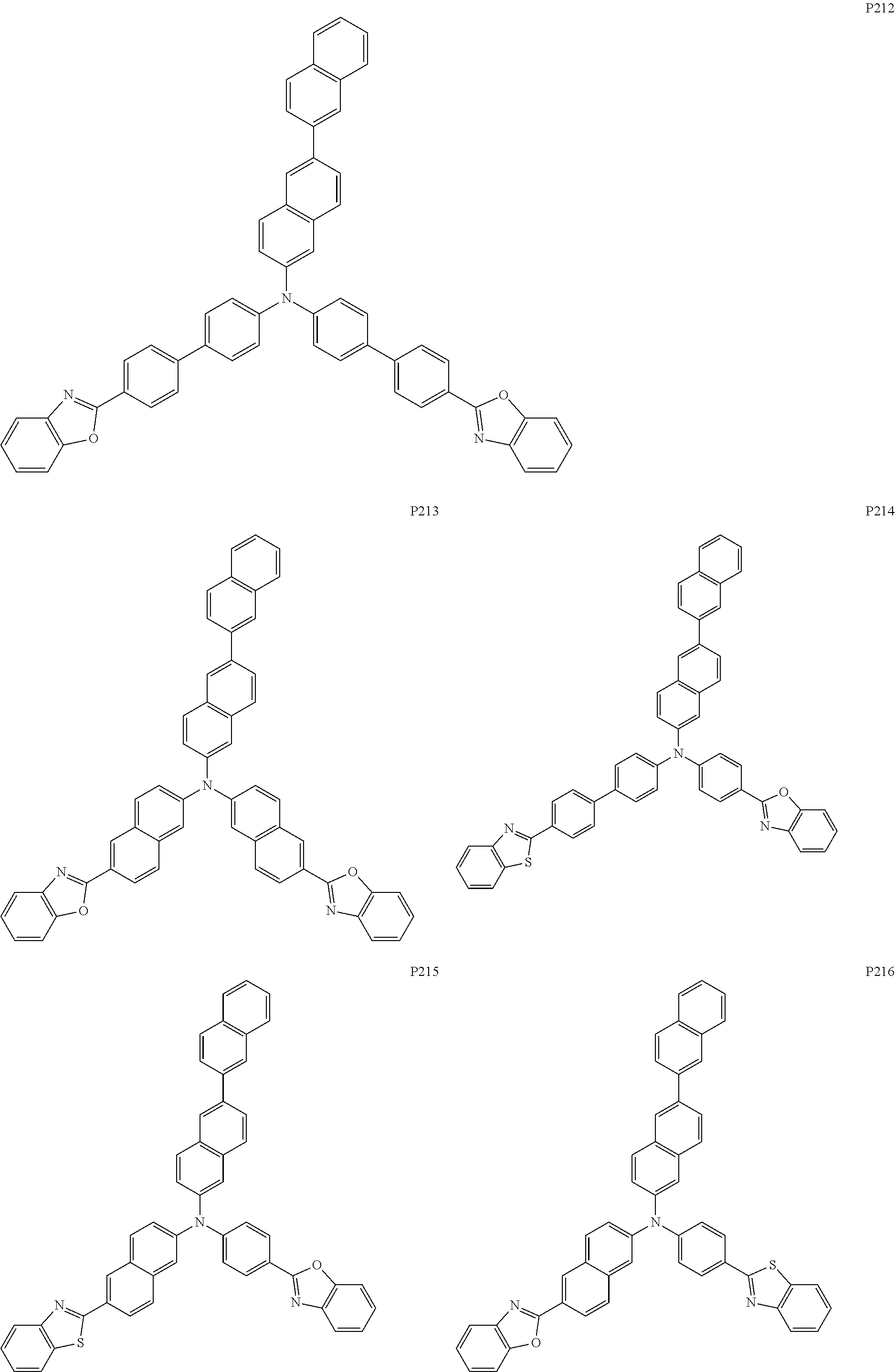 [SMILES: c1ccc2cc(-c3ccc4cc(N(c5ccc(-c6ccc(-c7nc8ccccc8o7)cc6)cc5)c5ccc(-c6ccc(-c7nc8ccccc8o7)cc6)cc5)ccc4c3)ccc2c1 ; c1ccc2cc(-c3ccc4cc(N(c5ccc6cc(-c7nc8ccccc8o7)ccc6c5)c5ccc6cc(-c7nc8ccccc8o7)ccc6c5)ccc4c3)ccc2c1 ; c1ccc2cc(-c3ccc4cc(N(c5ccc(-c6ccc(-c7nc8ccccc8s7)cc6)cc5)c5ccc(-c6nc7ccccc7o6)cc5)ccc4c3)ccc2c1 ; c1ccc2cc(-c3ccc4cc(N(c5ccc(-c6nc7ccccc7o6)cc5)c5ccc6cc(-c7nc8ccccc8s7)ccc6c5)ccc4c3)ccc2c1 ; c1ccc2cc(-c3ccc4cc(N(c5ccc(-c6nc7ccccc7s6)cc5)c5ccc6cc(-c7nc8ccccc8o7)ccc6c5)ccc4c3)ccc2c1]

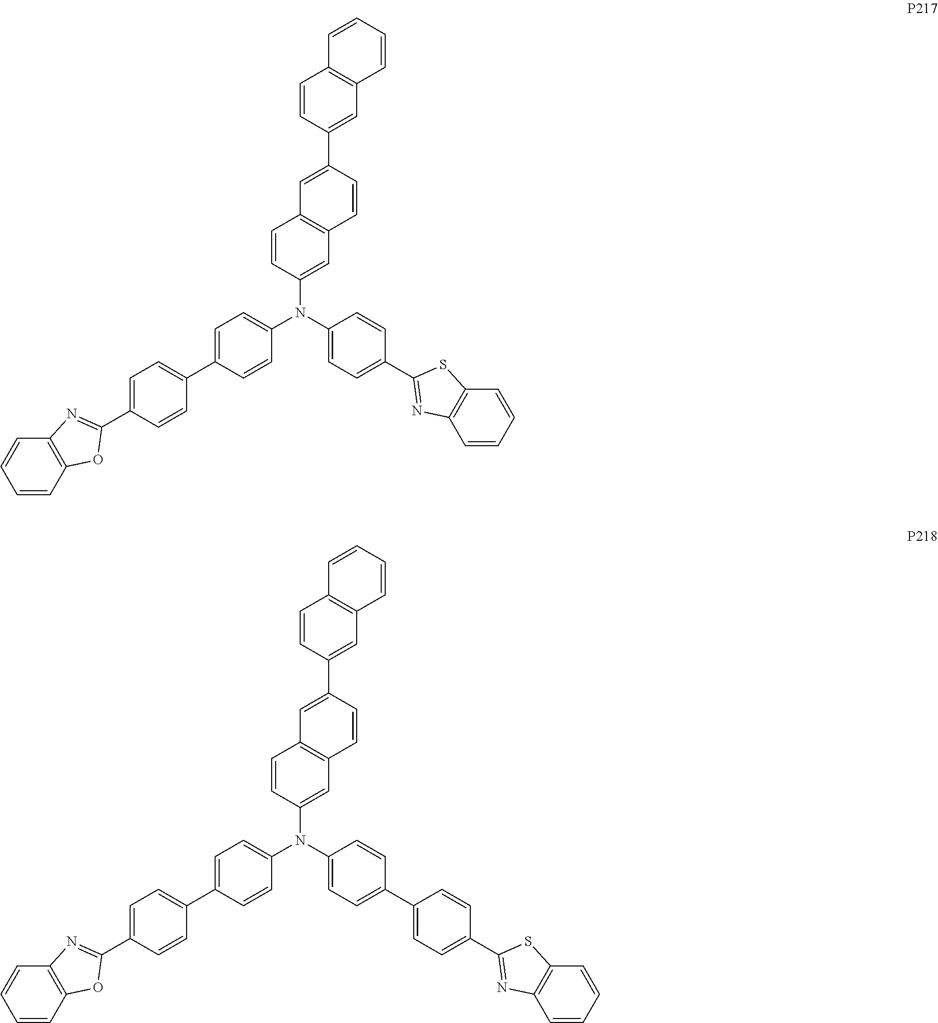 [SMILES: c1ccc2cc(-c3ccc4cc(N(c5ccc(-c6ccc(-c7nc8ccccc8o7)cc6)cc5)c5ccc(-c6nc7ccccc7s6)cc5)ccc4c3)ccc2c1 ; c1ccc2cc(-c3ccc4cc(N(c5ccc(-c6ccc(-c7nc8ccccc8o7)cc6)cc5)c5ccc(-c6ccc(-c7nc8ccccc8s7)cc6)cc5)ccc4c3)ccc2c1]

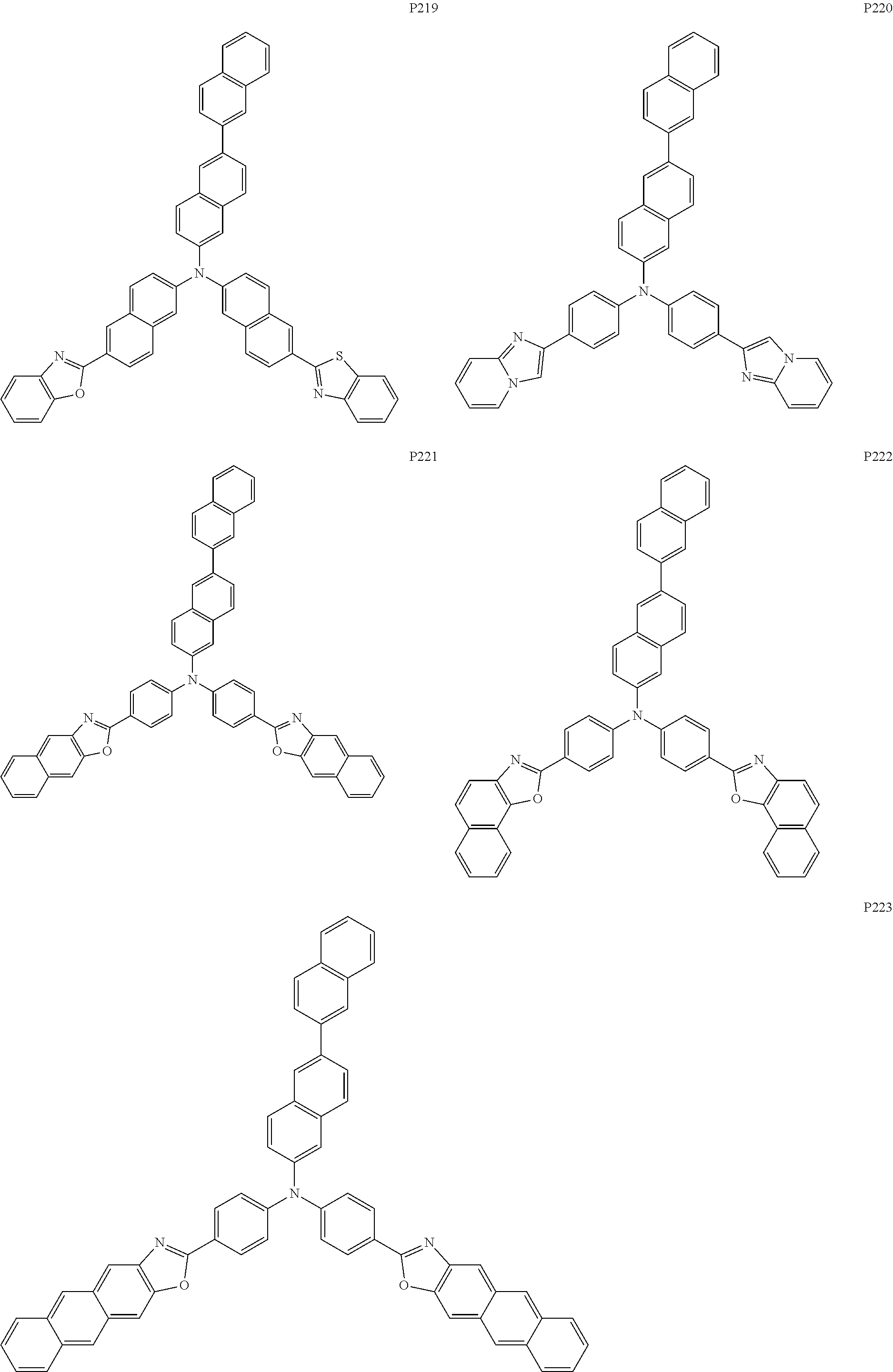 [SMILES: c1ccc2cc(-c3ccc4cc(N(c5ccc6cc(-c7nc8ccccc8o7)ccc6c5)c5ccc6cc(-c7nc8ccccc8s7)ccc6c5)ccc4c3)ccc2c1 ; c1ccc2cc(-c3ccc4cc(N(c5ccc(-c6cn7ccccc7n6)cc5)c5ccc(-c6cn7ccccc7n6)cc5)ccc4c3)ccc2c1 ; c1ccc2cc(-c3ccc4cc(N(c5ccc(-c6nc7cc8ccccc8cc7o6)cc5)c5ccc(-c6nc7cc8ccccc8cc7o6)cc5)ccc4c3)ccc2c1 ; c1ccc2cc(-c3ccc4cc(N(c5ccc(-c6nc7ccc8ccccc8c7o6)cc5)c5ccc(-c6nc7ccc8ccccc8c7o6)cc5)ccc4c3)ccc2c1 ; c1ccc2cc(-c3ccc4cc(N(c5ccc(-c6nc7cc8cc9ccccc9cc8cc7o6)cc5)c5ccc(-c6nc7cc8cc9ccccc9cc8cc7o6)cc5)ccc4c3)ccc2c1]

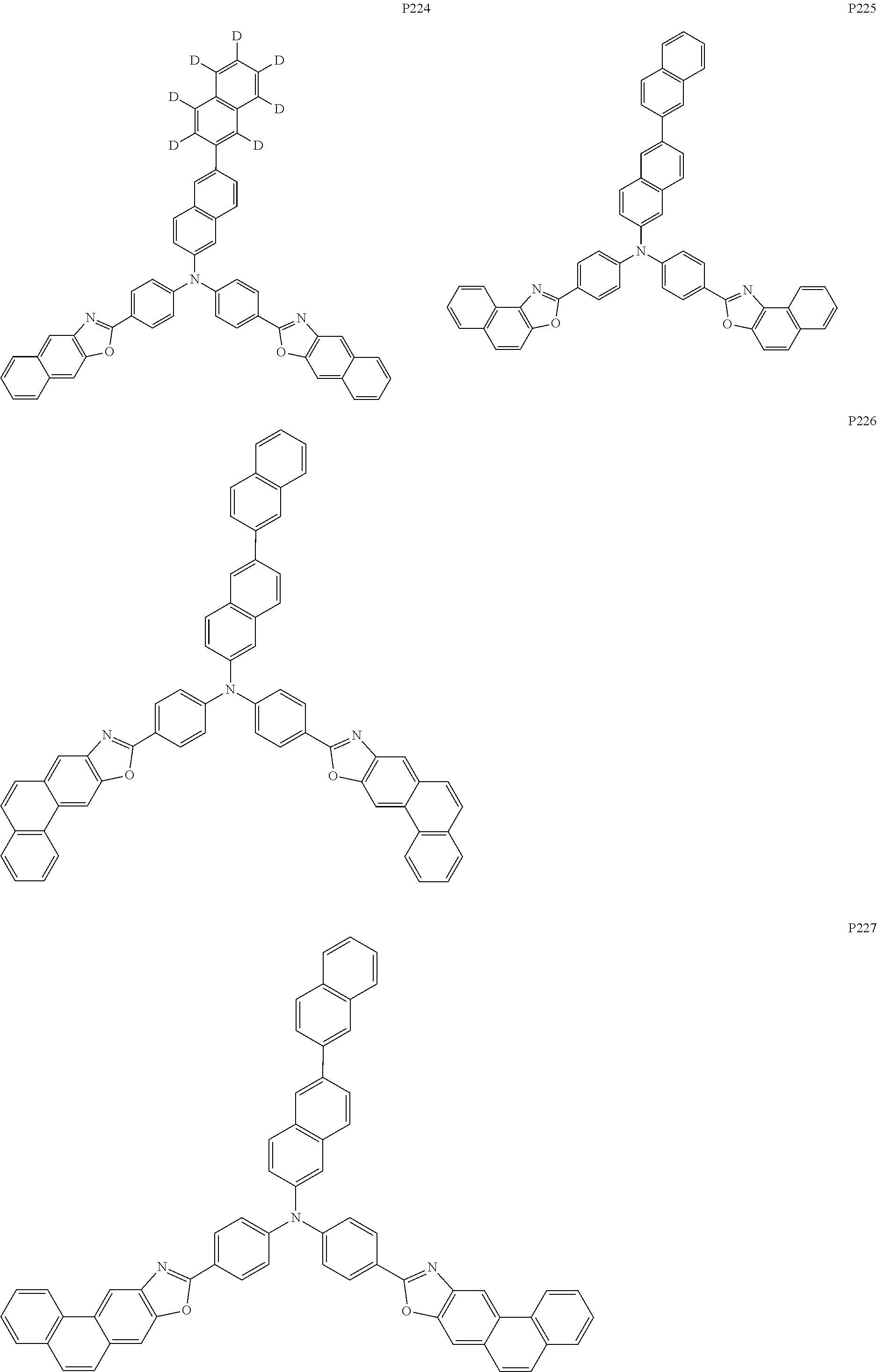 [SMILES: [2H]c1c([2H])c([2H])c2c([2H])c(-c3ccc4cc(N(c5ccc(-c6nc7cc8ccccc8cc7o6)cc5)c5ccc(-c6nc7cc8ccccc8cc7o6)cc5)ccc4c3)c([2H])c([2H])c2c1[2H] ; c1ccc2cc(-c3ccc4cc(N(c5ccc(-c6nc7c(ccc8ccccc87)o6)cc5)c5ccc(-c6nc7c(ccc8ccccc87)o6)cc5)ccc4c3)ccc2c1 ; c1ccc2cc(-c3ccc4cc(N(c5ccc(-c6nc7cc8ccc9ccccc9c8cc7o6)cc5)c5ccc(-c6nc7cc8ccc9ccccc9c8cc7o6)cc5)ccc4c3)ccc2c1 ; c1ccc2cc(-c3ccc4cc(N(c5ccc(-c6nc7cc8c(ccc9ccccc98)cc7o6)cc5)c5ccc(-c6nc7cc8c(ccc9ccccc98)cc7o6)cc5)ccc4c3)ccc2c1]

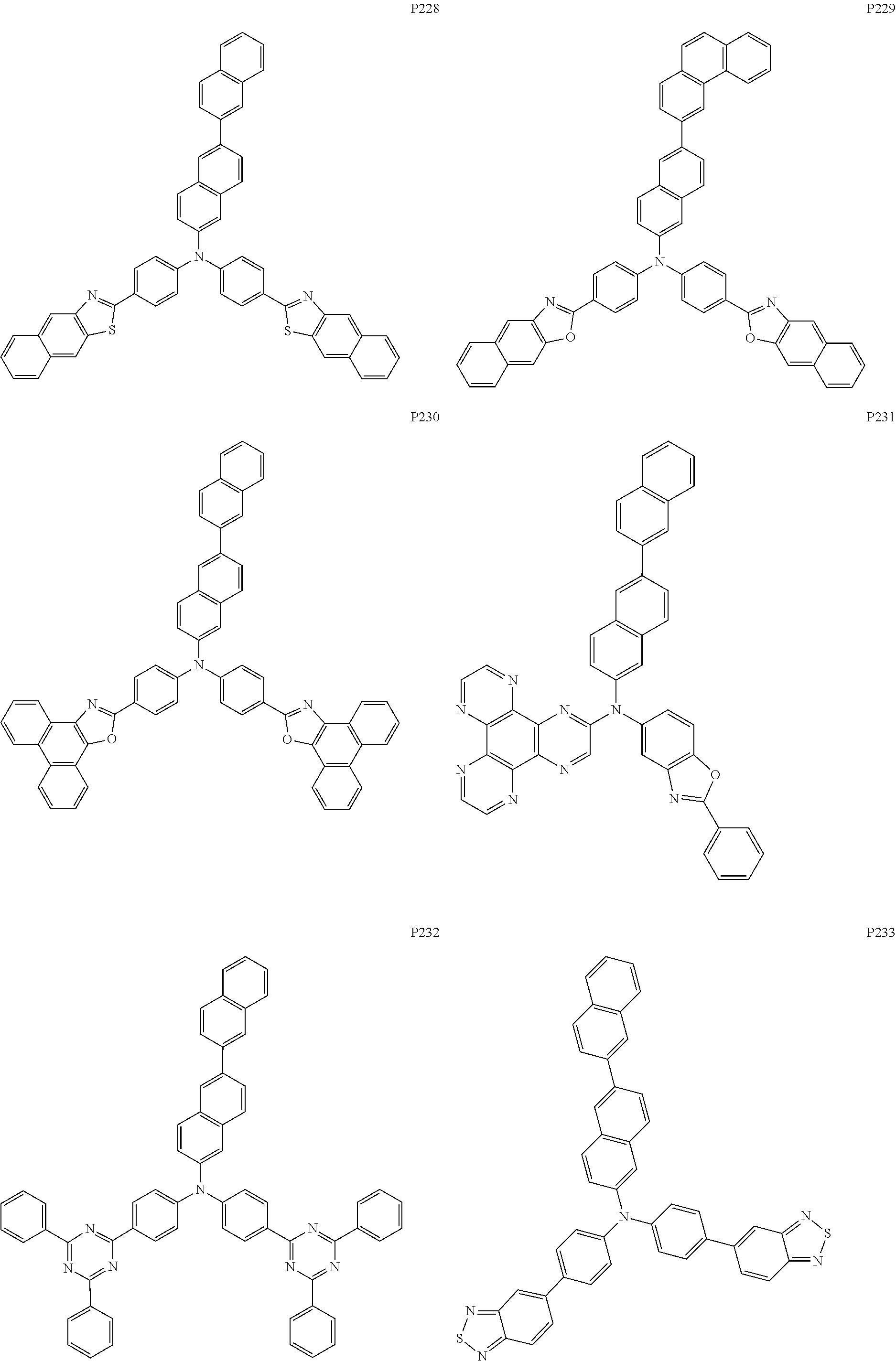

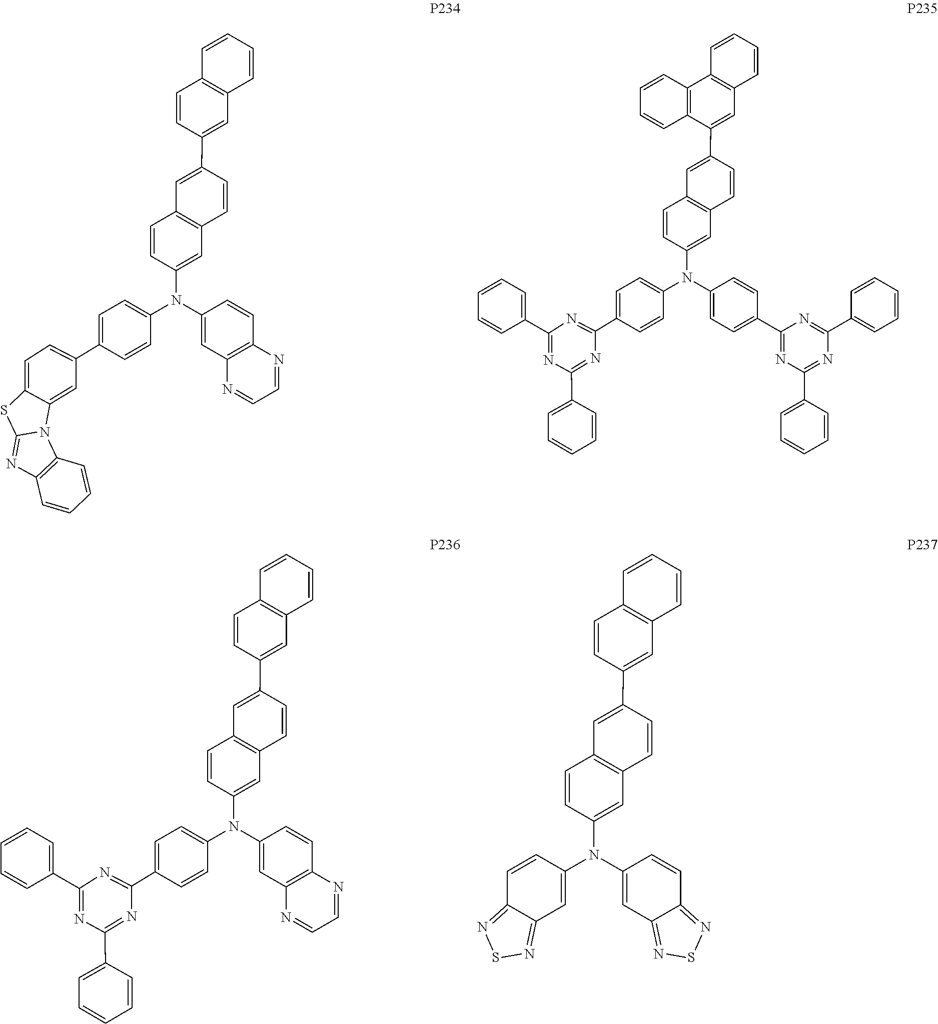 [SMILES: c1ccc2cc(-c3ccc4cc(N(c5ccc(-c6ccc7sc8nc9ccccc9n8c7c6)cc5)c5ccc6nccnc6c5)ccc4c3)ccc2c1 ; c1ccc(-c2nc(-c3ccccc3)nc(-c3ccc(N(c4ccc(-c5nc(-c6ccccc6)nc(-c6ccccc6)n5)cc4)c4ccc5cc(-c6cc7ccccc7c7ccccc67)ccc5c4)cc3)n2)cc1 ; c1ccc(-c2nc(-c3ccccc3)nc(-c3ccc(N(c4ccc5cc(-c6ccc7ccccc7c6)ccc5c4)c4ccc5nccnc5c4)cc3)n2)cc1 ; c1ccc2cc(-c3ccc4cc(N(c5ccc6nsnc6c5)c5ccc6nsnc6c5)ccc4c3)ccc2c1]

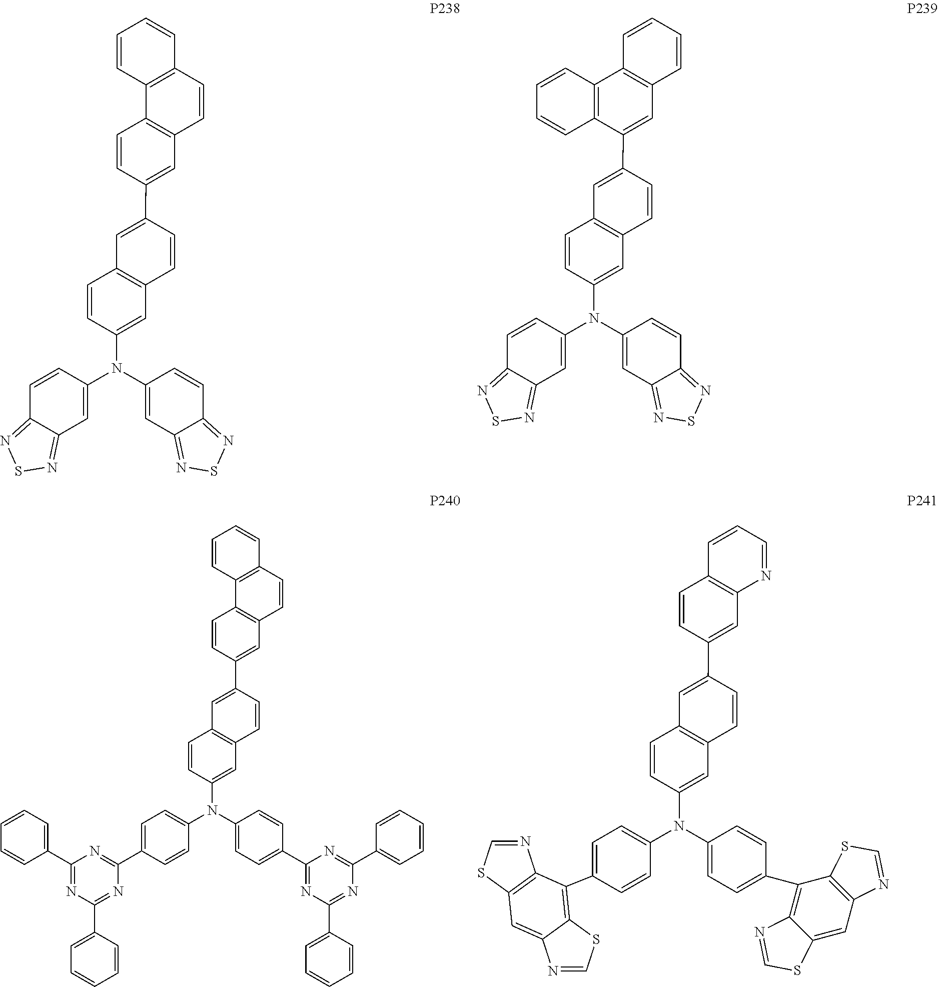 [SMILES: c1ccc2c(c1)ccc1cc(-c3ccc4cc(N(c5ccc6nsnc6c5)c5ccc6nsnc6c5)ccc4c3)ccc12 ; c1ccc2c(c1)cc(-c1ccc3cc(N(c4ccc5nsnc5c4)c4ccc5nsnc5c4)ccc3c1)c1ccccc12 ; c1ccc(-c2nc(-c3ccccc3)nc(-c3ccc(N(c4ccc(-c5nc(-c6ccccc6)nc(-c6ccccc6)n5)cc4)c4ccc5cc(-c6ccc7c(ccc8ccccc87)c6)ccc5c4)cc3)n2)cc1 ; c1cnc2cc(-c3ccc4cc(N(c5ccc(-c6c7ncsc7cc7ncsc67)cc5)c5ccc(-c6c7ncsc7cc7ncsc67)cc5)ccc4c3)ccc2c1]

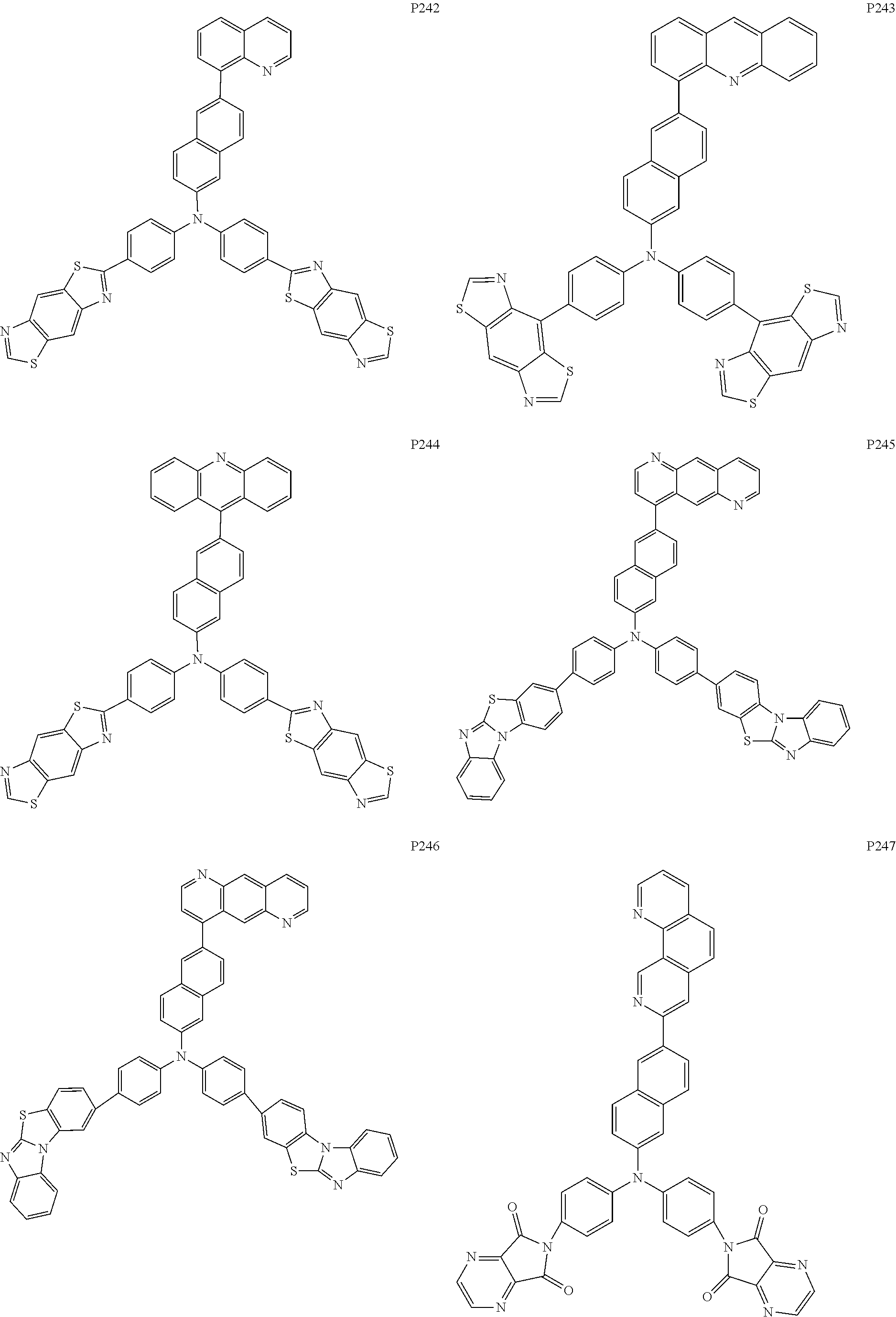

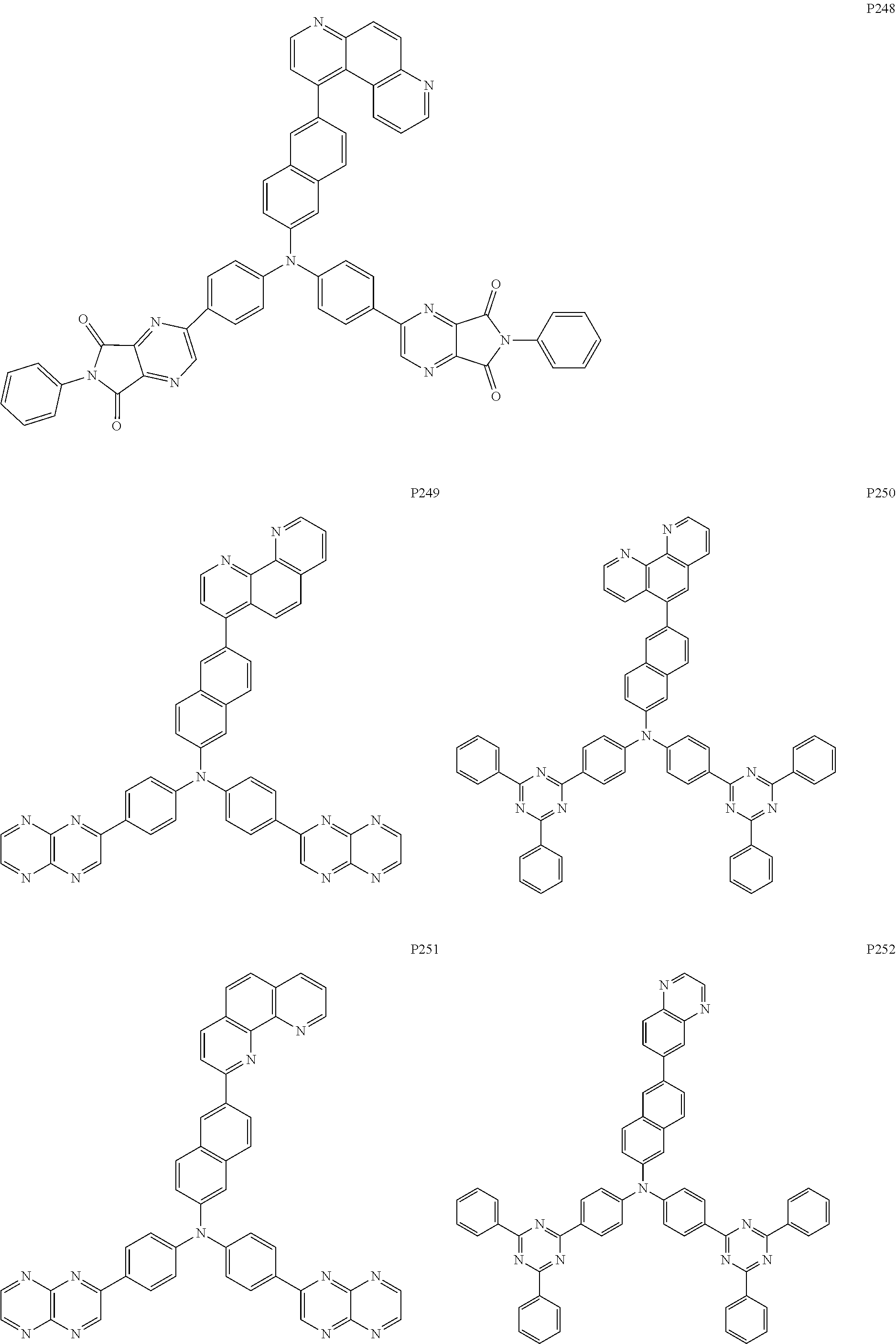

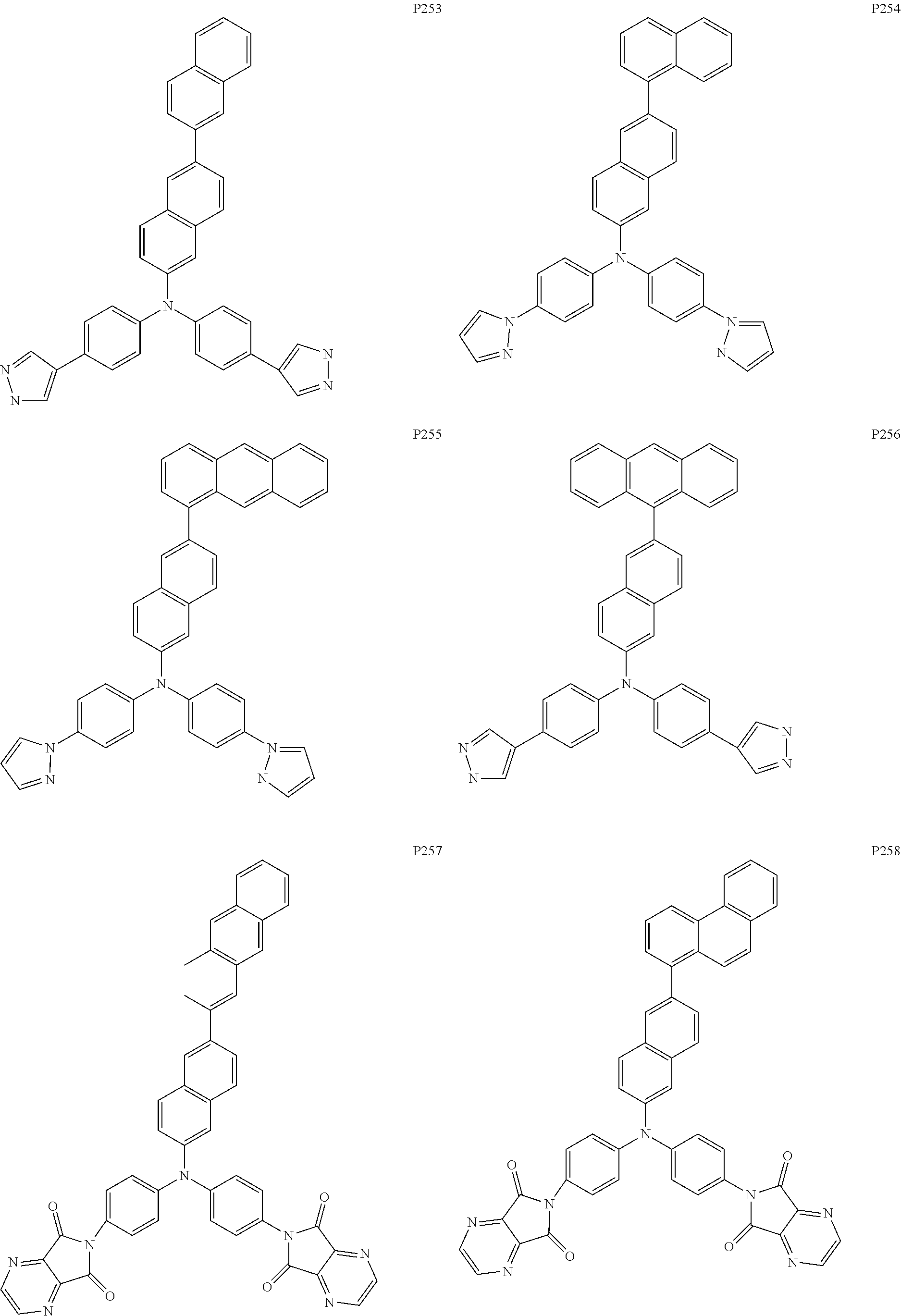

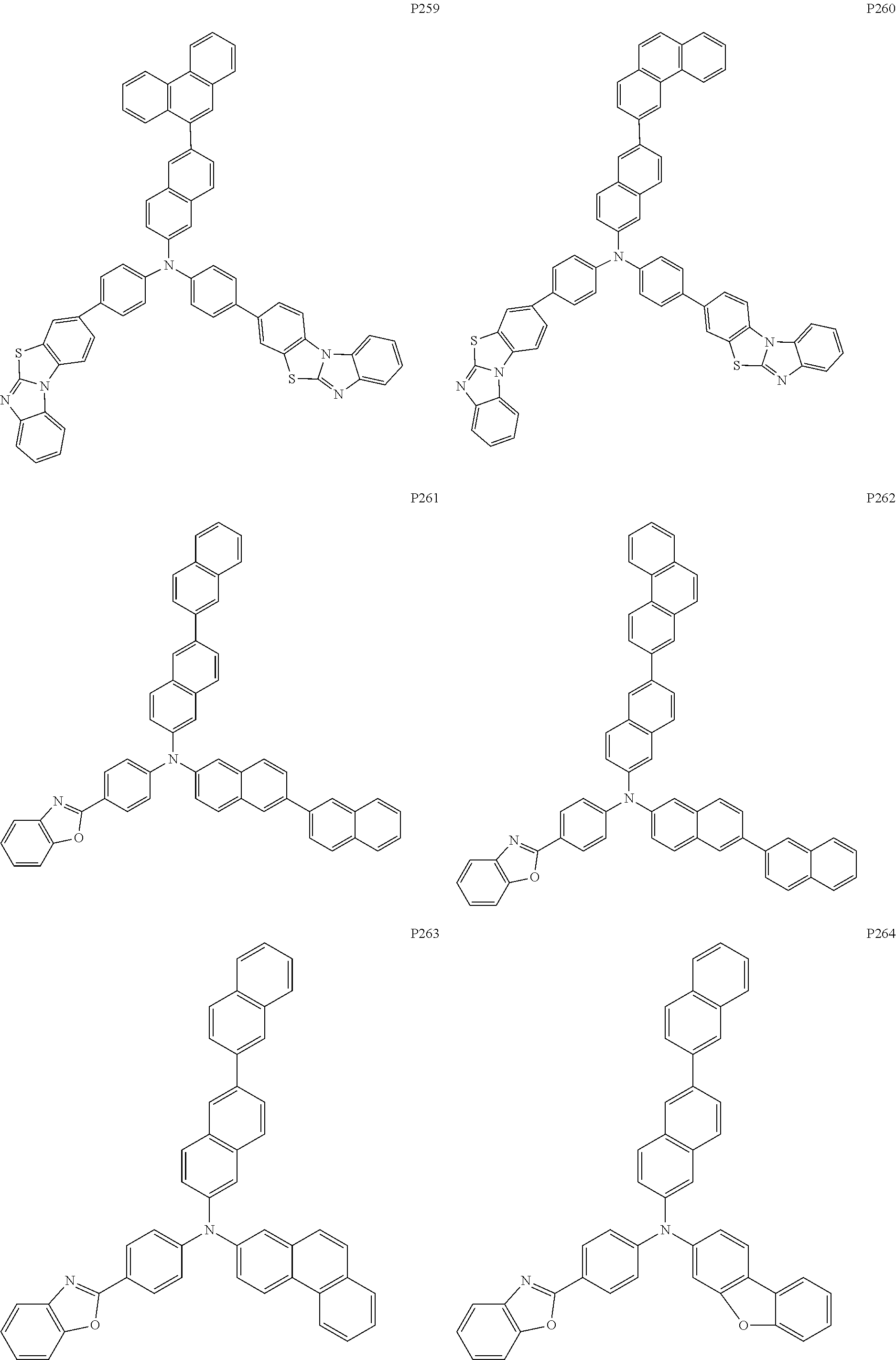

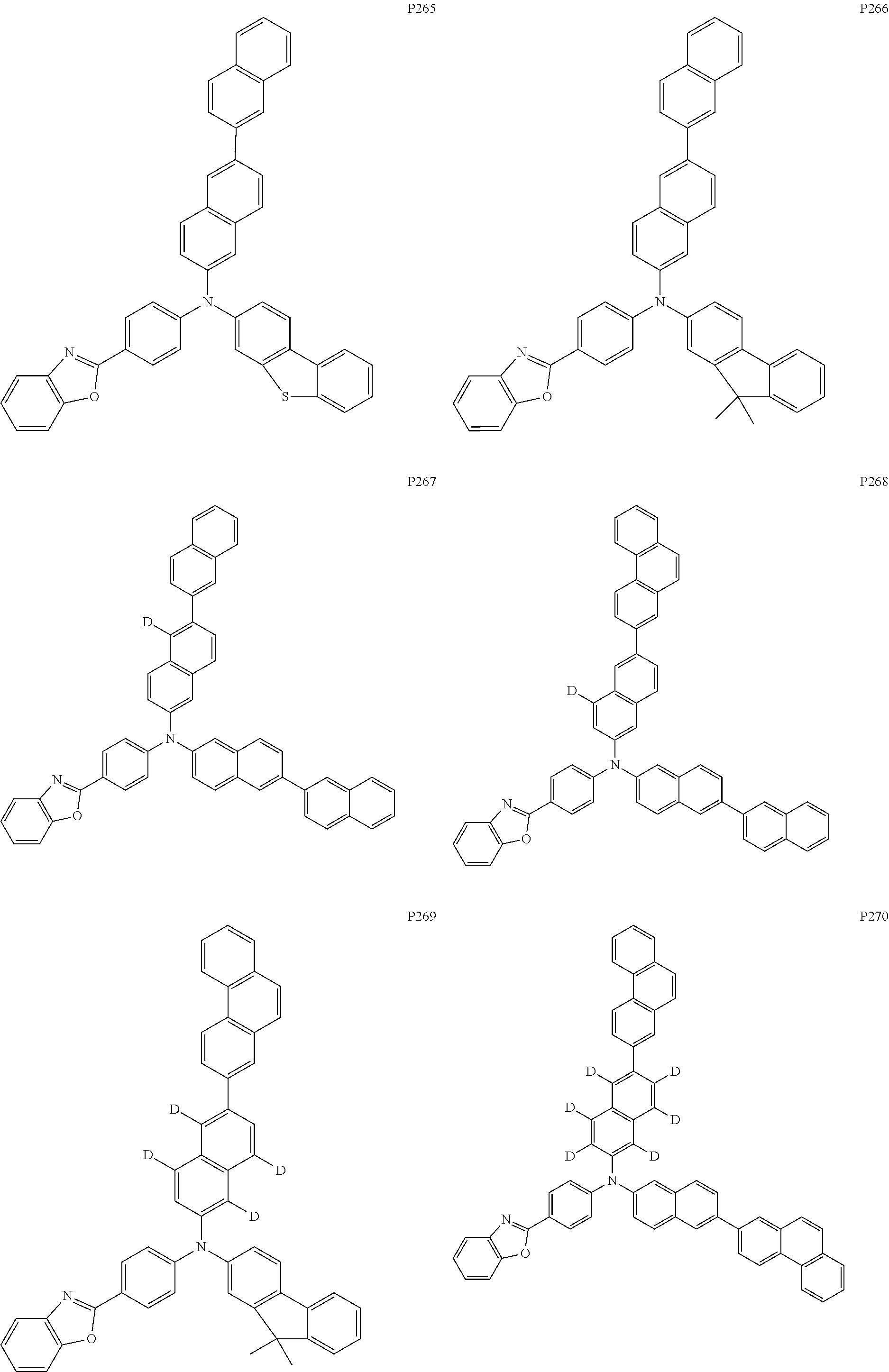

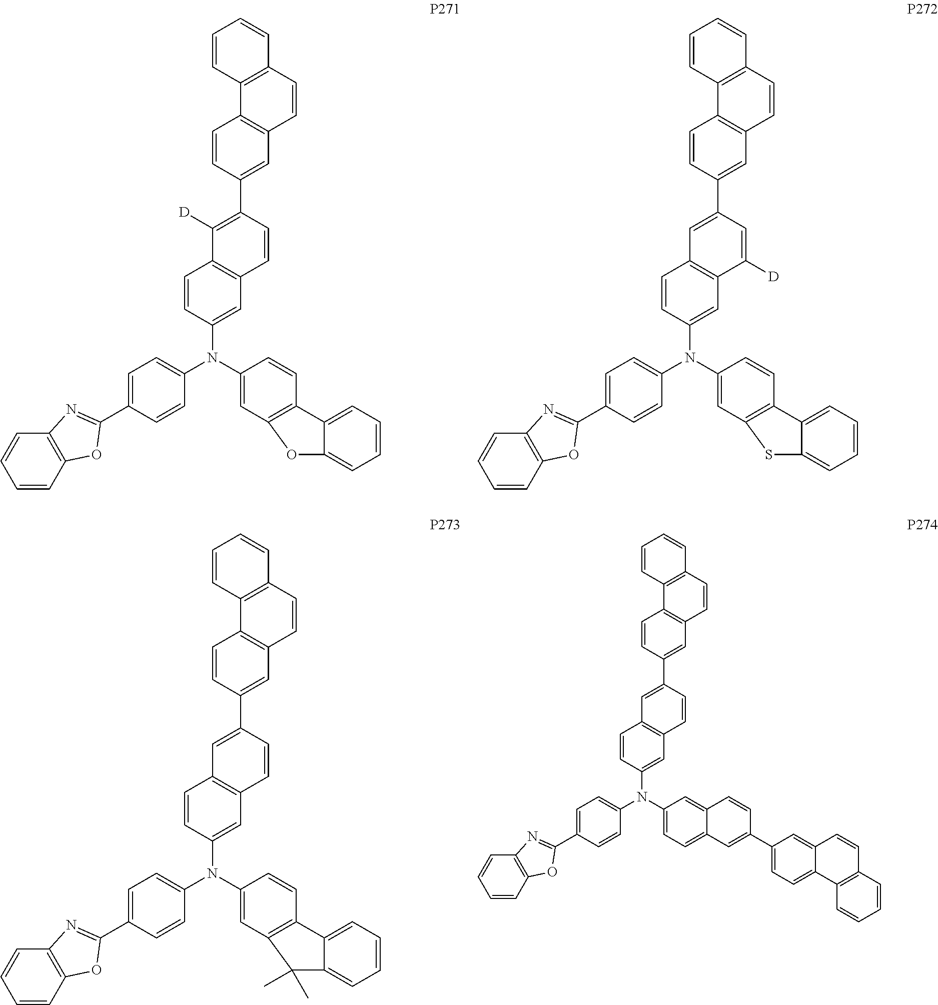 [SMILES: [2H]c1c(-c2ccc3c(ccc4ccccc43)c2)ccc2cc(N(c3ccc(-c4nc5ccccc5o4)cc3)c3ccc4c(c3)oc3ccccc34)ccc12 ; [2H]c1cc(-c2ccc3c(ccc4ccccc43)c2)cc2ccc(N(c3ccc(-c4nc5ccccc5o4)cc3)c3ccc4c(c3)sc3ccccc34)cc12 ; CC1(C)c2ccccc2-c2ccc(N(c3ccc(-c4nc5ccccc5o4)cc3)c3ccc4cc(-c5ccc6c(ccc7ccccc76)c5)ccc4c3)cc21 ; c1ccc2c(c1)ccc1cc(-c3ccc4cc(N(c5ccc(-c6nc7ccccc7o6)cc5)c5ccc6cc(-c7ccc8c(ccc9ccccc98)c7)ccc6c5)ccc4c3)ccc12]

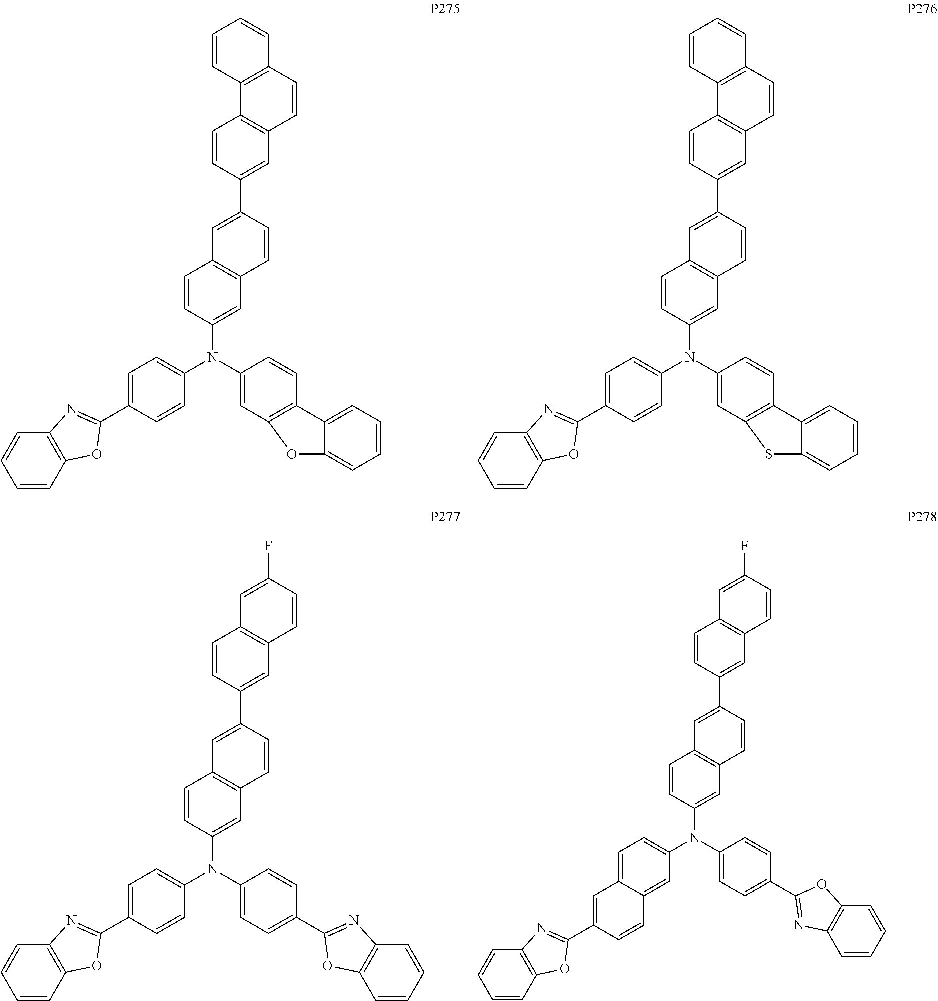 [SMILES: c1ccc2c(c1)ccc1cc(-c3ccc4cc(N(c5ccc(-c6nc7ccccc7o6)cc5)c5ccc6c(c5)oc5ccccc56)ccc4c3)ccc12 ; c1ccc2c(c1)ccc1cc(-c3ccc4cc(N(c5ccc(-c6nc7ccccc7o6)cc5)c5ccc6c(c5)sc5ccccc56)ccc4c3)ccc12 ; Fc1ccc2cc(-c3ccc4cc(N(c5ccc(-c6nc7ccccc7o6)cc5)c5ccc(-c6nc7ccccc7o6)cc5)ccc4c3)ccc2c1 ; Fc1ccc2cc(-c3ccc4cc(N(c5ccc(-c6nc7ccccc7o6)cc5)c5ccc6cc(-c7nc8ccccc8o7)ccc6c5)ccc4c3)ccc2c1]

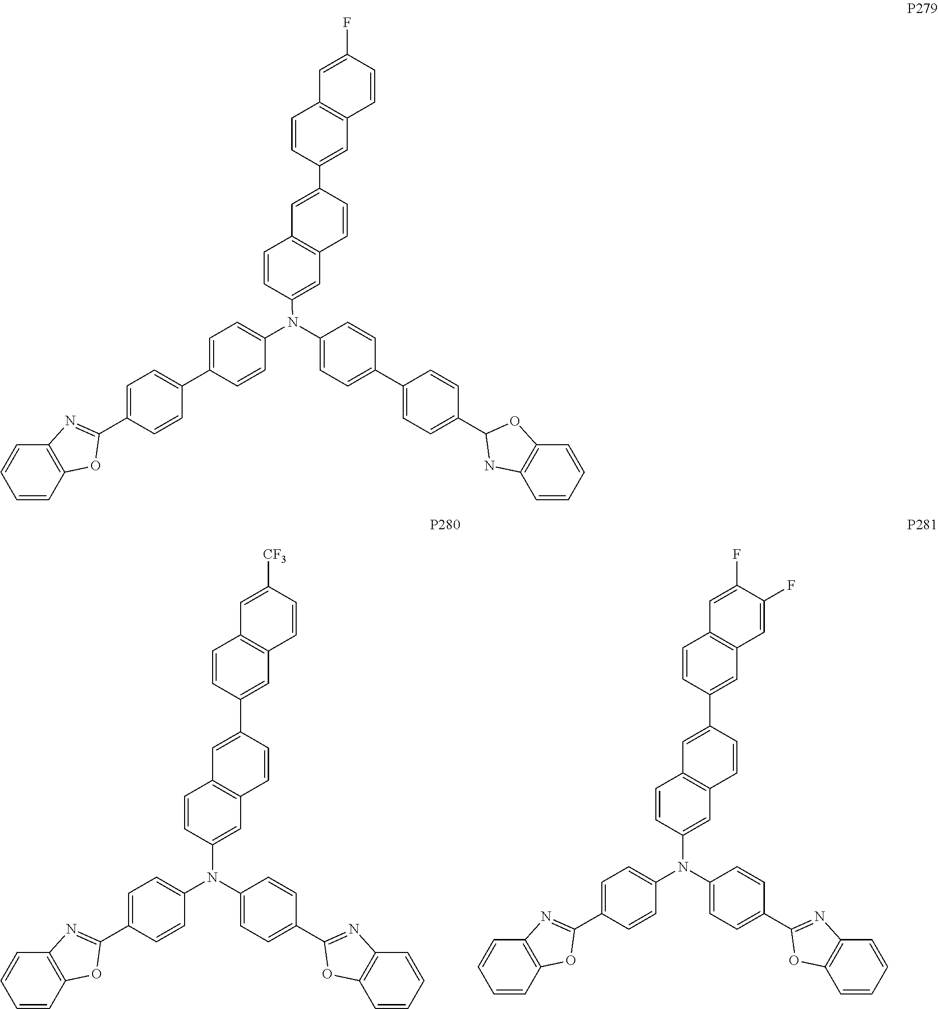 [SMILES: Fc1ccc2cc(-c3ccc4cc(N(c5ccc(-c6ccc(-c7nc8ccccc8o7)cc6)cc5)c5ccc(-c6ccc(C7Nc8ccccc8O7)cc6)cc5)ccc4c3)ccc2c1 ; FC(F)(F)c1ccc2cc(-c3ccc4cc(N(c5ccc(-c6nc7ccccc7o6)cc5)c5ccc(-c6nc7ccccc7o6)cc5)ccc4c3)ccc2c1 ; Fc1cc2ccc(-c3ccc4cc(N(c5ccc(-c6nc7ccccc7o6)cc5)c5ccc(-c6nc7ccccc7o6)cc5)ccc4c3)cc2cc1F]

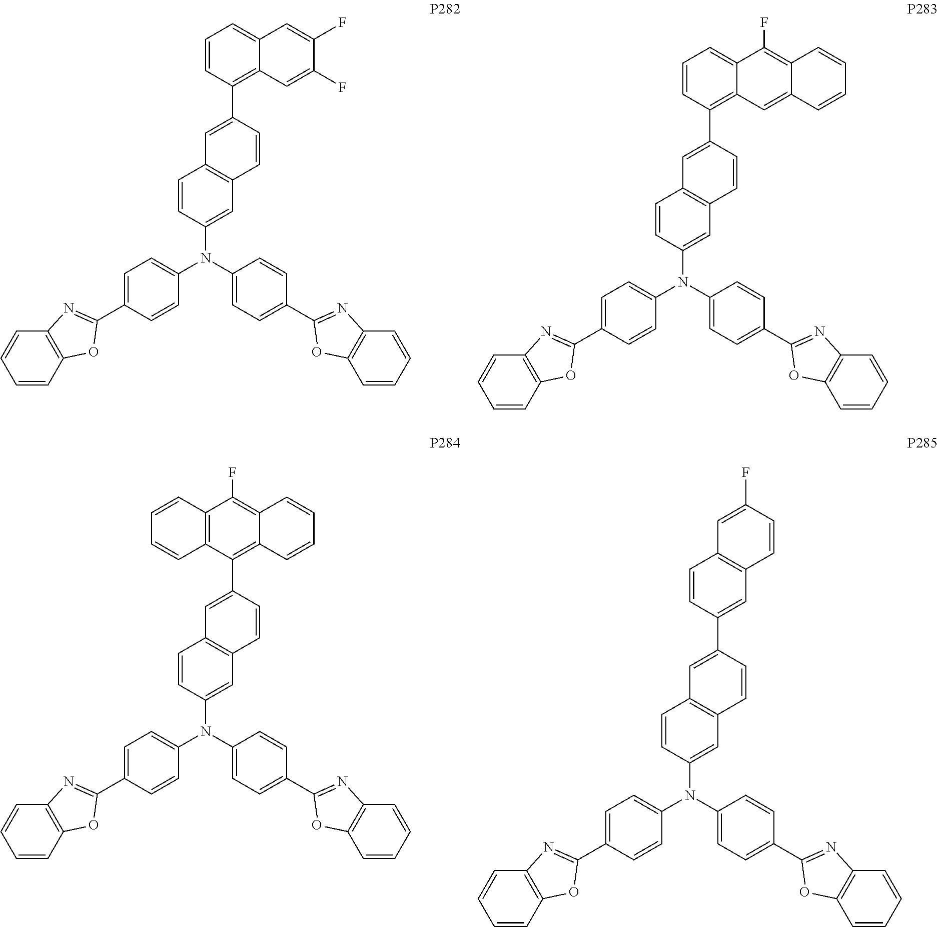 [SMILES: Fc1cc2cccc(-c3ccc4cc(N(c5ccc(-c6nc7ccccc7o6)cc5)c5ccc(-c6nc7ccccc7o6)cc5)ccc4c3)c2cc1F ; Fc1c2ccccc2cc2c(-c3ccc4cc(N(c5ccc(-c6nc7ccccc7o6)cc5)c5ccc(-c6nc7ccccc7o6)cc5)ccc4c3)cccc12 ; Fc1c2ccccc2c(-c2ccc3cc(N(c4ccc(-c5nc6ccccc6o5)cc4)c4ccc(-c5nc6ccccc6o5)cc4)ccc3c2)c2ccccc12 ; Fc1ccc2cc(-c3ccc4cc(N(c5ccc(-c6nc7ccccc7o6)cc5)c5ccc(-c6nc7ccccc7o6)cc5)ccc4c3)ccc2c1]

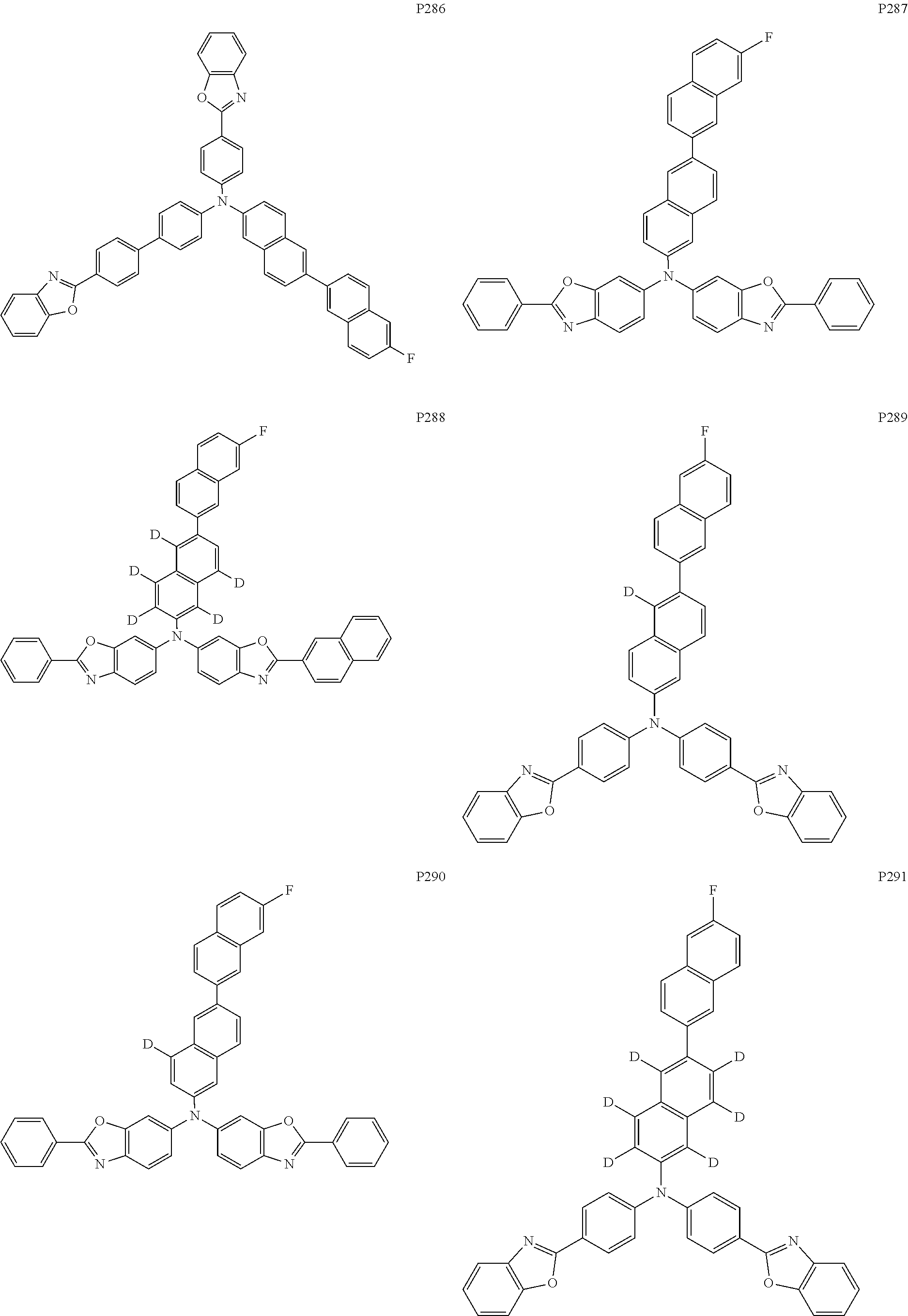

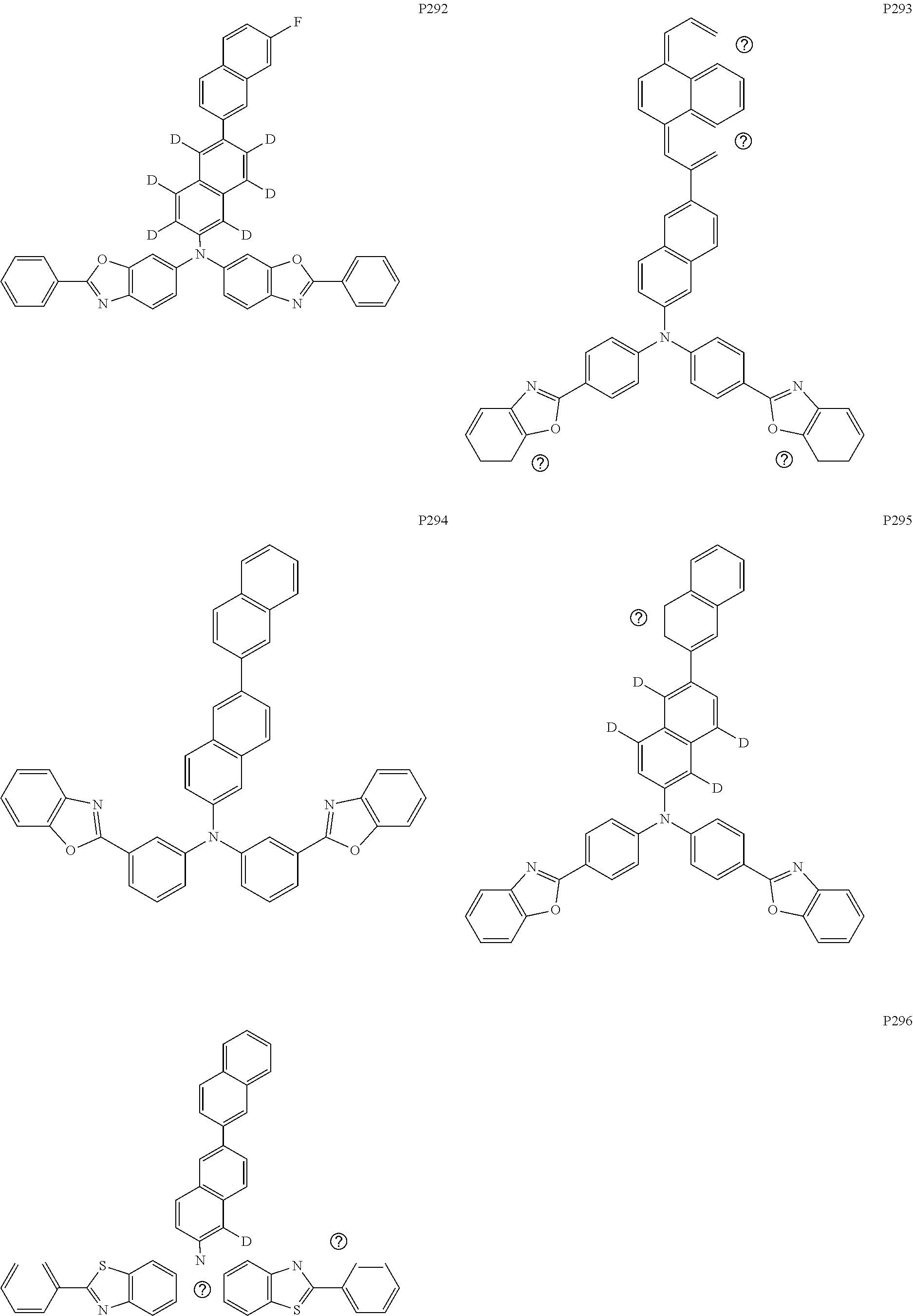

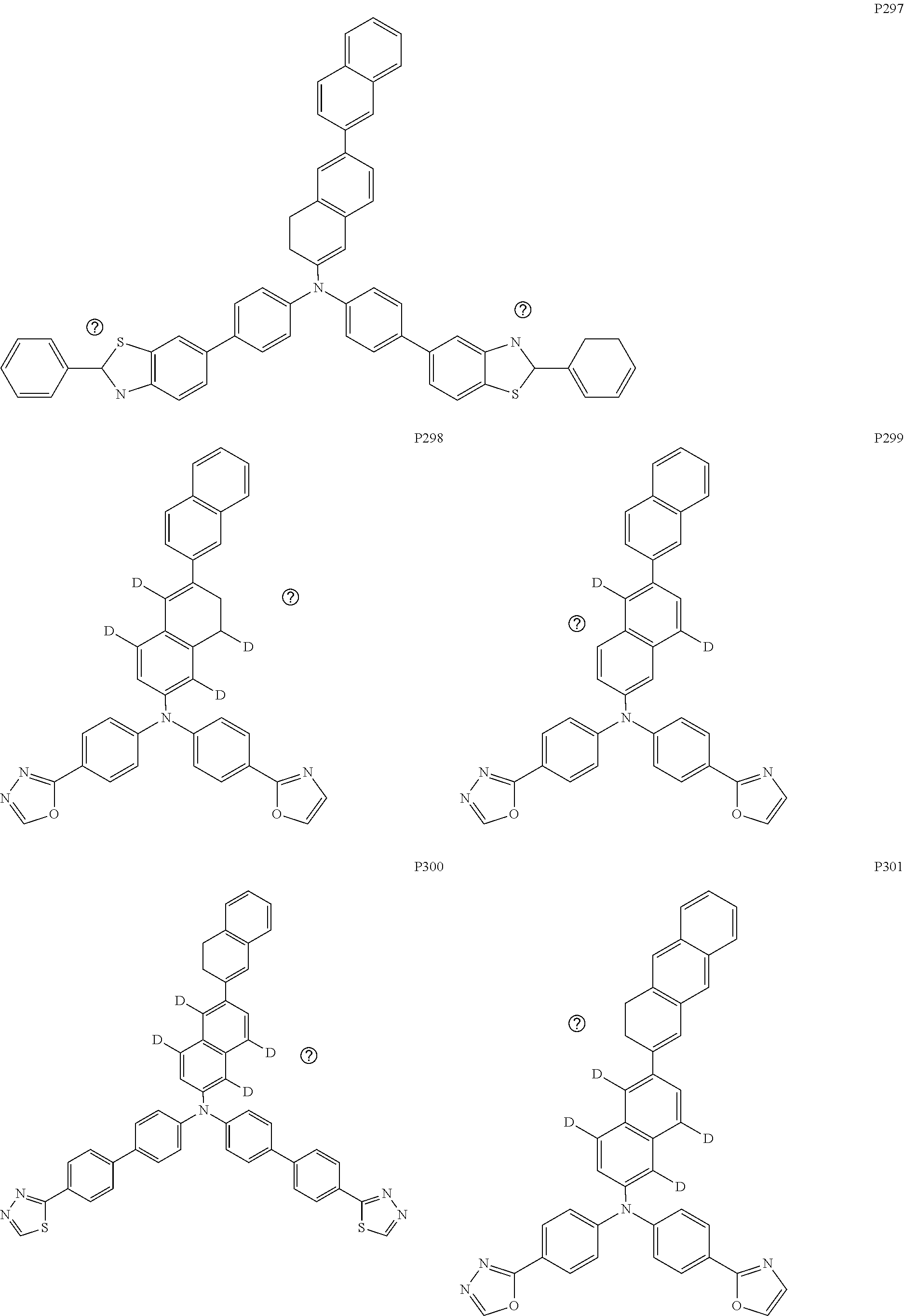

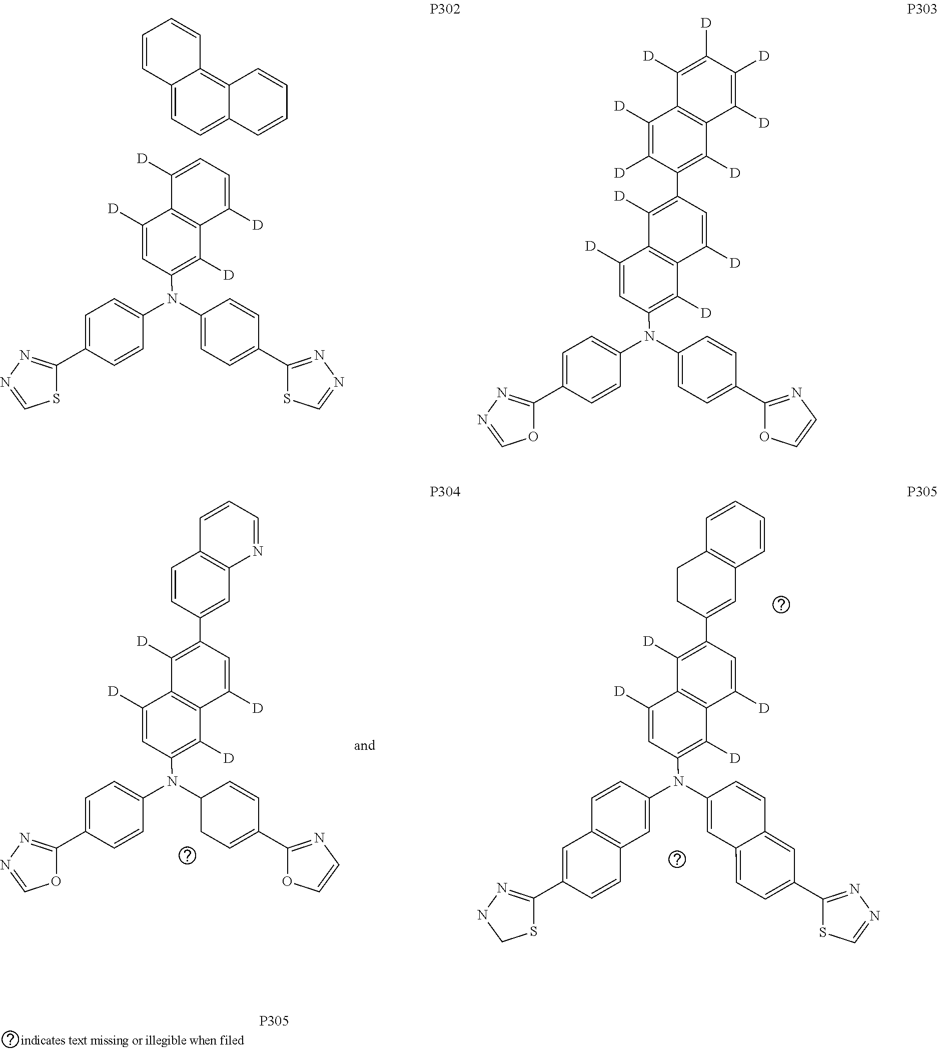

In accordance with one aspect of the present disclosure, a material for an organic electroluminescent device is provided. The material for an organic electroluminescent device includes any one or a combination of at least two of the compound as described herein.

In accordance with another aspect of the present disclosure, an organic electroluminescent device is provided. The organic electroluminescent device includes a first electrode layer, an organic function layer and a second electrode layer which are stacked in sequence.

The organic function layer includes the material as described above.

In accordance with another aspect of the present disclosure, another organic electroluminescent device is provided. The organic electroluminescent device includes a first capping layer, a first electrode layer, an organic function layer and a second electrode layer which are stacked in sequence.

The first capping layer includes the material as described above.

When the device is a top emitting device, the first electrode layer is a cathode layer and the second electrode layer is an anode layer; when the device is a bottom emitting device, the first electrode layer is an anode layer and the second electrode layer is a cathode layer.

The compound of the present disclosure can interact with a metal in a cathode (or anode) of the device, which reduces the coupling effect between free charges on the surface of the metal and electromagnetic radiation and improves photon extraction efficiency. Meanwhile, this modifies a metal electrode and reduces the possibility of film peeling.

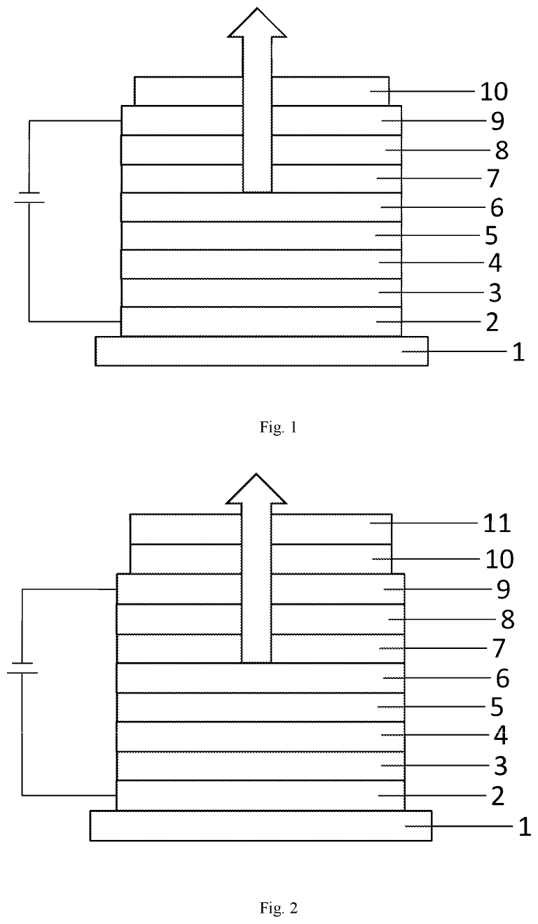

In an embodiment, an organic electroluminescent device provided by the present disclosure, as shown in FIG. 1, includes a substrate 1, an anode 2, a hole injection layer 3, a first hole transport layer 4, a second hole transport layer 5, a light-emitting layer 6, a first electron transport layer 7, a second electron transport layer 8, a cathode 9 and a first capping layer 10.

In an embodiment, the organic electroluminescent device further includes a second capping layer disposed on a side of the first capping layer facing away from the first electrode layer, where the second capping layer includes lithium fluoride and/or a material containing small organic molecules with a refractive index of 1.40-1.65 (for example, 1.42, 1.44, 1.46, 1.48, 1.5, 1.52, 1.54, 1.56, 1.58, 1.6, 1.62, 1.64 or the like).

The organic electroluminescent device provided by the present disclosure preferably includes two capping layers, and the compound provided by the present disclosure cooperates with lithium fluoride and/or a material containing small organic molecules with a refractive index of 1.40-1.65, which can alleviate the total reflection of light by a packaging glass, facilitate the transmission of visible light through the glass and improve the light extraction effect.

In an embodiment, the organic electroluminescent device provided by the present disclosure, as shown in FIG. 2, includes a substrate 1, an anode 2, a hole injection layer 3, a first hole transport layer 4, a second hole transport layer 5, a light-emitting layer 6, a first electron transport layer 7, a second electron transport layer 8, a cathode 9, a first capping layer 10 and a second capping layer 11.

In an embodiment, the material containing small organic molecules with a refractive index of 1.40-1.65 includes, but is not limited to, any one or a combination of at least two of polyfluorocarbons, boron-containing compounds, silicon-containing compounds, oxygen-containing silicon compounds and adamantane-containing alkane compounds.

In accordance with another aspect of the present disclosure, a display panel is provided. The display panel includes the organic electroluminescent device as described herein.

In an embodiment, the display panel is a foldable display panel.

When the compound provided by the present disclosure is used in the foldable display panel for display at multiple angles, light extraction Δn is small for RGB colors, which can effectively reduce a color cast.

In accordance with another aspect of the present disclosure, a display device is provided. The display device includes the display panel as described herein.

The method for preparing the compound provided by the present disclosure is known, and those skilled in the art can select a specific synthesis method according to conventional techniques. The present disclosure provides an exemplary synthesis route but is not so limited to such synthesis described below.

The representative synthesis route of the compound of Formula (1) provided by the present disclosure is as follows:

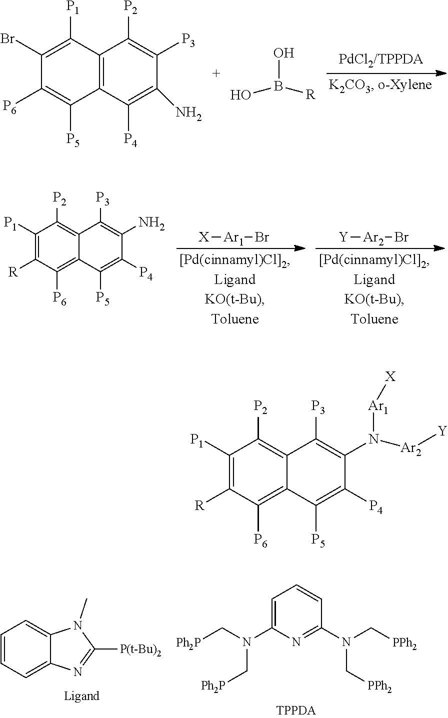

where o-Xylene represents ortho-xylene, Toluene represents toluene, KO(t-Bu) represents potassium tert-butoxide, and [Pd(cinnamyl)Cl]₂ represents palladium chloride (1-phenylallyl).

The following exemplary synthesis provides specific synthesis methods for a series of compounds. For compounds whose specific synthesis methods are not mentioned, these compounds may be synthesized by similar methods or other existing methods, which are not specifically limited in the present disclosure.

### Example 1

The synthesis route of Compound P91 is as follows:

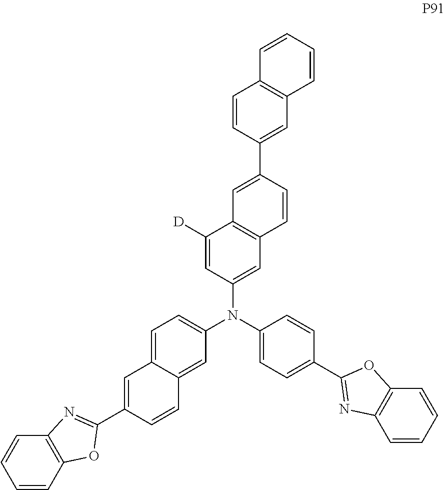 [SMILES: [2H]c1cc(N(c2ccc(-c3nc4ccccc4o3)cc2)c2ccc3cc(-c4nc5ccccc5o4)ccc3c2)cc2ccc(-c3ccc4ccccc4c3)cc12]

A specific preparation method includes steps described below.

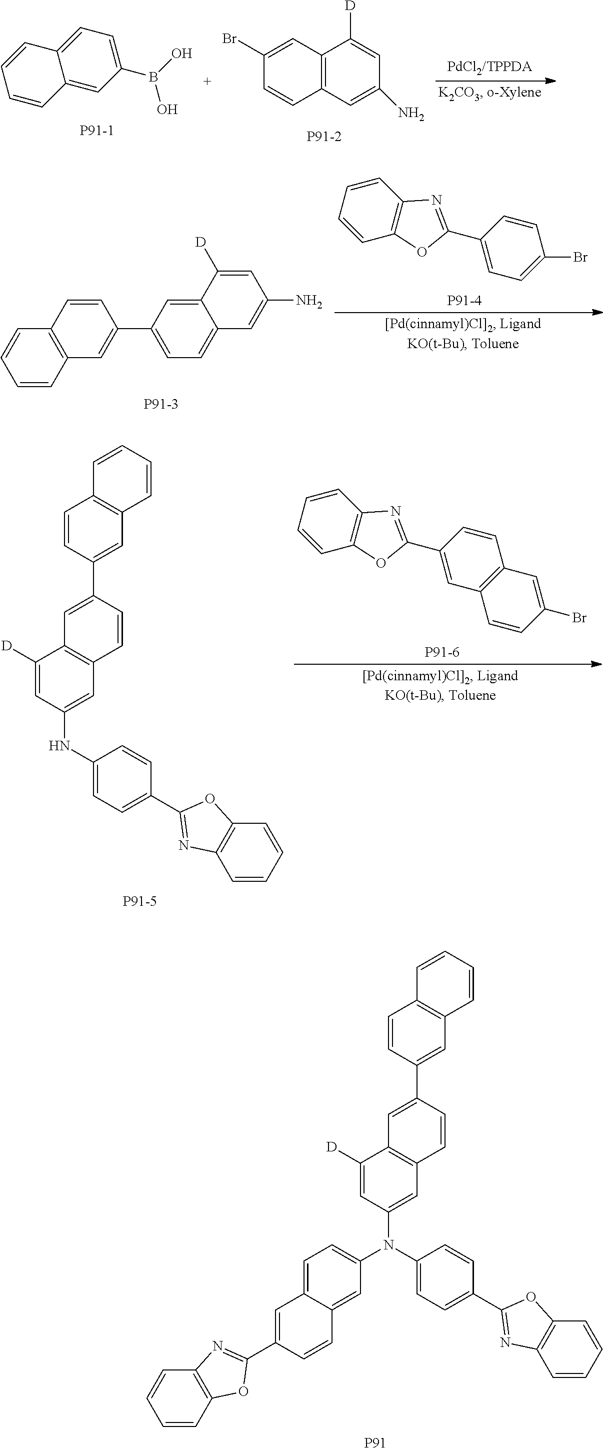

(1) P91-1 (0.5 mmol), P91-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 15 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P91-3 was obtained through column chromatography.

The structure of the target product P91-3 was tested through matrix-assisted laser desorption/ionization time-of-flight mass spectrometry (MALDI-TOF-MS) (m/z) to obtain C₂₀H₁₄DN whose calculated value was 270.1 and measured value was 270.0.

(2) P91-3 (0.5 mmol), P91-4 (0.45 mmol), KO(t-Bu) (0.5 mmol), [Pd(cinnamyl)Cl]₂ (1 mol %) and a ligand (1.0 mol %) were added to 3 mL of toluene and mixed into a solution, and the solution was put in a 50 mL flask and reacted at 60° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P91-5 was obtained through column chromatography.

The structure of the target product P91-5 was tested through matrix-assisted laser desorption/ionization time-of-flight mass spectrometry (MALDI-TOF-MS) (m/z) to obtain C₃₃H₂₁DN₂O whose calculated value was 463.2 and measured value was 463.0.

(3) P91-5 (0.5 mmol), P91-6 (0.65 mmol), KO(t-Bu) (0.65 mmol), [Pd(cinnamyl)Cl]₂ (1.5 mol %) and a ligand (1.5 mol %) were added to 3 mL of toluene and mixed into a solution, and the solution was put in a 50 mL flask and reacted at 80° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P91 was obtained through column chromatography.

The structure of the target product P91 was tested through matrix-assisted laser desorption/ionization time-of-flight mass spectrometry (MALDI-TOF-MS) (m/z) to obtain C₅₀H₃₀DN₃O₂ whose calculated value was 706.3 and measured value was 706.1.

Elemental analysis: theoretical value: C, 84.96, H, 4.56; N, 5.95; measured value: C, 84.95; H, 4.56; N, 5.96.

### Example 2

The synthesis route of Compound P92 is as follows:

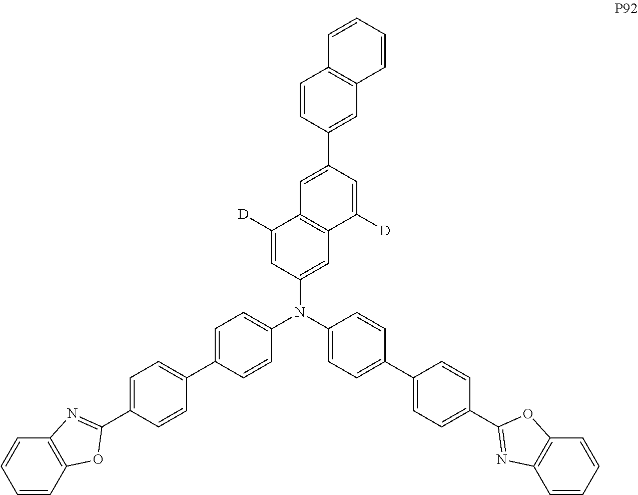 [SMILES: [2H]c1cc(N(c2ccc(-c3ccc(-c4nc5ccccc5o4)cc3)cc2)c2ccc(-c3ccc(-c4nc5ccccc5o4)cc3)cc2)cc2c([2H])cc(-c3ccc4ccccc4c3)cc12]

A specific preparation method includes steps described below.

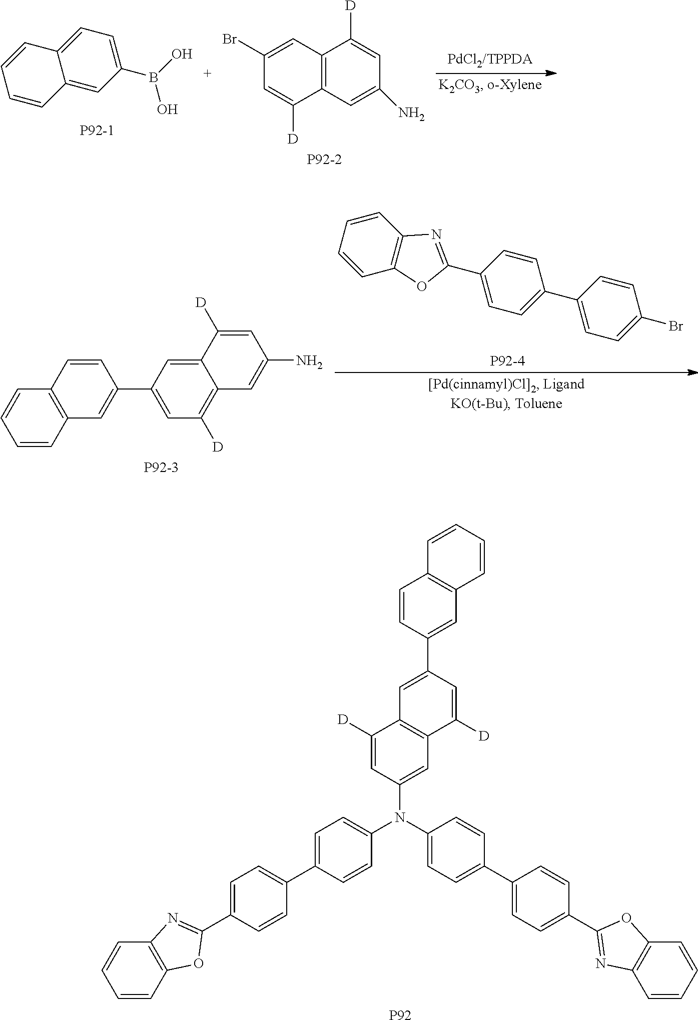

(1) P92-1 (0.5 mmol), P92-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 24 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P92-3 was obtained through column chromatography.

The structure of the target product P92-3 was tested through matrix-assisted laser desorption/ionization time-of-flight mass spectrometry (MALDI-TOF-MS) (m/z) to obtain C₂₀H₁₃D₂N whose calculated value was 271.1 and measured value was 271.0.

(2) P92-3 (0.5 mmol), P92-4 (1.5 mmol), KO(t-Bu) (0.75 mmol), [Pd(cinnamyl)Cl]₂ (2 mol %) and a ligand (1.5 mol %) were added to 3 mL of toluene and mixed into a solution, and the solution was put in a 50 mL flask and reacted at 80° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P92 was obtained through column chromatography.

The structure of the target product P92 was tested through matrix-assisted laser desorption/ionization time-of-flight mass spectrometry (MALDI-TOF-MS) (m/z) to obtain C₅₈H₃₅D₂N₃O₂ whose calculated value was 809.3 and measured value was 809.1.

Elemental analysis: theoretical value: C, 86.01, H, 4.85; N, 5.19; measured value: C, 86.00; H, 4.85; N, 5.19.

### Example 3

The synthesis route of Compound P100 is as follows:

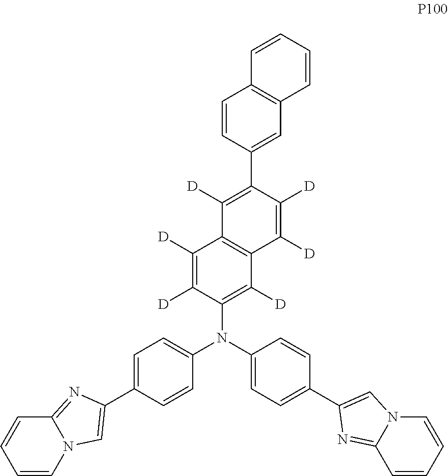 [SMILES: [2H]c1c(N(c2ccc(-c3cn4ccccc4n3)cc2)c2ccc(-c3cn4ccccc4n3)cc2)c([2H])c2c([2H])c([2H])c(-c3ccc4ccccc4c3)c([2H])c2c1[2H]]

A specific preparation method specifically includes steps described below.

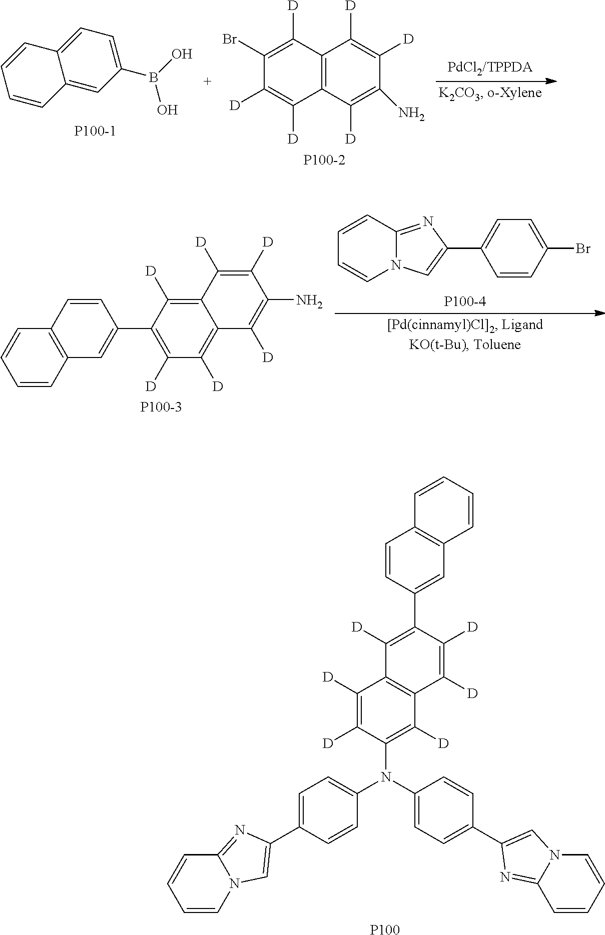

(1) P100-1 (0.5 mmol), P100-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 24 h. Then then solution was cooled to room temperature and slowly added with a saturated aqueous solution of MgSO₄ and ethyl acetate to be extracted for three times. Then, the organic layer is passed through a rotary evaporator for the solvent to be removed and the crude product P100-3 was obtained through column chromatography.

The structure of the target product P100-3 was tested through matrix-assisted laser desorption/ionization time-of-flight mass spectrometry (MALDI-TOF-MS) (m/z) to obtain C₂₀H₉D₆N whose calculated value was 275.2 and measured value was 275.1.

(2) P100-3 (0.5 mmol), P100-4 (1.5 mmol), KO(t-Bu) (0.75 mmol), [Pd(cinnamyl)Cl]₂ (2 mol %) and a ligand (1.5 mol %) were added to 3 mL of toluene and mixed into a solution, and the solution was put in a 50 mL flask and reacted at 80° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P100 was obtained through column chromatography.

The structure of the target product P100 was tested through matrix-assisted laser desorption/ionization time-of-flight mass spectrometry (MALDI-TOF-MS) (m/z) to obtain C₄₆H₂₅D₆N₅ whose calculated value was 659.3 and measured value was 659.2.

Elemental analysis: theoretical value: C, 83.74, H, 5.65; N, 10.61; measured value: C, 83.74; H, 5.65; N, 10.61.

### Example 4

The synthesis route of Compound P101 is as follows:

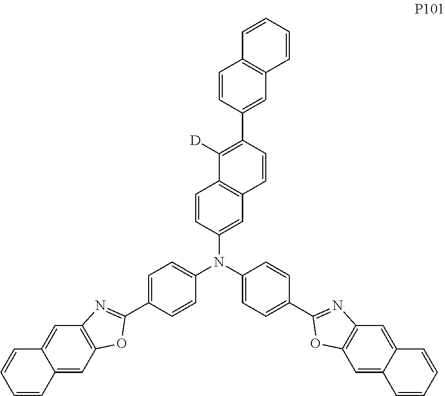 [SMILES: [2H]c1c(-c2ccc3ccccc3c2)ccc2cc(N(c3ccc(-c4nc5cc6ccccc6cc5o4)cc3)c3ccc(-c4nc5cc6ccccc6cc5o4)cc3)ccc12]

A specific preparation method includes steps described below.

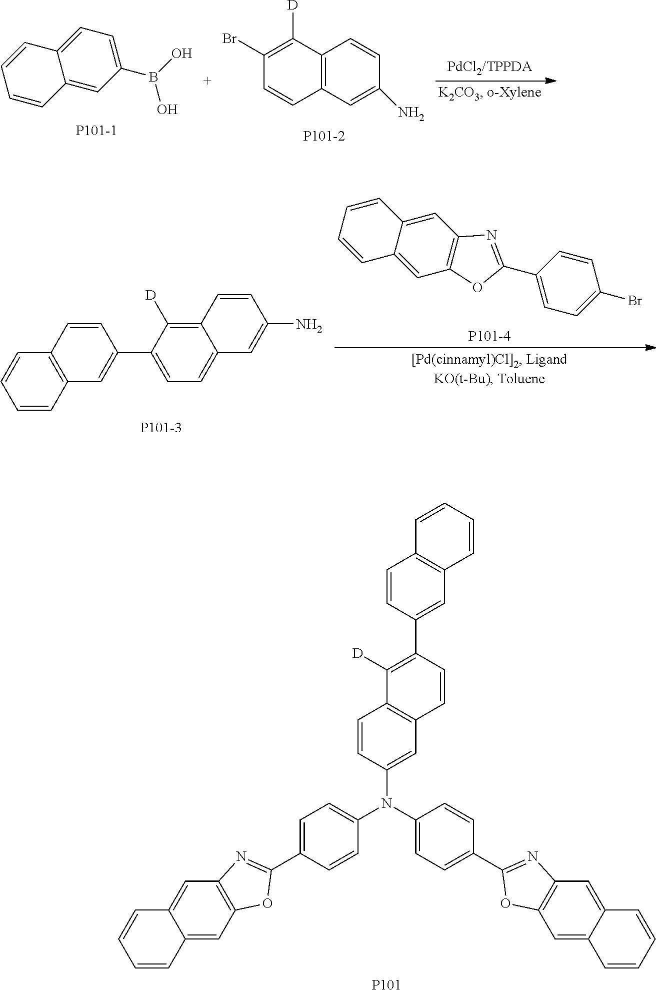

(1) P101-1 (0.5 mmol), P101-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 24 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P101-3 was obtained through column chromatography.

The structure of the target product P101-3 was tested through matrix-assisted laser desorption/ionization time-of-flight mass spectrometry (MALDI-TOF-MS) (m/z) to obtain C₂₀H₁₄DN whose calculated value was 270.1 and measured value was 270.1.

(2) P101-3 (0.5 mmol), P101-4 (1.5 mmol), KO(t-Bu) (0.75 mmol), [Pd(cinnamyl)Cl]₂ (2 mol %) and a ligand (1.5 mol %) were added to 3 mL of toluene and mixed into a solution, and the solution was put in a 50 mL flask and reacted at 80° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P101 was obtained through column chromatography.

The structure of the target product P101 was tested through matrix-assisted laser desorption/ionization time-of-flight mass spectrometry (MALDI-TOF-MS) (m/z) to obtain C₅₄H₃₂DN₃O₂ whose calculated value was 756.3 and measured value was 756.1.

Elemental analysis: theoretical value: C, 85.69, H, 4.53; N, 5.55; measured value: C, 85.68; H, 4.53; N, 5.56.

### Example 5

The synthesis route of Compound P197 is as follows:

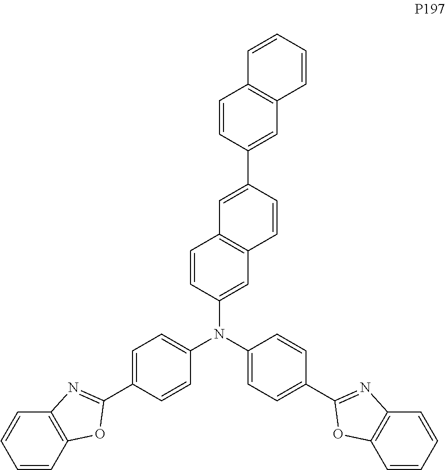 [SMILES: c1ccc2cc(-c3ccc4cc(N(c5ccc(-c6nc7ccccc7o6)cc5)c5ccc(-c6nc7ccccc7o6)cc5)ccc4c3)ccc2c1]

A specific preparation method includes steps described below.

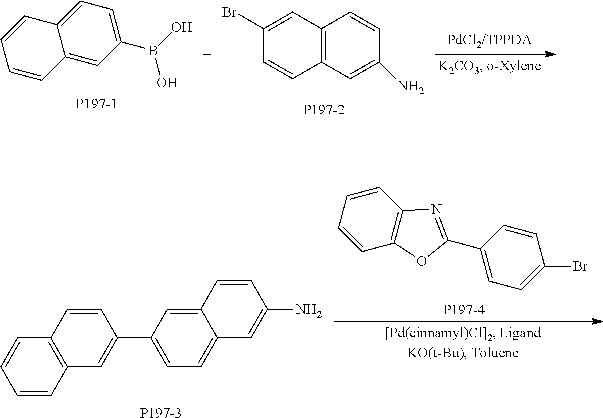

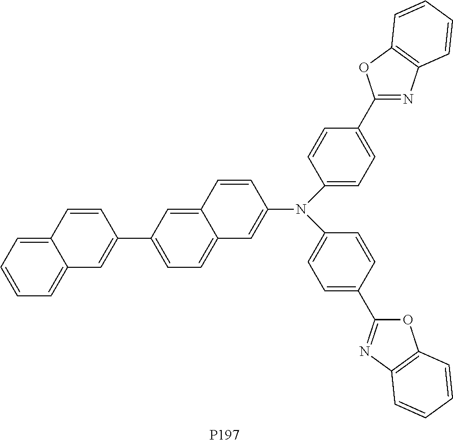 [SMILES: "P197" ; c1ccc2cc(-c3ccc4cc(N(c5ccc(-c6nc7ccccc7o6)cc5)c5ccc(-c6nc7ccccc7o6)cc5)ccc4c3)ccc2c1]

(1) P197-1 (0.5 mmol), P197-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 24 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P197-3 was obtained through column chromatography.

The structure of the target product P197-3 was tested through matrix-assisted laser desorption/ionization time-of-flight mass spectrometry (MALDI-TOF-MS) (m/z) to obtain C₂₀H₁₅N whose calculated value was 269.1 and measured value was 269.0.

(2) P197-3 (0.5 mmol), P197-4 (1.5 mmol), KO(t-Bu) (0.75 mmol), [Pd(cinnamyl)Cl]₂ (2 mol %) and a ligand (1.5 mol %) were added to 3 mL of toluene and mixed into a solution, and the solution was put in a 50 mL flask and reacted at 80° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P197 was obtained through column chromatography.

The structure of the target product P197 was tested through matrix-assisted laser desorption/ionization time-of-flight mass spectrometry (MALDI-TOF-MS) (m/z) to obtain C₄₆H₂₉N₃O₂ whose calculated value was 655.2 and measured value was 655.1.

Elemental analysis: theoretical value: C, 84.25, H, 4.46; N, 6.41; measured value: C, 84.24; H, 4.46; N, 6.42.

### Example 6

The synthesis route of Compound P205 is as follows:

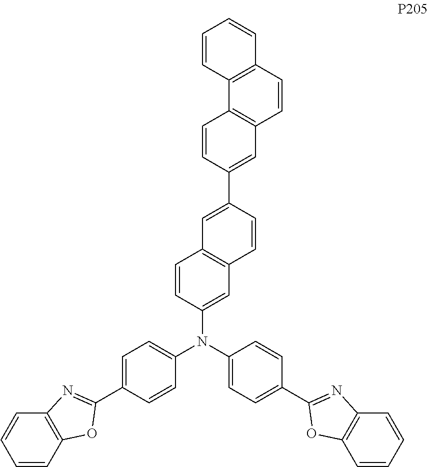 [SMILES: c1ccc2c(c1)ccc1cc(-c3ccc4cc(N(c5ccc(-c6nc7ccccc7o6)cc5)c5ccc(-c6nc7ccccc7o6)cc5)ccc4c3)ccc12]

A specific preparation method includes steps described below.

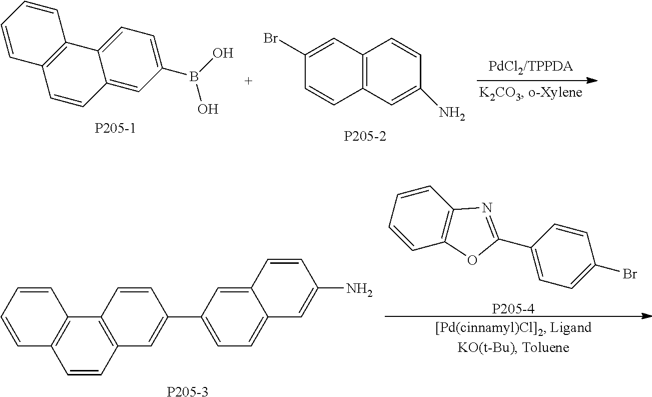

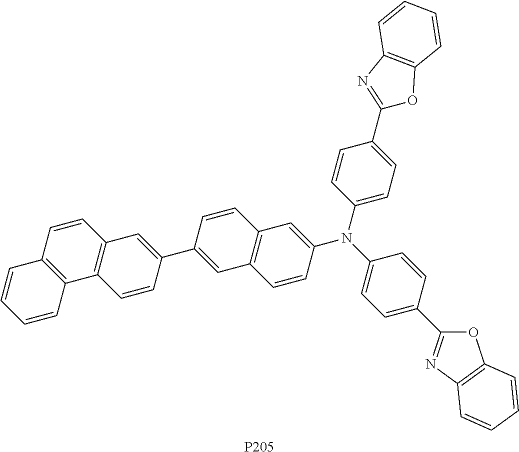 [SMILES: "P205" ; c1ccc2c(c1)ccc1cc(-c3ccc4cc(N(c5ccc(-c6nc7ccccc7o6)cc5)c5ccc(-c6nc7ccccc7o6)cc5)ccc4c3)ccc12]

(1) P205-1 (0.5 mmol), P205-2 (0.75 mmol), K₂CO₃ (0.5 mmol), PdCl₂ (5×10⁻⁴ mmol) and TPPDA (5×10⁻⁴ mmol) were added to 3 mL of o-xylene and mixed into a solution, and the solution was put in a 50 mL flask and reacted at 100° C. for 24 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P205-3 was obtained through column chromatography.

The structure of the target product P205-3 was tested through matrix-assisted laser desorption/ionization time-of-flight mass spectrometry (MALDI-TOF-MS) (m/z) to obtain C₂₄H₁₇N whose calculated value was 319.1 and measured value was 319.0.

(2) P205-3 (0.5 mmol), P205-4 (1.5 mmol), KO(t-Bu) (0.75 mmol), [Pd(cinnamyl)Cl]₂ (2 mol %) and a ligand (1.5 mol %) were added to 3 mL of toluene and mixed into a solution, and the solution was put in a 50 mL flask and reacted at 80° C. for 12 h. Then the solution was cooled to room temperature and slowly added with a saturated aqueous solution of MgSO₄ and ethyl acetate to be extracted for three times. Then, the organic layer is passed through a rotary evaporator for the solvent to be removed and the crude product P205 was obtained through column chromatography.

The structure of the target product P205 was tested through matrix-assisted laser desorption/ionization time-of-flight mass spectrometry (MALDI-TOF-MS) (m/z) to obtain C₅₀H₃₁N₃O₂ whose calculated value was 705.2 and measured value was 705.1.

Elemental analysis: theoretical value: C, 85.09, H, 4.43; N, 5.95; measured value: C, 85.08; H, 4.43; N, 5.94.

The preparation methods for other compounds of the present disclosure are all similar to the preceding methods and not repeated herein. Only the characterization results of mass spectrometry and elemental analysis of some other compounds of the present disclosure are provided, which are shown in Table 1-1.

TABLE 1-1
Characterization results of compounds

| | Result of Mass | | | |
|---|---|---|---|---|
| | Spectrometry | | | |
| | Calculated | Measured | Result of Elemental Analysis | |
| Compound | Value | Value | Theoretical Value | Measured Value |
| P1 | 558.2 | 558.3 | C, 77.40; H, 4.33; N, 12.54; | C, 77.40; H, 4.32; N, 12.55; |
| P11 | 526.2 | 526.1 | C, 82.11; H, 4.59; N, 13.30; | C, 82.10; H, 4.59; N, 13.30; |
| P145 | 687.2 | 687.1 | C, 80.32; H, 4.25; N, 6.11; | C, 80.32; H, 4.25; N, 6.10; |
| P157 | 688.2 | 688.1 | C, 78.46; H, 4.10; N, 8.13; | C, 78.45; H, 4.10; N, 8.14; |
| P169 | 655.2 | 655.1 | C, 84.25; H, 4.46; N, 6.41; | C, 84.25; H, 4.45; N, 6.42; |
| P198 | 655.2 | 655.3 | C, 84.25; H, 4.46; N, 6.41; | C, 84.25; H, 4.46; N, 6.40; |
| P200 | 705.2 | 705.1 | C, 85.09; H, 4.43; N, 5.95; | C, 85.09; H, 4.44; N, 5.95; |
| P201 | 705.2 | 705.0 | C, 85.09; H, 4.43; N, 5.95; | C, 85.09; H, 4.43; N, 5.95; |
| P203 | 705.2 | 705.1 | C, 85.09; H, 4.43; N, 5.95; | C, 85.08; H, 4.44; N, 5.95; |
| P206 | 705.2 | 705.2 | C, 85.09; H, 4.43; N, 5.95; | C, 85.09; H, 4.42; N, 5.95; |
| P207 | 662.3 | 662.4 | C, 83.36; H, 5.47; N, 6.34; | C, 83.36; H, 5.46; N, 6.34; |
| P208 | 670.3 | 670.2 | C, 82.36; H, 6.61; N, 6.26; | C, 82.36; H, 6.60; N, 6.26; |
| P230 | 855.3 | 855.1 | C, 87.00; H, 4.36; N, 4.91; | C, 87.01; H, 4.37; N, 4.90; |
| P231 | 694.2 | 684.1 | C, 77.80; H, 3.77; N, 16.13; | C, 77.80; H, 3.78; N, 16.13; |
| P232 | 883.3 | 883.2 | C, 84.23; H, 4.67; N, 11.09; | C, 84.23; H, 4.66; N, 11.09; |
| P277 | 673.2 | 673.1 | C, 82.00; H, 4.19; N, 6.24; | C, 82.00; H, 4.19; N, 6.25; |
| P280 | 723.2 | 723.1 | C, 78.00; H, 3.90; N, 5.81; | C, 78.00; H, 3.91; N, 5.80; |
| P293 | 729.2 | 729.1 | C, 85.58; H, 4.28; N, 5.76; | C, 85.59; H, 4.28; N, 5.75; |
| P294 | 655.2 | 655.1 | C, 84.25; H, 4.46; N, 6.41; | C, 84.25; H, 4.47; N, 6.40; |
| P295 | 659.3 | 659.2 | C, 83.74; H, 5.04; N, 6.37; | C, 83.74; H, 5.05; N, 6.37; |
| P296 | 687.2 | 687.1 | C, 80.32; H, 4.25; N, 6.11; | C, 80.32; H, 4.26; N, 6.11; |
| P297 | 839.2 | 839.1 | C, 82.92; H, 4.44; N, 5.00; | C, 82.92; H, 4.45; N, 5.00; |

Characterization data of the compounds through proton nuclear magnetic resonance are provided in Table 1-2.

TABLE 1-2

| Compound | NMR Data |
|---|---|
| P1 | ¹H-NMR (400 MHz, DMSO) δ (ppm) 8.38 (d, 2H), 8.27 |
| | (s, 1H), 8.19-7.95 (m, 11H), 7.87 (s, 2H), 7.80-7.71 (m, |
| | 4H), 7.58-7.51 (m, 2H); |
| P11 | ¹H-NMR (400 MHz, DMSO) δ (ppm) 8.55 (s, 2H),8.40 |
| | (d, 2H), 8.29-7.97 (m, 12H), 7.82-7.70 (m, 4H), 7.48-7.40 |
| | (m, 2H); |
| P157 | ¹H-NMR (400 MHz, DMSO) δ (ppm) 8.80 (d, 1H), 8.27 |
| | (s, 1H), 8.39-7.99 (m, 5H), 7.90-7.81 (m, 8H), 7.68-7.61 |
| | (m, 4H), 7.53-7.40 (m, 9H); |
| P169 | ¹H-NMR (400 MHz, DMSO) δ (ppm) 8.41 (d, 2H), 8.20- |
| | 7.96 (m, 12H), 7.81-7.73 (m, 4H), 7.56-7.51 (m, 2H), |
| | 7.43-7.32 (m, 7H), 6.99 (s, 2H); |
| P197 | ¹H-NMR (400 MHz, DMSO) δ (ppm) 8.40 (d, 2H), 8.19- |
| | 7.95 (m, 12H), 7.80-7.71 (m, 4H), 7.58-7.51 (m, 2H), |
| | 7.43-7.31 (m, 9H); |
| P198 | ¹H-NMR (400 MHz, DMSO) δ (ppm) 8.42 (d, 1H), 8.36 |
| | (d, 1H), 8.22 (d, 1H), 8.18-7.95 (m, 11H), 7.84-7.70 (m, |
| | 4H),7.58-7.51 (m, 2H), 7.42-7.30 (m, 9H); |
| P200 | ¹H-NMR (400 MHz, CDCl₃) δ (ppm) 8.54 (s, 1H), 8.49 |
| | (s, 1H), 8.35 (s, 1H), 8.24-8.21 (m, 5H), 8.16 (d, 1H), |
| | 8.07-8.04 (m, 2H), 7.97-7.95 (m, 2H), 7.92-7.90 (m, |
| | 1H),7.85-7.83 (m, 1H), 7.79-7.78 (m, 2H), 7.72-7.71 (m, |
| | 1H), 7.61-7.59 (m, 2H), 7.52-7.50 (m, 2H) , 7.42-7.28 (m, |
| | 9H); |
| P201 | ¹H-NMR (400 MHz, CDCl₃) δ (ppm) 8.44 (s, 1H), 8.39 |
| | (d, 1H), 8.34 (d, 1H),8.24-8.21 (m, 5H), 8.16 (d, 1H), |
| | 8.07-7.90 (m, 5H),7.85-7.83 (m, 1H), 7.79-7.59 (m, 5H), |
| | 7.52-7.50 (m, 2H) , 7.42-7.28 (m, 9H); |
| P203 | ¹H-NMR (400 MHz, CDCl₃) δ (ppm) 8.84 (d, 1H), 8.78 |
| | (d, 1H), 8.23 (d, 4H), 8.05 (s, 1H), 8.01-7.95(m, 3H), 7.81 |
| | (d, 1H), 7.73-7.56 (m, 11H), 7.46 (d, 1H), 7.37-7.35 (m, |
| | 8H); |
| P205 | ¹H-NMR (400 MHz, CDCl₃) δ (ppm) 8.81 (d, 1H), 8.84 |
| | (d, 1H), 8.82-8.20 (m, 6H), 8.06 (d, 1H), 8.05-8.04 (m, |
| | 3H), 7.99-7.93 (m, 5H), 7.87-7.80 (m, 2H), 7.79-7.66 (m, |
| | 3H), 7.60-7.27 (m, 9H); |
| P206 | ¹H-NMR (400 MHz, CDCl₃) δ (ppm) 8.72 (d, 2H),8.83- |
| | 8.20 (m, 6H), 8.08 (d, 1H), 8.05-8.06 (m, 3H), 7.99-7.93 |
| | (m, 5H), 7.87-7.66 (m, 5H), 7.61-7.27 (m, 9H); |
| P207 | ¹H-NMR (400 MHz, CDCl₃) δ (ppm) 8.83-8.20 (m, 5H), |
| | 7.99-7.93 (m, 4H), 7.87-7.66 (m, 5H), 7.61-7.27 (m, 8H); |
| P208 | ¹H-NMR (400 MHz, CDCl₃) δ (ppm) 7.85-7.83 (m, 1H), |
| | 7.79-7.78 (m, 2H), 7.72-7.71 (m, 1H), 7.61-7.59 (m, 2H), |
| | 7.52-7.50 (m, 2H), 7.42-7.28 (m, 6H); |
| P232 | ¹H-NMR (400 MHz, CDCl₃) δ (ppm) 8.43 (s, 1H), 8.32 |
| | (s, 1H), 8.26-8.21 (m, 5H), 8.17 (d, 1H), 8.07-7.90 (m, |
| | 20H), 7.85-7.59 (m, 4H), 7.41-7.12 (m, 9H); |
| P277 | ¹H-NMR (400 MHz, CDCl₃) δ (ppm) 8.74 (s, 1H), 8.49 |
| | (s, 1H), 8.25-8.21 (m, 4H), 8.07-7.91 (m, 5H), 7.86-7.83 |
| | (m, 1H), 7.76-7.58 (m, 5H), 7.53-7.50 (m, 2H), 7.41-7.28 |
| | (m, 9H); |
| P280 | ¹H-NMR (400 MHz, CDCl₃) δ (ppm) 8.81 (s, 1H), 8.50 |
| | (s, 1H), 8.26-8.21 (m, 4H), 8.06-7.91 (m, 5H), 7.86-7.82 |
| | (m, 1H), 7.76-7.57 (m, 5H), 7.53-7.50 (m, 2H), 7.41-7.27 |
| | (m, 9H); |
| P294 | ¹H-NMR (400 MHz, DMSO) δ (ppm) 8.39 (d, 2H), 8.18- |
| | 7.94 (m, 12H), 7.80-7.61 (m, 4H), 7.58-7.50 (m, 2H), |
| | 7.43-7.30 (m, 7H), 7.28 (s, 2H); |
| P295 | ¹H-NMR (400 MHz, DMSO) δ (ppm) 8.40 (d, 2H), 8.19- |
| | 7.95 (m, 11H), 7.90 (s, 1H), 7.80-7.71 (m, 3H), 7.58 (s, |
| | 1H), 7.43-7.31 (m, 7H); |

Performance Test I Characterization of Refractive Indexes of Materials

The refractive indexes of the compounds at wavelengths of 460 nm, 530 nm and 620 nm were tested by an ellipsometer. A difference Δn₁ between the refractive index at the wavelength of 460 nm and the refractive index at the wavelength of 530 nm, a difference Δn₂ between the refractive index at the wavelength of 530 nm and the refractive index at the wavelength of 620 nm, and a difference Δn₃ between the refractive index at the wavelength of 460 nm and the refractive index at the wavelength of 620 nm were calculated.

The results of the preceding test are shown in Table 2.

TABLE 2

| Compound | n_{460 nm} | n_{530 nm} | n_{620 nm} | Δn₁ | Δn₂ | Δn₃ |
|---|---|---|---|---|---|---|
| P1 | 2.19 | 2.09 | 2.04 | 0.10 | 0.05 | 0.15 |
| P11 | 2.18 | 2.07 | 2.01 | 0.11 | 0.06 | 0.17 |
| P145 | 2.30 | 2.20 | 2.15 | 0.10 | 0.05 | 0.15 |
| P157 | 2.32 | 2.21 | 2.18 | 0.11 | 0.03 | 0.14 |
| P169 | 2.20 | 2.10 | 2.15 | 0.10 | 0.05 | 0.15 |
| P197 | 2.24 | 2.12 | 2.04 | 0.12 | 0.08 | 0.20 |
| P198 | 2.10 | 1.98 | 1.89 | 0.12 | 0.09 | 0.21 |
| P200 | 2.21 | 2.06 | 1.97 | 0.15 | 0.09 | 0.24 |
| P201 | 2.35 | 2.20 | 2.11 | 0.15 | 0.08 | 0.23 |
| P203 | 2.23 | 2.11 | 2.04 | 0.12 | 0.07 | 0.19 |
| P205 | 2.32 | 2.22 | 2.16 | 0.10 | 0.06 | 0.16 |
| P206 | 2.22 | 2.07 | 1.99 | 0.15 | 0.08 | 0.23 |
| P207 | 2.25 | 2.13 | 2.05 | 0.12 | 0.08 | 0.20 |
| P208 | 2.26 | 2.14 | 2.07 | 0.12 | 0.07 | 0.19 |
| P230 | 2.35 | 2.20 | 2.12 | 0.15 | 0.08 | 0.23 |
| P231 | 2.30 | 2.19 | 2.12 | 0.11 | 0.07 | 0.18 |
| P232 | 2.18 | 2.08 | 2.05 | 0.10 | 0.03 | 0.13 |
| P277 | 2.25 | 2.14 | 2.09 | 0.11 | 0.05 | 0.16 |
| P280 | 2.25 | 2.14 | 2.10 | 0.11 | 0.04 | 0.15 |
| P293 | 2.25 | 2.09 | 2.01 | 0.16 | 0.08 | 0.24 |
| P294 | 2.19 | 2.08 | 2.03 | 0.11 | 0.05 | 0.16 |
| P295 | 2.25 | 2.13 | 2.07 | 0.12 | 0.06 | 0.18 |
| P296 | 2.27 | 2.14 | 2.06 | 0.13 | 0.08 | 0.21 |
| P297 | 2.25 | 2.13 | 2.07 | 0.12 | 0.06 | 0.18 |
| C1 | 2.18 | 2.00 | 1.93 | 0.18 | 0.07 | 0.25 |
| C2 | 2.20 | 2.05 | 1.94 | 0.15 | 0.11 | 0.26 |

Comparative compounds C1 and C2 have the following structures:

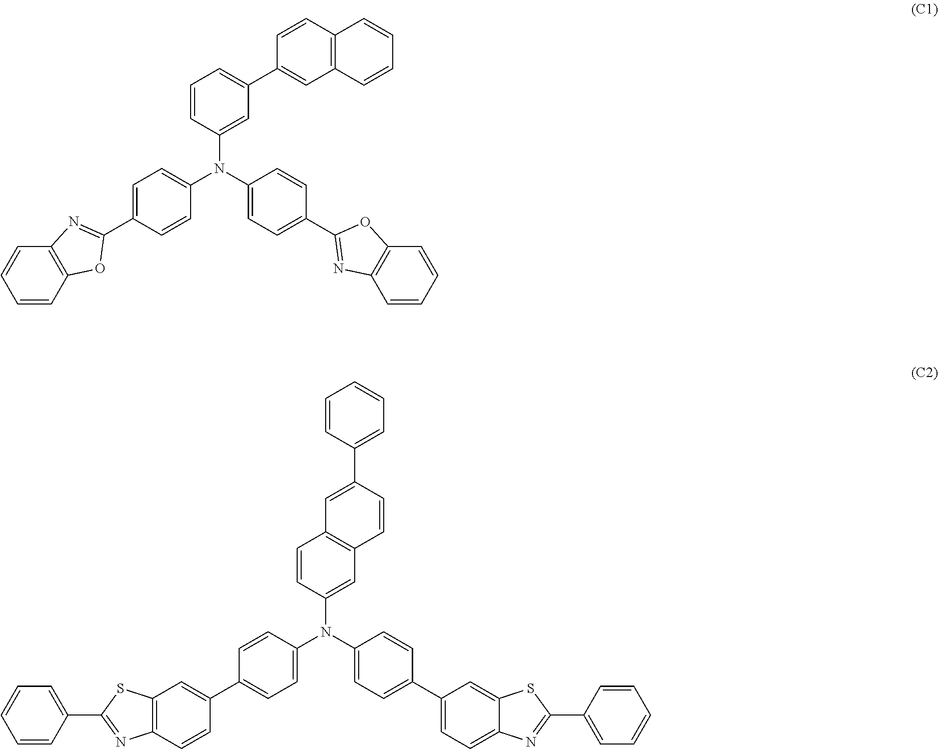 [SMILES: c1cc(-c2ccc3ccccc3c2)cc(N(c2ccc(-c3nc4ccccc4o3)cc2)c2ccc(-c3nc4ccccc4o3)cc2)c1 ; c1ccc(-c2ccc3cc(N(c4ccc(-c5ccc6nc(-c7ccccc7)sc6c5)cc4)c4ccc(-c5ccc6nc(-c7ccccc7)sc6c5)cc4)ccc3c2)cc1]

It can be seen from Table 1 that the compounds provided by the present application satisfy that the difference between the refractive index at the wavelength of 460 nm and the refractive index at the wavelength of 530 nm is 0.10-0.17, the difference between the refractive index at the wavelength of 530 nm and the refractive index at the wavelength of 620 nm is 0.03-0.10, and the difference between the refractive index at the wavelength of 460 nm and the refractive index at the wavelength of 620 nm is 0.15-0.40. When used in the organic electroluminescent device, especially as a material for a capping layer, the compound provided by the present application can improve the color cast while achieving display at multiple angles compared with compounds C1 and C2.

Performance Test II Characterization of Absorbance of Materials

The compounds of the present disclosure were made into films with a thickness of 700 angstroms, and the absorbances of the films at a wavelength of 250 nm and at a wavelength of 380 nm were measured by an ultraviolet spectrophotometer.

The results of the preceding test are shown in Table 3.

TABLE 3

| | Absorbance (at | Absorbance (at |
|---|---|---|
| | a wavelength | a wavelength |
| Compound | of 250 nm) | of 380 nm) |
| P1 | 0.57 | 0.34 |
| P11 | 0.47 | 0.56 |
| P145 | 0.95 | 0.96 |
| P157 | 0.83 | 0.87 |
| P169 | 0.80 | 0.79 |
| P197 | 0.85 | 0.86 |
| P198 | 0.74 | 0.63 |
| P200 | 0.85 | 0.64 |
| P201 | 0.85 | 0.53 |
| P203 | 0.95 | 0.85 |
| P205 | 1.00 | 0.75 |
| P206 | 0.75 | 0.63 |
| P207 | 0.86 | 0.86 |
| P208 | 0.87 | 0.85 |
| P230 | 0.56 | 0.88 |
| P231 | 0.75 | 0.56 |
| P232 | 0.95 | 0.76 |
| P277 | 0.85 | 0.86 |
| P280 | 0.86 | 0.86 |
| P293 | 0.52 | 1.15 |
| P294 | 0.83 | 0.86 |
| P295 | 0.85 | 0.86 |
| P296 | 0.89 | 0.90 |
| P297 | 0.82 | 0.86 |
| C1 | 0.24 | 0.85 |
| C2 | 0.23 | 0.96 |

It can be seen from Table 3 that the film prepared from the compound provided by the present disclosure has an absorbance greater than 0.3 at the wavelength of 250 nm and at the wavelength of 380 nm so that the efficient absorption of light at the wavelengths of 250 nm and 380 nm is achieved and damage to human eyes is avoided.

For a better understanding of the present disclosure, application examples of the compounds of the present disclosure are listed below. It is understood that the examples described herein are used for a better understanding of the present disclosure and are not to be construed as specific limitations to the present disclosure.

### Application Example 1

This application example provides an organic electroluminescent device which has a structure shown in FIG. 1 and is prepared through specific steps described below.

(1) A glass substrate with an indium tin oxide (ITO) anode layer 2 (with a thickness of 15 nm) was cut into a size of 50 mm×50 mm×0.7 mm, sonicated in isopropyl alcohol and deionized water for 30 min separately, and cleaned under ozone for 10 min. The cleaned substrate 1 was installed onto a vacuum deposition device.

(2) A material for a hole injection layer, Compound 2, and a p-doped material, Compound 1, were co-deposited at a doping ratio of 3% (mass ratio) by means of vacuum evaporation on the ITO anode layer 2 as a hole injection layer 3 with a thickness of 5 nm.

(3) A material for a hole transport layer, Compound 2, was deposited by means of vacuum evaporation on the hole injection layer 3 as a first hole transport layer 4 with a thickness of 100 nm.

(4) A hole transport material, Compound 3, was deposited by means of vacuum evaporation on the first hole transport layer 4 as a second hole transport layer 5 with a thickness of 5 nm.

(5) A light-emitting layer 6 with a thickness of 30 nm was deposited by means of vacuum evaporation on the second hole transport layer 5, where Compound 4 was doped as a host material with Compound 5 as a doping material at a ratio of 3% (mass ratio).

(6) An electron transport material, Compound 6, was deposited by means of vacuum evaporation on the light-emitting layer 6 as a first electron transport layer 7 with a thickness of 30 nm.

(7) An electron transport material, Compound 7, and a n-doping material, Compound 8, were co-deposited at a doping mass ratio of 1:1 by means of vacuum evaporation on the first electron transport layer 7 as a second electron transport layer 8 with a thickness of 5 nm.

(8) A magnesium-silver electrode was deposited at a ratio of 9:1 by means of vacuum evaporation on the second electron transport layer 8 as a cathode 9 with a thickness of 10 nm.

(9) Compound P1 of the present disclosure was deposited by means of vacuum evaporation on the cathode 9 as a capping layer 10 with a thickness of 100 nm.

The compounds used in the preceding steps have the following structures:

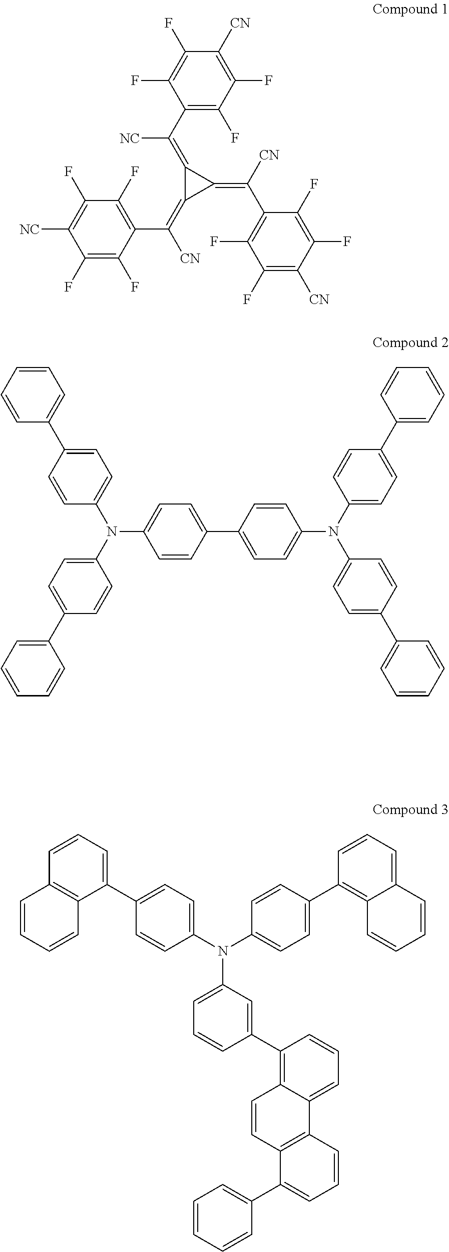 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc3c2ccc2c(-c4cccc(N(c5ccc(-c6cccc7ccccc67)cc5)c5ccc(-c6cccc7ccccc67)cc5)c4)cccc23)cc1]

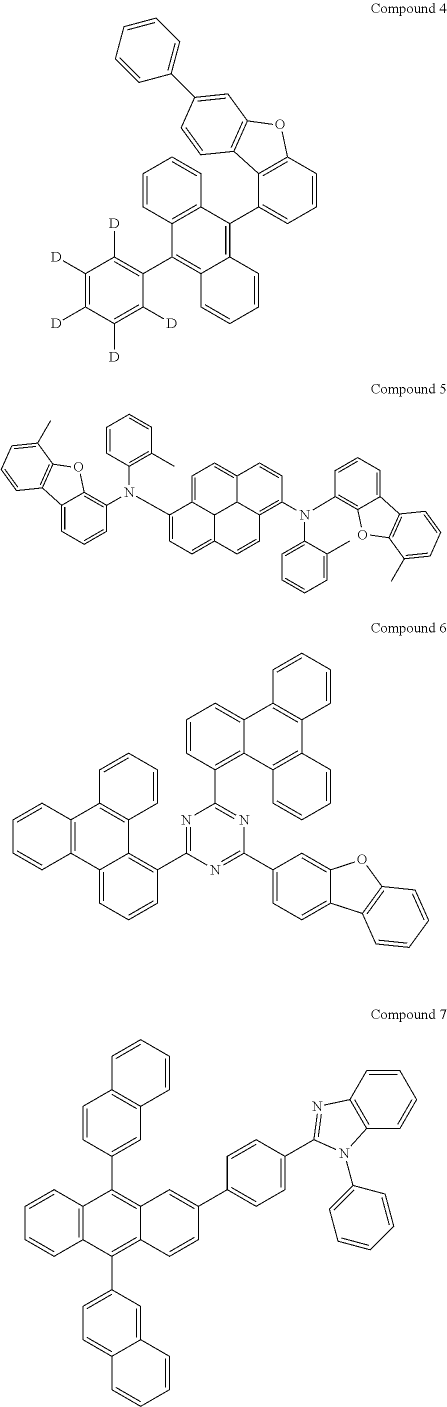 [SMILES: [2H]c1c([2H])c([2H])c(-c2c3ccccc3c(-c3cccc4oc5cc(-c6ccccc6)ccc5c34)c3ccccc23)c([2H])c1[2H] ; Cc1ccccc1N(C1=CC=C2C=CC3=C(N(c4ccccc4C)c4cccc5c4oc4c(C)cccc45)C=CC4=CC=C1C2C43)c1cccc2c1oc1c(C)cccc12 ; c1ccc2c(c1)oc1cc(-c3nc(-c4cccc5c6ccccc6c6ccccc6c45)nc(-c4cccc5c6ccccc6c6ccccc6c45)n3)ccc12 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

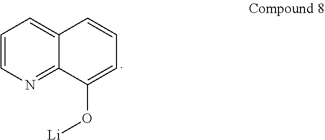

Application Examples 2-24 and Comparative Application Examples 1-2 differ from Application Example 1 only in that Compound P1 in step (9) was replaced with Compounds P11, P145, P157, P169, P197, P198, P200, P201, P203, P205, P206, P207, P208, P230, P231, P232, P277, P280, P293, P294, P295, P296, P297, C1 and C2 respectively for preparing the capping layer. All the other preparation steps are the same. For details, see Table 4.

Performance Test III Characterization of Device Performance

A performance test was performed on organic electroluminescent devices provided in Application Examples 2-24 and Comparative Application Examples 1-2 as follows.

Currents were measured with Keithley 2365A digital nanovoltmeter at different voltages for the organic electroluminescent devices and then divided by a light-emitting area so that the current densities of the organic optoelectronic devices at different voltages were obtained. The brightness and radiation energy flux density of each of the organic electroluminescent devices manufactured according to application examples and comparative application examples at different voltages were tested with Konicaminolta CS-2000 spectrometer. According to the current densities and brightness of the organic electroluminescent devices at different voltages, an operating voltage Vₒₙ (V), a current efficiency CE (cd/A), an external quantum efficiency EQE₍ₘₐₓ₎, a color cast JNCD (30/45/60° C.) and a lifetime LT95 (which is obtained by measuring time taken for the organic electroluminescent device to reach 95% of initial brightness (under a condition of 50 mA/cm²)) at the same current density (10 mA/cm²) were obtained. The results are shown in Table 4.

TABLE 4

| | | | CE_{(10mA/cm}_{²}₎ | | EQE₍ₘₐₓ₎ | Lifetime |
|---|---|---|---|---|---|---|
| No. | Compound | Vₒₙ (V) | (cd A⁻¹) | JNCD | (%) | LT95(h) |
| Application | P1 | 3.52 | 6.99 | 4/2/1 | 16.9 | 66 |
| Example 1 | | | | | | |
| Application | P11 | 3.50 | 7.32 | 4/4/1 | 17.3 | 74 |
| Example 2 | | | | | | |
| Application | P145 | 3.43 | 7.89 | 4/1/1 | 18.1 | 70 |
| Example 3 | | | | | | |
| Application | P157 | 3.46 | 7.90 | 3/2/1 | 18.2 | 69 |
| Example 4 | | | | | | |
| Application | P169 | 3.47 | 7.70 | 5/2/1 | 17.9 | 70 |
| Example 5 | | | | | | |
| Application | P197 | 3.42 | 7.86 | 4/3/1 | 18.1 | 71 |
| Example 6 | | | | | | |
| Application | P198 | 3.45 | 7.05 | 4/4/2 | 17.3 | 70 |
| Example 7 | | | | | | |
| Application | P200 | 3.45 | 7.85 | 5/3/1 | 18.0 | 68 |
| Example 8 | | | | | | |
| Application | P201 | 3.46 | 8.01 | 5/2/1 | 18.6 | 68 |
| Example 9 | | | | | | |
| Application | P203 | 3.43 | 7.95 | 4/3/2 | 18.1 | 71 |
| Example 10 | | | | | | |
| Application | P205 | 3.44 | 8.01 | 3/2/1 | 19.1 | 72 |
| Example 11 | | | | | | |
| Application | P206 | 3.45 | 7.81 | 4/3/3 | 18.0 | 73 |
| Example 12 | | | | | | |
| Application | P207 | 3.41 | 7.93 | 4/3/1 | 18.2 | 84 |
| Example 13 | | | | | | |
| Application | P208 | 3.40 | 8.04 | 3/3/1 | 18.2 | 89 |
| Example 14 | | | | | | |
| Application | P230 | 3.45 | 7.65 | 4/3/2 | 17.6 | 69 |
| Example 15 | | | | | | |
| Application | P231 | 3.52 | 7.54 | 4/2/1 | 17.0 | 68 |
| Example 16 | | | | | | |
| Application | P232 | 3.45 | 6.93 | 5/3/1 | 16.4 | 70 |
| Example 17 | | | | | | |
| Application | P277 | 3.42 | 7.87 | 4/3/1 | 18.1 | 70 |
| Example 18 | | | | | | |
| Application | P280 | 3.42 | 7.87 | 4/3/1 | 18.1 | 71 |
| Example 19 | | | | | | |
| Application | P293 | 3.49 | 7.86 | 4/4/1 | 17.6 | 65 |
| Example 20 | | | | | | |
| Application | P294 | 3.46 | 6.88 | 4/3/1 | 16.0 | 70 |
| Example 21 | | | | | | |
| Application | P295 | 3.42 | 7.87 | 4/2/1 | 18.1 | 94 |
| Example 22 | | | | | | |
| Application | P296 | 3.45 | 7.69 | 4/2/1 | 16.1 | 69 |
| Example 23 | | | | | | |
| Application | P297 | 3.46 | 7.58 | 5/1/1 | 15.9 | 68 |
| Example 24 | | | | | | |
| Comparative | C1 | 3.45 | 6.89 | 6/3/2 | 14.3 | 70 |
| Application | | | | | | |
| Example 1 | | | | | | |
| Comparative | C2 | 3.48 | 6.84 | 4/7/8 | 13.4 | 69 |
| Application | | | | | | |
| Example 2 | | | | | | |

It can be seen from Table 4 that when used as a material in the capping layer of the organic electroluminescent device, the compound of the present disclosure effectively reduces the color cast of the device and improves the current efficiency and the external quantum efficiency.

Compound P197 differs from Compound C1 only in that both an arylamino group and a fused ring aryl group are present as substituents on a naphthalene ring, and data shows that the organic electroluminescent device whose capping layer is made of P197 has a higher current efficiency, a higher external quantum efficiency, and a lower color cast value. Compound P296 differs from Compound C2 only in that a fused ring aryl group is present as a substituent at position 6 of the naphthalene ring, and data shows that the organic electroluminescent device whose capping layer is made of Compound P296 has a higher current efficiency, a higher external quantum efficiency, and a lower color cast value.

Compound P207 differs from Compound P197 only in that a deuterated substituent is present at position 6 of the naphthalene ring, and data shows that the organic electroluminescent device whose capping layer is made of Compound P207 has a higher lifetime.

Differences between Compounds P197, P198, P200, P201, P203, P205 and P206 are that the naphthalene ring, the phenanthrene ring or the anthracene ring have different linkage sites, and the organic electroluminescent devices whose capping layers are made of Compounds P197, P201, P203 and P205 have better device efficiency.

### Application Example 25

This application example provides an organic electroluminescent device which has a structure shown in FIG. 2 and is prepared through specific steps described below.

(1) A glass substrate with an indium tin oxide (ITO) anode layer 2 (with a thickness of 15 nm) was cut into a size of 50 mm×50 mm×0.7 mm, sonicated in isopropyl alcohol and deionized water for 30 min separately, and cleaned under ozone for 10 min. The cleaned substrate 1 was installed onto a vacuum deposition device.

(2) A material for a hole injection layer, Compound 2, and a p-doping material, Compound 1, were co-deposited at a doping ratio of 3% (mass ratio) by means of vacuum evaporation on the ITO anode layer 2 as a hole injection layer 3 with a thickness of 5 nm.

(3) A material for a hole transport layer, Compound 2, was deposited by means of vacuum evaporation on the hole injection layer 3 as a first hole transport layer 4 with a thickness of 100 nm.

(4) A hole transport material, Compound 3, was deposited by means of vacuum evaporation on the first hole transport layer 4 as a second hole transport layer 5 with a thickness of 5 nm.

(5) A light-emitting layer 6 with a thickness of 30 nm was deposited by means of vacuum evaporation on the second hole transport layer 5, where Compound 4 was doped as a host material with Compound 5 as a doping material at a ratio of 3% (mass ratio).

(6) An electron transport material, Compound 6, was deposited by means of vacuum evaporation on the light-emitting layer 6 as a first electron transport layer 7 with a thickness of 30 nm.

(7) An electron transport material, Compound 7, and an n-doping material, Compound 8, were co-deposited at a doping mass ratio of 1:1 by means of vacuum evaporation on the first electron transport layer 7 as a second electron transport layer 8 with a thickness of 5 nm.

(8) A magnesium-silver electrode was deposited at a ratio of 9:1 by means of vacuum evaporation on the second electron transport layer 8 as a cathode 9 with a thickness of 10 nm.

(9) Compound P197 of the present disclosure was deposited by means of vacuum evaporation on the cathode 9 as a first capping layer 10 with a thickness of 100 nm.

(10) A small organic molecule D1 with a low refractive index was deposited by means of vacuum evaporation on the first capping layer 10 as a second capping layer 11 with a thickness of 20 nm.

Some small organic molecules with low refractive indexes have the following structures:

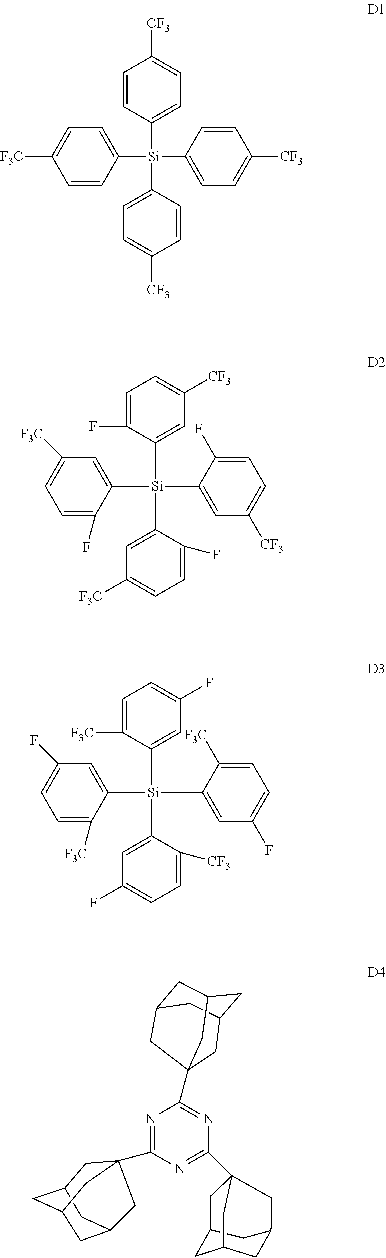 [SMILES: FC(F)(F)c1ccc([Si](c2ccc(C(F)(F)F)cc2)(c2ccc(C(F)(F)F)cc2)c2ccc(C(F)(F)F)cc2)cc1 ; Fc1ccc(C(F)(F)F)cc1[Si](c1cc(C(F)(F)F)ccc1F)(c1cc(C(F)(F)F)ccc1F)c1cc(C(F)(F)F)ccc1F ; Fc1ccc(C(F)(F)F)c([Si](c2cc(F)ccc2C(F)(F)F)(c2cc(F)ccc2C(F)(F)F)c2cc(F)ccc2C(F)(F)F)c1 ; C1C2CC3CC1CC(c1nc(C45CC6CC(CC(C6)C4)C5)nc(C45CC6CC(CC(C6)C4)C5)n1)(C2)C3]

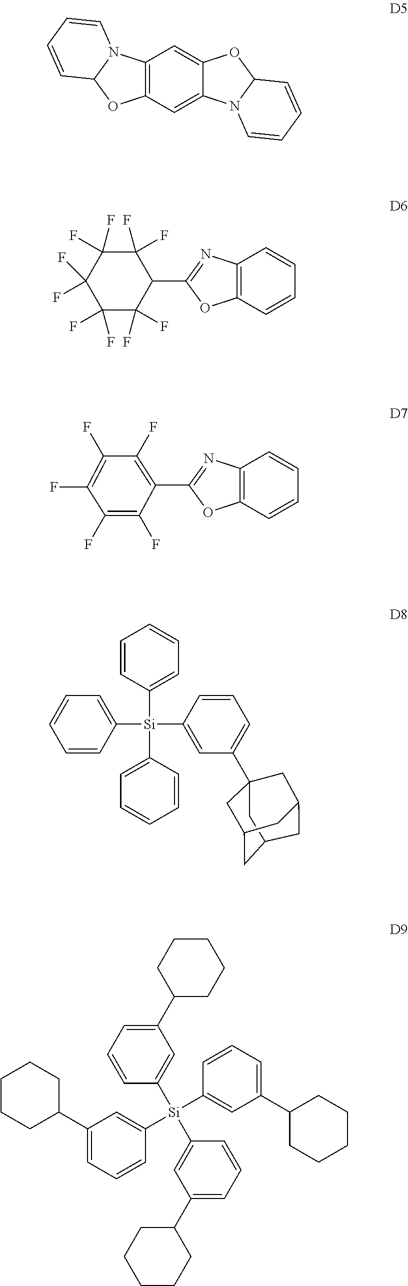 [SMILES: C1=CC2Oc3cc4c(cc3N2C=C1)OC1C=CC=CN41 ; FC1(F)C(c2nc3ccccc3o2)C(F)(F)C(F)(F)C(F)(F)C1(F)F ; Fc1c(F)c(F)c(-c2nc3ccccc3o2)c(F)c1F ; c1ccc([Si](c2ccccc2)(c2ccccc2)c2cccc(C34CC5CC(CC(C5)C3)C4)c2)cc1 ; c1cc(C2CCCCC2)cc([Si](c2cccc(C3CCCCC3)c2)(c2cccc(C3CCCCC3)c2)c2cccc(C3CCCCC3)c2)c1]

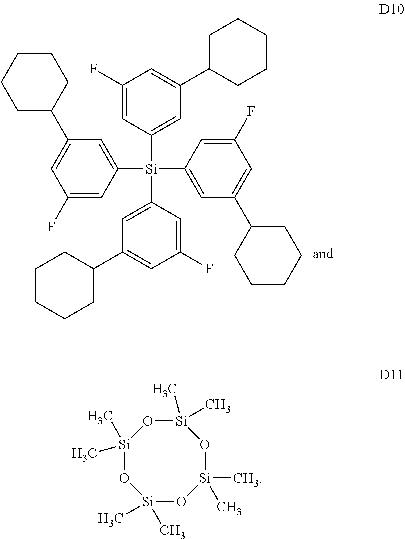 [SMILES: Fc1cc(C2CCCCC2)cc([Si](c2cc(F)cc(C3CCCCC3)c2)(c2cc(F)cc(C3CCCCC3)c2)c2cc(F)cc(C3CCCCC3)c2)c1 ; C[Si]1(C)O[Si](C)(C)O[Si](C)(C)O[Si](C)(C)O1]

Application Examples 26-35 differ from Application Example 25 only in that the small organic molecule D1 in step (10) was replaced with D2, D3, D4, D5, D6, D7, D8, D9, D10 and D11 respectively for preparing the second capping layer. All the other preparation steps are the same. Application Examples 36-38 and Comparative Application Examples 3-4 differ from Application Example 25 only in that Compound P197 in step (9) was replaced with P195, P295, P277, C1 and C2 respectively for preparing the first capping layer. For details, see Table 5.

The performance test was performed on organic electroluminescent devices provided in Application Examples 25-38 and Comparative Application Examples 3-4 by the same test method described above. The results are shown in Table 5.

TABLE 5

| | Material for | Material for | | |
|---|---|---|---|---|
| | First Capping | Second | CE_{(10 mA/cm}_{²}₎ | EQE₍ₘₐₓ₎ |
| No. | Layer | Capping Layer | (cd A⁻¹) | (%) |
| Application | P197 | D1 | 7.87 | 19.1 |
| Example 25 | | | | |
| Application | P197 | D2 | 7.88 | 19.4 |
| Example 26 | | | | |
| Application | P197 | D3 | 7.89 | 19.5 |
| Example 27 | | | | |
| Application | P197 | D4 | 7.86 | 19.1 |
| Example 28 | | | | |
| Application | P197 | D5 | 7.86 | 19.1 |
| Example 29 | | | | |
| Application | P197 | D6 | 7.86 | 19.0 |
| Example 30 | | | | |
| Application | P197 | D7 | 7.89 | 19.5 |
| Example 31 | | | | |
| Application | P197 | D8 | 7.87 | 19.1 |
| Example 32 | | | | |
| Application | P197 | D9 | 7.85 | 19.0 |
| Example 33 | | | | |
| Application | P197 | D10 | 7.96 | 19.8 |
| Example 34 | | | | |
| Application | P197 | D11 | 8.01 | 20.1 |
| Example 35 | | | | |
| Application | P195 | D1 | 7.91 | 19.1 |
| Example 36 | | | | |
| Application | P295 | D1 | 7.89 | 18.9 |
| Example 37 | | | | |
| Application | P277 | D1 | 7.89 | 18.9 |
| Example 38 | | | | |
| Comparative | C1 | D1 | 6.91 | 14.9 |
| Application | | | | |
| Example 3 | | | | |
| Comparative | C2 | D1 | 6.94 | 14.4 |
| Application | | | | |
| Example 4 | | | | |

It can be seen from Table 5 that compared with the use of Compound C1 or C2 with the material containing small organic molecules with a low refractive index used in the second capping layer, the use of the compound provided by the present disclosure as the material in the first capping layer with the material containing small organic molecules with a low refractive index used in the second capping layer is more conducive to improving the device efficiency, especially in terms of improving the external quantum efficiency.

It is understood that the present disclosure is not limited to the detailed method described above, which means that the implementation of the present disclosure does not depend on the detailed method described above. It should be apparent to those skilled in the art that any improvements made to the present disclosure, equivalent substitutions of various raw materials of the product, the addition of adjuvant ingredients, and the selection of specific manners, etc. in the present disclosure all fall within the protection scope and the disclosure scope of the present disclosure.

What is claimed is:

1. A compound having a structure represented by Formula (1): 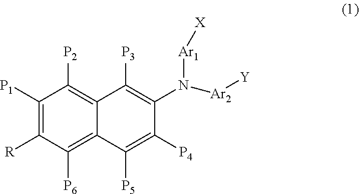
wherein, R is selected from the group consisting of substituted or unsubstituted C10-C60 fused-ring aryl and substituted or unsubstituted C6-C60 fused-ring heteroaryl;
wherein, each of P₁, P₂, P₃, P₄, P₅ and P₆ are is selected from the group consisting of a hydrogen atom, a deuterium atom, CD₃, CD₂CH₃ and CD₂CD₃;
wherein, each of Ar₁ and Ar₂ is selected from the group consisting of a single bond, substituted or unsubstituted C6-C60 arylene, substituted or unsubstituted C3-C60 heteroarylene, substituted or unsubstituted C10-C60 fused-ring arylene and substituted or unsubstituted C6-C60 fused-ring heteroarylene;
wherein, each of X and Y is selected from the group consisting of substituted or unsubstituted C6-C60 aryl and substituted or unsubstituted C3-C60 heteroaryl, and at least one of X and Y is selected from substituted or unsubstituted C3-C60 electron withdrawing heteroaryl; and
wherein each of substituted groups in R, Ar₁, Ar₂, X and Y is selected from the group consisting of protium, deuterium, tritium, halogen, C1-C10 alkyl, C1-C10 haloalkyl, C1-C10 alkoxy, C6-C60 aryl, C3-C60 heteroaryl, and a combination of at least two selected therefrom.

2. The compound according to claim 1, wherein R is selected from groups represented by Formula (2), Formula (3) and Formula (4): 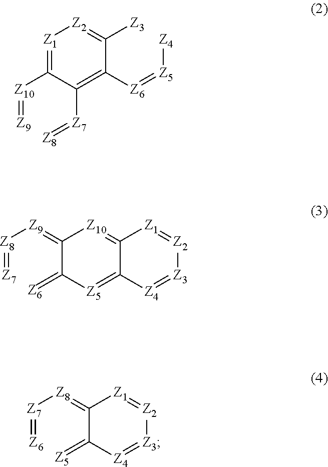
wherein each of Z₁ to Z₁₀ is selected from the group consisting of a N atom, CH and CR₁, wherein R₁ is selected from the group consisting of protium, deuterium, tritium, halogen, C1-C10 alkyl, C1-C10 haloalkyl, C1-C10 alkoxy, C6-C60 aryl, C3-C60 heteroaryl, and a combination of at least two selected therefrom.

3. The compound according to claim 2, wherein each of Z₁ to Z₁₀ is selected from the group consisting of CH and CR₁.

4. The compound according to claim 3, wherein R is selected from the group consisting of the following groups: 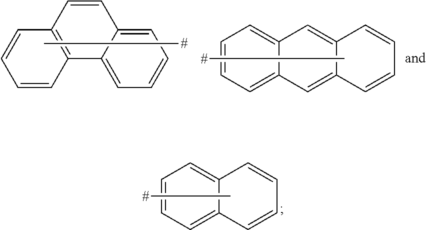
wherein # represents a linkage site of the group.

5. The compound according to claim 4, wherein R is selected from the group consisting of the following groups: 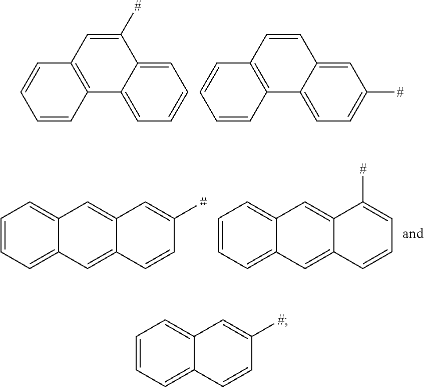
wherein # represents a linkage site of the group.

6. The compound according to claim 5, wherein R is selected from the group consisting of the following groups: 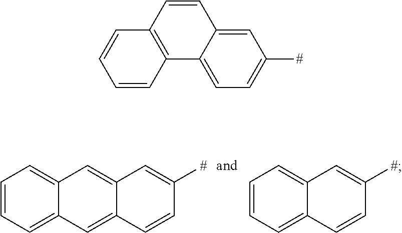
wherein # represents a linkage site of the group.

7. The compound according to claim 2, wherein in Formula (2), at least one of Z₁ to Z₁₀ is selected from N;
and where in in Formula (3), at least one of Z₁ to Z₁₀ is selected from N; and
in Formula (4), at least one of Z₁ to Z₈ is selected from N.

8. The compound according to claim 7, wherein R is selected from the group consisting of the following groups: 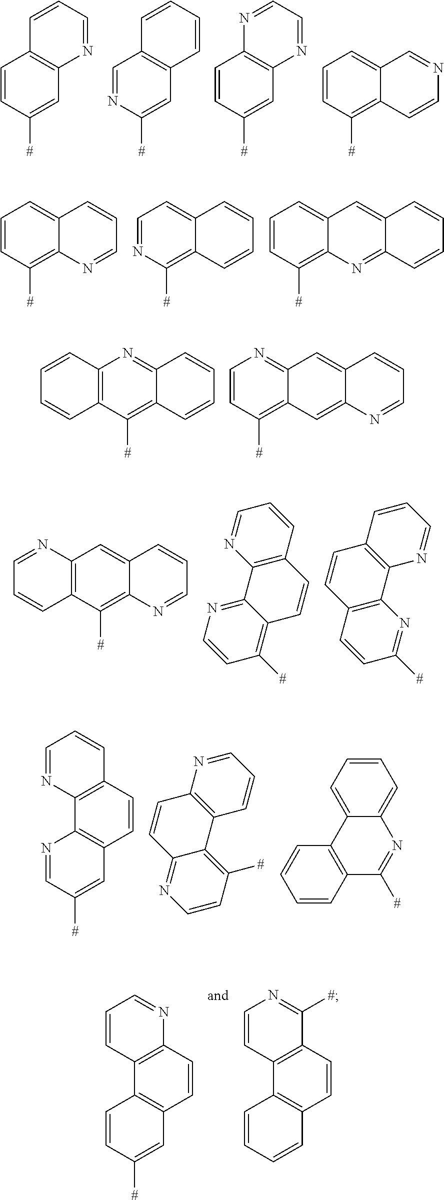
wherein # represents a linkage site of the group.

9. The compound according to claim 2, wherein in Formula (2), at least one of Z₁ to Z₁₀ is CR₁, and wherein R₁ is selected from the group consisting of deuterium, halogen, C1-C10 haloalkyl, and a combination of at least two selected therefrom;
wherein in Formula (3), at least one of Z₁ to Z₁₀ is CR₁, and wherein R₁ is selected from the group consisting of deuterium, halogen, C1-C10 haloalkyl, and a combination of at least two selected therefrom; and
wherein in Formula (4), at least one of Z₁ to Z₈ is CR₁, and wherein R₁ is selected from the group consisting of deuterium, halogen, C1-C10 haloalkyl, and a combination of at least two selected therefrom.

10. The compound according to claim 9, wherein R is selected from the group consisting of the following groups: 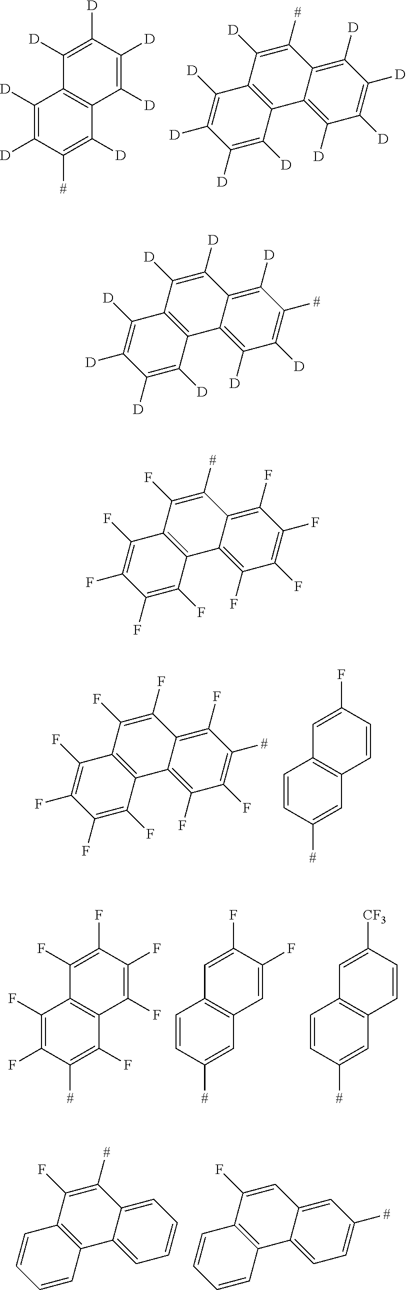 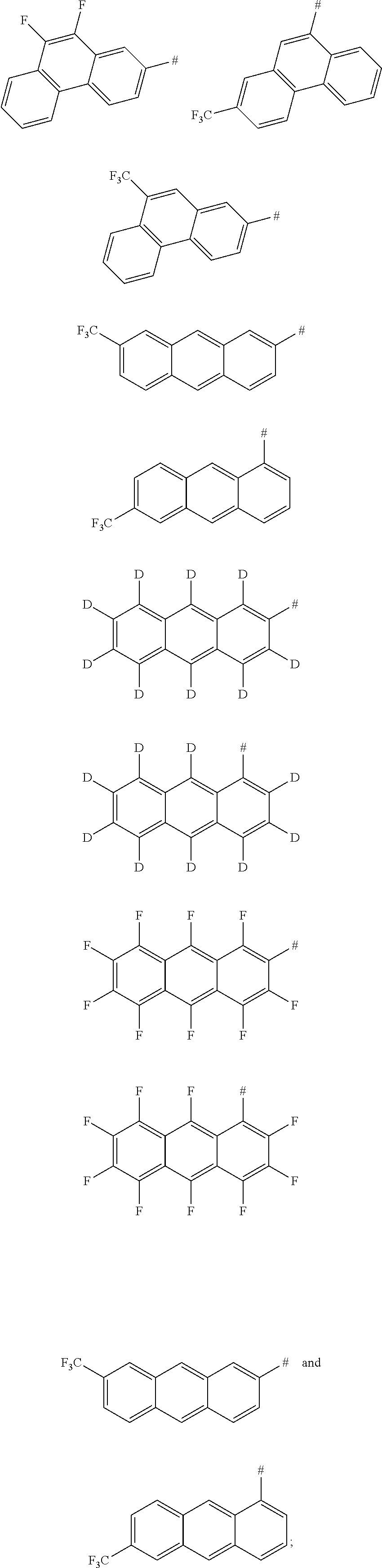
wherein # represents a linkage site of the group.

11. The compound according to claim 1, wherein each of X and Y is selected from substituted or unsubstituted C3-C60 electron withdrawing heteroaryl.

12. The compound according to claim 1, wherein each of X and Y is selected from the group consisting of the following substituted or unsubstituted groups: 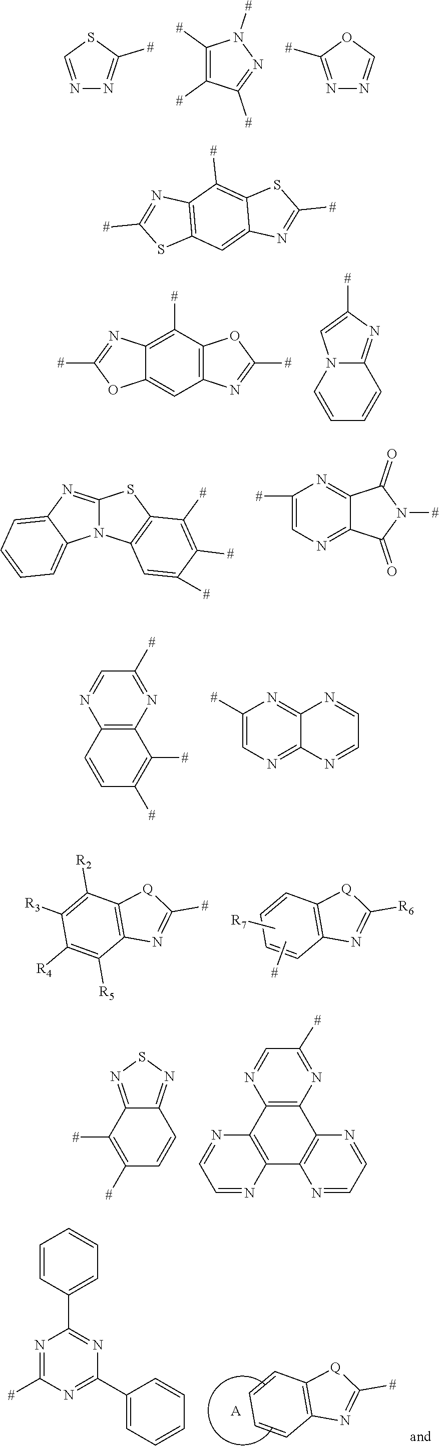 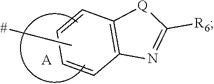
wherein # represents a linkage site of the group;
wherein Q is selected from the group consisting of an O atom, a S atom and NR₈;
wherein each of R₂ to R₇ is selected from the group consisting of hydrogen, protium, deuterium, tritium, halogen, C1-C10 alkyl, C1-C10 alkoxy, C6-C60 aryl, and C3-C60 heteroaryl; and
wherein the ring A is fused at any position of a benzene ring where the ring A is able to be fused, and wherein the ring A is selected from the group consisting of substituted or unsubstituted C6-C30 aromatic rings and substituted or unsubstituted C3-C30 heteroaromatic rings.

13. The compound according to claim 12, wherein each of X and Y is selected from the group consisting of the following groups: 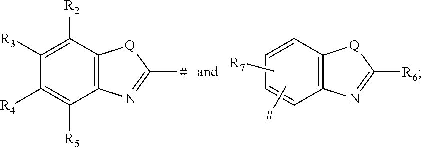
wherein # represents a linkage site of the group; and
wherein Q and R₂ to R₇ each have the same ranges as defined in claim 12.

14. The compound according to claim 13, wherein each of X and Y is selected from the group consisting of the following groups: 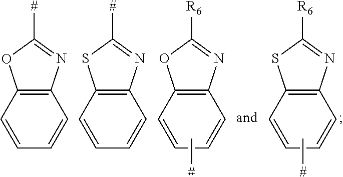
wherein # represents a linkage site of the group; and
wherein R₆ has the same range as defined in claim 13.

15. The compound according to claim 14, wherein Ar₁ is selected from the group consisting of substituted or unsubstituted C6-C60 arylene, substituted or unsubstituted C3-C60 heteroarylene, substituted or unsubstituted C10-C60 fused-ring arylene and substituted or unsubstituted C6-C60 fused-ring heteroarylene, and wherein X is selected from the group consisting of 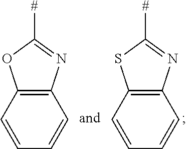
and/or
wherein Ar₂ is selected from the group consisting of substituted or unsubstituted C6-C60 arylene, substituted or unsubstituted C3-C60 heteroarylene, substituted or unsubstituted C10-C60 fused-ring arylene and substituted or unsubstituted C6-C60 fused-ring heteroarylene, and wherein Y is selected from the group consisting of 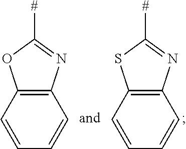
wherein # represents a linkage site of the group.

16. The compound according to claim 14, wherein Ar₁ is selected from a single bond, and wherein X is selected from the group consisting of 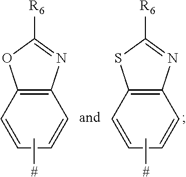
and/or wherein Ar₂ is selected from a single bond, and Y is selected from the group consisting of 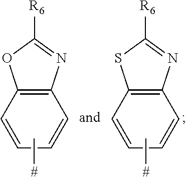
wherein R₆ has the same range as defined in claim 14; and
wherein # represents a linkage site of the group.

17. The compound according to claim 1, wherein each of Ar₁ and Ar₂ is selected from the group consisting of phenylene, biphenylene, terphenylene, naphthylene, anthrylene, phenanthrylene, pyridinylene, pyrimidinylene, triazinylene, furylene, pyrrolidene, thienylene, quinolylene, isoquinolylene, benzofurylene and benzothienylene.

18. The compound according to claim 15, wherein each of Ar₁ and Ar₂ is selected from

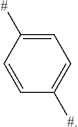

19. The compound according to claim 1, wherein each of Ar₁ and Ar₂ is selected from the group consisting of naphthylene and biphenylene.

20. The compound according to claim 19, wherein each of Ar₁ and Ar₂ is selected from the group consisting of 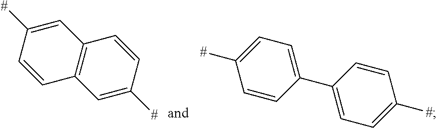
wherein # represents a linkage site of the group.

21. The compound according to claim 1, wherein the compound has a structure represented by Formula (2): 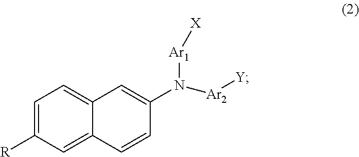
wherein R, Ar₁, Ar₂, X and Y each have the same ranges as defined in Formula (1).

22. The compound according to claim 1, wherein at least one of P₁, P₂, P₃, P₄, P₅ and P₆ is selected from a deuterium atom.

23. The compound according to claim 22, wherein each of P₂, P₃, P₅ and P₆ is selected from a deuterium atom.

24. The compound according to claim 1, wherein the difference between the refractive index of the compound at a wavelength of 460 nm and the refractive index of the compound at a wavelength of 530 nm is 0.10-0.17, wherein the difference between the refractive index of the compound at a wavelength of 530 nm and the refractive index of the compound at a wavelength of 620 nm is 0.03-0.10, and wherein the difference between the refractive index of the compound at a wavelength of 460 nm and the refractive index of the compound at a wavelength of 620 nm is 0.15-0.40.

25. The compound according to claim 1, wherein a film with a thickness of 700 angstroms and prepared from the compound has an absorbance greater than 0.3 at a wavelength of 250 nm and at a wavelength of 380 nm.

26. The compound according to claim 1, wherein the compound has any one of the following structures represented by P1 to P305:

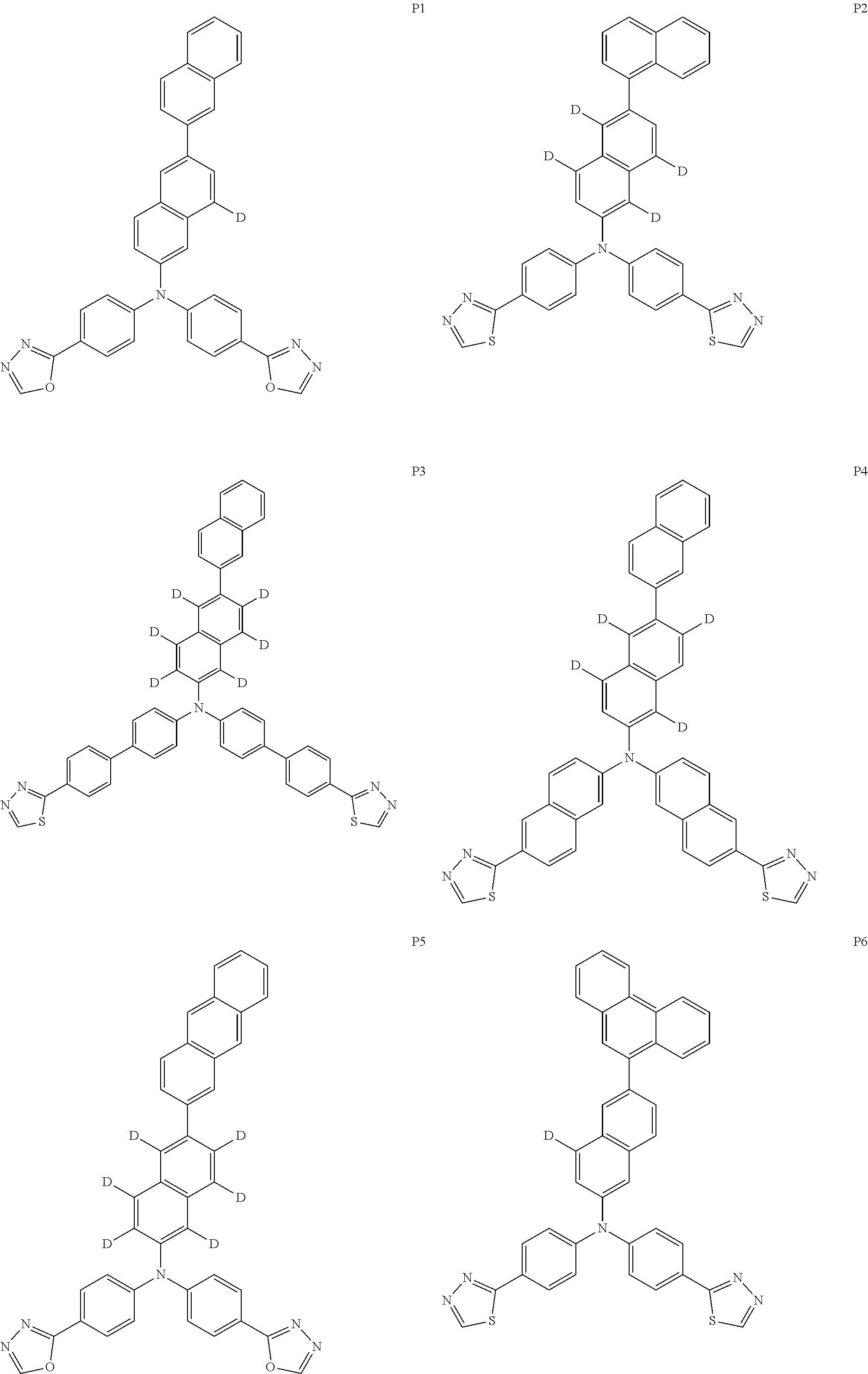

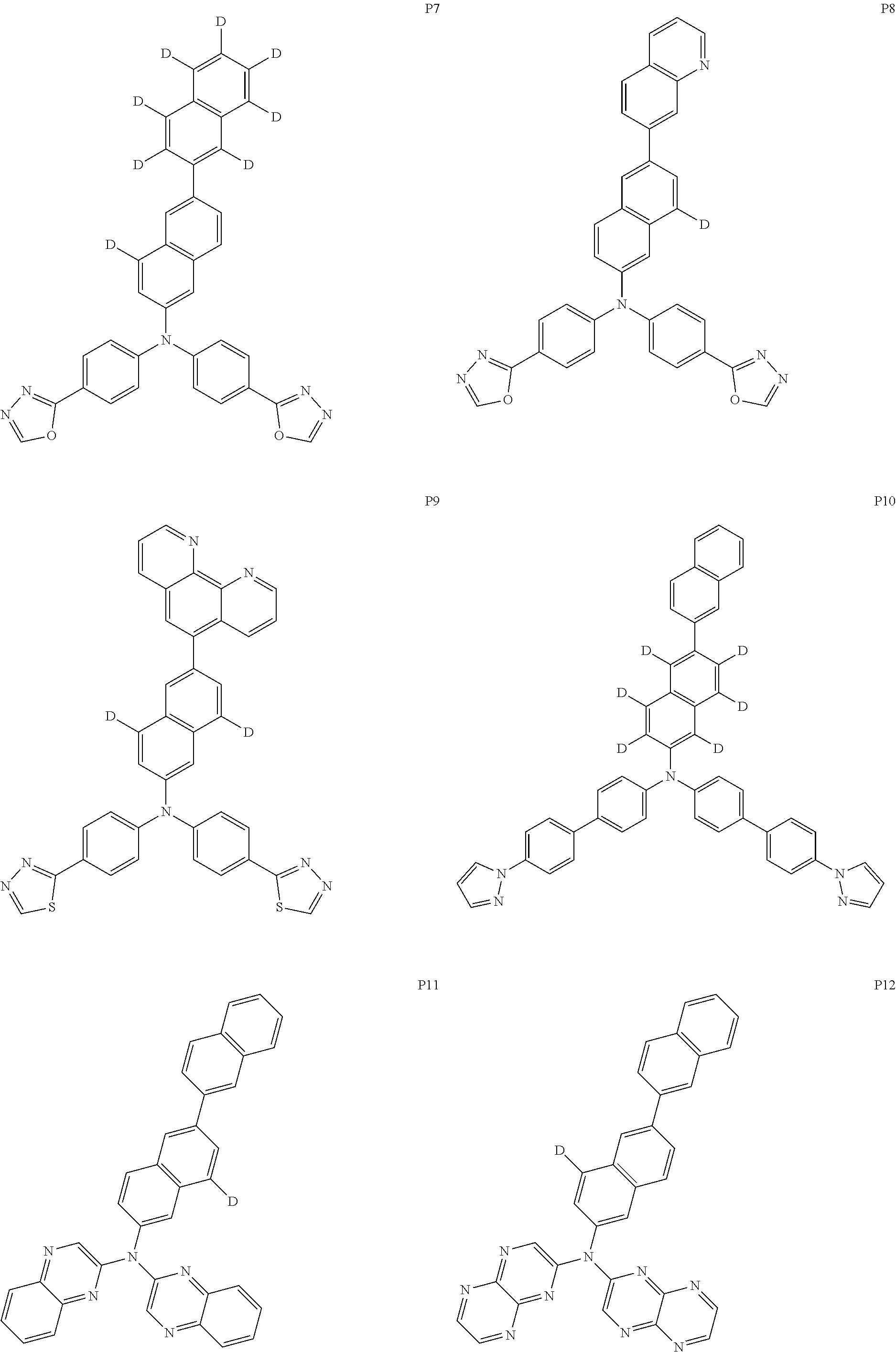

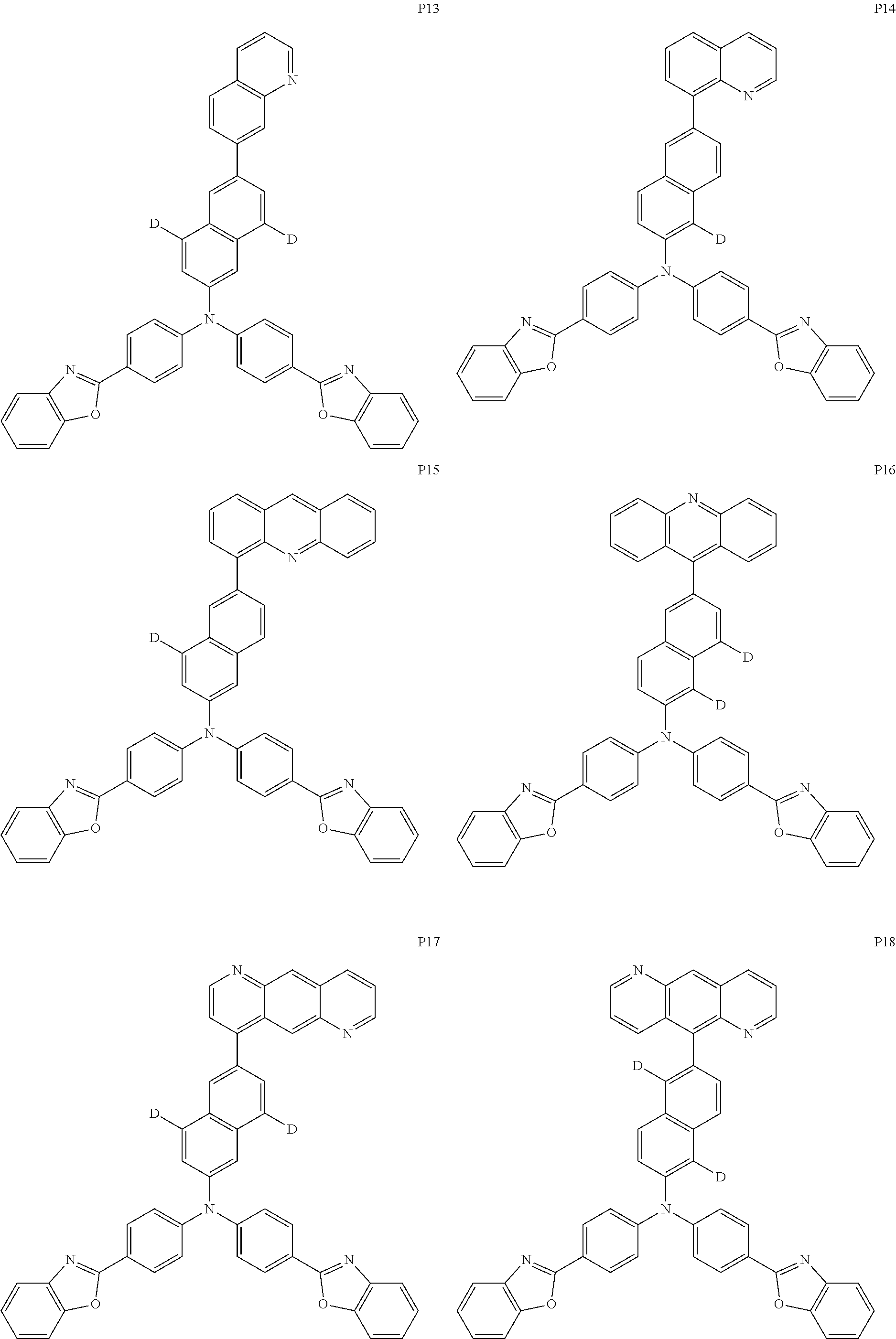

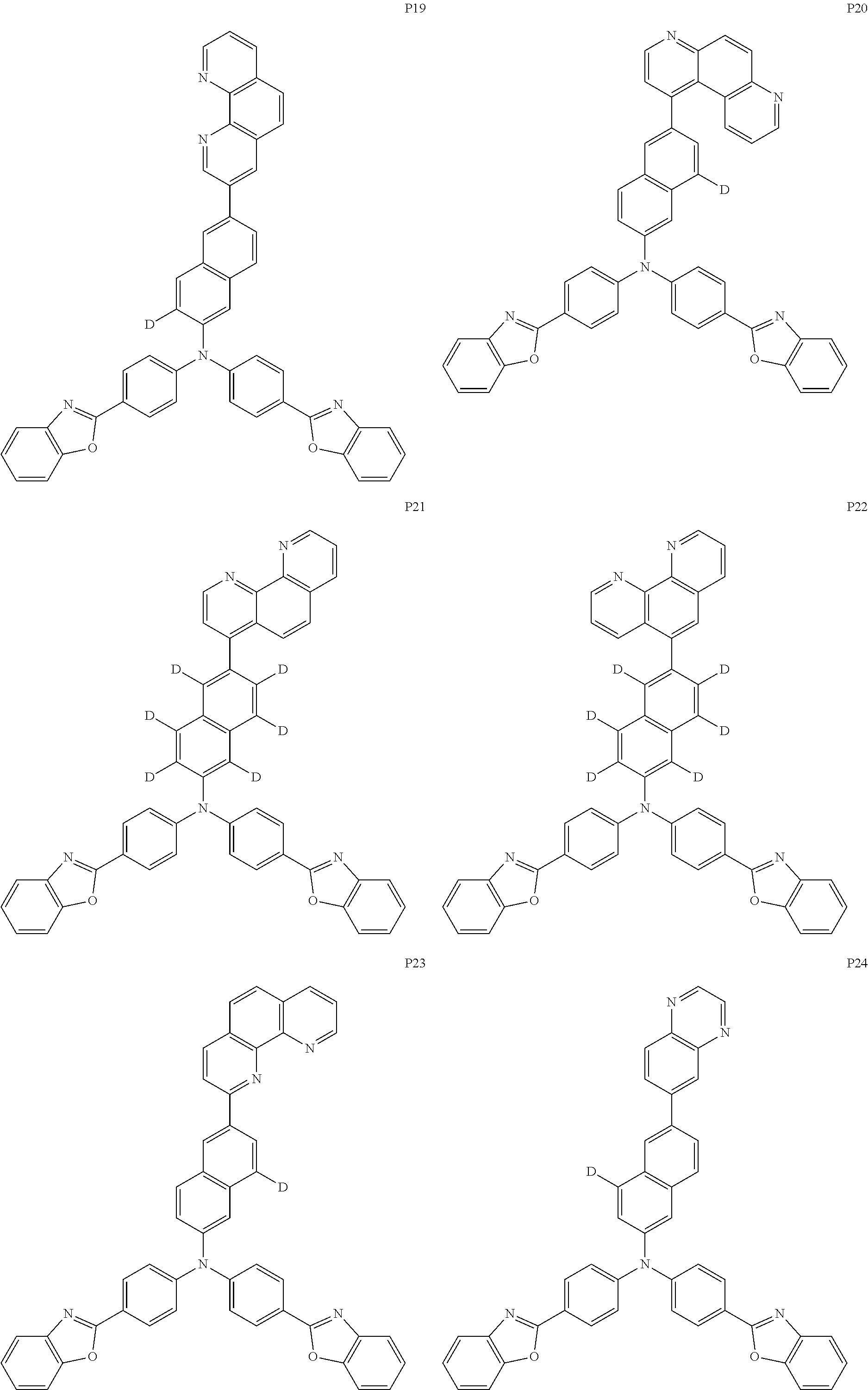

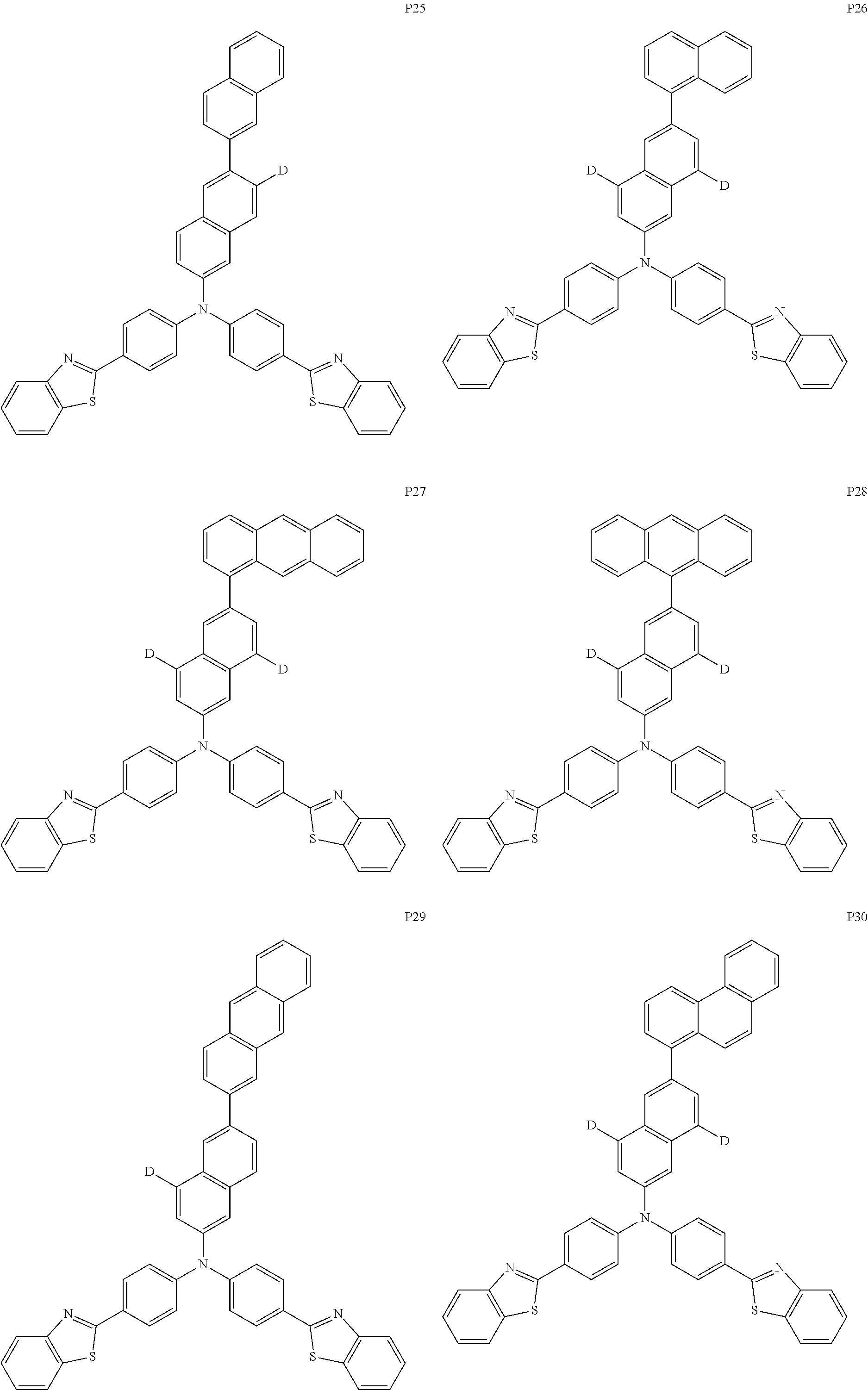

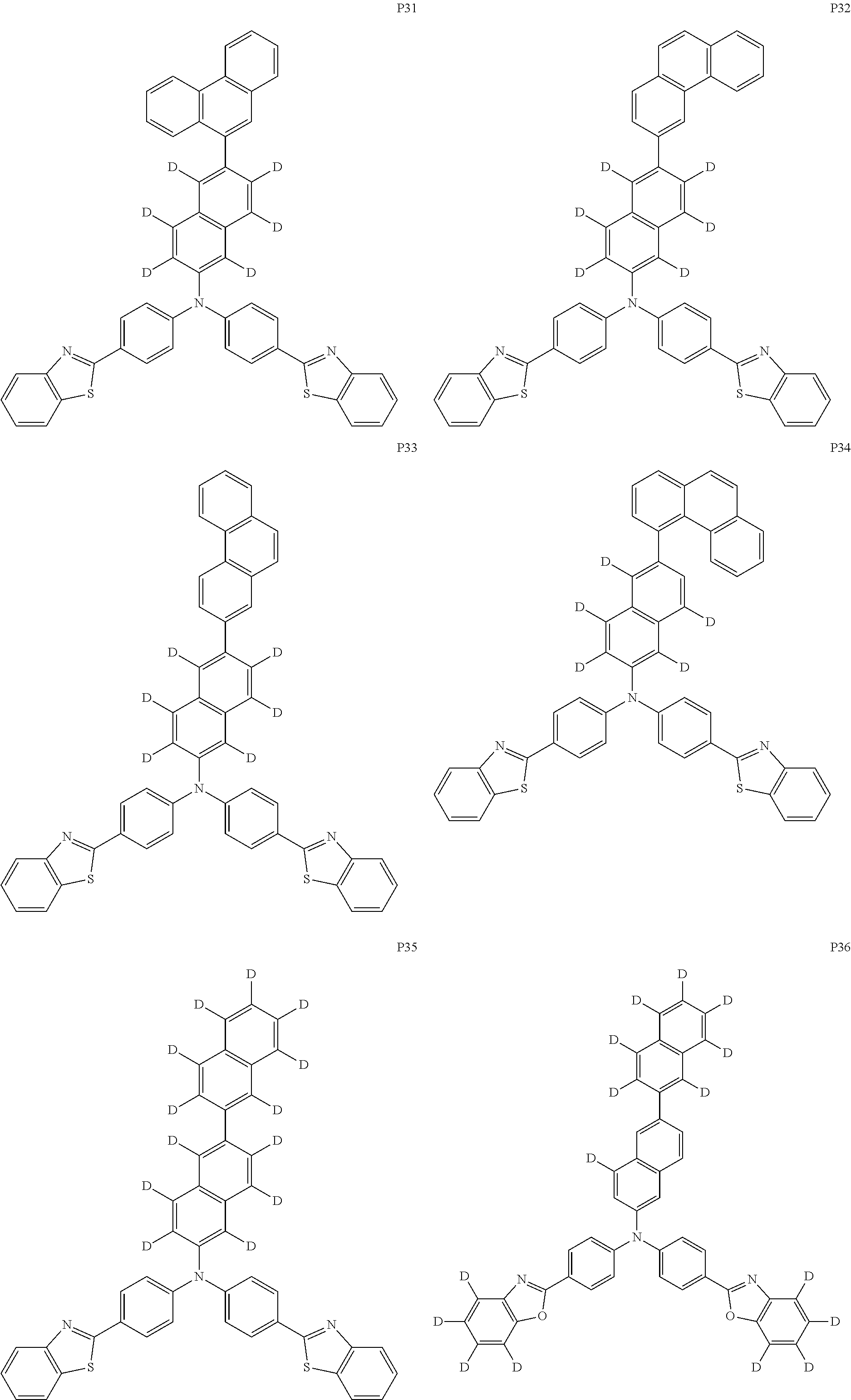

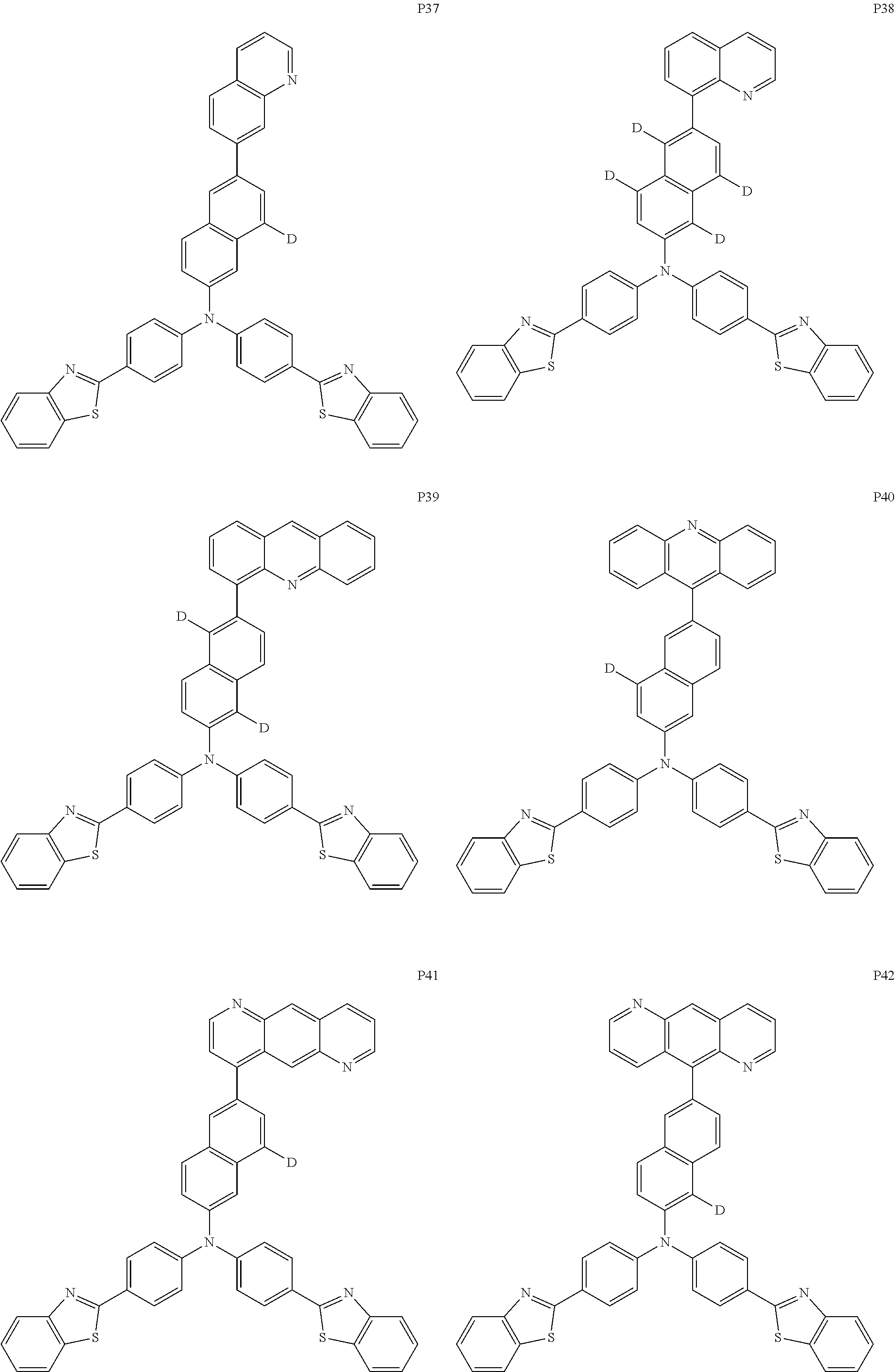

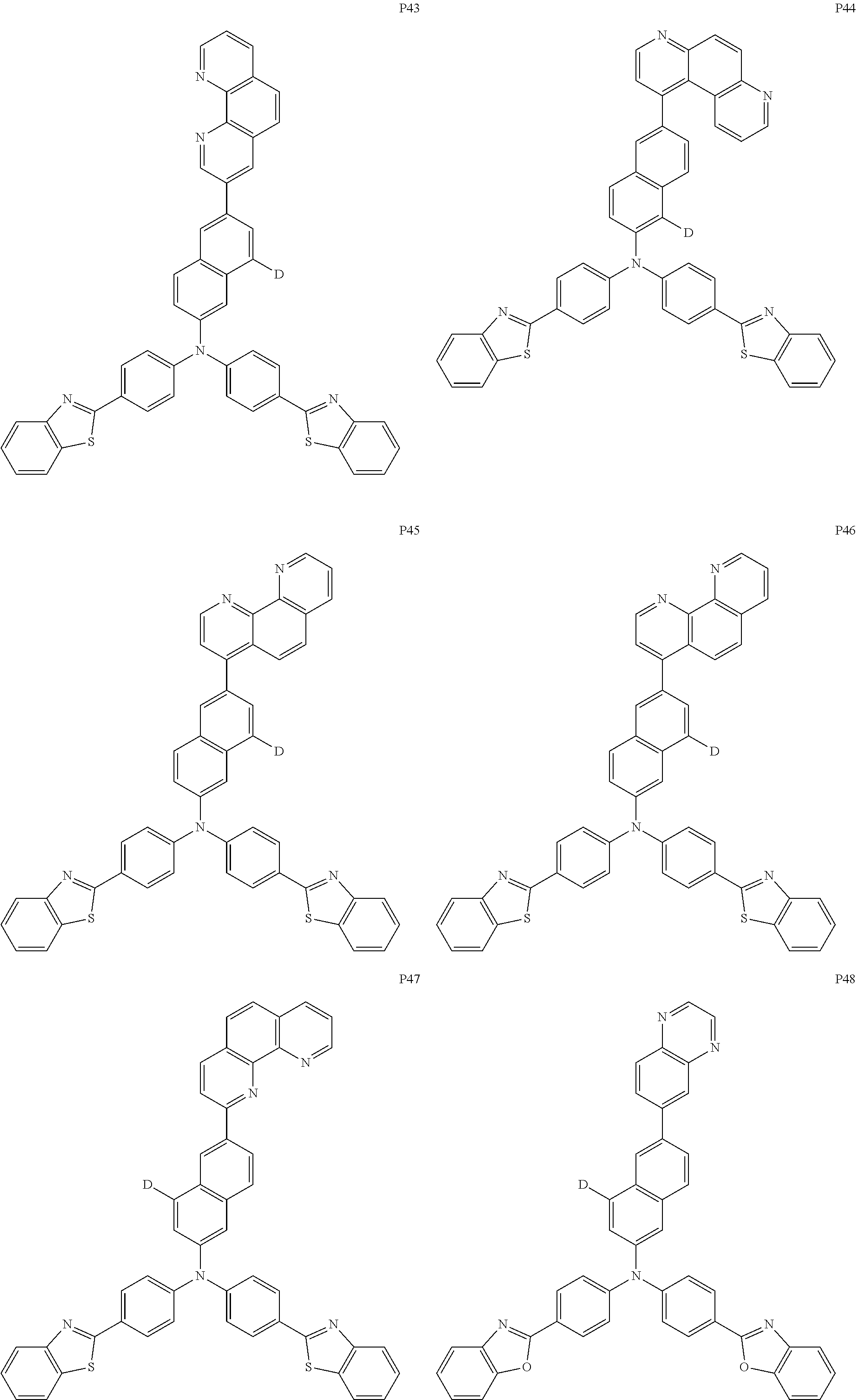

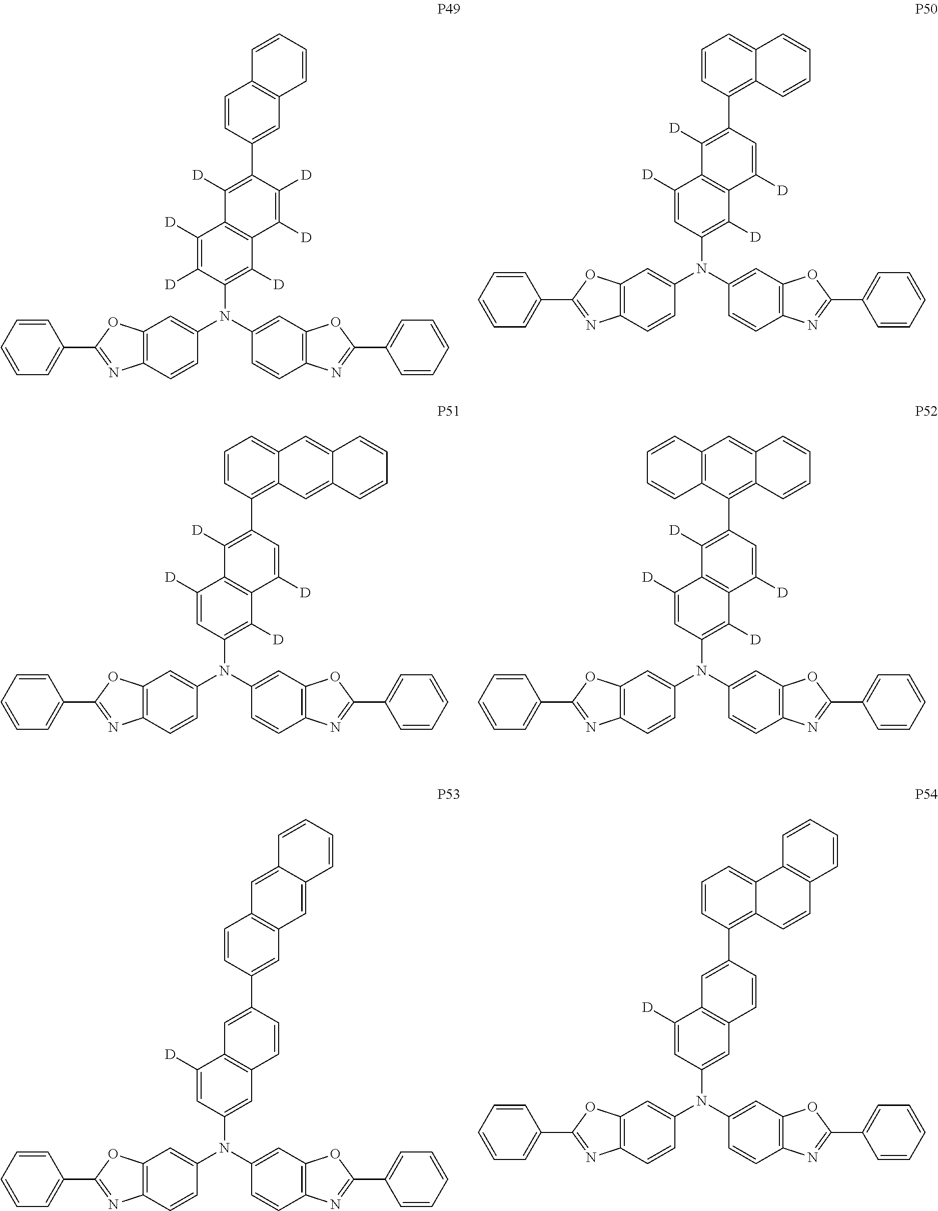

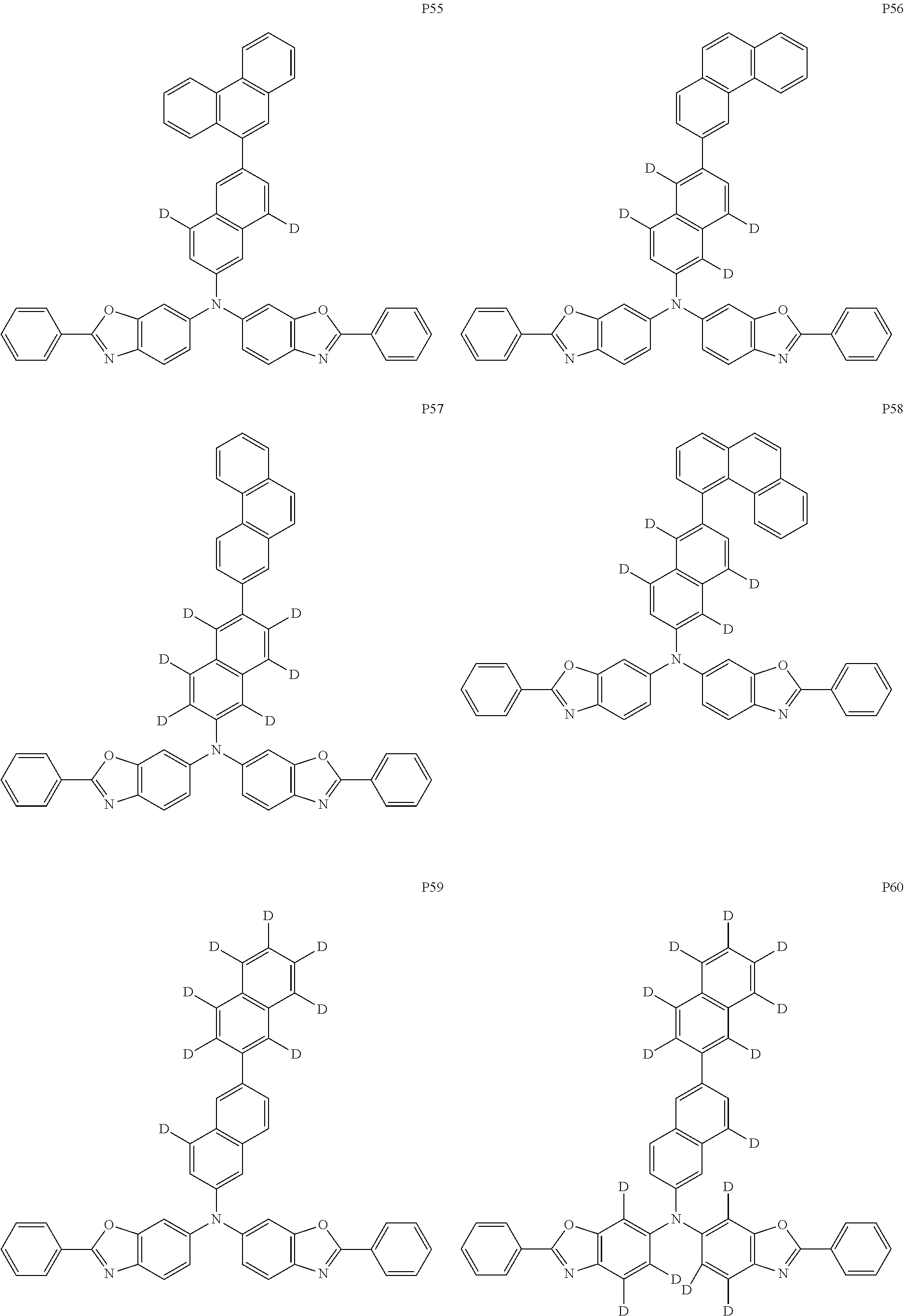

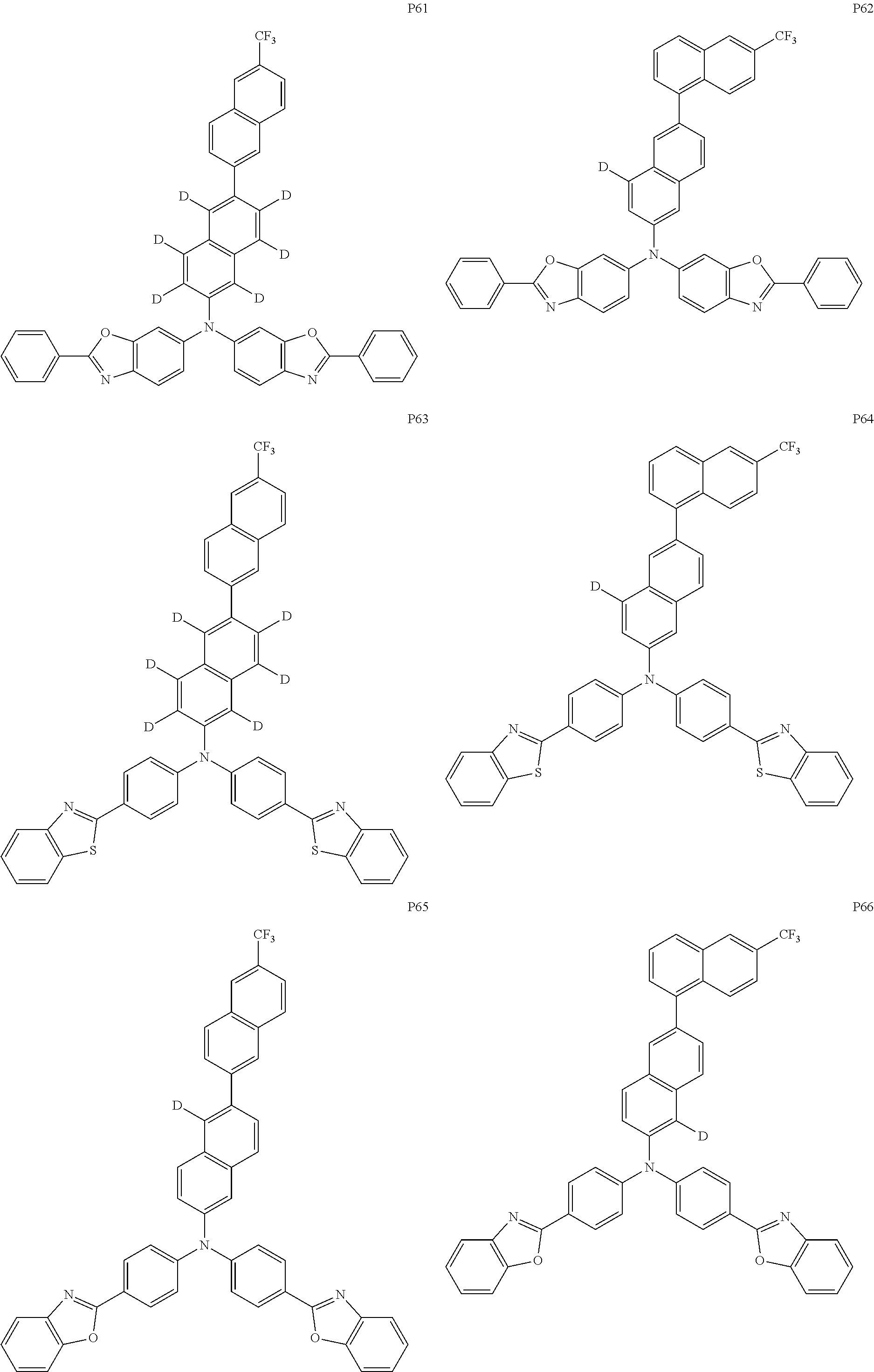

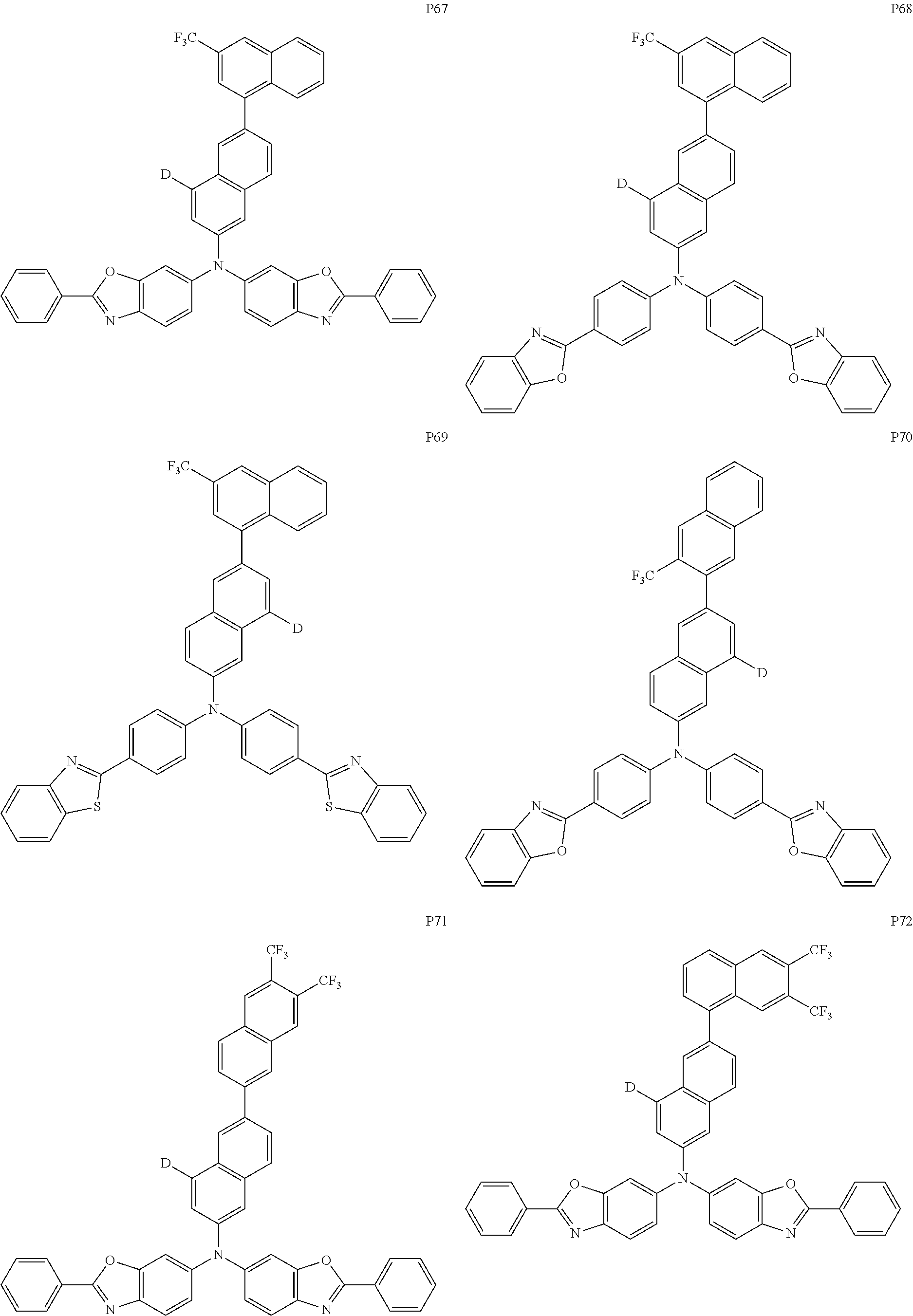

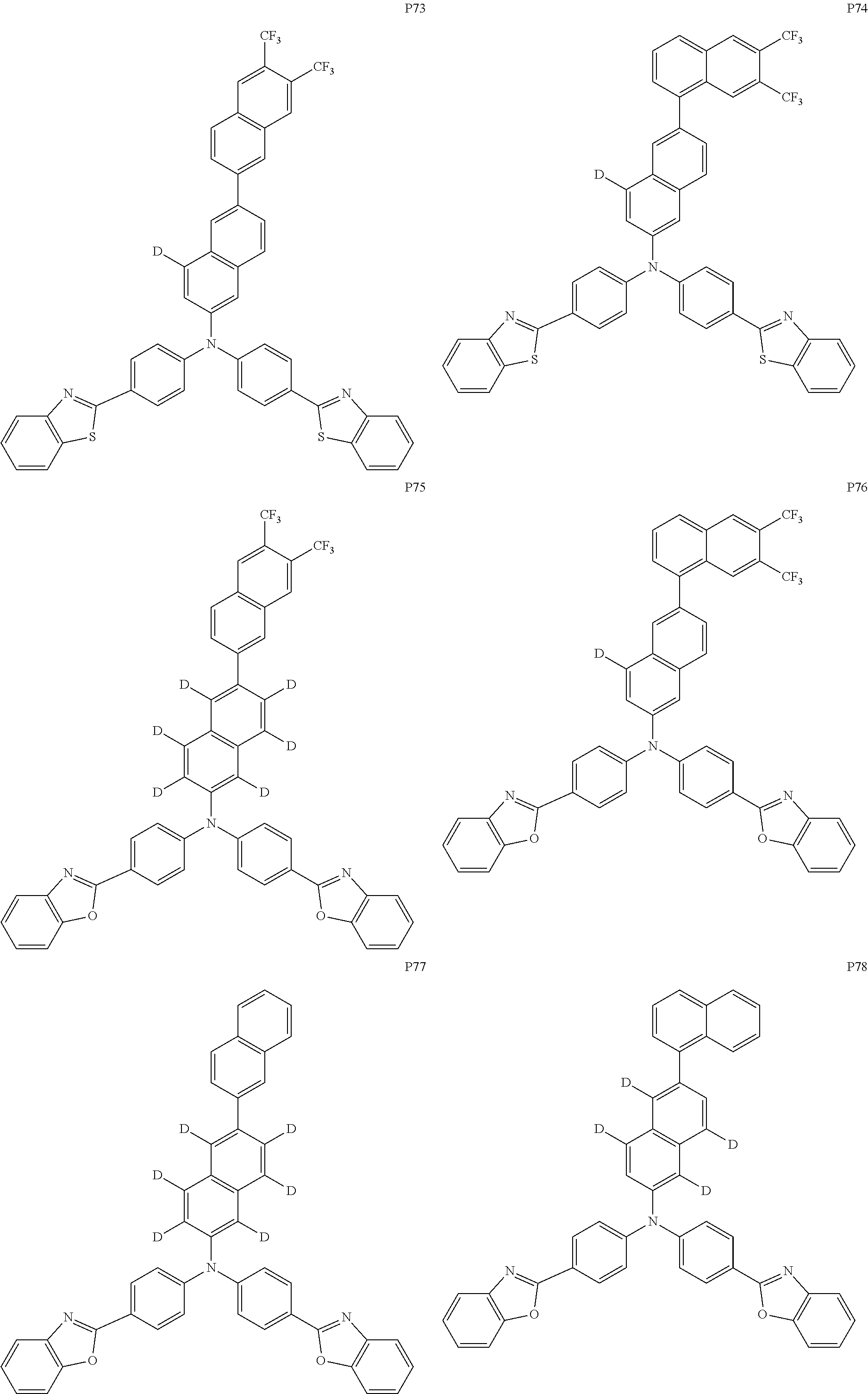

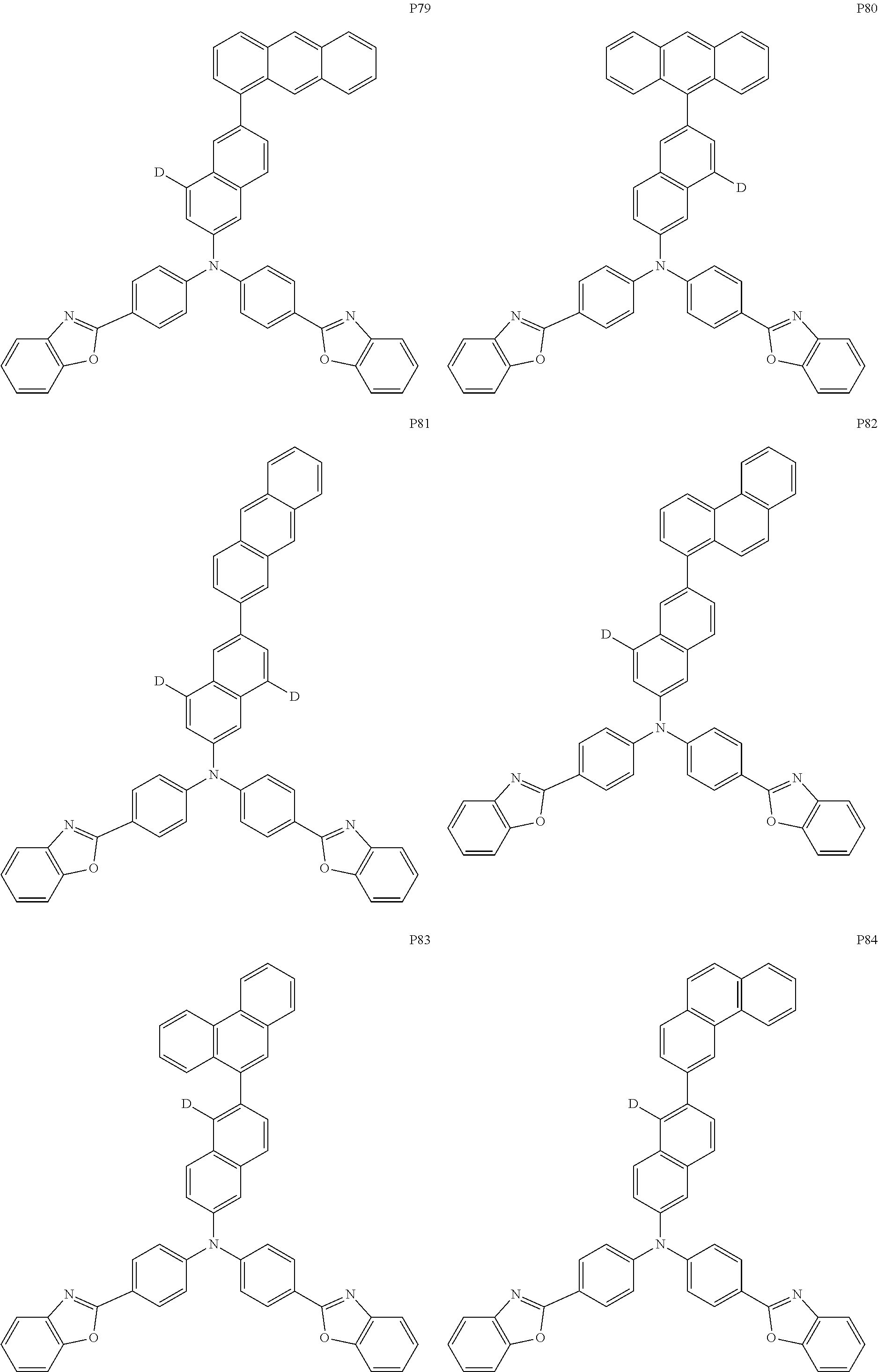

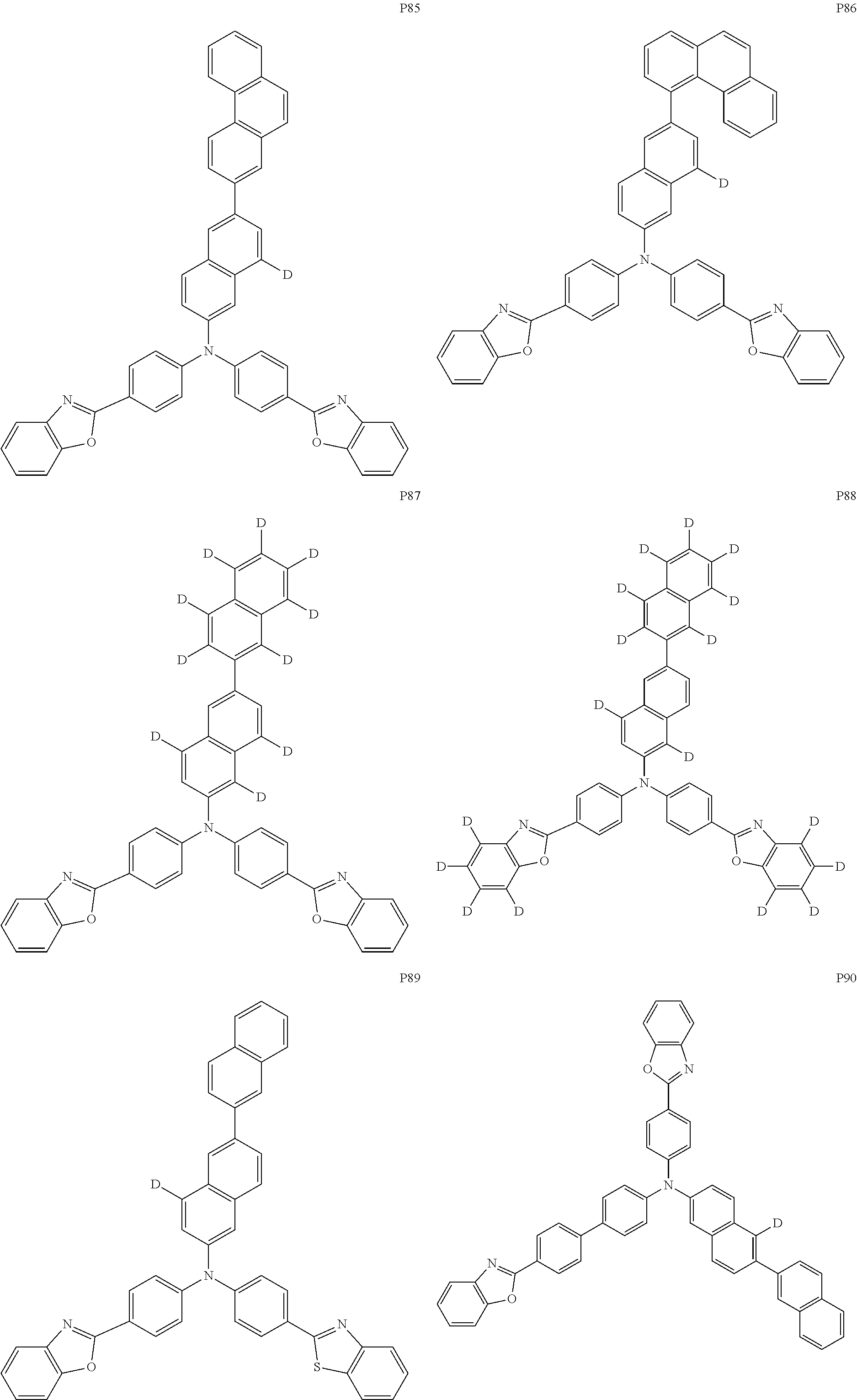

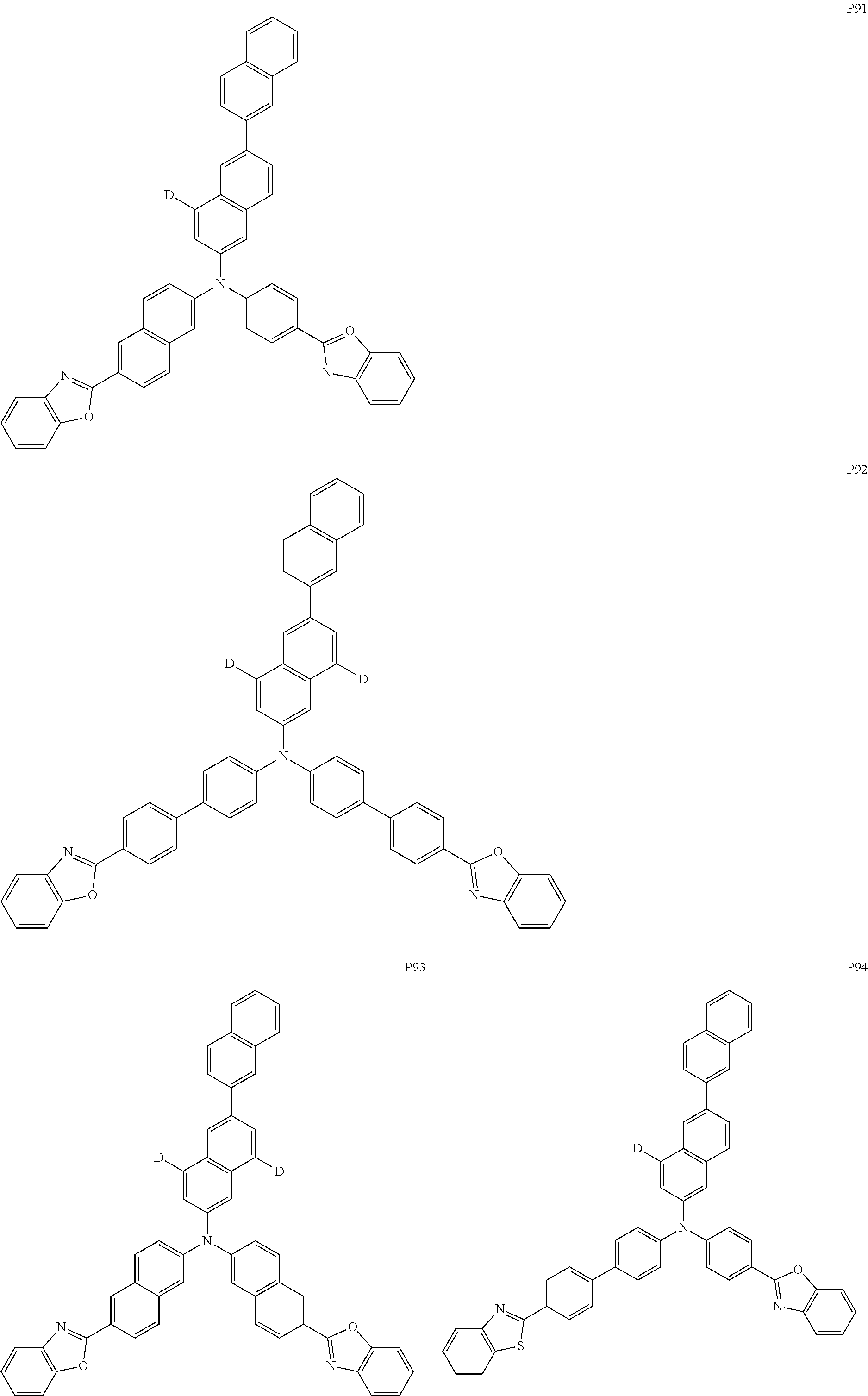

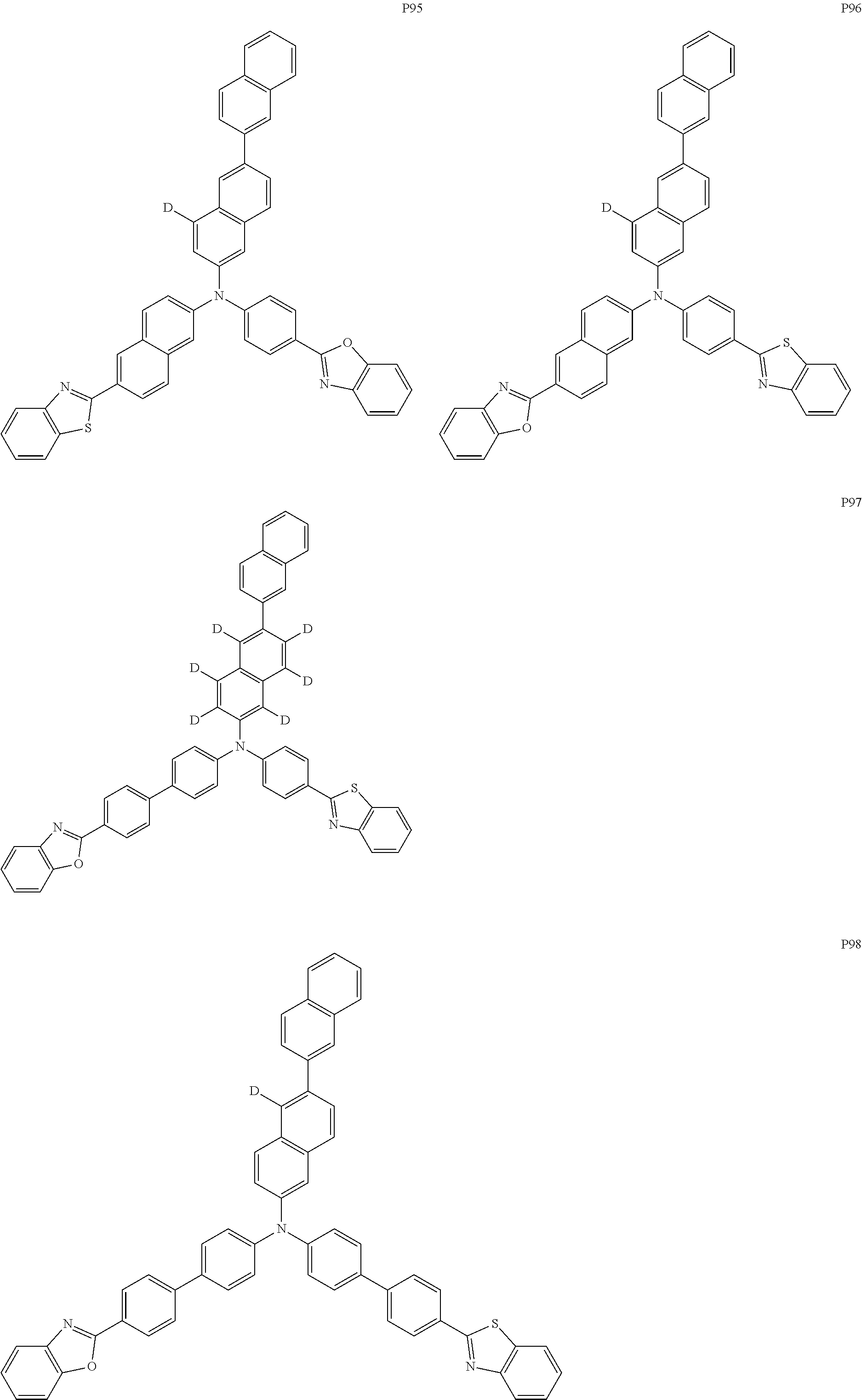 [SMILES: [2H]c1cc(N(c2ccc(-c3nc4ccccc4o3)cc2)c2ccc3cc(-c4nc5ccccc5s4)ccc3c2)cc2ccc(-c3ccc4ccccc4c3)cc12 ; [2H]c1cc(N(c2ccc(-c3nc4ccccc4s3)cc2)c2ccc3cc(-c4nc5ccccc5o4)ccc3c2)cc2ccc(-c3ccc4ccccc4c3)cc12 ; [2H]c1c(N(c2ccc(-c3ccc(-c4nc5ccccc5o4)cc3)cc2)c2ccc(-c3nc4ccccc4s3)cc2)c([2H])c2c([2H])c([2H])c(-c3ccc4ccccc4c3)c([2H])c2c1[2H] ; [2H]c1c(-c2ccc3ccccc3c2)ccc2cc(N(c3ccc(-c4ccc(-c5nc6ccccc6o5)cc4)cc3)c3ccc(-c4ccc(-c5nc6ccccc6s5)cc4)cc3)ccc12]

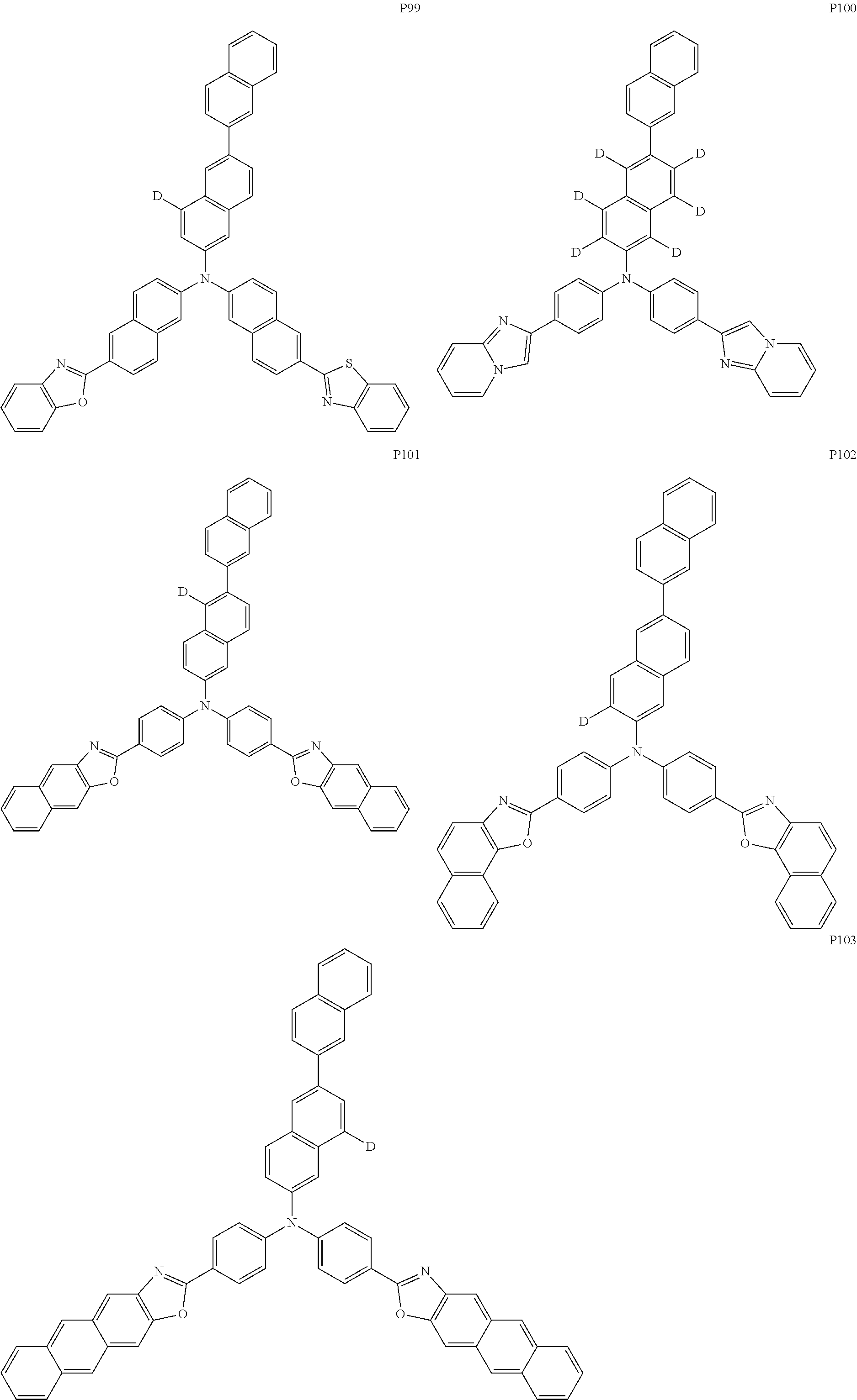

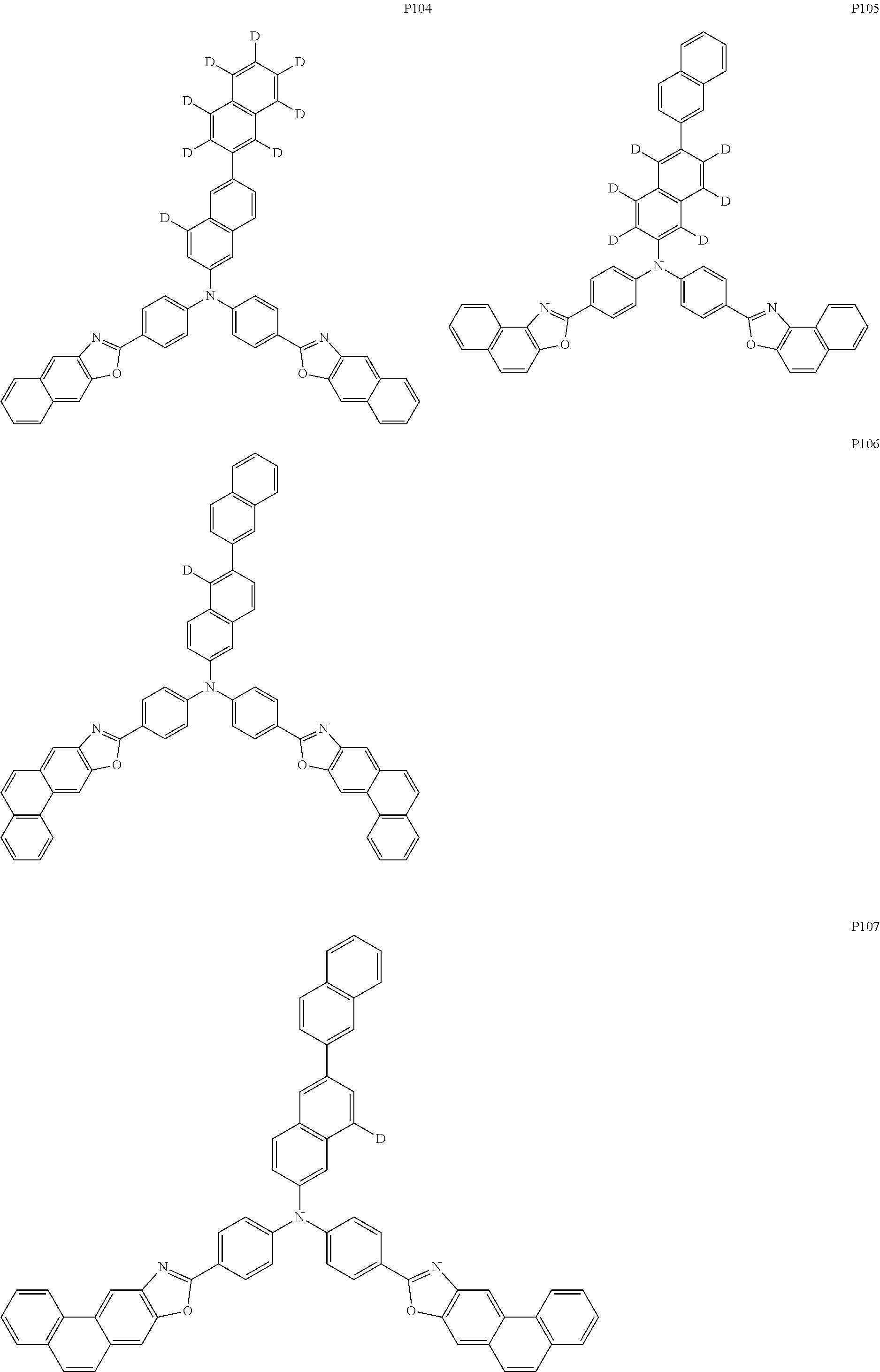

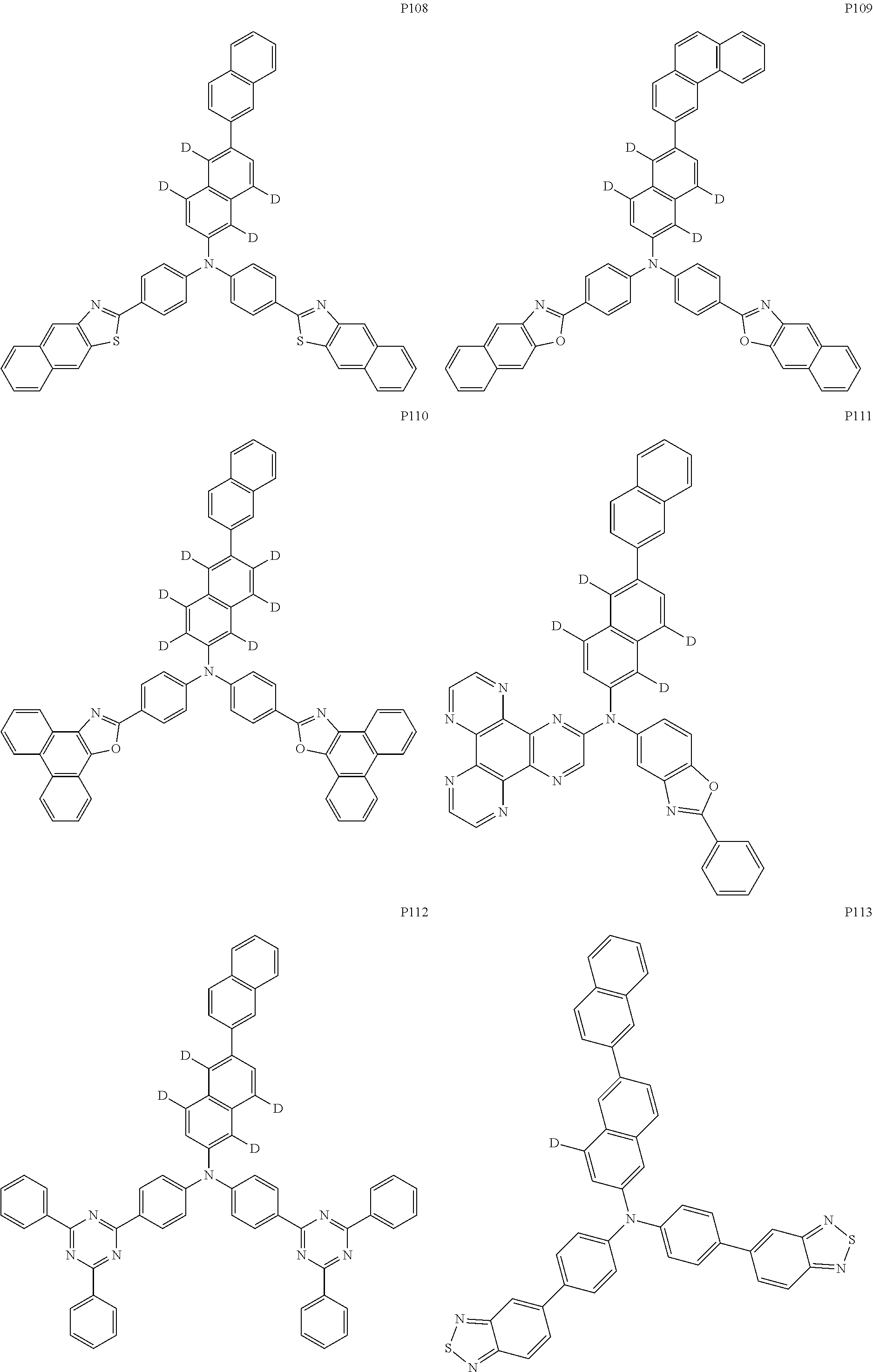

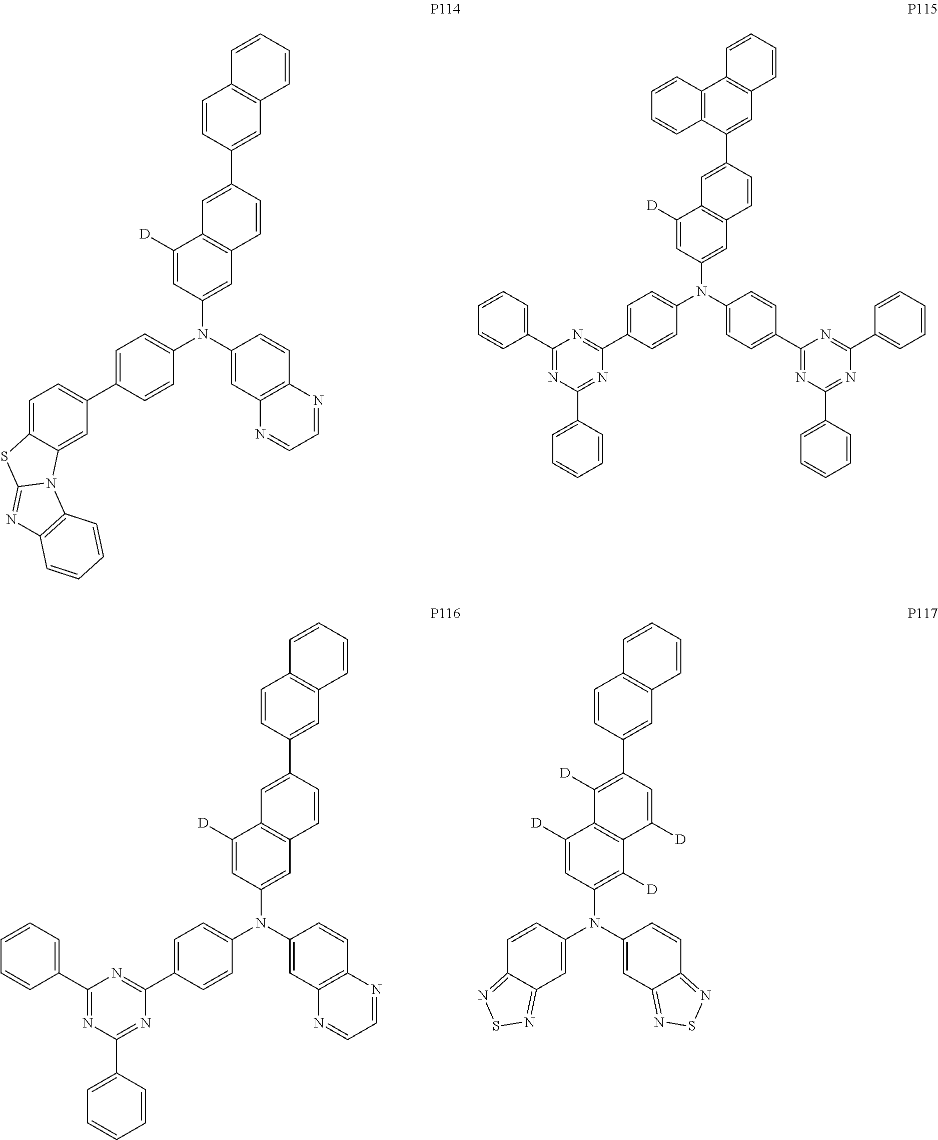 [SMILES: [2H]c1cc(N(c2ccc(-c3ccc4sc5nc6ccccc6n5c4c3)cc2)c2ccc3nccnc3c2)cc2ccc(-c3ccc4ccccc4c3)cc12 ; [2H]c1cc(N(c2ccc(-c3nc(-c4ccccc4)nc(-c4ccccc4)n3)cc2)c2ccc(-c3nc(-c4ccccc4)nc(-c4ccccc4)n3)cc2)cc2ccc(-c3cc4ccccc4c4ccccc34)cc12 ; [2H]c1cc(N(c2ccc(-c3nc(-c4ccccc4)nc(-c4ccccc4)n3)cc2)c2ccc3nccnc3c2)cc2ccc(-c3ccc4ccccc4c3)cc12 ; [2H]c1cc(N(c2ccc3nsnc3c2)c2ccc3nsnc3c2)c([2H])c2c([2H])cc(-c3ccc4ccccc4c3)c([2H])c12]

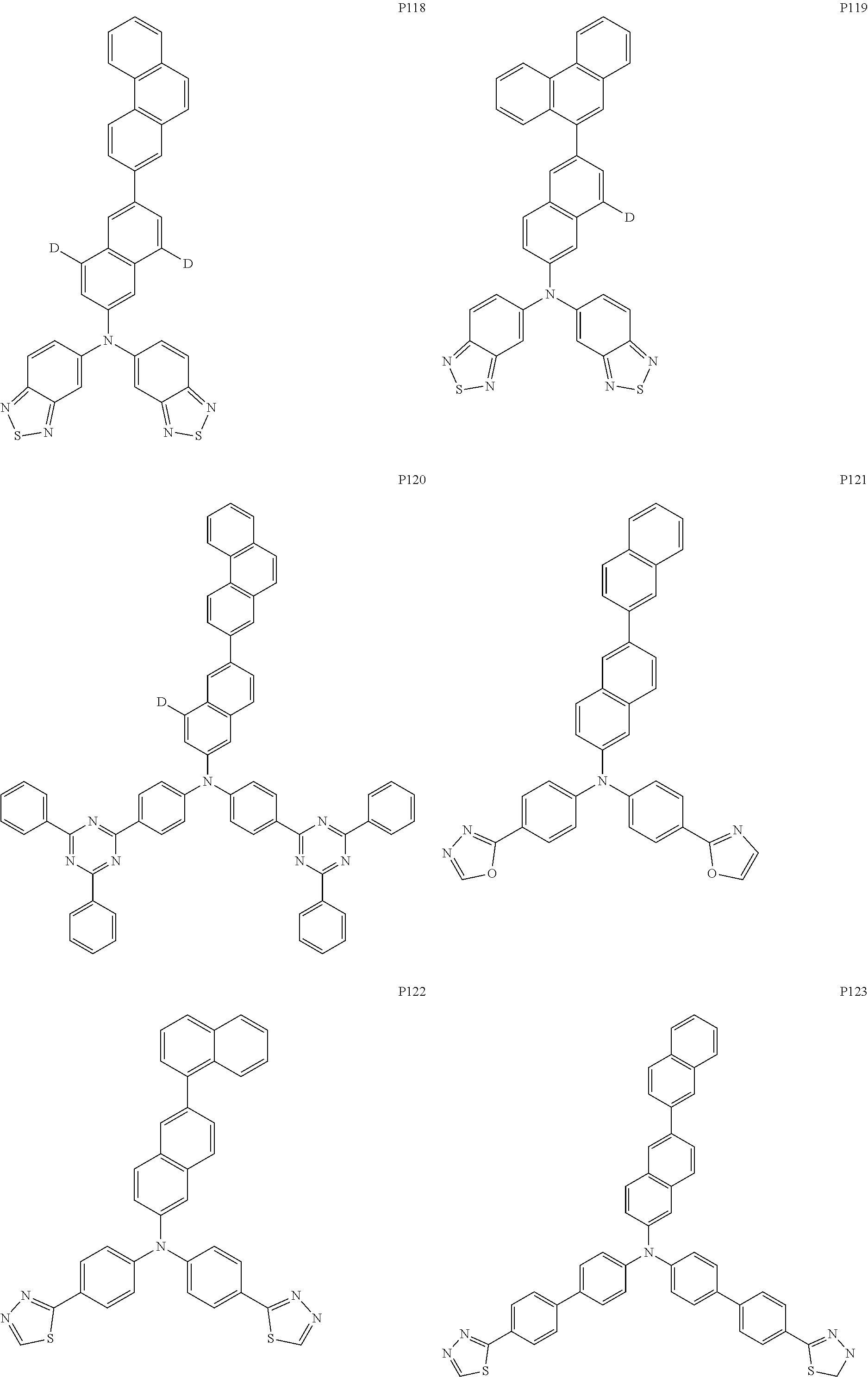

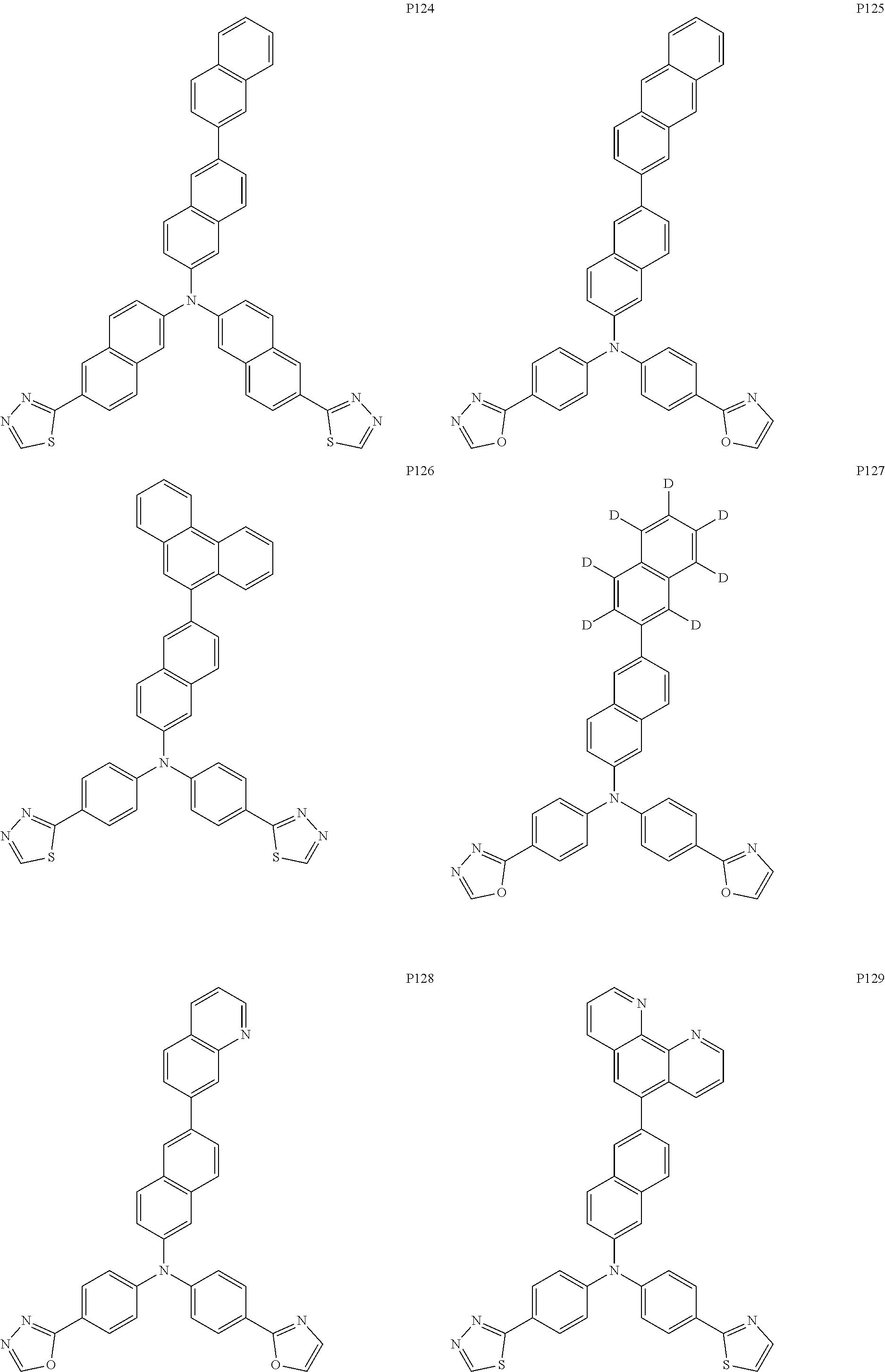

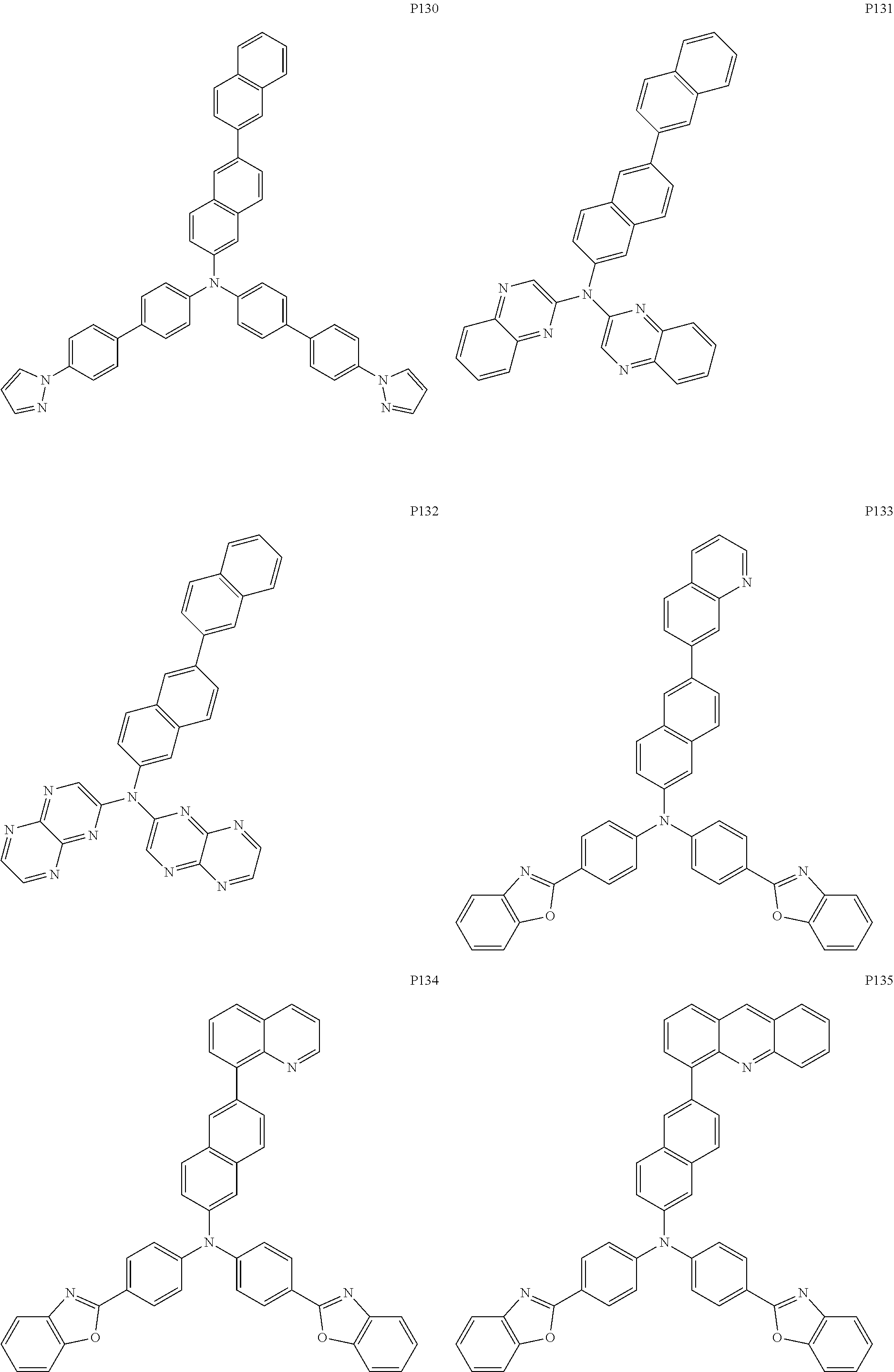

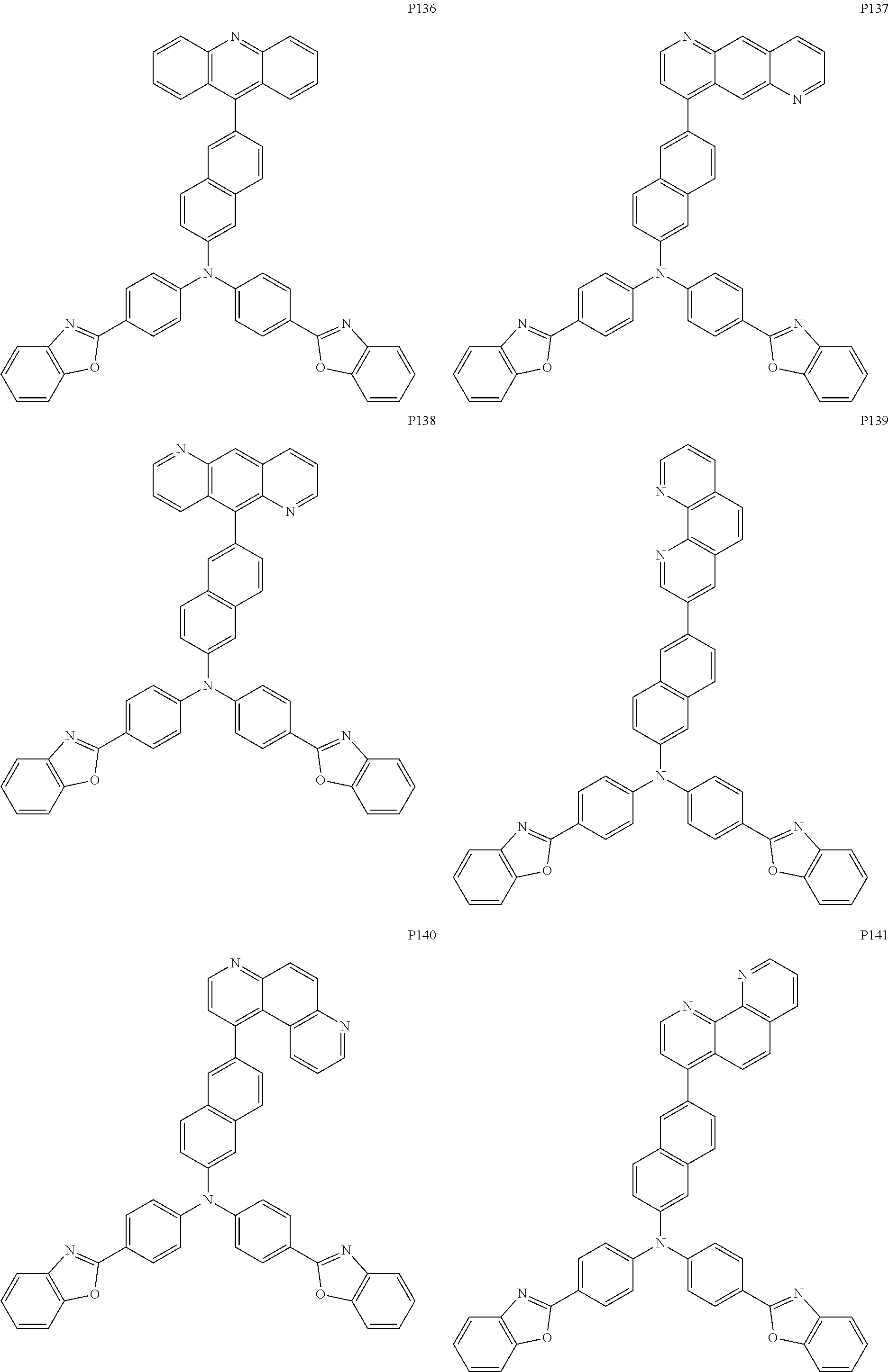

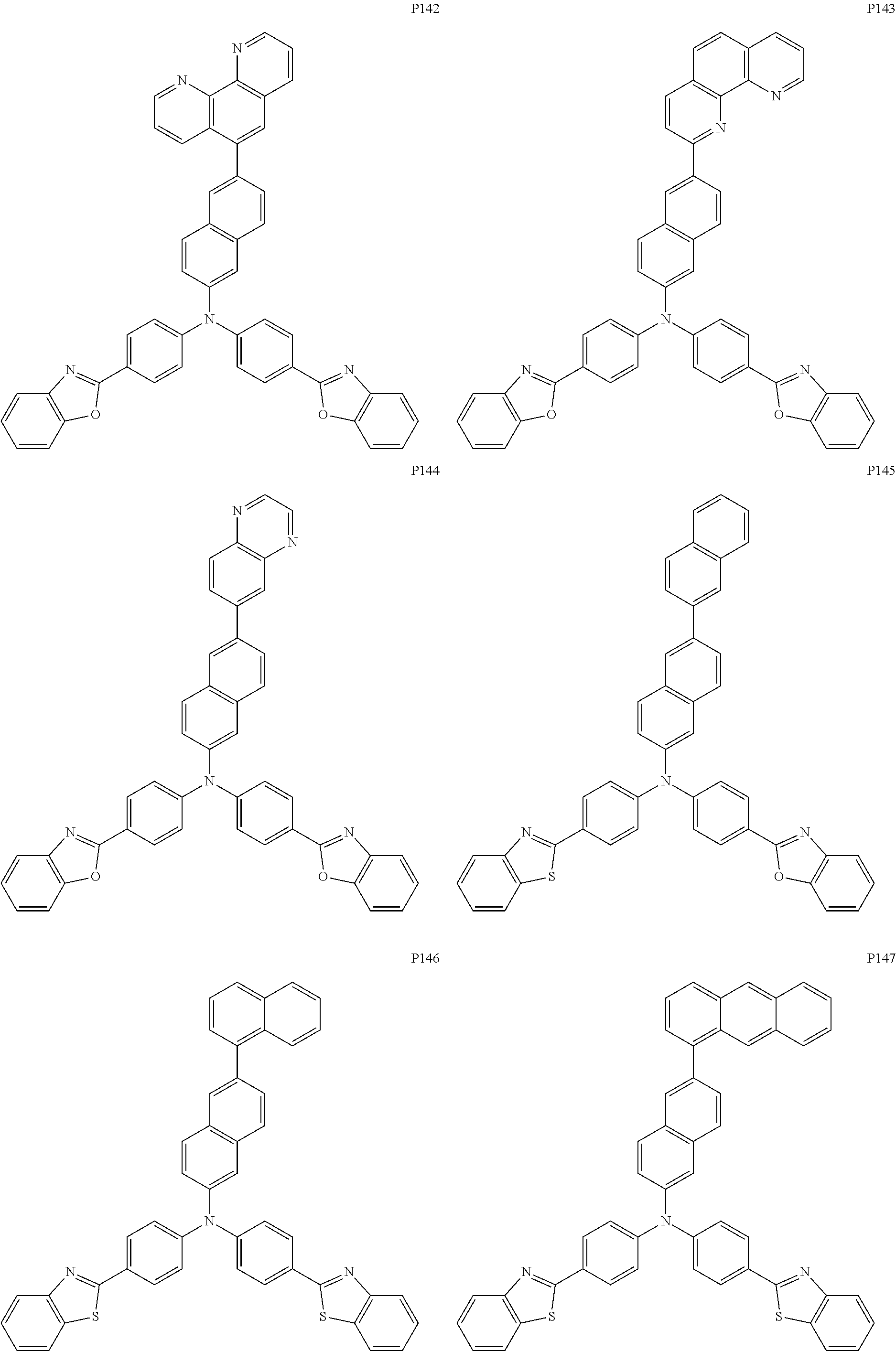

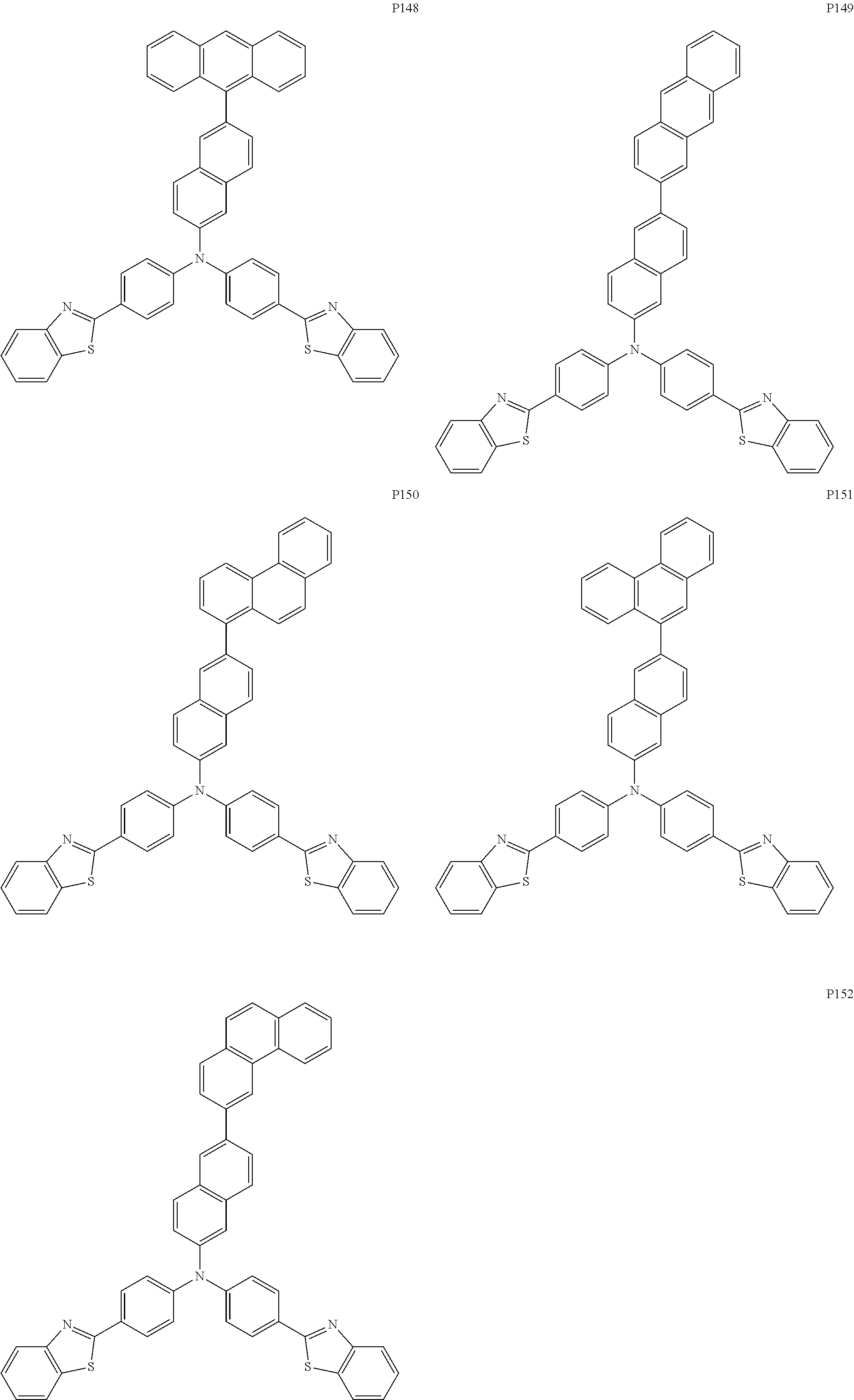

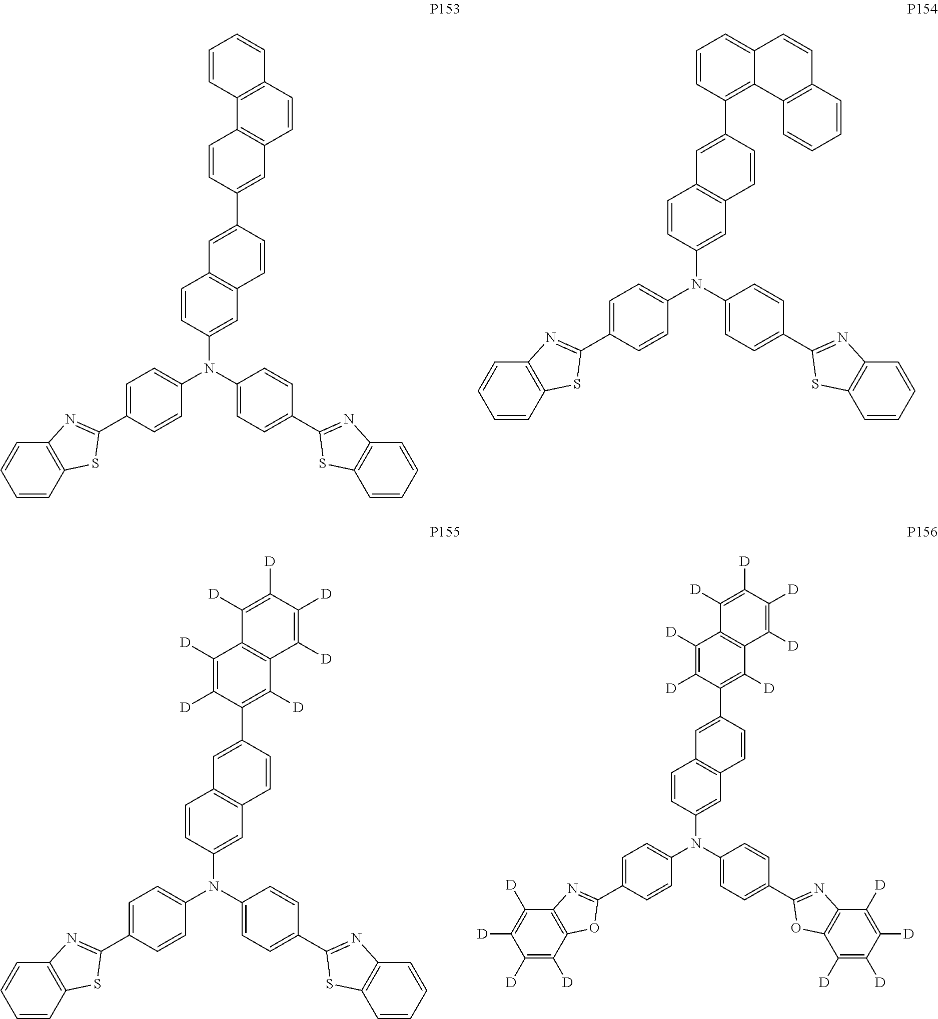 [SMILES: c1ccc2c(c1)ccc1cc(-c3ccc4cc(N(c5ccc(-c6nc7ccccc7s6)cc5)c5ccc(-c6nc7ccccc7s6)cc5)ccc4c3)ccc12 ; c1ccc2c(c1)ccc1cccc(-c3ccc4cc(N(c5ccc(-c6nc7ccccc7s6)cc5)c5ccc(-c6nc7ccccc7s6)cc5)ccc4c3)c12 ; [2H]c1c([2H])c([2H])c2c([2H])c(-c3ccc4cc(N(c5ccc(-c6nc7ccccc7s6)cc5)c5ccc(-c6nc7ccccc7s6)cc5)ccc4c3)c([2H])c([2H])c2c1[2H] ; [2H]c1c([2H])c([2H])c2oc(-c3ccc(N(c4ccc(-c5nc6c([2H])c([2H])c([2H])c([2H])c6o5)cc4)c4ccc5cc(-c6c([2H])c([2H])c7c([2H])c([2H])c([2H])c([2H])c7c6[2H])ccc5c4)cc3)nc2c1[2H]]

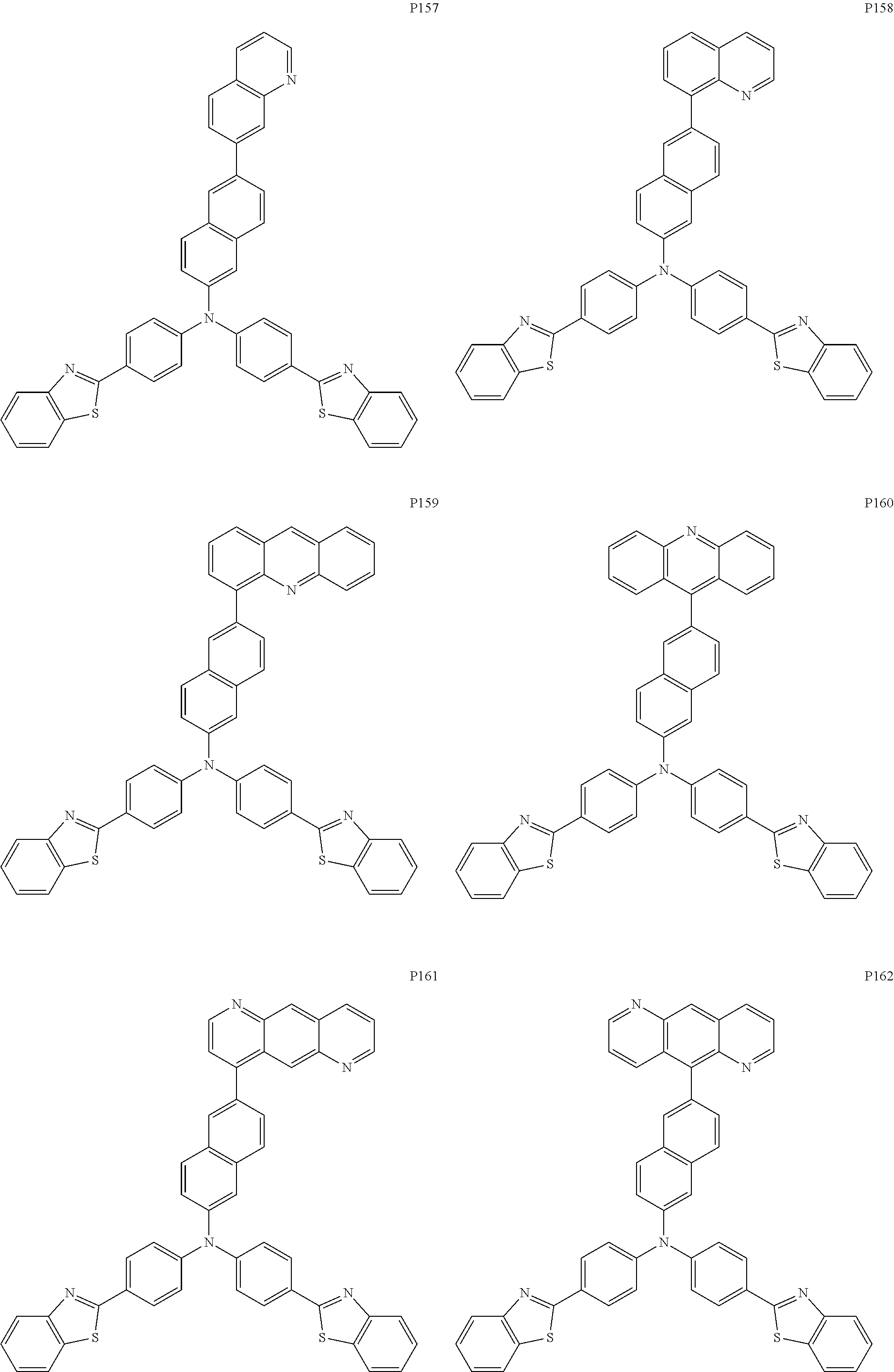

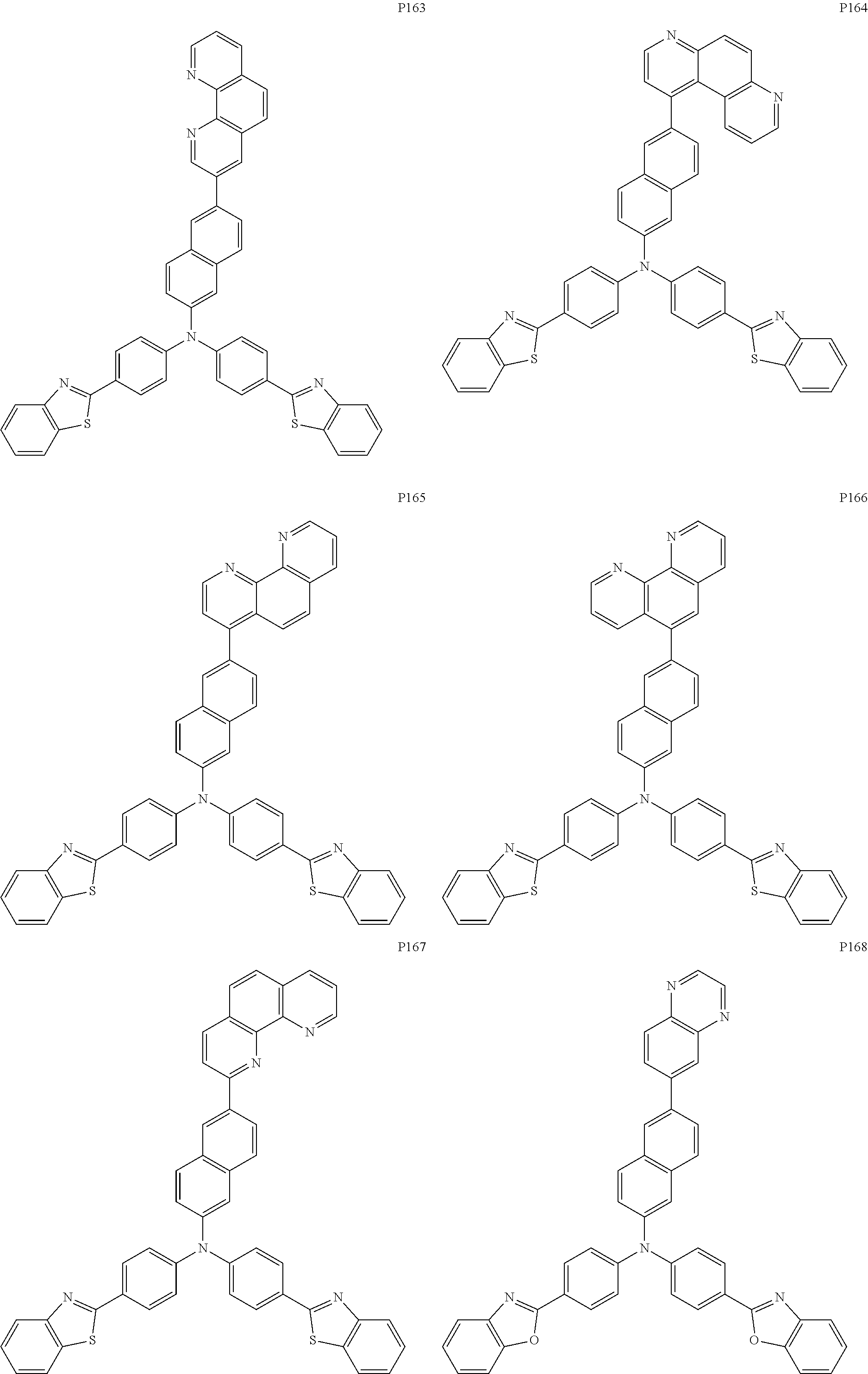

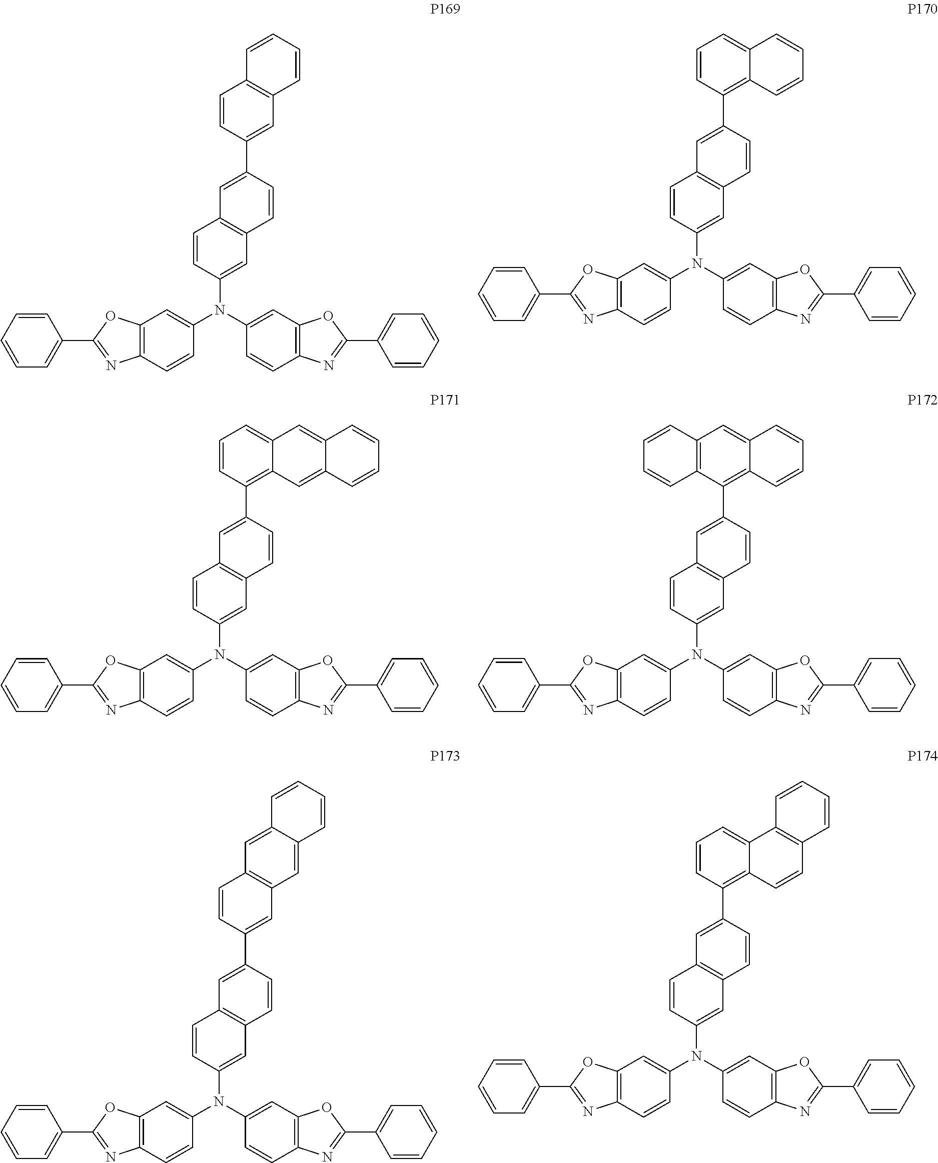

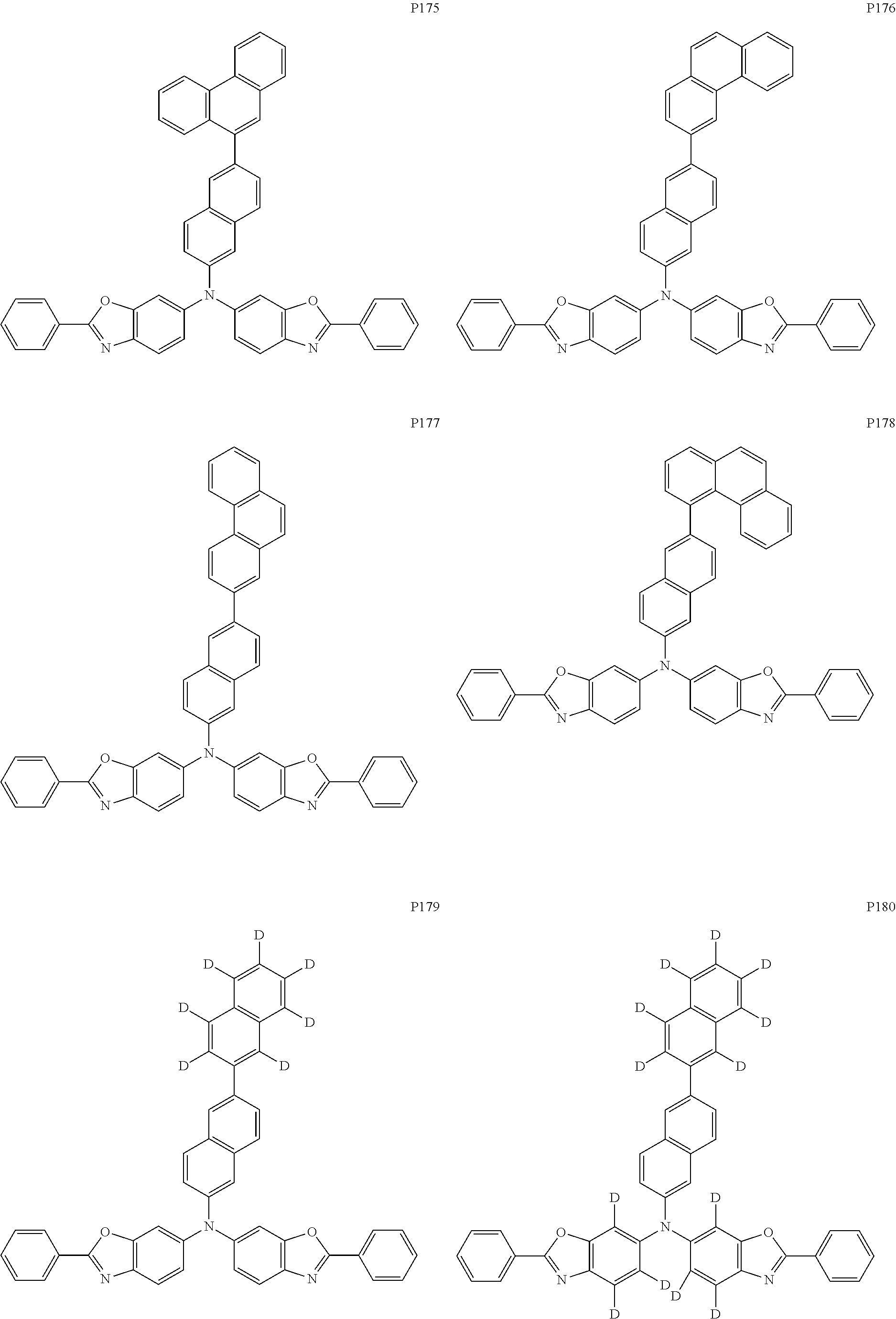

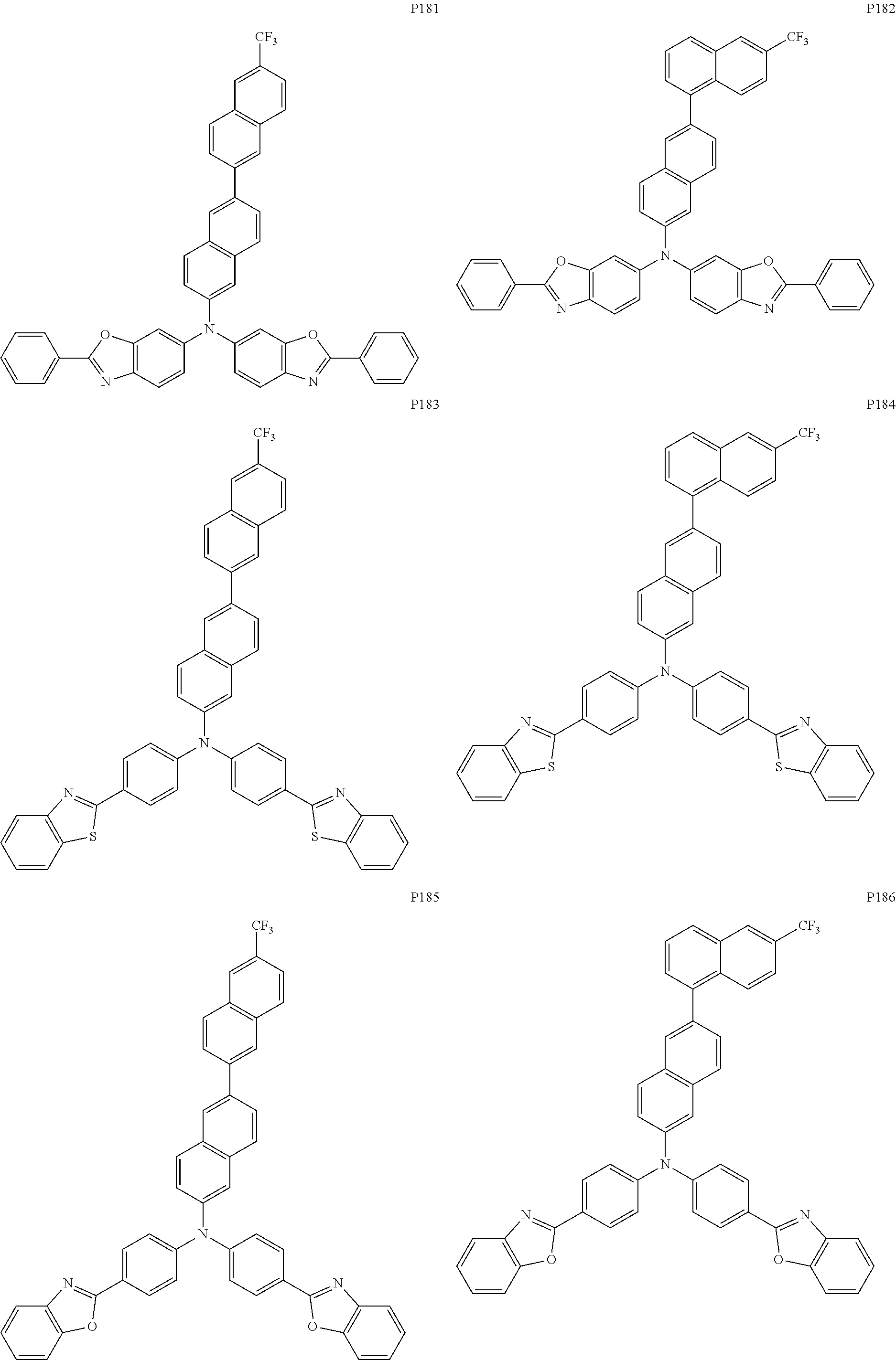

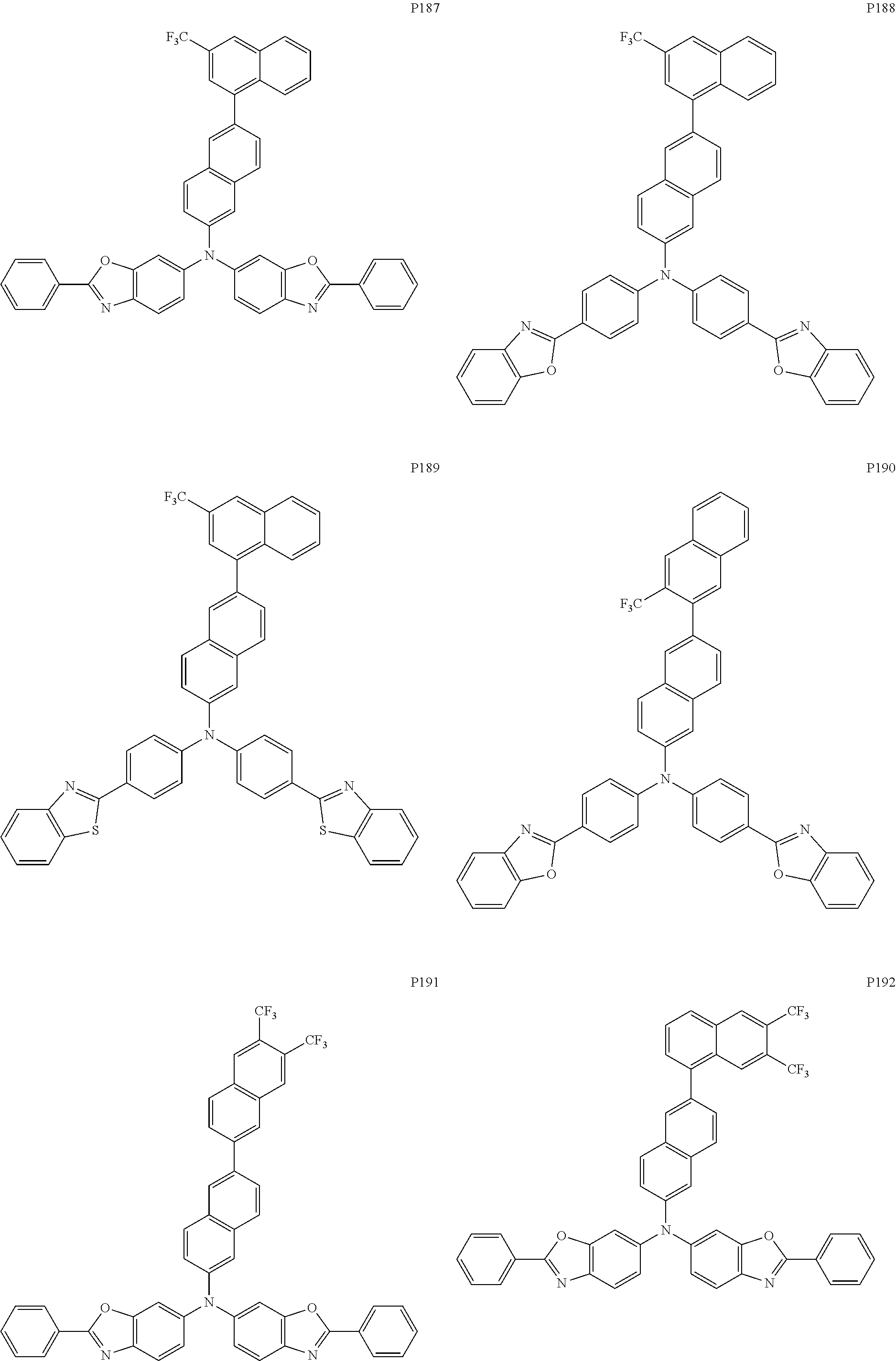

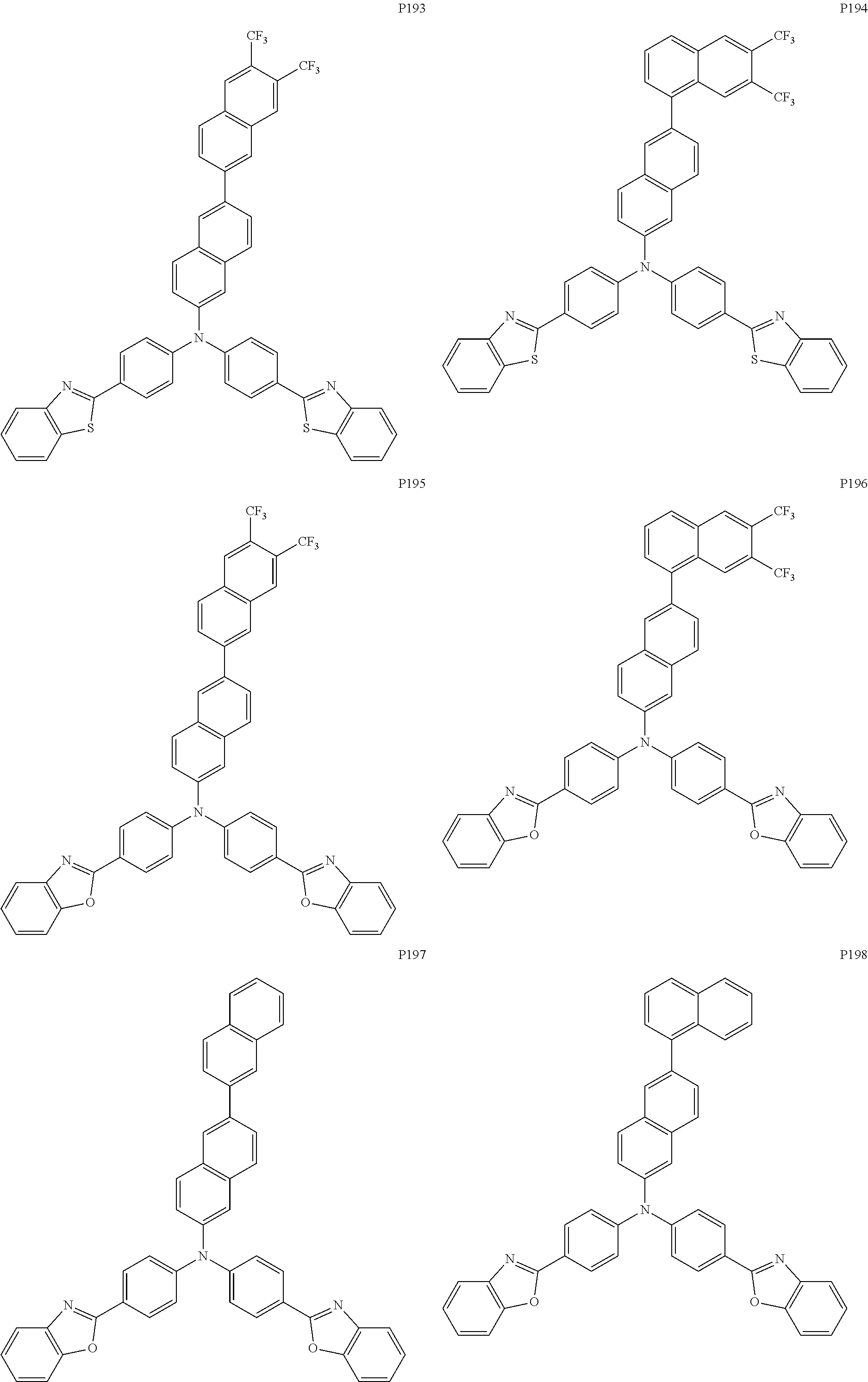

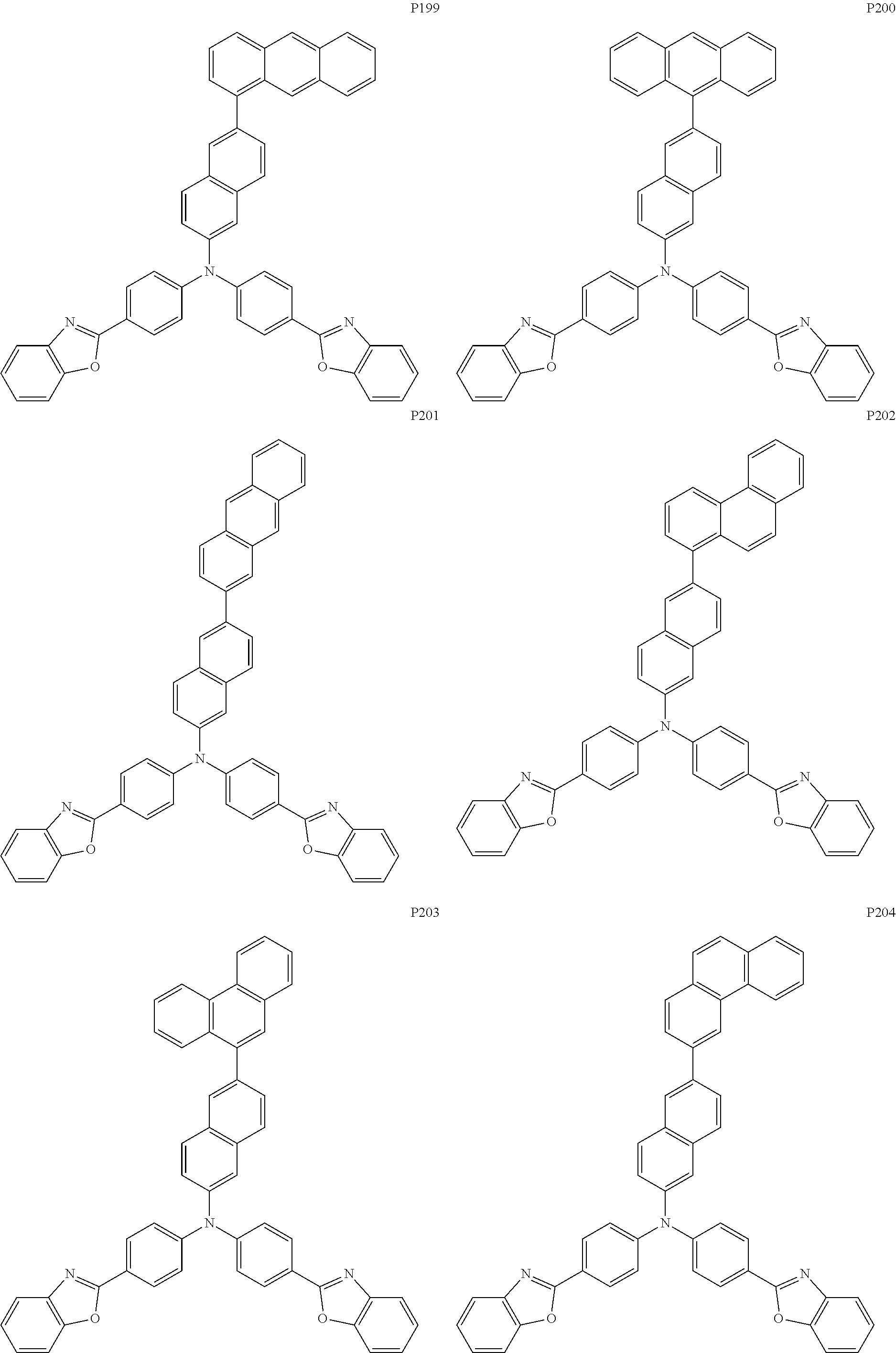

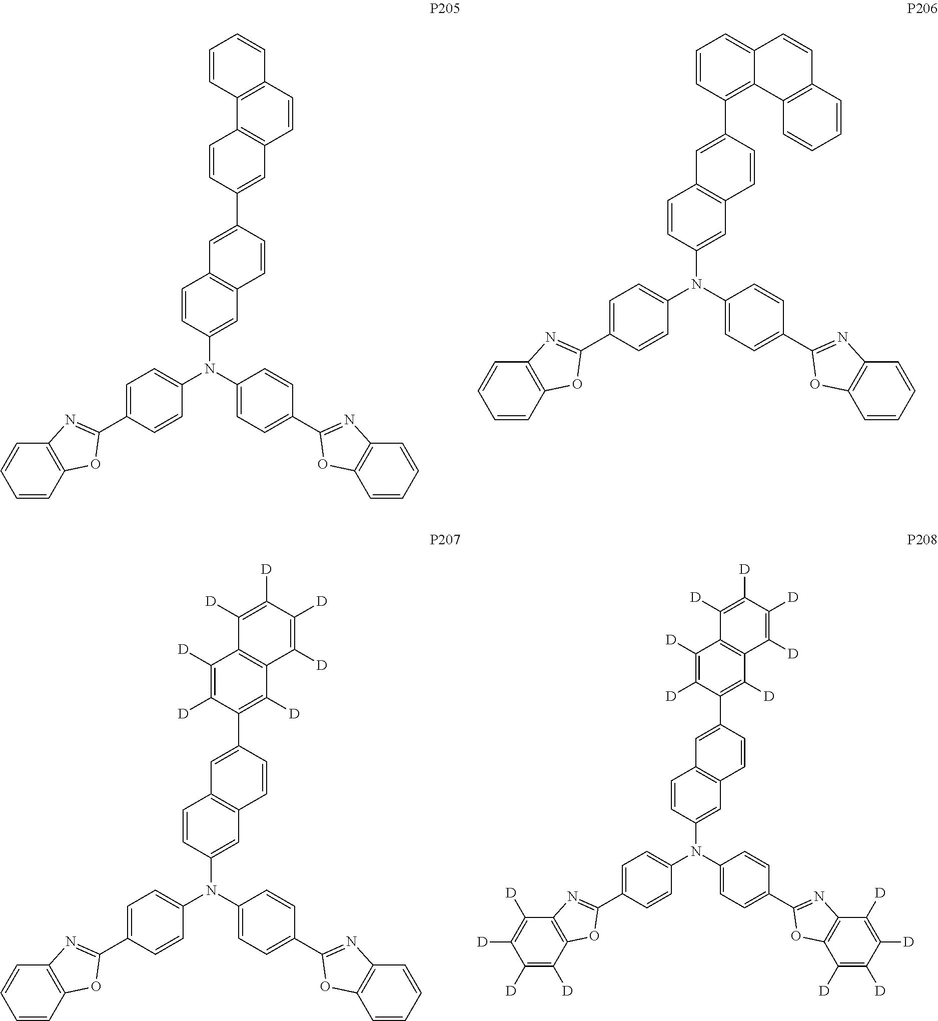 [SMILES: c1ccc2c(c1)ccc1cc(-c3ccc4cc(N(c5ccc(-c6nc7ccccc7o6)cc5)c5ccc(-c6nc7ccccc7o6)cc5)ccc4c3)ccc12 ; c1ccc2c(c1)ccc1cccc(-c3ccc4cc(N(c5ccc(-c6nc7ccccc7o6)cc5)c5ccc(-c6nc7ccccc7o6)cc5)ccc4c3)c12 ; [2H]c1c([2H])c([2H])c2c([2H])c(-c3ccc4cc(N(c5ccc(-c6nc7ccccc7o6)cc5)c5ccc(-c6nc7ccccc7o6)cc5)ccc4c3)c([2H])c([2H])c2c1[2H] ; [2H]c1c([2H])c([2H])c2oc(-c3ccc(N(c4ccc(-c5nc6c([2H])c([2H])c([2H])c([2H])c6o5)cc4)c4ccc5cc(-c6c([2H])c([2H])c7c([2H])c([2H])c([2H])c([2H])c7c6[2H])ccc5c4)cc3)nc2c1[2H]]

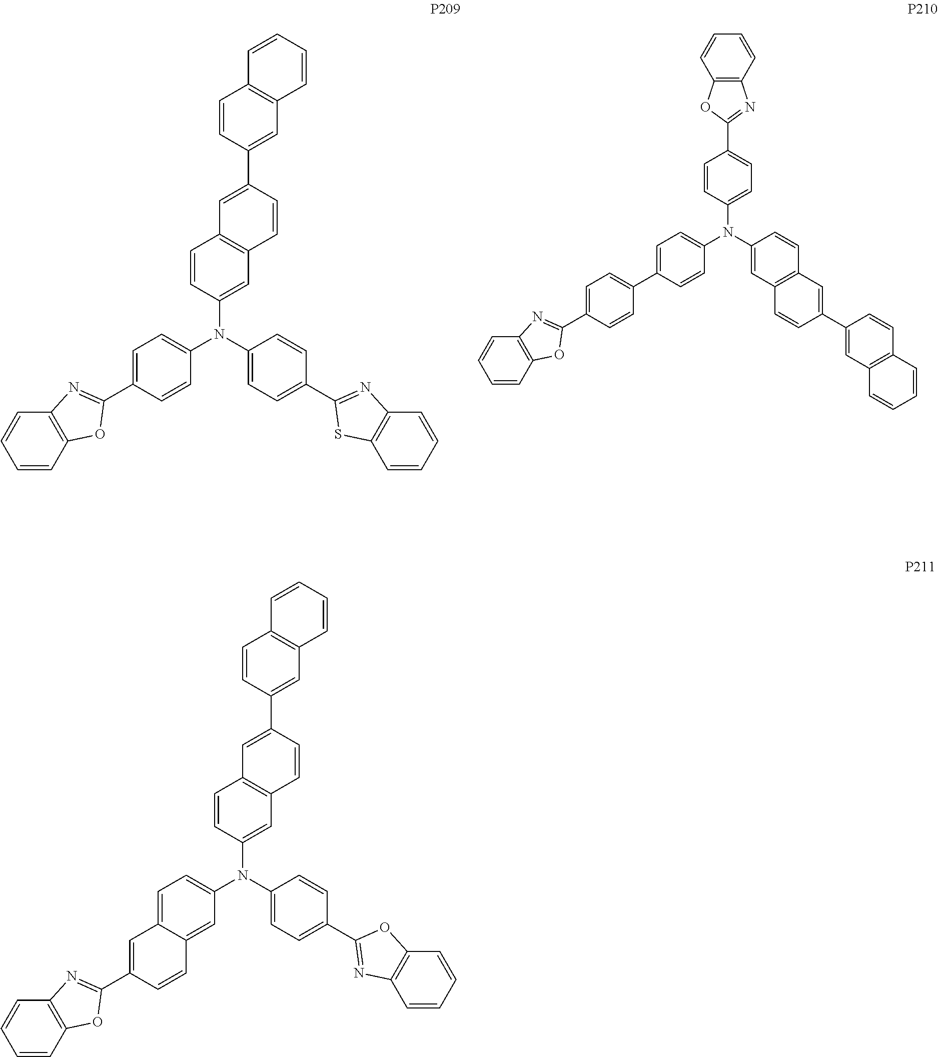 [SMILES: c1ccc2cc(-c3ccc4cc(N(c5ccc(-c6nc7ccccc7o6)cc5)c5ccc(-c6nc7ccccc7s6)cc5)ccc4c3)ccc2c1 ; c1ccc2cc(-c3ccc4cc(N(c5ccc(-c6nc7ccccc7o6)cc5)c5ccc6cc(-c7nc8ccccc8o7)ccc6c5)ccc4c3)ccc2c1 ; c1ccc2cc(-c3ccc4cc(N(c5ccc(-c6ccc(-c7nc8ccccc8o7)cc6)cc5)c5ccc(-c6nc7ccccc7o6)cc5)ccc4c3)ccc2c1 ; "P211"]

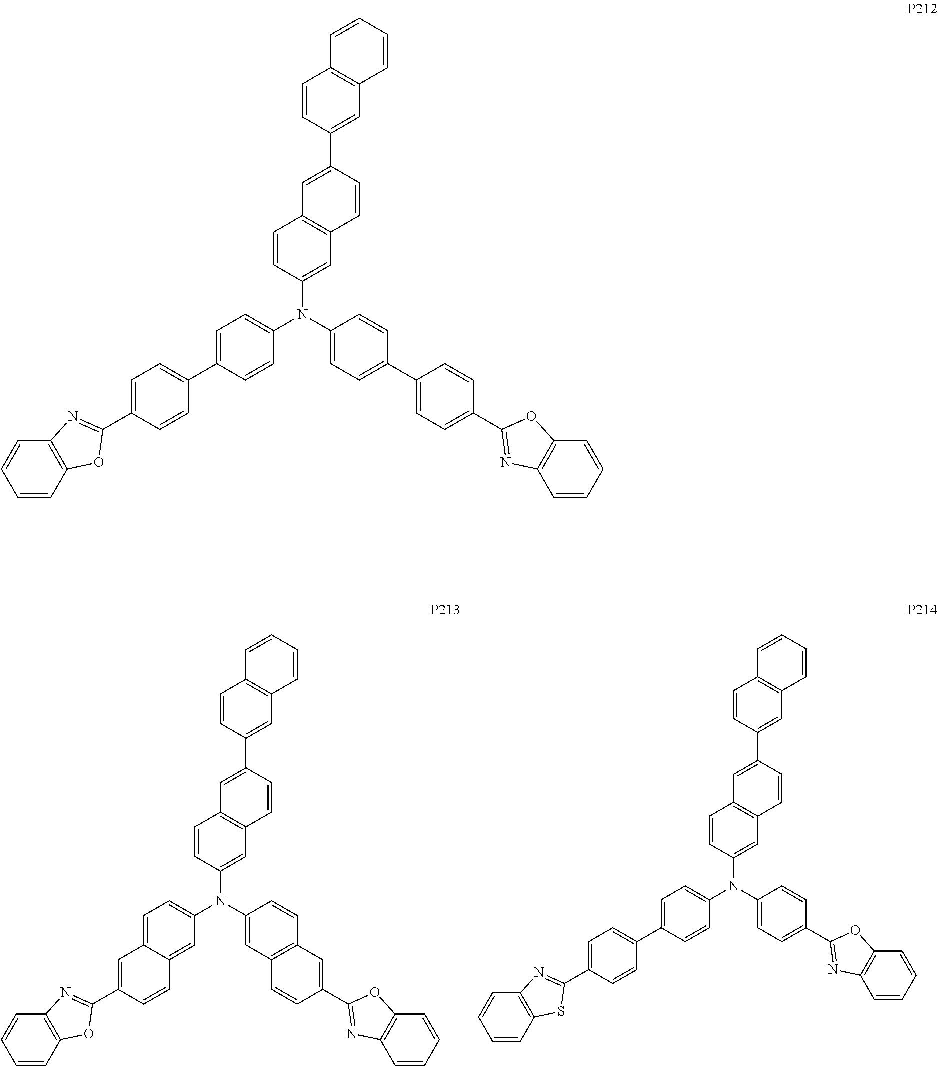 [SMILES: c1ccc2cc(-c3ccc4cc(N(c5ccc(-c6ccc(-c7nc8ccccc8o7)cc6)cc5)c5ccc(-c6ccc(-c7nc8ccccc8o7)cc6)cc5)ccc4c3)ccc2c1 ; c1ccc2cc(-c3ccc4cc(N(c5ccc6cc(-c7nc8ccccc8o7)ccc6c5)c5ccc6cc(-c7nc8ccccc8o7)ccc6c5)ccc4c3)ccc2c1 ; c1ccc2cc(-c3ccc4cc(N(c5ccc(-c6ccc(-c7nc8ccccc8s7)cc6)cc5)c5ccc(-c6nc7ccccc7o6)cc5)ccc4c3)ccc2c1]

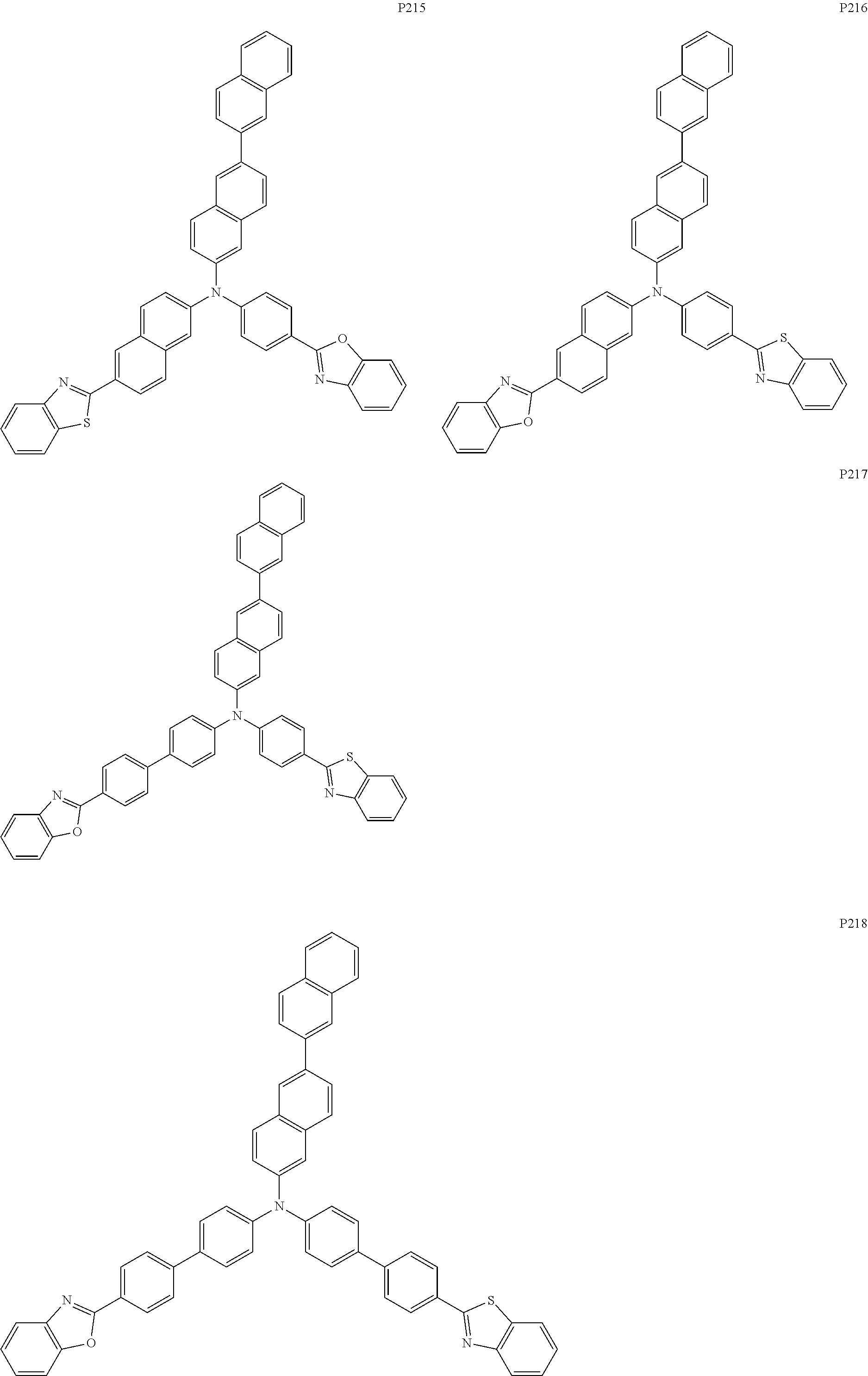 [SMILES: c1ccc2cc(-c3ccc4cc(N(c5ccc(-c6nc7ccccc7o6)cc5)c5ccc6cc(-c7nc8ccccc8s7)ccc6c5)ccc4c3)ccc2c1 ; c1ccc2cc(-c3ccc4cc(N(c5ccc(-c6nc7ccccc7s6)cc5)c5ccc6cc(-c7nc8ccccc8o7)ccc6c5)ccc4c3)ccc2c1 ; c1ccc2cc(-c3ccc4cc(N(c5ccc(-c6ccc(-c7nc8ccccc8o7)cc6)cc5)c5ccc(-c6nc7ccccc7s6)cc5)ccc4c3)ccc2c1 ; c1ccc2cc(-c3ccc4cc(N(c5ccc(-c6ccc(-c7nc8ccccc8o7)cc6)cc5)c5ccc(-c6ccc(-c7nc8ccccc8s7)cc6)cc5)ccc4c3)ccc2c1]

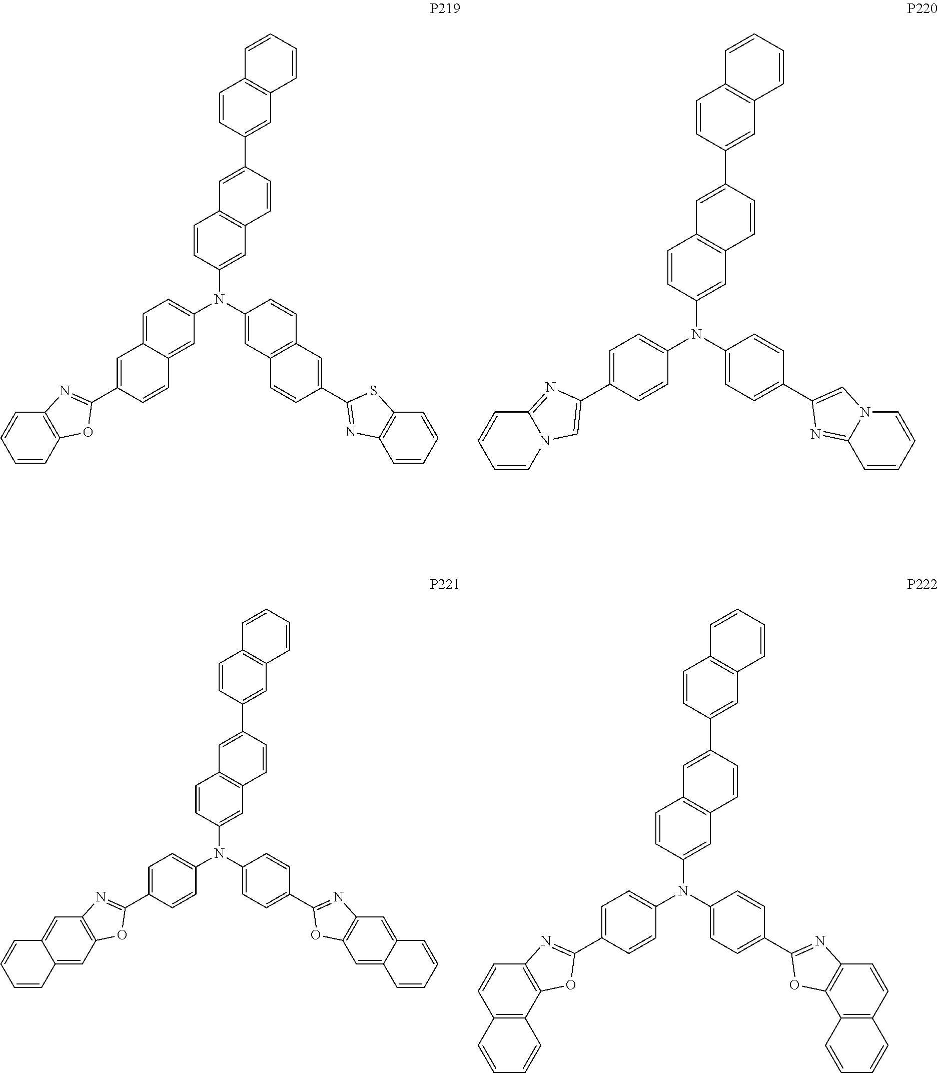

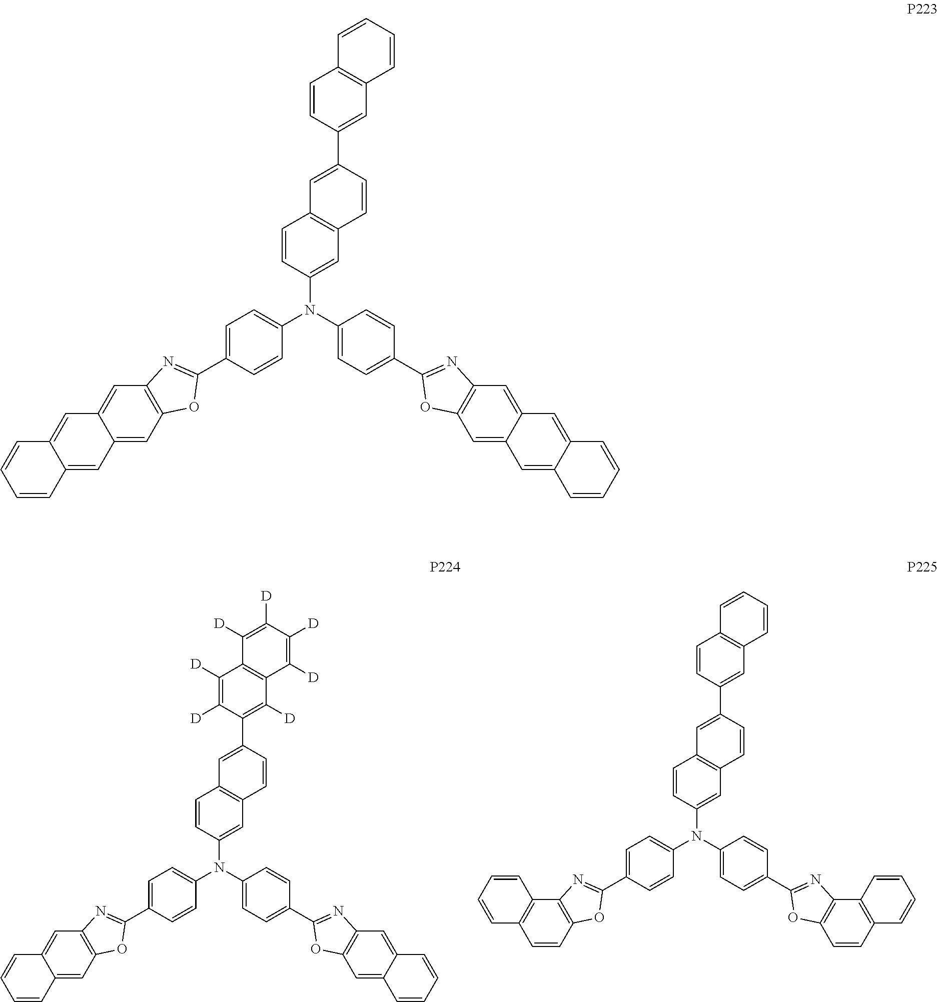 [SMILES: c1ccc2cc(-c3ccc4cc(N(c5ccc(-c6nc7cc8cc9ccccc9cc8cc7o6)cc5)c5ccc(-c6nc7cc8cc9ccccc9cc8cc7o6)cc5)ccc4c3)ccc2c1 ; [2H]c1c([2H])c([2H])c2c([2H])c(-c3ccc4cc(N(c5ccc(-c6nc7cc8ccccc8cc7o6)cc5)c5ccc(-c6nc7cc8ccccc8cc7o6)cc5)ccc4c3)c([2H])c([2H])c2c1[2H] ; c1ccc2cc(-c3ccc4cc(N(c5ccc(-c6nc7c(ccc8ccccc87)o6)cc5)c5ccc(-c6nc7c(ccc8ccccc87)o6)cc5)ccc4c3)ccc2c1]

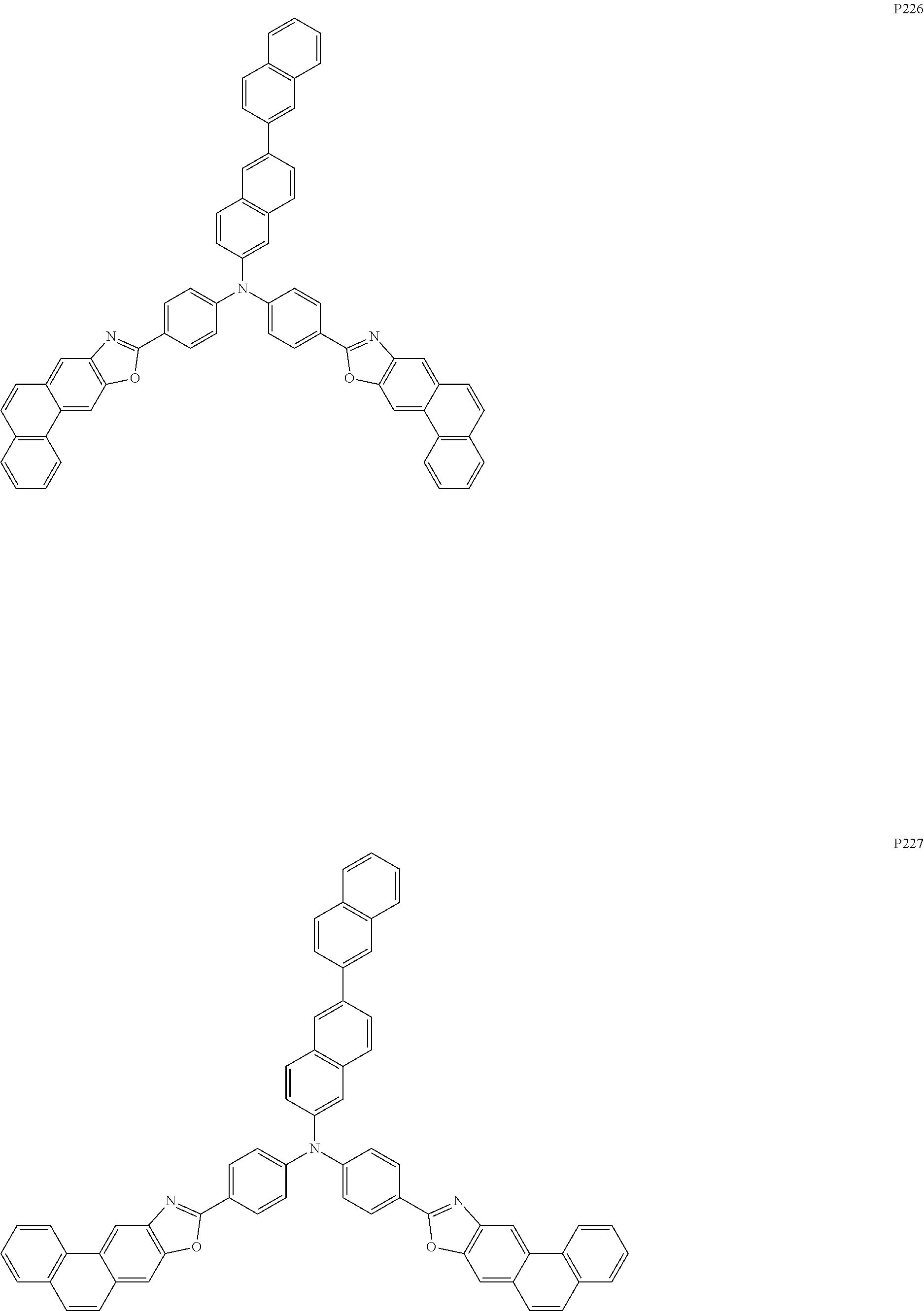 [SMILES: c1ccc2cc(-c3ccc4cc(N(c5ccc(-c6nc7cc8ccc9ccccc9c8cc7o6)cc5)c5ccc(-c6nc7cc8ccc9ccccc9c8cc7o6)cc5)ccc4c3)ccc2c1 ; c1ccc2cc(-c3ccc4cc(N(c5ccc(-c6nc7cc8c(ccc9ccccc98)cc7o6)cc5)c5ccc(-c6nc7cc8c(ccc9ccccc98)cc7o6)cc5)ccc4c3)ccc2c1]

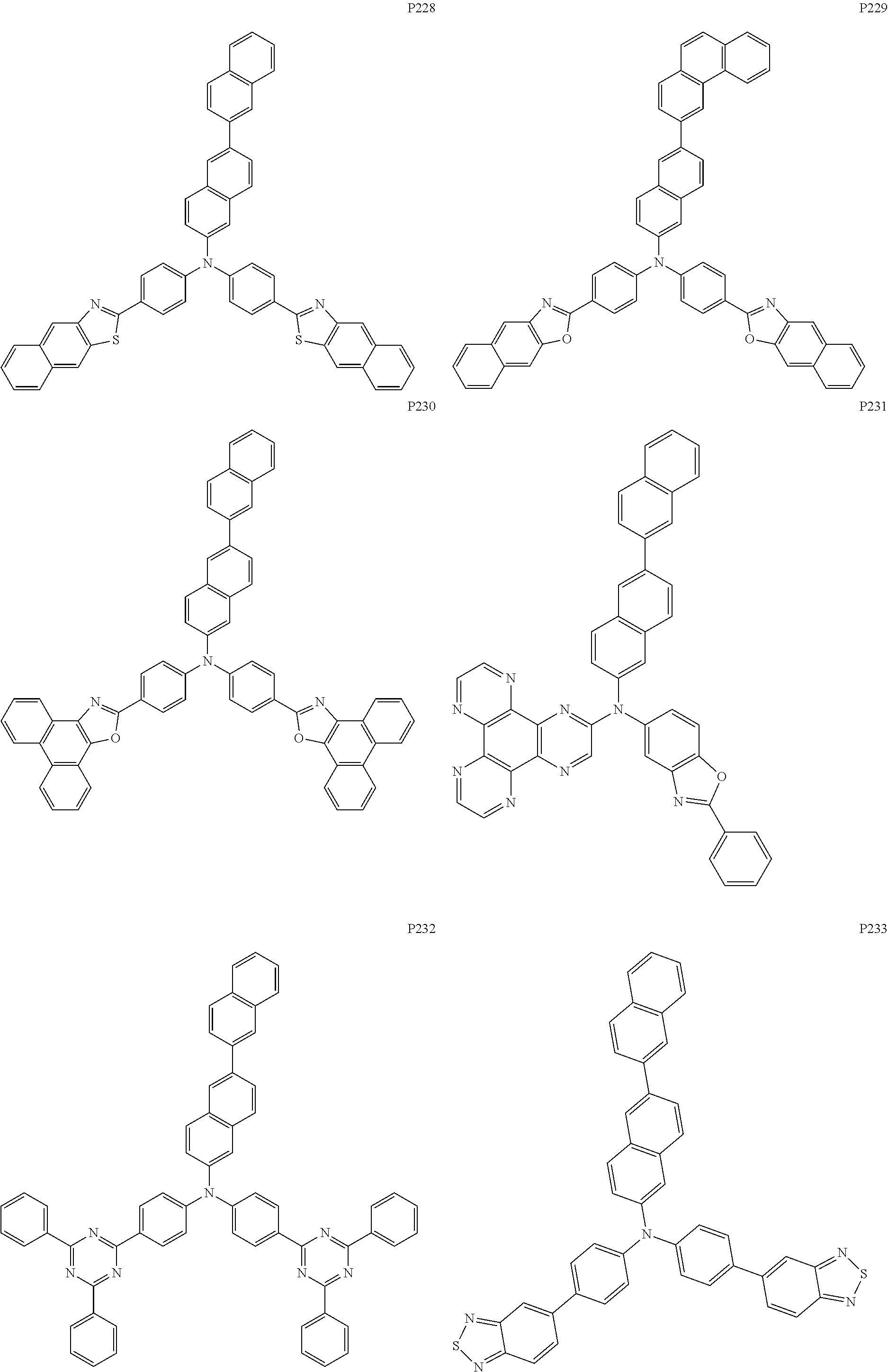

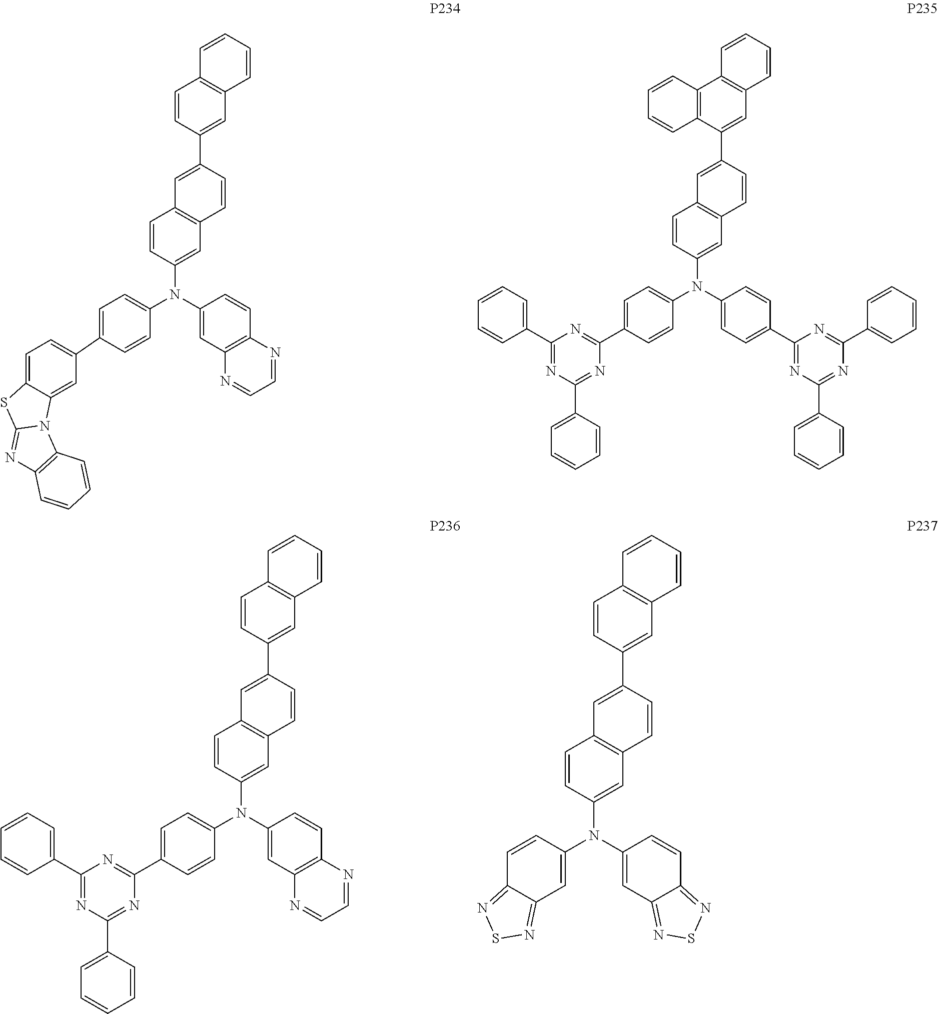 [SMILES: c1ccc2cc(-c3ccc4cc(N(c5ccc(-c6ccc7sc8nc9ccccc9n8c7c6)cc5)c5ccc6nccnc6c5)ccc4c3)ccc2c1 ; c1ccc(-c2nc(-c3ccccc3)nc(-c3ccc(N(c4ccc(-c5nc(-c6ccccc6)nc(-c6ccccc6)n5)cc4)c4ccc5cc(-c6cc7ccccc7c7ccccc67)ccc5c4)cc3)n2)cc1 ; c1ccc(-c2nc(-c3ccccc3)nc(-c3ccc(N(c4ccc5cc(-c6ccc7ccccc7c6)ccc5c4)c4ccc5nccnc5c4)cc3)n2)cc1 ; c1ccc2cc(-c3ccc4cc(N(c5ccc6nsnc6c5)c5ccc6nsnc6c5)ccc4c3)ccc2c1]

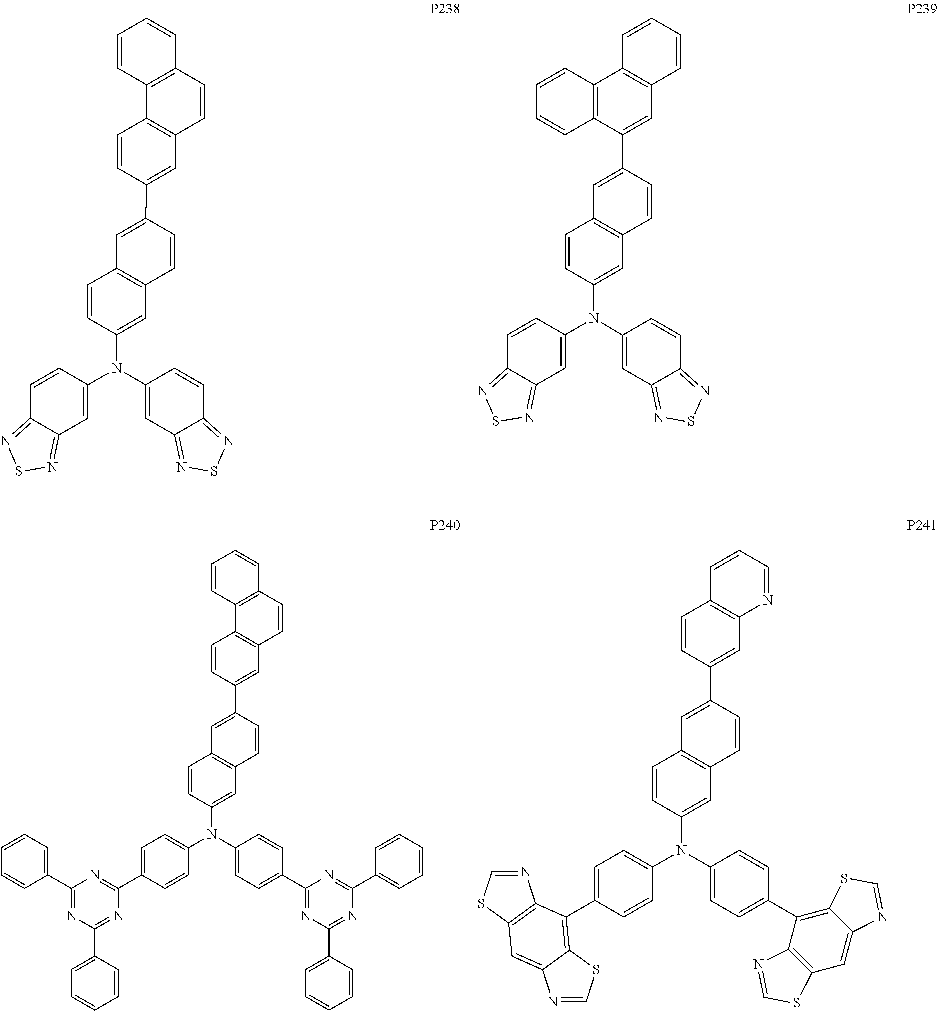 [SMILES: c1ccc2c(c1)ccc1cc(-c3ccc4cc(N(c5ccc6nsnc6c5)c5ccc6nsnc6c5)ccc4c3)ccc12 ; c1ccc2c(c1)cc(-c1ccc3cc(N(c4ccc5nsnc5c4)c4ccc5nsnc5c4)ccc3c1)c1ccccc12 ; c1ccc(-c2nc(-c3ccccc3)nc(-c3ccc(N(c4ccc(-c5nc(-c6ccccc6)nc(-c6ccccc6)n5)cc4)c4ccc5cc(-c6ccc7c(ccc8ccccc87)c6)ccc5c4)cc3)n2)cc1 ; c1cnc2cc(-c3ccc4cc(N(c5ccc(-c6c7ncsc7cc7ncsc67)cc5)c5ccc(-c6c7ncsc7cc7ncsc67)cc5)ccc4c3)ccc2c1]

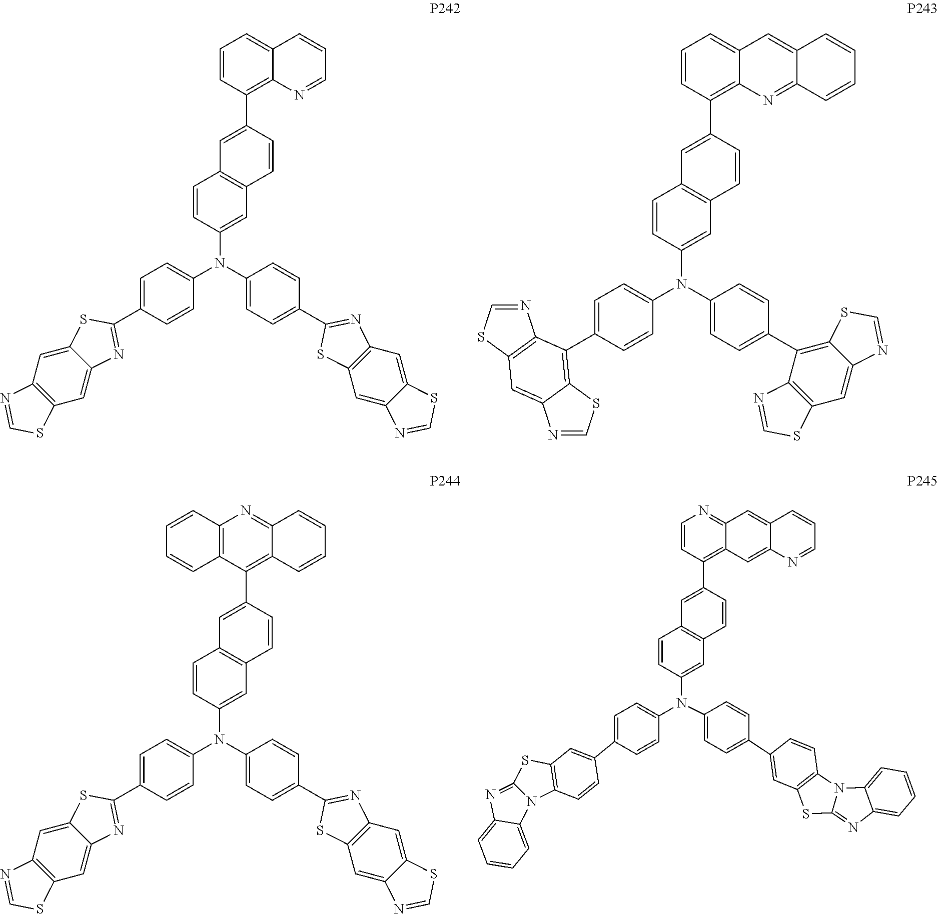 [SMILES: c1cnc2c(-c3ccc4cc(N(c5ccc(-c6nc7cc8scnc8cc7s6)cc5)c5ccc(-c6nc7cc8scnc8cc7s6)cc5)ccc4c3)cccc2c1 ; c1ccc2nc3c(-c4ccc5cc(N(c6ccc(-c7c8ncsc8cc8ncsc78)cc6)c6ccc(-c7c8ncsc8cc8ncsc78)cc6)ccc5c4)cccc3cc2c1 ; c1ccc2c(-c3ccc4cc(N(c5ccc(-c6nc7cc8scnc8cc7s6)cc5)c5ccc(-c6nc7cc8scnc8cc7s6)cc5)ccc4c3)c3ccccc3nc2c1 ; c1cnc2cc3c(-c4ccc5cc(N(c6ccc(-c7ccc8c(c7)sc7nc9ccccc9n78)cc6)c6ccc(-c7ccc8c(c7)sc7nc9ccccc9n78)cc6)ccc5c4)ccnc3cc2c1]

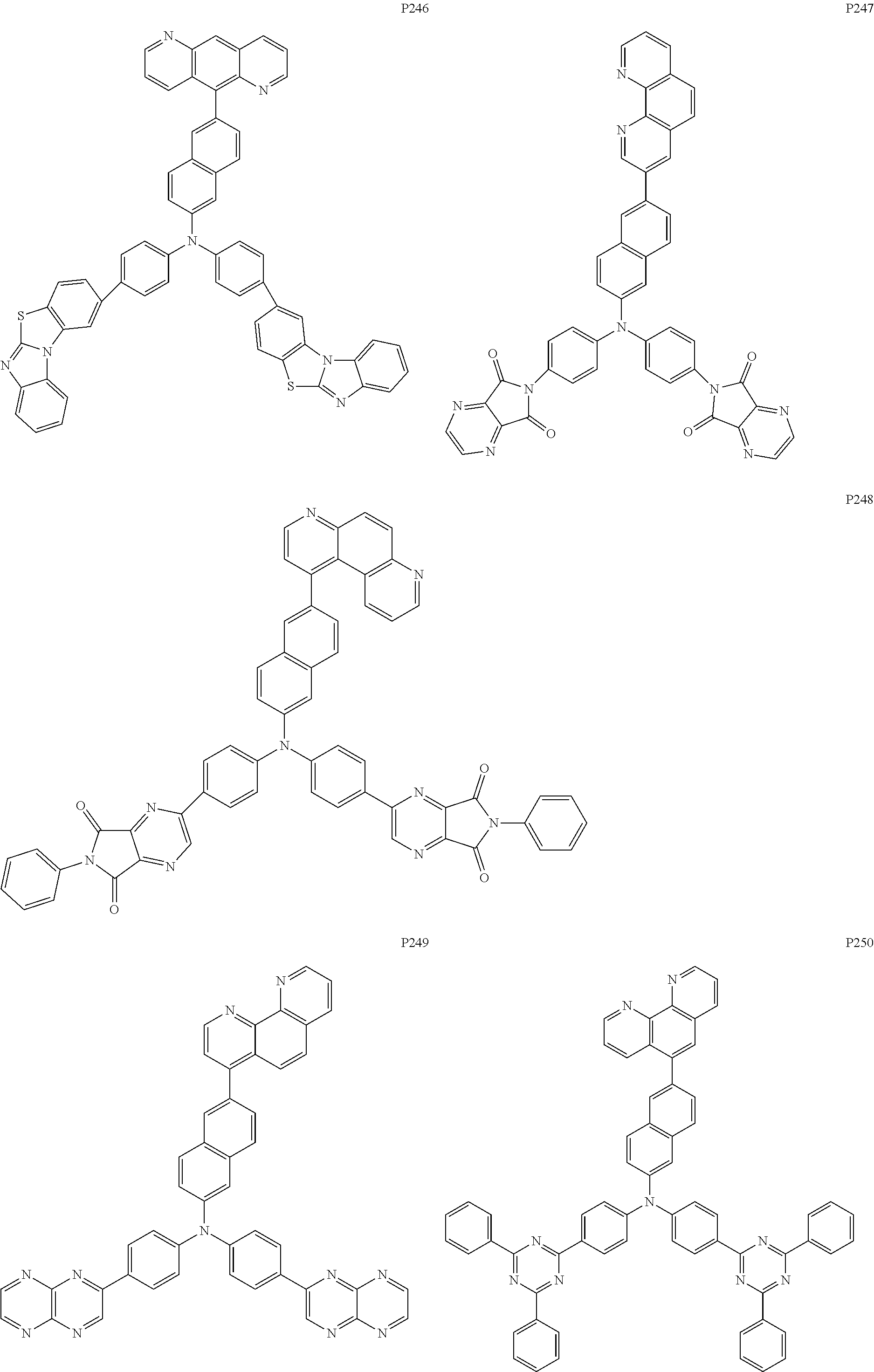

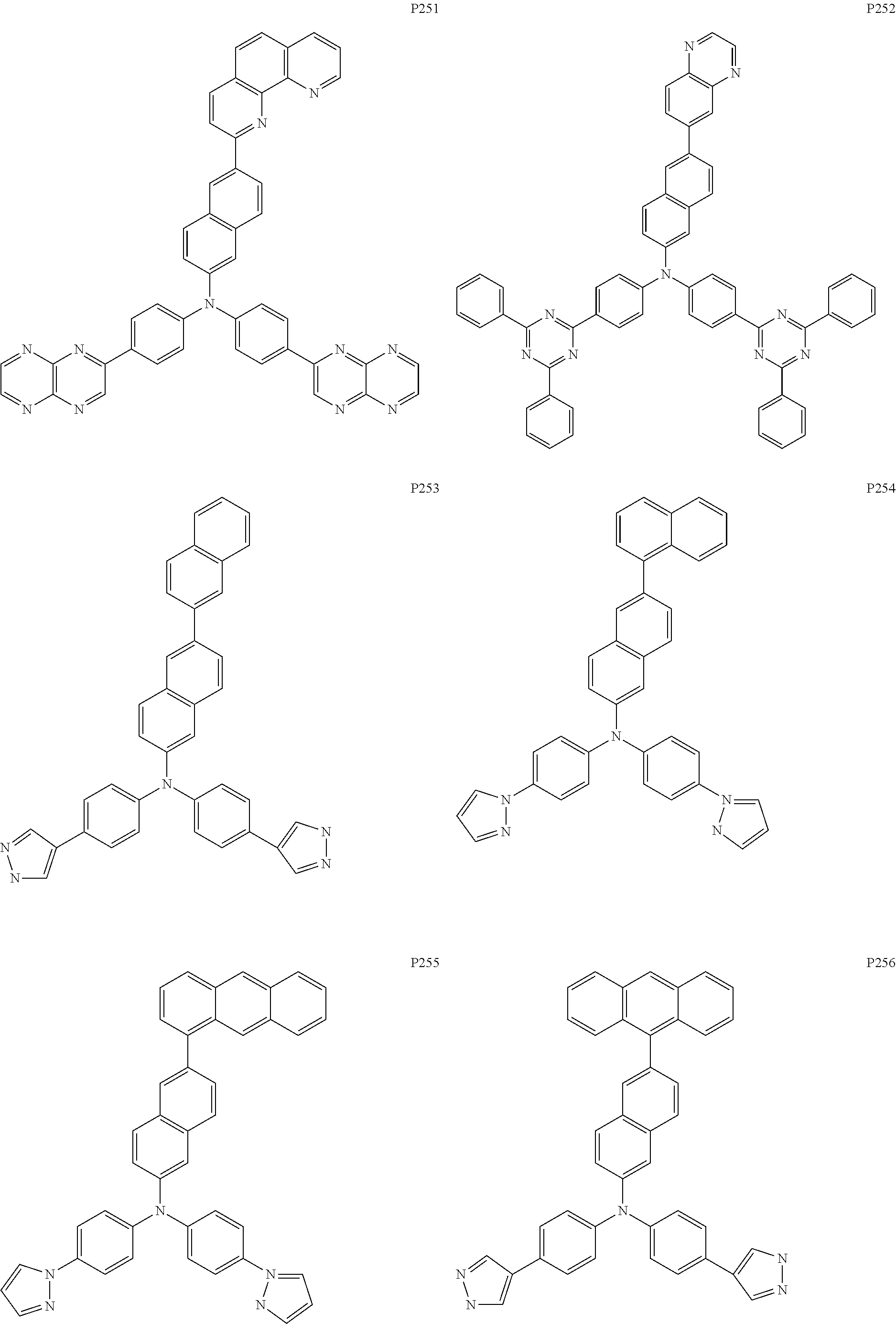

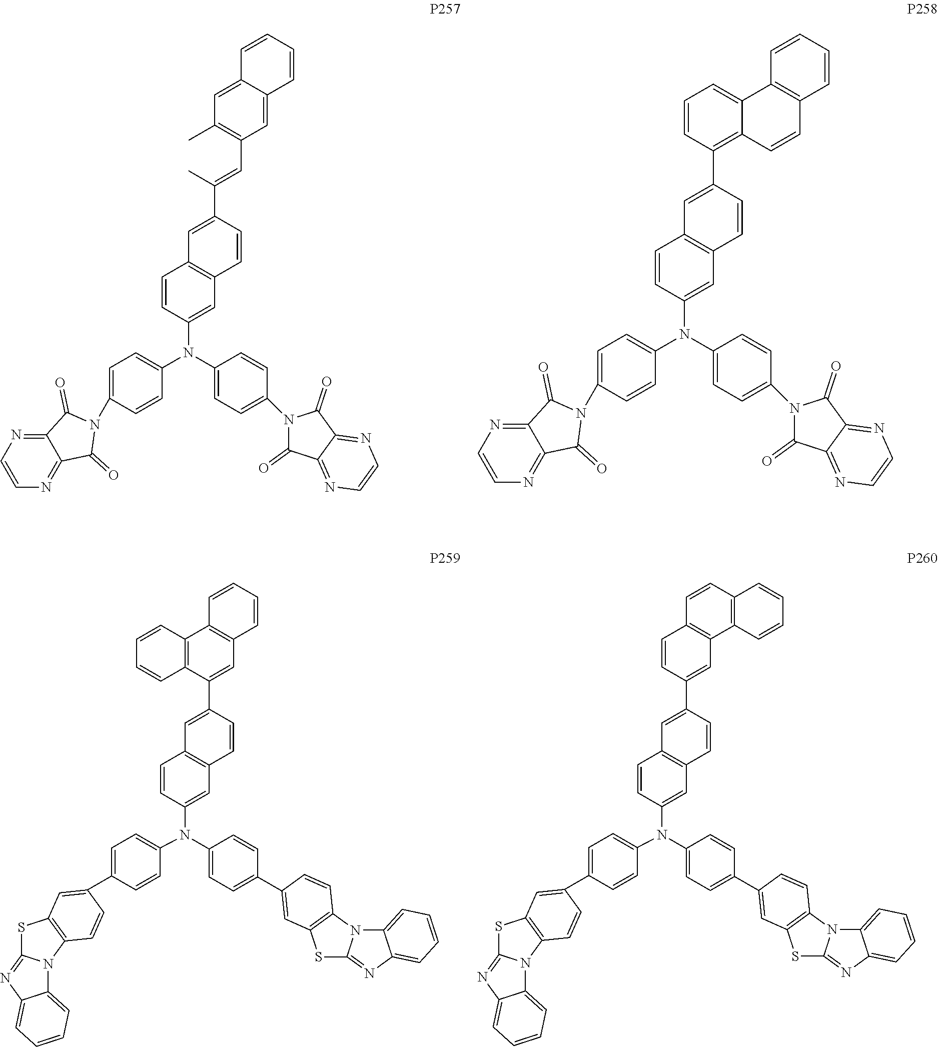 [SMILES: C/C(=C\c1cc2ccccc2cc1C)c1ccc2cc(N(c3ccc(N4C(=O)c5nccnc5C4=O)cc3)c3ccc(N4C(=O)c5nccnc5C4=O)cc3)ccc2c1 ; O=C1c2nccnc2C(=O)N1c1ccc(N(c2ccc(N3C(=O)c4nccnc4C3=O)cc2)c2ccc3cc(-c4cccc5c4ccc4ccccc45)ccc3c2)cc1 ; c1ccc2c(c1)cc(-c1ccc3cc(N(c4ccc(-c5ccc6c(c5)sc5nc7ccccc7n56)cc4)c4ccc(-c5ccc6c(c5)sc5nc7ccccc7n56)cc4)ccc3c1)c1ccccc12 ; c1ccc2c(c1)ccc1ccc(-c3ccc4cc(N(c5ccc(-c6ccc7c(c6)sc6nc8ccccc8n67)cc5)c5ccc(-c6ccc7c(c6)sc6nc8ccccc8n67)cc5)ccc4c3)cc12]

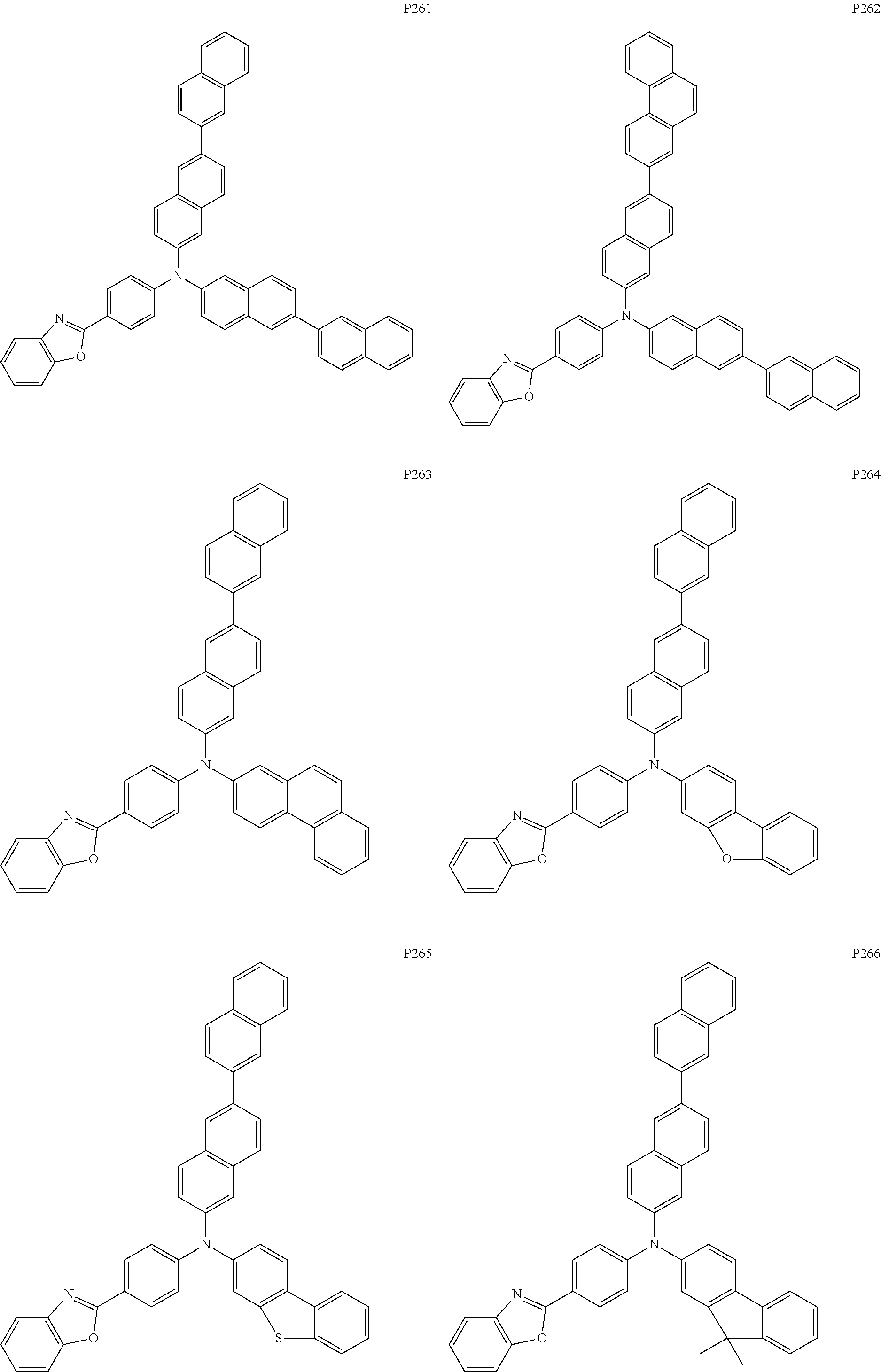

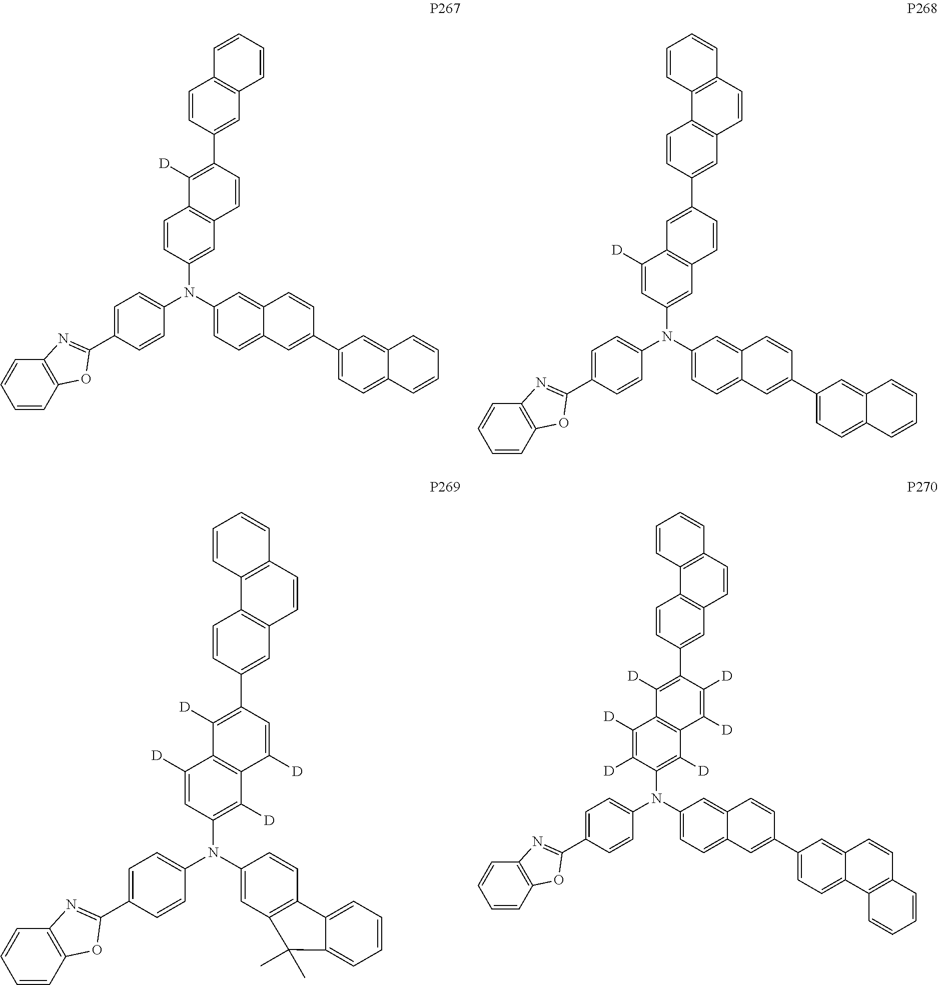 [SMILES: [2H]c1c(-c2ccc3ccccc3c2)ccc2cc(N(c3ccc(-c4nc5ccccc5o4)cc3)c3ccc4cc(-c5ccc6ccccc6c5)ccc4c3)ccc12 ; [2H]c1cc(N(c2ccc(-c3nc4ccccc4o3)cc2)c2ccc3cc(-c4ccc5ccccc5c4)ccc3c2)cc2ccc(-c3ccc4c(ccc5ccccc54)c3)cc12 ; [2H]c1cc(N(c2ccc(-c3nc4ccccc4o3)cc2)c2ccc3c(c2)C(C)(C)c2ccccc2-3)c([2H])c2c([2H])cc(-c3ccc4c(ccc5ccccc54)c3)c([2H])c12 ; [2H]c1c(N(c2ccc(-c3nc4ccccc4o3)cc2)c2ccc3cc(-c4ccc5c(ccc6ccccc65)c4)ccc3c2)c([2H])c2c([2H])c([2H])c(-c3ccc4c(ccc5ccccc54)c3)c([2H])c2c1[2H]]

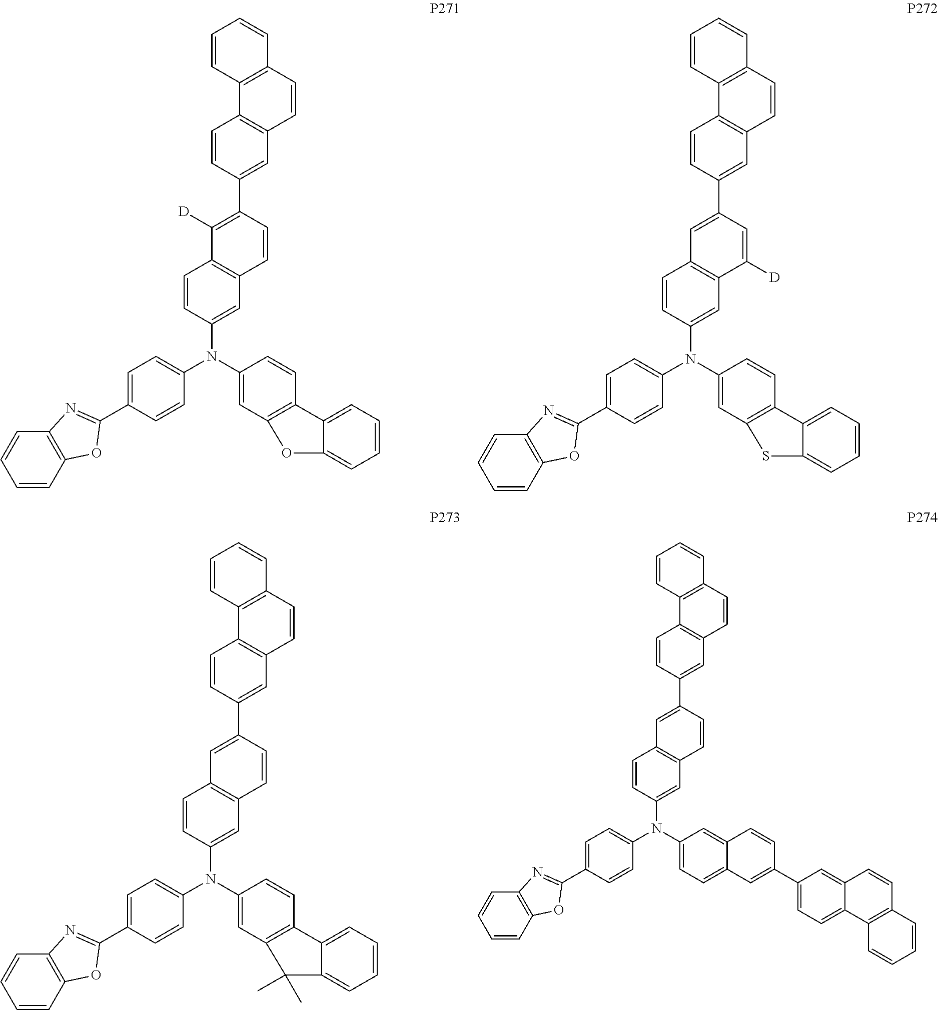 [SMILES: [2H]c1c(-c2ccc3c(ccc4ccccc43)c2)ccc2cc(N(c3ccc(-c4nc5ccccc5o4)cc3)c3ccc4c(c3)oc3ccccc34)ccc12 ; [2H]c1cc(-c2ccc3c(ccc4ccccc43)c2)cc2ccc(N(c3ccc(-c4nc5ccccc5o4)cc3)c3ccc4c(c3)sc3ccccc34)cc12 ; CC1(C)c2ccccc2-c2ccc(N(c3ccc(-c4nc5ccccc5o4)cc3)c3ccc4cc(-c5ccc6c(ccc7ccccc76)c5)ccc4c3)cc21 ; c1ccc2c(c1)ccc1cc(-c3ccc4cc(N(c5ccc(-c6nc7ccccc7o6)cc5)c5ccc6cc(-c7ccc8c(ccc9ccccc98)c7)ccc6c5)ccc4c3)ccc12]

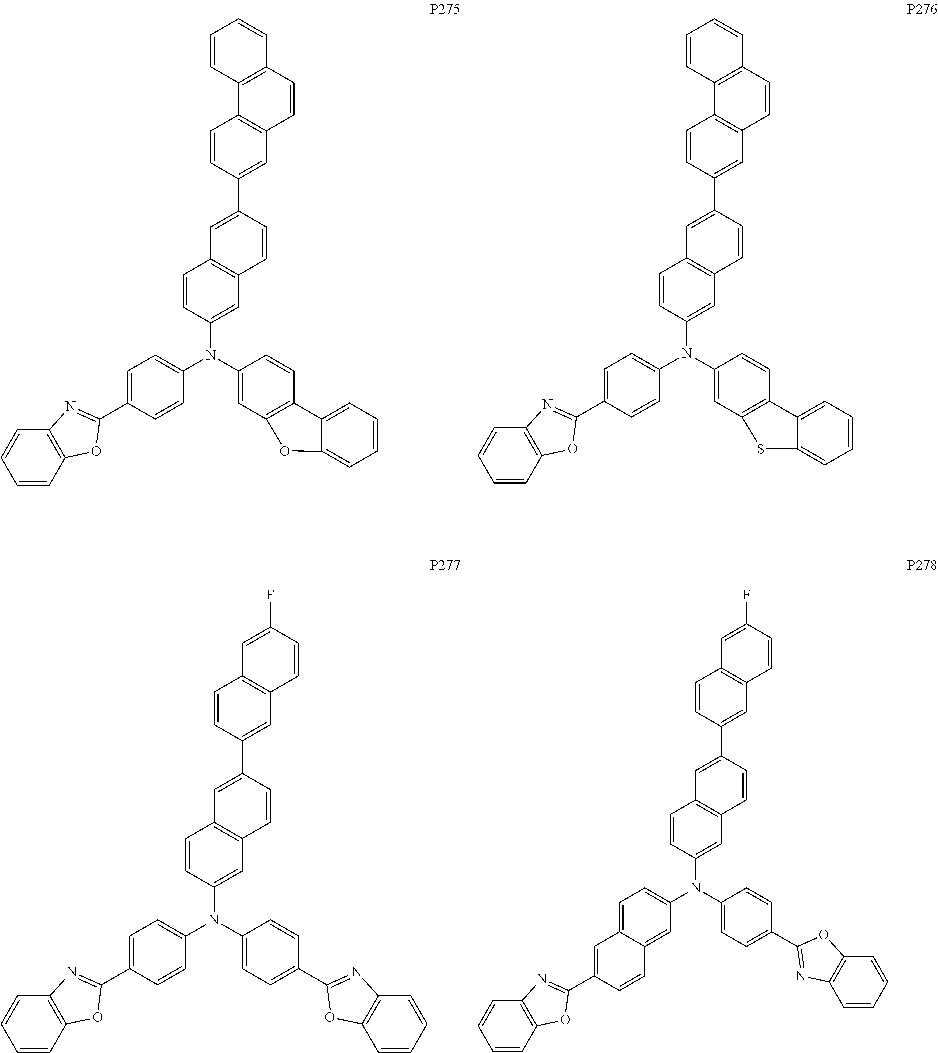 [SMILES: c1ccc2c(c1)ccc1cc(-c3ccc4cc(N(c5ccc(-c6nc7ccccc7o6)cc5)c5ccc6c(c5)oc5ccccc56)ccc4c3)ccc12 ; c1ccc2c(c1)ccc1cc(-c3ccc4cc(N(c5ccc(-c6nc7ccccc7o6)cc5)c5ccc6c(c5)sc5ccccc56)ccc4c3)ccc12 ; Fc1ccc2cc(-c3ccc4cc(N(c5ccc(-c6nc7ccccc7o6)cc5)c5ccc(-c6nc7ccccc7o6)cc5)ccc4c3)ccc2c1 ; Fc1ccc2cc(-c3ccc4cc(N(c5ccc(-c6nc7ccccc7o6)cc5)c5ccc6cc(-c7nc8ccccc8o7)ccc6c5)ccc4c3)ccc2c1]

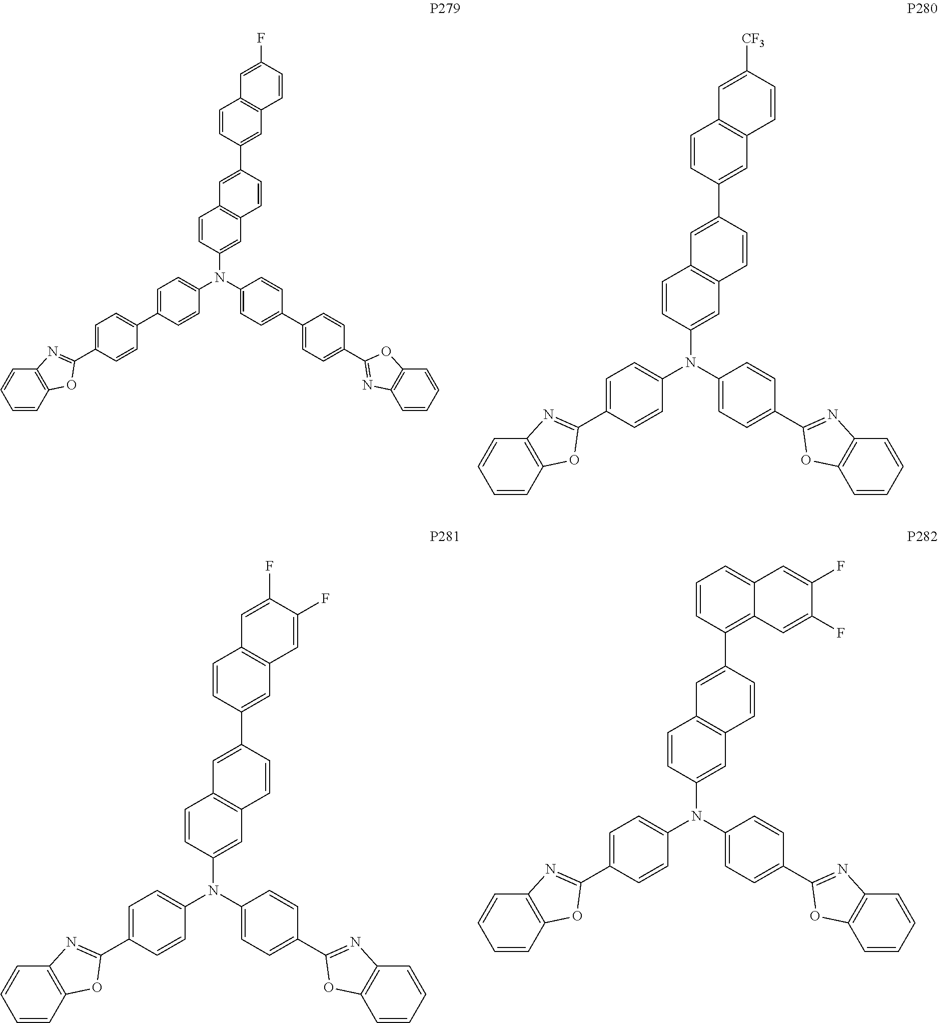 [SMILES: Fc1ccc2cc(-c3ccc4cc(N(c5ccc(-c6ccc(-c7nc8ccccc8o7)cc6)cc5)c5ccc(-c6ccc(-c7nc8ccccc8o7)cc6)cc5)ccc4c3)ccc2c1 ; FC(F)(F)c1ccc2cc(-c3ccc4cc(N(c5ccc(-c6nc7ccccc7o6)cc5)c5ccc(-c6nc7ccccc7o6)cc5)ccc4c3)ccc2c1 ; Fc1cc2ccc(-c3ccc4cc(N(c5ccc(-c6nc7ccccc7o6)cc5)c5ccc(-c6nc7ccccc7o6)cc5)ccc4c3)cc2cc1F ; Fc1cc2cccc(-c3ccc4cc(N(c5ccc(-c6nc7ccccc7o6)cc5)c5ccc(-c6nc7ccccc7o6)cc5)ccc4c3)c2cc1F]

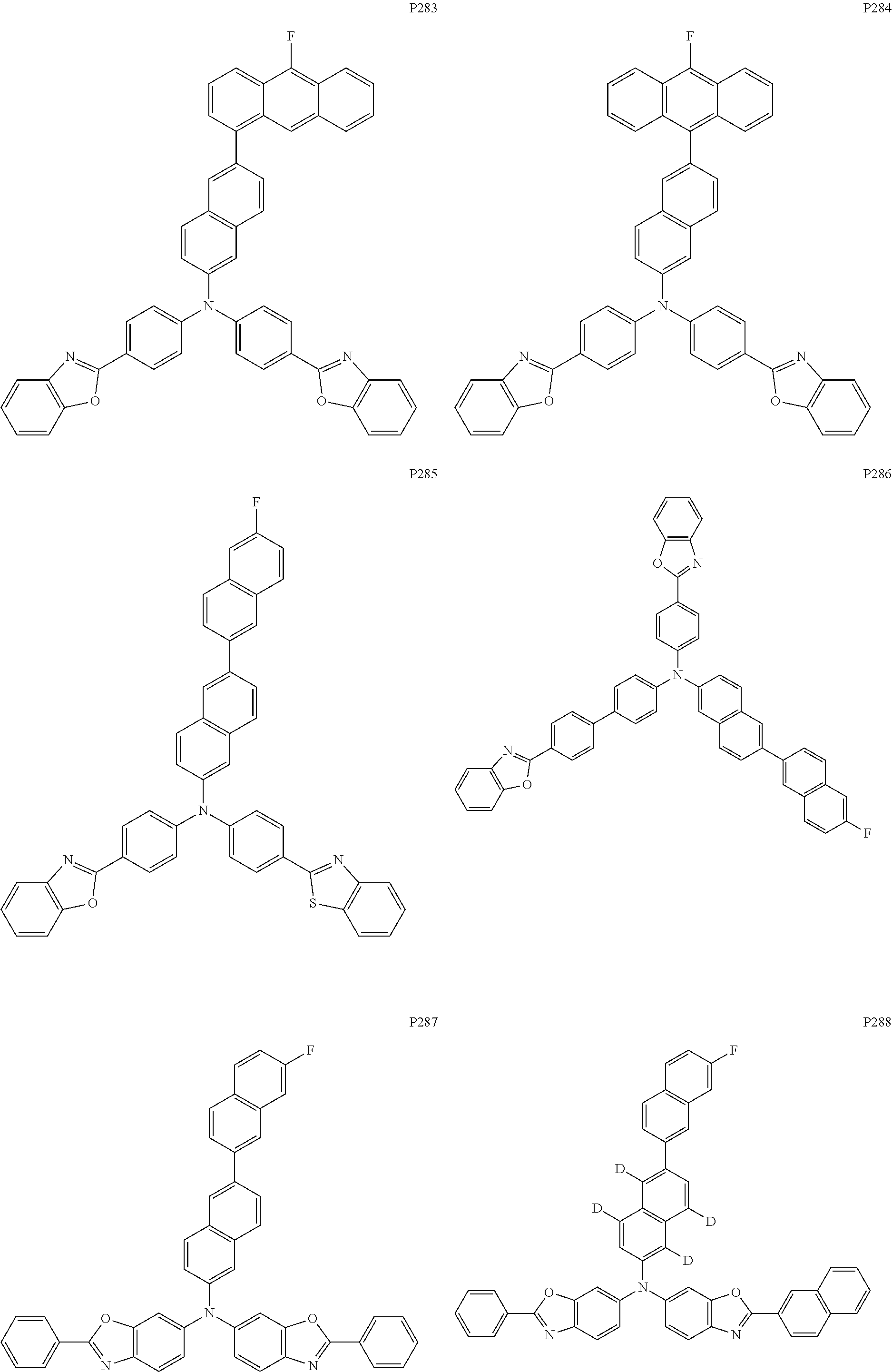

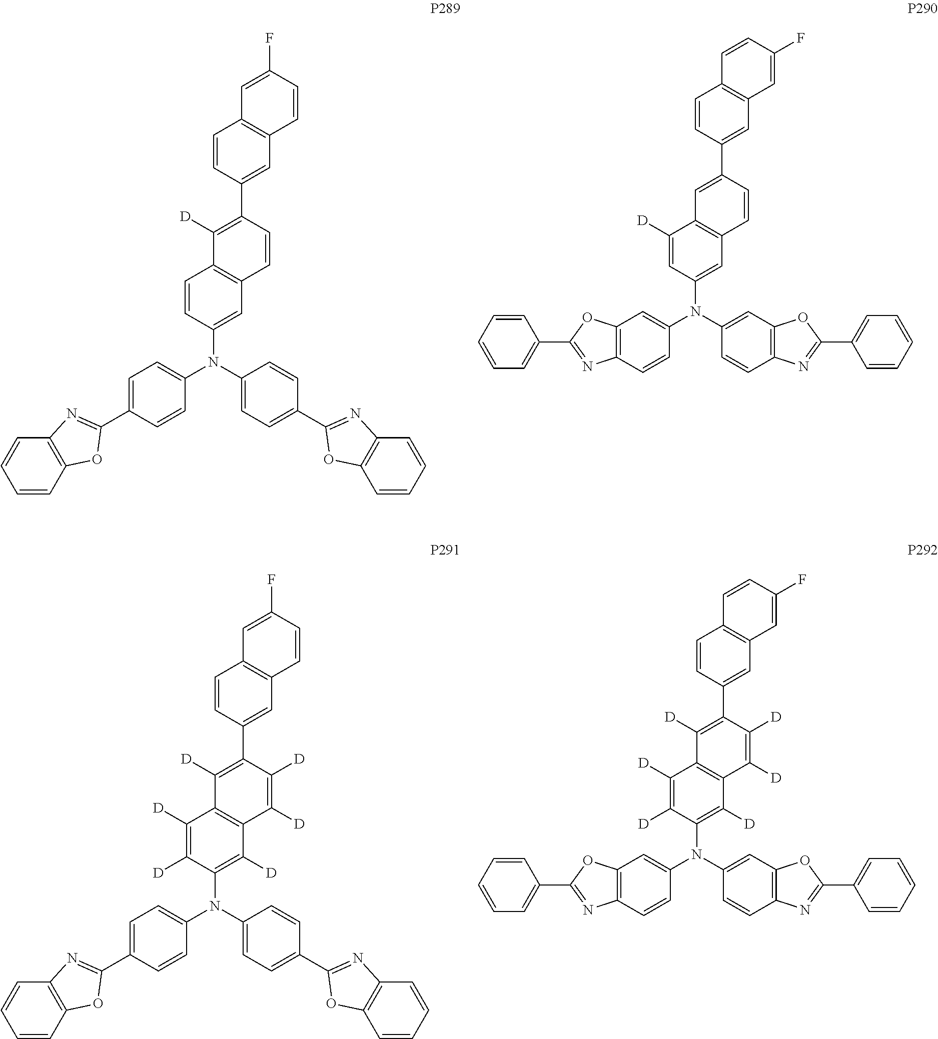 [SMILES: [2H]c1c(-c2ccc3cc(F)ccc3c2)ccc2cc(N(c3ccc(-c4nc5ccccc5o4)cc3)c3ccc(-c4nc5ccccc5o4)cc3)ccc12 ; [2H]c1cc(N(c2ccc3nc(-c4ccccc4)oc3c2)c2ccc3nc(-c4ccccc4)oc3c2)cc2ccc(-c3ccc4ccc(F)cc4c3)cc12 ; [2H]c1c(N(c2ccc(-c3nc4ccccc4o3)cc2)c2ccc(-c3nc4ccccc4o3)cc2)c([2H])c2c([2H])c([2H])c(-c3ccc4cc(F)ccc4c3)c([2H])c2c1[2H] ; [2H]c1c(N(c2ccc3nc(-c4ccccc4)oc3c2)c2ccc3nc(-c4ccccc4)oc3c2)c([2H])c2c([2H])c([2H])c(-c3ccc4ccc(F)cc4c3)c([2H])c2c1[2H]]

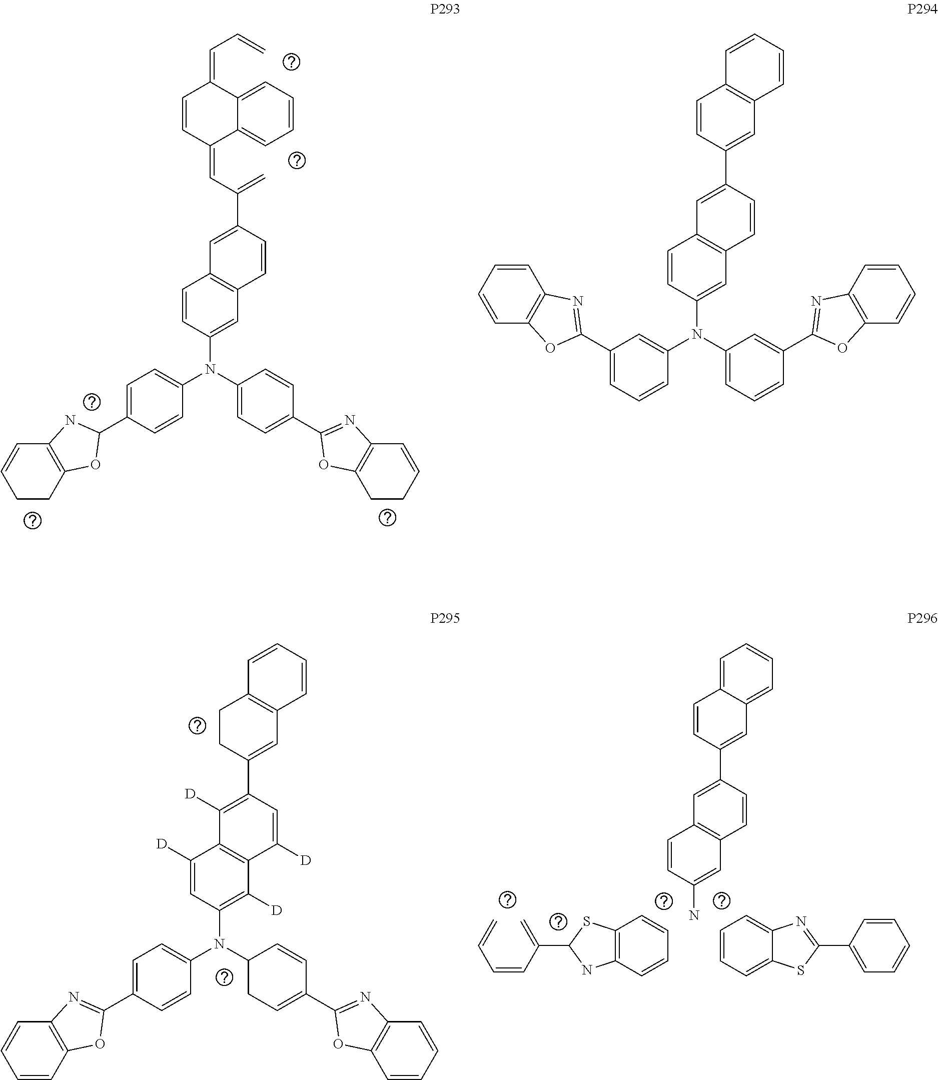

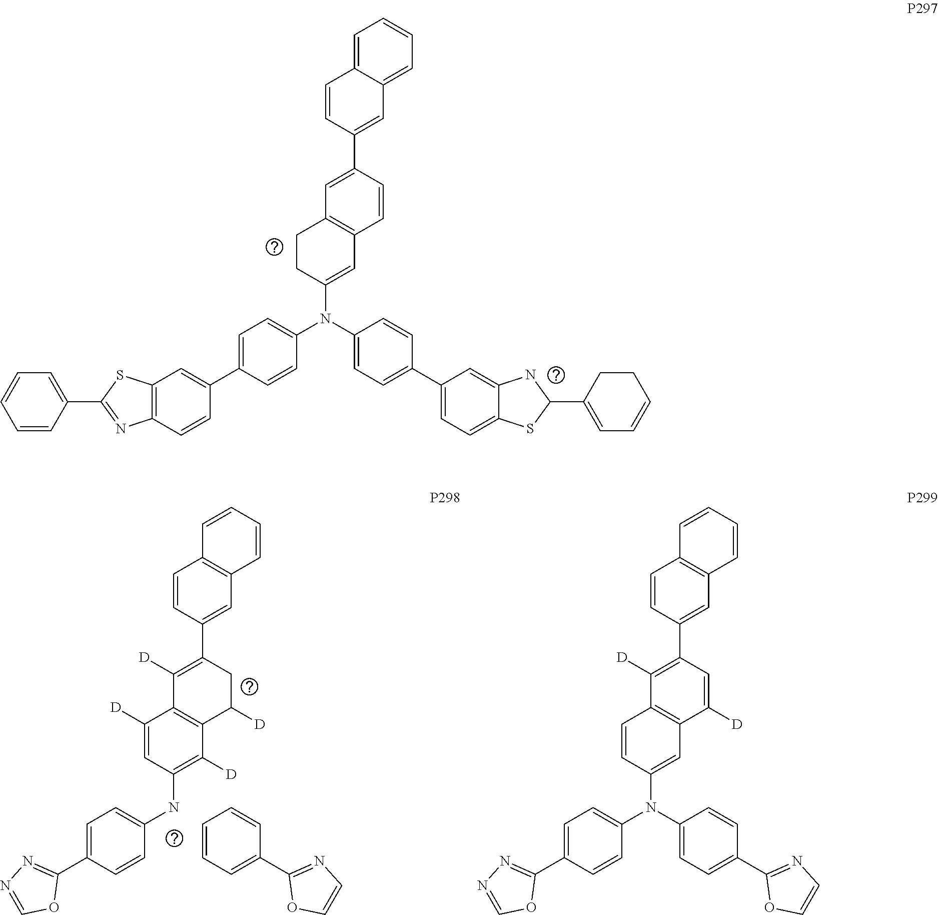

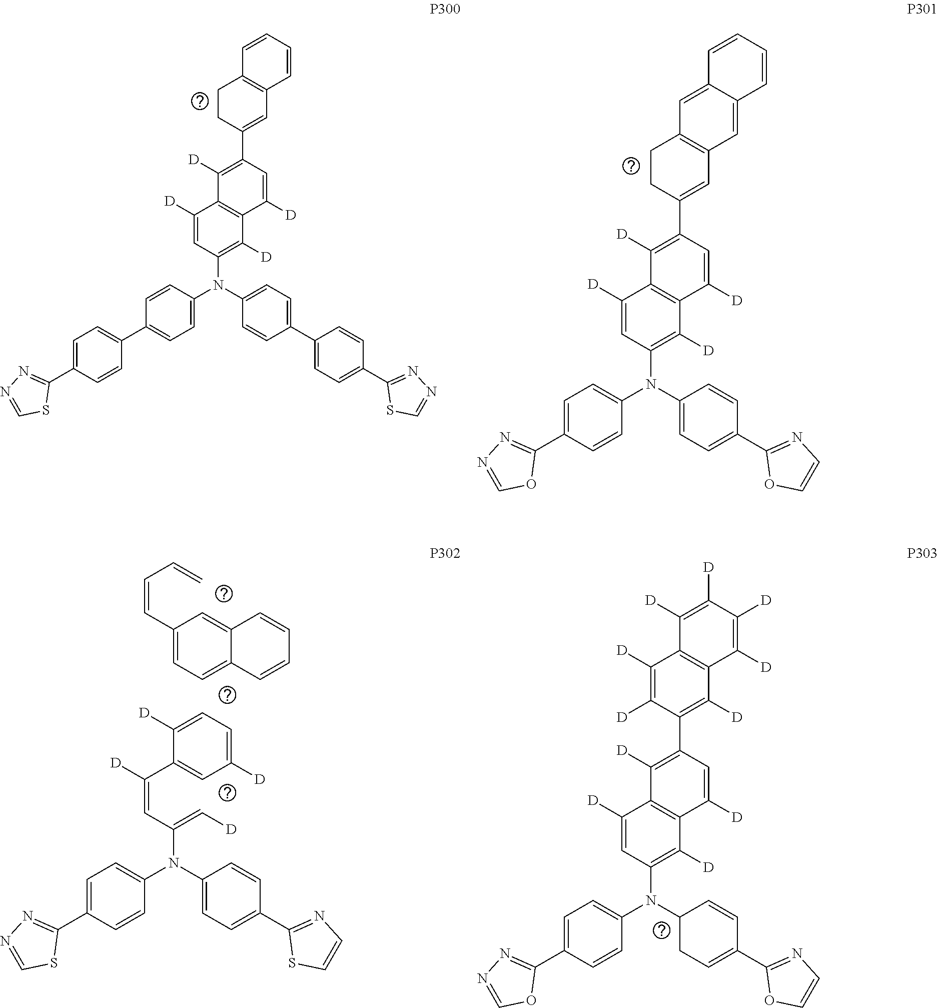

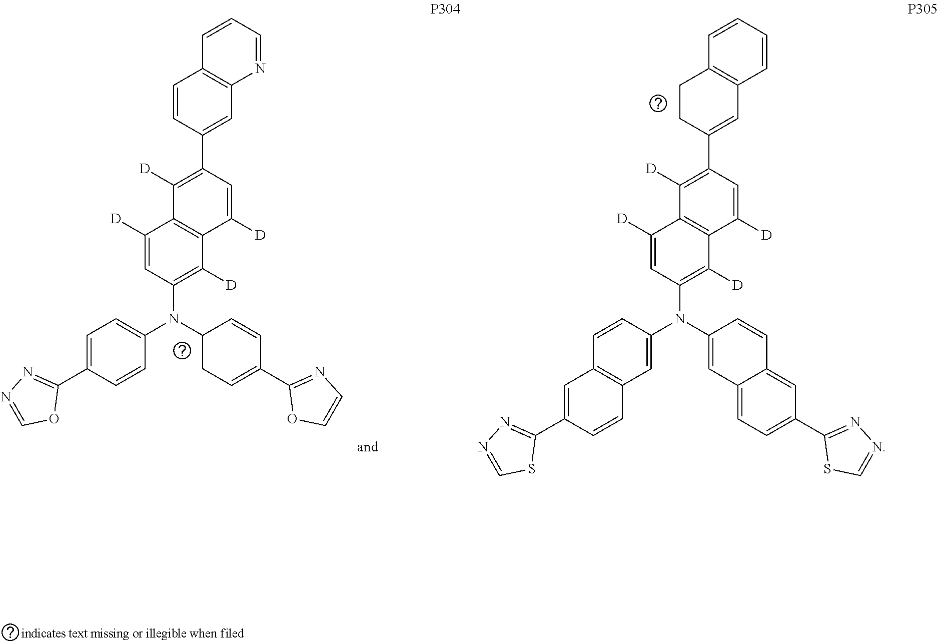 [SMILES: [2H]c1cc(N(c2ccc(-c3nnco3)cc2)C2C=CC(c3ncco3)=CC2)c([2H])c2c([2H])cc(-c3ccc4cccnc4c3)c([2H])c12 ; [2H]c1cc(N(c2ccc3cc(-c4nncs4)ccc3c2)c2ccc3cc(-c4nncs4)ccc3c2)c([2H])c2c([2H])cc(C3=Cc4ccccc4CC3)c([2H])c12]

27. A material for an organic electroluminescent device, comprising any one or a combination of at least two of the compound according to claim 1.

28. An organic electroluminescent element, comprising a first electrode layer, an organic function layer and a second electrode layer which are stacked in sequence;
wherein the organic function layer comprises the material according to claim 27.

29. An organic electroluminescent element, comprising a first capping layer, a first electrode layer, an organic function layer and a second electrode layer which are stacked in sequence;
wherein the first capping layer comprises the material according to claim 27.

30. A display panel, comprising the organic electroluminescent element according to claim 28, wherein the display panel is optionally used in a display device

31. A display panel, comprising the organic electroluminescent element according to claim 29, wherein the display panel is optionally used in a display device.
